(19) United States
(12) Patent Application Publication
(10) Pub. No.: US 2010/0144672 A1
(43) Pub. Date: Jun. 10, 2010

(54) PYRIMIDINYL PYRAZOLES AS INSECTICIDES AND PARASITICIDE ACTIVE AGENTS

(75) Inventors: Jens Frackenpohl, Frankfurt (DE); Olaf Gebauer, Leverkusen (DE); Silvia Cerezo-Galvez, Langenfeld (DE); Mazen Es-Sayed, Langenfeld (DE); Ulrich Görgens, Ratingen (DE); Eva-Maria Franken, Lyon (FR); Olga Malsam, Rosrath (DE); Stefan Schnatterer, Hattersheim (DE); Christian Arnold, Langenfeld (DE); Peter Lümmen, Idstein (DE); Hans-Georg Schwarz, Langenfeld (DE); Achim Hense, Leverkusen (DE); Stefan Werner, Monheim am Rhein (DE)

Correspondence Address: Baker Donelson Bearman, Caldwell & Berkowitz, PC, 555 Eleventh Street, NW, Sixth Floor, Washington, DC, 20004, US

(73) Assignee: BAYER CROPSCIENCE AG, Monheim (DE)

(21) Appl. No.: 12/520,477

(22) PCT Filed: Dec. 12, 2007
(86) PCT No.: PCT/EP2007/010839

§ 371 Date: Jan. 25, 2010

(30) Foreign Application Priority Data
Dec. 20, 2006 (DE) 10 2006 060 230.7
Jan. 20, 2007 (DE) 10 2007 003 036.5

(51) Int. Cl. A01N 43/54; C07D 403/04; C07F 7/10; A01N 55/10; A01P 7/04

(52) U.S. Cl. 514/63; 544/331; 544/229; 514/275

(57) ABSTRACT

The present invention relates to pyrimidinylpyrazoles and their use as insecticides, and also to processes for their preparation and to compositions comprising such arylpyrazoles.

The present invention relates to pyrimidinylpyrazoles and their use as insecticides and/or parasiticides, and also to processes for their preparation and to compositions comprising such arylpyrazoles.

U.S. Pat. No. 5,201,938 describes arylpyrazoles as intermediates in the synthesis of herbicides. An inherent activity of the arylpyrazoles (as herbicides or with any other biological activity) is neither disclosed nor suggested. WO 93/19054, too, discloses arylpyrazoles. Here, they are used as intermediates, this time in the synthesis of fungicides. Here, too, an inherent activity is not disclosed.

WO 02/68413 discloses insecticidally active pyrimidinylpyrazoles which, however, contain a 4-pyrimidinyl radical. This publication does not mention the activity of the pyrimidinylpyrazoles according to the invention.

WO 95/22530 discloses N-pyrazolylanilines and N-pyrazolylaminopyridines for whose preparation use is likewise made of pyrimidinylpyrazole intermediates which generically include the compounds disclosed in the present application. However, none of the compounds embraced by the claims of the present application is disclosed explicitly. Also, a biological action is not ascribed to the compounds disclosed therein.

Aggerwal et al. (Bioorganic & Medicinal Chemistry, 14 (2006), 1785-1791) describe antibacterially active phenylpyrimidinylpyrazol-2-yls; however, they only contain unsubstituted phenyl and 4,6-dimethylated pyrimidinyl. Other substitution patterns or a different biological action are neither disclosed or suggested.

Accordingly, the present invention provides novel pyrimidinylpyrazoles of the formula (I)

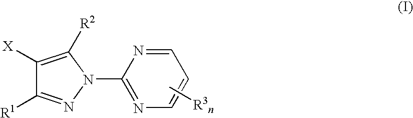

in which
X represents phenyl, 2-pyridyl, 3-pyridyl or 2-pyrimidinyl, in each case substituted by one or more substituents selected from the group consisiting of halogen, alkyl, haloalkyl, alkoxy, alkoxyalkyl, alkoxyalkoxy, cycloalkyl, alkenoxy, alkynoxy, benzyloxy, cycloalkylalkoxy, haloalkoxy, haloalkoxyalkyl, alkylsulphanyl, haloalkylsulphanyl, alkylsulphinyl, haloalkylsulphinyl, alkylsulphonyl, haloalkylsulphonyl, cyano, alkylcarbonyl, alkoxycarbonyl, alkoxycarbonylalkyl, carboxyl, carboxamide, dialkylcarboxamide, trialkylsilyl, nitro, amino, alkylamino, dialkylamino, alkylsulphonylamino, dialkylsulphonylamino, —CH═NO—H, —CH═NO-alkyl, —CH═NO-haloalkyl, —C(CH₃)═NO—H, —C(CH₃)═NO-alkyl, —C(CH₃)═NO-haloalkyl, formyl, benzyl (for its part optionally substituted by one or more halogen atoms), phenoxy or pyrid-2-yloxy (the latter two optionally substituted by one or more substituents selected from the group consisting of alkyl, halogen and haloalkyl) and phenyl (optionally substituted by one or more halogen atoms), where vicinal alkyl, haloalkyl, alkoxy and/or haloalkoxy groups together with the carbon atoms to which they are attached may form a five- to six-membered cyclic system which contains 0 to 2 oxygen atoms and whose alkyl moiety may optionally be substituted by one or more fluorine atoms,
R¹ represents alkyl (optionally mono- or disubstituted by substituents independently of one another selected from the group consisting of alkoxy, haloalkoxy, alkylsulphanyl, haloalkylsulphanyl, alkylsulphinyl, haloalkylsulphinyl, alkylsulphonyl, haloalkylsulphonyl, alkylcarbonyl, alkoxycarbonyl, hydroxyl, cycloalkyl, phenyl (for its part optionally substituted by one or more halogen atoms) and heterocyclyl), cycloalkyl (optionally substituted by one or more haloalkyl groups and/or halogen atoms), haloalkyl (optionally substituted by alkoxy), cyano, formyl, ‘3CH═NO—H, —CH═NO-alkyl, —CH═NO-haloalkyl, —C(CH₃)═NO—H, —C(CH₃)═NO-alkyl, —C(CH₃)═NO-halolkyl or benzyl (optionally mono- or polysubstituted by one or more substituents independently of one another selected from the group consisting of halogen, alkyl, haloalkyl and alkoxy),
R² represent amino and
R² furthermore represents amino which is monosubstituted by alkyl, alkylcarbonyl, cycloalkyl, cycloalkylalkyl, alkenyl, alkoxycarbonyl, alkenyloxycarbonyl, alkynyloxycarbonyl, alkoxycarbonylalkyl, alkoxycarbonylcarbonyl, benzyl or phenylcarbonyl (where the phenyl ring in benzyl and phenylcarbonyl for its part is optionally substituted by one or more halogen atoms) and
R² furthermore represents amino which is disubstituted by two substituents independently of one another selected from the group consisting of benzyl, alkyl, alkoxycarbonyl, alkenyloxycarbonyl, alkynyloxycarbonyl and alkylcarbonyl, and
R² furthermore represents piperidine, pyrrolidine or morpholine which are optionally mono- or disubstituted by alkyl and
R² furthermore represents halogen,
R³ represents halogen, alkyl, haloalkyl, alkoxy or dialkylamino and
n represents 0 or 1,
where the compounds of the general formula (I) also include N-oxides and salts.

Finally, it has been found that the compounds of the formula (I) according to the invention have very good insecticidal and parasiticidal properties and can be used in crop protection, in veterinary hygiene and in the protection of materials for controlling unwanted pests, such as insects, endo- or ectoparasites.

If appropriate, the compounds according to the invention can be present as mixtures of different possible isomeric forms, in particular stereoisomers, such as, for example, E and Z, threo and erythro, and also optical isomers, and, if appropriate, also of tautomers. What is claimed are both the E and the Z isomers, and the threo and erythro, and also the optical isomers, any mixtures of these isomers, and also the possible tautomeric forms.

The formula (I) provides a general definition of the pyrimidinylpyrazoles according to the invention. Preferred, particularly preferred and very particularly preferred radical definitions of the formulae mentioned above and below are given below. These definitions apply to the end products of the formulae (I) and likewise to all intermediates.
X preferably represents phenyl, 2-pyridyl, 3-pyridyl or 2-pyrimidinyl, in each case substituted by one or more substituents selected from the group consisting of fluorine, chlorine, bromine, iodine, C₁-C₆-alkyl, C₁-C₆-haloalkyl, C₁-C₆-alkoxy-C₁-C₆-alkyl, C₁-C₆-alkoxy, C₁-C₆-haloalkoxy, C₁-C₆-haloalkoxy-C₁-C₆-alkyl, C₁-C₆-alkylsulphanyl, halo-C₁-C₆-alkylsulphanyl, C₁-C₆-alkylsulphinyl, halo-C₁-C₆-alkylsulphinyl, C₁-C₆-alkylsulphonyl, halo-C₁-C₆-alkylsulphonyl, cyano, C₁-C₆-alkylcarbonyl, C₁-C₆-alkoxycarbonyl, C₁-C₆-alkoxycarbonyl-C₁-C₆-alkyl, nitro, amino, C₁-C₆-alkylamino, di(C₁-C₆-alkyl)amino, C₁-C₆-alkylsulphonylamino, di(C₁-C₆-alkyl)sulphonylamino, —CH═NO—H, —CH═NO—C₁-C₆-alkyl, —CH═NO-halo-C₁-C₆-alkyl, —C(CH₃)═NO—H, —C(CH₃)═NO—C₁-C₆-alkyl, —C(CH₃)═NO-halo-C₁-C₆-alkyl, formyl, benzyl (for its part optionally substituted by one or more halogen atoms), phenoxy or pyrid-2-yloxy (the latter two optionally substituted by one or more halogen atoms and/or halo-C₁-C₆-alkyl groups) and phenyl (optionally substituted by one or more halogen atoms), where vicinal alkyl, haloalkyl, alkoxy and/or haloalkoxy groups together with the carbon atoms to which they are attached may form a five- to six-membered cyclic system which contains 0 to 2 oxygen atoms and whose alkyl moiety may optionally be substituted by one or more fluorine atoms.
X particularly preferably represents phenyl, 2-pyridyl, 3-pyridyl or 2-pyrimidinyl, in each case substituted by one or more substituents selected from the group consisting of fluorine, chlorine, bromine, iodine, C₁-C₄-alkyl, C₁-C₄-haloalkyl, C₁-C₄-alkoxy-C₁-C₄-alkyl, C₁-C₄-alkoxy, C₁-C₄-haloalkoxy, C₁-C₄-haloalkoxy-C₁-C₄-alkoxy-C₁-alkylsulphanyl, halo-C₁-C₄-alkylsulphanyl, C₁-C₄-alkylsulphinyl, halo-C₁-C₄-alkylsulphinyl, C₁-C₄-alkylsulphonyl, halo-C₁-C₄-alkylsulphonyl, cyano, C₁-C₄-alkylcarbonyl, C₁-C₄-alkoxycarbonyl, C₁-C₄-alkoxycarbonyl-C₁-C₄-alkyl, nitro, amino, C₁-C₄-alkylamino, di(C₁-C₄-alkyl)amino, C₁-C₄-alkylsulphonylamino, di(C₁-C₄-alkyl)sulphonylamino, —CH═NO—H, —CH═NO—C₁-C₄-alkyl, —CH═NO-halo-C₁-C₄-alkyl, —C(CH₃)═NO—H, —C(CH₃)═NO—C₁-C₄-alkyl, —C(CH₃)═NO-halo-C₁-C₄-alkyl, formyl, benzyl (for its part optionally substituted by one or more halogen atoms), phenoxy or pyrid-2-yloxy (the latter two optionally substituted by one or more halogen atoms and/or halo-C₁-C₄-alkyl groups) and phenyl (optionally substituted by one or more halogen atoms), where vicinal alkyl, haloalkyl, alkoxy and/or haloalkoxy groups together with the carbon atoms to which they are attached may form a five- to six-membered cyclic system which contains 0 to 2 oxygen atoms and whose alkyl moiety may optionally be substituted by one or more fluorine atoms.
X very particularly preferably represents phenyl, 2-pyridyl, 3-pyridyl or 2-pyrimidinyl, in each case substituted by one or more substituents selected from the group consisting of fluorine, chlorine, bromine, iodine, methyl, ethyl, isopropyl, tert-butyl, —(CH₂)₄—, —O—C₂H₄—O—, difluoromethyl, trifluoromethyl, C₁-C₄-alkoxy-C₁-C₄-alkyl, methoxy, ethoxy, isopropyloxy, n-propyloxy, trifluoromethoxy, 1,1,2,3,3,3-hexafluoropropyloxy, difluorochloromethoxy, difluoromethoxy, 1,1,2-trifluoro-2-chloroethoxy, 2,2,2-trifluoroethoxy, methylsulphanyl, methylsulphinyl, methylsulphonyl, trifluoromethylsulphanyl, cyano, nitro, formyl, amino, dimethylamino, diethylamino, di(n-propyl)amino, di(isopropyl)amino, methylamino, ethylamino, n-propylamino, iso-propylamino, methylsulphonylamino, ethylsulphonylamino, n-propylsulphonylamino, isopropylsulphonylamino, —CH═NO—H, —CH═NO—CH₃, —CH═NO—C₂H₅, —CH═NO—CH(CH₃)₂, —CH═NO—CH₂CH₂CH₃, —C(CH₃)═NO—H, —C(CH₃)═NO—CH₃, —C(CH₃)═NO—C₂H₅, —C(CH₃)═NO—CH(CH₃)₂, —C(CH₃)═NO—CH₂CH₂CH₃, benzyl, phenoxy or pyrid-2-yloxy (the latter two optionally substituted by one or more substituents selected from the group consisting of chlorine and trifluoromethyl) and phenyl (optionally substituted by chlorine).
X especially preferably represents phenyl, 2-pyridyl, 3-pyridyl or 2-pyrimidinyl, in each case substituted by one or more substituents selected from the group consisting of fluorine, chlorine, bromine, iodine, methyl, ethyl, isopropyl, tert-butyl, —(CH₂)₄—, —O—C₂H₄—O—, trifluoromethyl, C₁-C₄-alkoxy-C₁-C₄-alkyl, methoxy, ethoxy, isopropyloxy, n-propyloxy, trifluoromethoxy, 1,1,2,3,3,3-hexafluoropropyloxy, difluorochloro-methoxy, difluoromethoxy, 1,1,2-trifluoro-2-chloroethoxy, 2,2,2-trifluoroethoxy, methylsulphanyl, methylsulphinyl, methylsulphonyl, trifluoromethylsulphanyl, cyano, benzyl, phenoxy or pyrid-2-yloxy (the latter two optionally substituted by one or more substituents selected from the group consisting of chlorine and trifluoromethyl) and phenyl (optionally substituted by chlorine).

R¹ preferably represents C₁-C₆-alkyl (optionally mono- or disubstituted by substituents independently of one another selected from the group consisting of C₁-C₆-alkoxy, halo-C₁-C₆-alkoxy, C₁-C₆-alkylsulphanyl, halo-C₁-C₆-alkylsulphanyl, C₁-C₆-alkylsulphinyl, halo-C₁-C₆-alkylsulphinyl, C₁-C₆-alkylsulphonyl, halo-C₁-C₆-alkylsulphonyl, C₁-C₆-alkylcarbonyl, C₁-C₆-alkoxycarbonyl, hydroxyl, C₃-C₆-cycloalkyl, phenyl and heterocyclyl), C₃-C₆-cycloalkyl (optionally substituted by one or more halogen atoms), halo-C₁-C₆-alkyl (optionally substituted by C₁-C₆-alkoxy), cyano, formyl, —CH═NO—H, —CH═NO—C₁-C₆-alkyl, —CH═NO-halo-C₁-C₆-alkyl, —C(CH₃)═NO—H, —C(CH₃)═NO—C₁-C₆-alkyl, —C(CH₃)═NO-halo-C₁-C₆-alkyl or benzyl (optionally mono- or polysubstituted by one or more substituents independently of one another selected from the group consisting of halogen, C₁-C₆-alkyl and C₁-C₆-alkoxy).

R¹ particularly preferably represents C₁-C₄-alkyl (optionally mono- or disubstituted by substituents independently of one another selected from the group consisting of C₁-C₄-alkoxy, C₁-C₄-alkylsulphanyl, C₁-C₄-alkylcarbonyl, C₁-C₄-alkoxycarbonyl, hydroxyl, C₃-C₆-cycloalkyl, phenyl and heterocyclyl), C₃-C₅-cycloalkyl (optionally substituted by one or more halogen atoms), halo-C₁-C₄-alkyl (optionally substituted by C₁-C₄-alkoxy), cyano, formyl, —CH═NO—H, —CH═NO—C₁-C₄-alkyl, —CH═NO-halo-C₁-C₄-alkyl, —C(CH₃)═NO—H, —C(CH₃)═NO—C₁-C₄-alkyl, —C(CH₃)═NO-halo-C₁-C₄-alkyl or benzyl (optionally mono- or polysubstituted by one or more substituents independently of one another selected from the group consisting of halogen, C₁-C₄-alkyl and C₁-C₄-alkoxy).
R¹ very particularly preferably represents methyl, ethyl, n-propyl, isopropyl, n-butyl or isobutyl (in each case optionally mono- or disubstituted by substituents independently of one another selected from the group consisting of methoxy, ethoxy, methylthio, methoxycarbonyl, trifluoromethyl, hydroxyl, cyclopropyl, cyclohexyl, phenyl and optionally substituted 1-pyrazolyl); represents cyclopropyl, cyclobutyl or cyclopentyl (in each case optionally substituted by one or more substituents selected from the group consisting of fluorine and chlorine); represents fluoromethyl, chlorofluoromethyl, difluoromethyl, 1-fluoro-n-propyl, 1-fluoro-n-butyl, difluorobromomethyl, difluorochloromethyl, dichlorofluoromethyl, 2-fluoroisopropyl, perfluoroethyl, 1,1,2,2-tetrafluoro ethyl, 1,1-difluoroethyl, 2,2-difluoro-1-bromoethyl, 2,2,2-trifluoro-1-methoxyethyl or trifluoromethyl; represents cyano; represents —CH═NO—H, —CH═NO—CH₃ or —CH═NO—C₂H₅; or represents benzyl (optionally mono- or polysubstituted by one or more substituents independently of one another selected from the group consisting of fluorine, bromine, chlorine, methyl and methoxy).
R¹ especially preferably represents methyl, ethyl, n-propyl, isopropyl, n-butyl or isobutyl (in each case optionally mono- or disubstituted by substituents independently of one another selected from the group consisting of methoxy, ethoxy, methylthio, methoxycarbonyl, trifluoromethyl, hydroxyl, cyclopropyl, cyclohexyl and phenyl); represents cyclopropyl, cyclobutyl or cyclopentyl (in each case optionally substituted by one or more substituents selected from the group consisting of fluorine and chlorine); represents fluoromethyl, chlorofluoromethyl, difluoromethyl, 1-fluoro-n-propyl, 1-fluoro-n-butyl, difluorobromomethyl, difluorochloromethyl, dichlorofluoromethyl, 2-fluoroisopropyl, perfluoroethyl, 1,1,2,2-tetrafluoroethyl, 1,1-difluoroethyl, 2,2-difluoro-1-bromoethyl, 2,2,2-trifluoro-1-methoxyethyl or trifluoromethyl; represents cyano; represents —CH═NO—H, —CH═NO—CH₃ or —CH═NO—C₂H₅; or represents benzyl (optionally mono- or polysubstituted by one or more substituents independently of one another selected from the group consisting of fluorine, bromine, chlorine, methyl and methoxy).
R² preferably represents amino which is optionally monosubstituted by C₁-C₆-alkyl, C₁-C₆-alkylcarbonyl, C₁-C₆-alkoxycarbonyl, C₃-C₆-alkenyloxycarbonyl, C₃-C₆-alkynyloxycarbonyl,C₁-C₆-alkoxycarbonyl-C₁-C₆-alkyl, C₃-C₅-cycloalkyl, C₃-C₅-cycloalkyl-C₁-C₃-alkyl, C₁-C₆-alkenyl, C₁-C₆-alkoxycarbonyl, C₁-C₆-alkoxycarbonylcarbonyl, benzyl or phenylcarbonyl (where the phenyl ring in benzyl and phenylcarbonyl for its part is optionally substituted by one or more halogen atoms) and
R² furthermore preferably represents amino which is disubstituted by two substituents independently of one another selected from the group consisting of benzyl, C₁-C₆-alkyl and C₁-C₆-alkylcarbonyl, C₁-C₆-alkoxycarbonyl, C₃-C₆-alkenyloxycarbonyl and C₃-C₆-alkynyloxycarbonyl, and
R² furthermore preferably represents piperidine, pyrrolidine or morpholine which are optionally substituted by one or two C₁-C₆-alkyl and
R² furthermore preferably represents fluorine, chlorine, bromine or iodine.
R² particularly preferably represents amino, optionally monosubstituted by methyl, ethyl, isopropyl, isobutyl, n-propyl, n-butyl, 2′-isopentyl, methylcarbonyl, cyclopropylmethyl, cyclopentyl, prop-1-en-3-yl, 2-methylbut-2-en-4-yl, methoxycarbonylcarbonyl, ethoxycarbonylcarbonyl, methoxycarbonyl, ethoxycarbonyl, 2-propenyloxycarbonyl, propargyloxycarbonyl, ethoxycarbonylmethyl, benzyl or phenylcarbonyl (where the phenyl ring in benzyl and phenylcarbonyl for its part is optionally substituted by one or more fluorine, bromine and/or chlorine atoms) and
R² furthermore particularly preferably represents amino which is disubstituted by two substituents independently of one another selected from the group consisting of benzyl, methyl, ethyl, methoxycarbonyl, ethoxycarbonyl, 2-propenyloxycarbonyl and propargyloxycarbonyl, and
R² furthermore particularly preferably represents piperidine, pyrrolidine or morpholine which are optionally mono- or disubstituted by C₁-C₆-alkyl and
R² furthermore particularly preferably represents chlorine, bromine or iodine.
R² very particularly preferably represents amino, optionally monosubstituted by methyl, ethyl, isopropyl, isobutyl, n-propyl, n-butyl, 2′-isopentyl, methylcarbonyl, cyclopropylmethyl, cyclopentyl, prop-1-en-3-yl, 2-methylbut-2-en-4-yl, methoxycarbonylcarbonyl, methoxycarbonyl, ethoxycarbonyl, 2-propenyloxycarbonyl, propargyloxycarbonyl, ethoxycarbonylcarbonyl, ethoxycarbonylmethyl, benzyl or phenylcarbonyl (where the phenyl ring in benzyl and phenylcarbonyl for its part is optionally substituted by one or more fluorine, bromine and/or chlorine atoms) and
R² furthermore very particularly preferably represents amino which is disubstituted by two identical substituents selected from the group consisting of benzyl, methyl, ethyl, methoxycarbonyl, ethoxycarbonyl, 2-propenyloxycarbonyl and propargyloxycarbonyl, and
R² furthermore very particularly preferably represents 4-methylpiperidine, pyrrolidine, morpholine, 2-6-dimethylmorpholine or piperidine and
R² furthermore very particularly preferably represents chlorine, bromine or iodine.
R² especially preferably represents amino, optionally monosubstituted by methyl, ethyl, isopropyl, isobutyl, n-propyl, n-butyl, 2′-isopentyl, methylcarbonyl, cyclopropylmethyl, cyclopentyl, prop-1-en-3-yl, 2-methylbut-2-en-4-yl, methoxycarbonylcarbonyl, methoxycarbonyl, ethoxycarbonyl, propargyloxycarbonyl, ethoxycarbonylcarbonyl, ethoxycarbonylmethyl, benzyl or phenylcarbonyl (where the phenyl ring in benzyl and phenylcarbonyl for its part is optionally substituted by one or more bromine and/or chlorine atoms) and
R² furthermore especially preferably represents amino which is disubstituted by two identical substituents selected from the group consisting of benzyl, methyl, ethyl, methoxycarbonyl, ethoxycarbonyl and propargyloxycarbonyl, and
R² furthermore especially preferably represents 4-methylpiperidine and
R² furthermore especially preferably represents chlorine, bromine or iodine.
R³ preferably represents fluorine, chlorine, bromine, C₁-C₆-alkyl, halo-C₁-C₆-alkyl, C₁-C₆-alkoxy or di-Cᵢ-C₆-alkylamino.
R³ particularly preferably represents fluorine, chlorine, methyl, ethyl, isopropyl, n-propyl, perhalo-C₁-C₃-alkyl, methoxy, ethoxy, n-propoxy, isopropoxy, dimethylamino or diethylamino.
R³ very particularly preferably represents fluorine, chlorine, methyl, trifluoromethyl, methoxy or dimethylamino.
n very particularly preferably represents 0.

A further preferred embodiment of the present invention are compounds of the formula (I) where X, R¹, R², R³ and n are as defined above, where, if X represents phenyl substituted by C₁-C₅-alkyl, Cl, F, Br, CF₃, C₂-C₃-alkenyl, C₂-C₃-alkynyl, C₁-C₄-alkoxy, alkoxyalkyl, phenyl, benzyl, O-phenyl or O-benzyl, and R² represents NH₂ and R¹ represents CF₃, C₁-C₃-alkyl or C₁-C₃-alkoxy-C₁-C₃-alkyl and n represents 0, then X is at least disubstituted, and if X is substituted by two identical substituents, these two substituents are located neither in positions 2 and 4 nor in positions 3 and 4.

A further preferred embodiment of the present invention are compounds of the formula (I) selected from the group consisting of Tables 1 to 43, where
a) if the compounds are selected from Table 1, then, if X is monosubstituted, the substituent of X does not represent F, Cl or Br, and, if X is substituted by two identical substituents selected from the group consisting of F, Cl or Br, then these two substituents are located neither in positions 2 and 4 nor in positions 3 and 4;
b) if the compounds are selected from Table 3, then, if X is monosubstituted, the substituent is not CF₃, and, if X is disubstituted by CF₃, these two substituents are located neither in positions 2 and 4 nor in positions 3 and 4;
c) if the compounds are selected from Table 8, then, if X is substituted by two identical substituents selected from the group consisting of methyl and methoxy, these two substituents are located neither in positions 2 and 4 nor in positions 3 and 4;
d) if the compounds are selected from Table 16 where R¹ is C₁-C₃-alkyl, then, if X is monosubstituted, the substituent of X does not represent F, Cl or Br, and, if X is disubstituted by Cl, these two substituents are located neither in positions 2 and 3 nor in positions 3 and 4; and
e) if the compounds are selected from Table 17 where R¹ is CH₂OMe, CH₂OEt or CH(OMe)Me, then, if X is monosubstituted, the substituent of X does not represent F, Cl or Br, and, if X is disubstituted by two identical substituents selected from the group consisting of F, Cl and Br, then these two substituents are located neither in positions 2 and 4 nor in positions 3 and 4.

A further preferred embodiment of the present invention are compounds of the formula (I), where X, R¹, R², R³ and n are as defined above, where X is disubstituted by two non-identical substituents.

A further preferred embodiment of the present invention are compounds of the formula (I), where X, R¹, R², R³ and n are as defined above, where X is disubstituted and the substituents are located in positions 3 and 5.

A further preferred embodiment of the present invention are compounds of the formula (I), where X, R¹, R², R³ and n are as defined above, where X is at least trisubstituted.

A further preferred embodiment of the present invention are compounds of the formula (I), where X, R¹, R², R³ and n are as defined above, where X is at least trisubstituted and the substituents are located in positions 3, 4 and 5 or positions 2, 3 and 5.

A further preferred embodiment of the present invention are compounds of the formula (I), where X, R¹, R², R³ and n are as defined above, where R¹ is selected from the group consisting of fluoromethyl, chlorofluoromethyl, difluoromethyl, 1-fluoro-n-propyl, 1-fluoro-n-butyl, difluorobromomethyl, difluorochloromethyl, dichlorofluoromethyl, 2-fluoroisopropyl, perfluoroethyl, 1,1,2,2-tetrafluoroethyl, 1,1-difluoroethyl, 2,2-difluoro-1-bromoethyl or 2,2,2-trifluoro-1-methoxyethyl.

Halogen-substituted radicals, for example haloalkyl, are mono- or polyhalogenated up to the highest possible number of substituents. In the case of polyhalogenation, the halogen atoms can be identical or different. Here, halogen represents fluorine, chlorine, bromine or iodine, in particular fluorine, chlorine or bromine.

Preferred, particularly preferred, very particularly preferred and especially preferred are compounds carrying in each case the substituents mentioned as being preferred, particularly preferred, very particularly preferred and especially preferred.

Saturated or unsaturated hydrocarbon radicals, such as alkyl or alkenyl, can in each case be straight-chain or branched as far as this is possible, including in combination with heteroatoms, such as, for example, in alkoxy.

Optionally substituted radicals can be mono- or polysubstituted, where in the case of polysubstitution the substituents can be identical or different.

However, the general or preferred radical definitions or illustrations given above can also be combined with one another as desired, i.e. between the respective ranges and preferred ranges. The definitions apply both to the end products and, correspondingly, to the precursors and intermediates.

Depending on their substituent R², the compounds of the formula (I) can be classed into the compounds of the formulae (IA) and (IB):

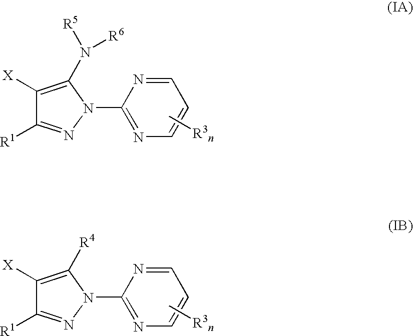

where
X, R¹, R³ and n have the meanings given above,
R⁴ represents halogen,
R⁵ represents hydrogen, alkyl, alkylcarbonyl, alkoxycarbonyl, alkenyloxycarbonyl, alkynyloxycarbonyl, cycloalkyl, cycloalkylalkyl, alkenyl, alkoxycarbonylcarbonyl, benzyl or phenylcarbonyl (where the phenyl ring in benzyl and phenylcarbonyl for its part is optionally substituted by one or more fluorine, bromine and/or chlorine atoms),
R⁶ represents hydrogen,
R⁶ furthermore, if R⁵ represents benzyl, alkyl, alkylcarbonyl alkoxycarbonyl, alkenyloxycarbonyl or alkynyloxycarbonyl, may also represent benzyl, alkyl, alkylcarbonyl, alkoxycarbonyl, alkenyloxycarbonyl or alkynyloxycarbonyl, or
R⁵ and R⁶ together with the nitrogen atom to which they are attached may form a piperidine, pyrrolidine or morpholine radical.

Hereinbelow, compounds of the formula (IA) in which R⁵ and R⁶ both represent hydrogen are referred to as compounds of the formula (IA-1):

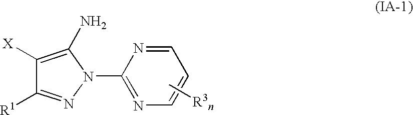

where X, R¹, R³ and n have the meanings given above.

Process (A)

Compounds of the formula (IA-1) are, for example, synthesized by process (A) below:

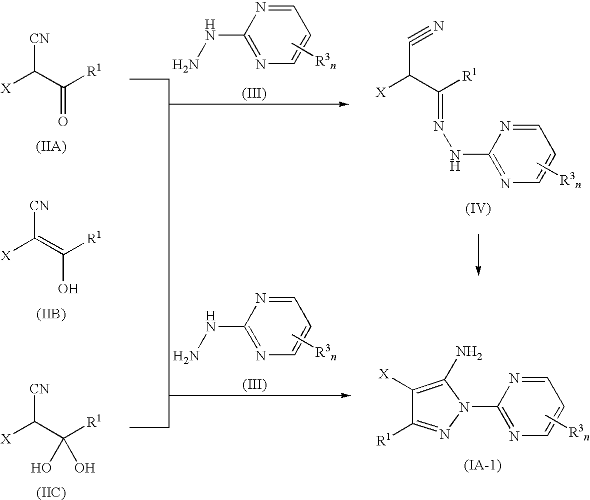

where X, R¹, R³ and n have the meanings given above.

In the process (A) according to the invention for preparing the compounds of the formula (IA-1), ketonitriles, their tautomers or hydrates of the formulae (IIA), (IIB) and (IIC) are condensed with pyrimidinylhydrazines of the formula (III), which initially gives hydrazones of the formula (IV) as intermediates and where, if the reaction time is prolonged and the temperature elevated, there is ring closure giving the aminopyrazole of the formula (IA). Here, acids may be added as catalyst, where inorganic acids such as hydrochloric acid and organic acids such as sulphonic acids or acetic acid may be suitable.

The synthesis of structurally related aminopyrazoles is described in
R. Aggarwal et al, Bioorg. Med. Chem. 14 (2006), 6, 1785-1791
S. P. Singh et al, Eur. J. Med. Chem. 40 (2005), 922-927
DE-A 2643640
U.S. Pat. No. 3,041,342

The ketonitriles can be present in the tautomeric forms (IIA) and (IIB) and as hydrate (IIC). Starting materials can also be employed in the form of their salts; for example, the ketonitriles can be used in the form of their alkali metal salts and the pyrimidinylhydrazines can be used in the form of their hydrochlorides.

The ketonitriles of the formula (II) can be prepared by known methods:
W. R. Nes, Alfred Burger, J. Amer. Chem. Soc. 72 (1950), 5409-5413

Some of the pyrimidinylhydrazines of the formula (III) are commercially available. The preparation of pyrimidinylhydrazines of the formula (III) is carried out according to the methods described in:
Methoden der Organischen Chemie [Methods of Organic Chemistry] (Houben-Weyl), Organische Stickstoff-Verbindungen [Organic Nitrogen Compounds], Volume E 16a, Part 1, pp. 678-775, Georg Thieme Verlag Stuttgart-New York, 1990.

Process (B)

Alternatively, compounds of the formula (IA-1) can be synthesized according to the process (B) according to the invention:

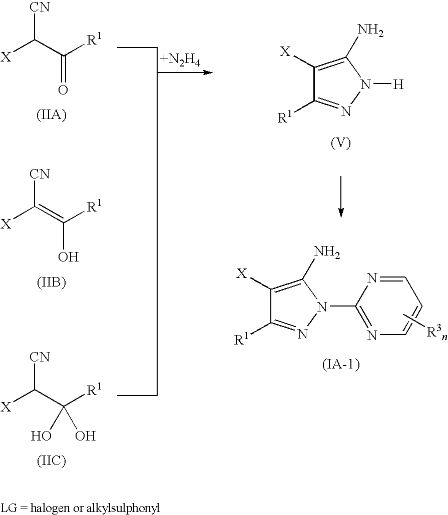

In the process (B) according to the invention for preparing the compounds of the formula (IA-1), 1-H-aminopyrazoles of the formula (V) are reacted with pyrimidinyl halides or alkylsulphones of the formula (VI) in the presence of a base in organic solvents, where a particular isomer, the aminopyrazole of the formula (IA-1), is formed with preference.

The 1-H-aminopyrazoles of the formula (V) can be prepared according to known methods:
DE-A 2643640
C. Chen et al, Bioorg. Med. Chem. Lett., 14 (2004), 14, 3669
Gilligan, Paul J. et al, Bioorg. Med. Chem., 8, (2000), 1, 181-190

Some of the pyrimidinyl halides or alkylsulphones of the formula (VI) are commercially available, or they can be synthesized according to methods known to the person skilled in the art.

Process (C)

Compounds of the formula (IA) according to the invention in which R⁵ or R⁶ or R⁵ and R⁶ does/do not represent hydrogen can be synthesized according to the process (C) according to the invention:

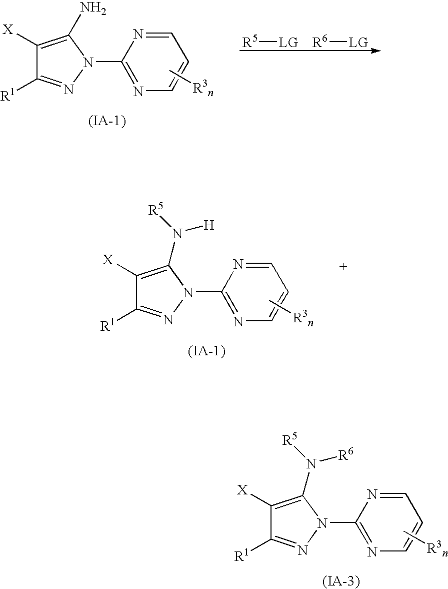

X, R¹, R³, R⁵, R⁶ and n may have the meanings given above (however, R⁵ and R⁶ do not represent hydrogen) and LG represents halogen or alkylsulphonyl.

In the process (C) according to the invention for preparing the compounds of the formula (IC) and (ID), compounds of the formula (IA-1) are reacted with one or two alkylating agents or acylating agents R⁵-LG and/or R⁶-LG, where aminopyrazoles of the formulae (IA-2) and (IA-3) are formed by monosubstitution and disubstitution, respectively. Suitable alkylating agents are alkyl bromides, alkyl dibromides, alkyl iodides, alkyl diiodides, dialkyl sulphates and alkylsulphonates. Suitable for use as acylating agents are carboxylic anhydrides and carbonyl chlorides.

Process (D)

Compounds of the formula (IB) according to the invention are synthesized, for example, according to process (D):

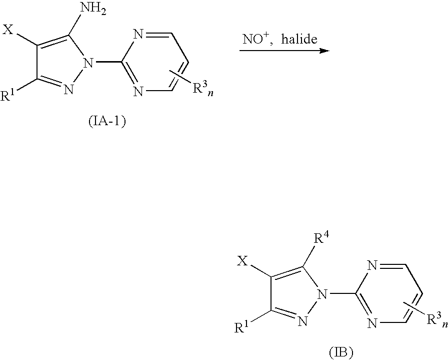

where X, R¹, R³, R⁴ and n have the meanings described above.

In the process (D) according to the invention for preparing the compounds of the formula (TB), aminopyrazoles of the formula (IA-1) are reacted with nitrosyl species in the presence of suitable halides, where after the formation of a diazo intermediate a 5-halopyrazole of the formula (TB) is formed. Suitable sources of nitrosyl species are alkali metal nitrites plus acids and also esters of nitrous acid, for example butyl nitrite. Suitable for use as halides are alkali metal halides and also organic halides, for example bromoform.

The synthesis of structurally related 5-halopyrazoles is described in
WO 2003/074492, p. 34, Bayer CropScience
Beck, James R. et al., J. Heterocycl. Chem., 24 (1987), 267-270

Process (E)

Compounds of the formula (IA-1) according to the invention in which R¹ represents an oxime, which compounds are referred to hereinbelow as (IA-1-2), are synthesized, for example, according to process (E) from the acetales of the formula (IA-1-1):

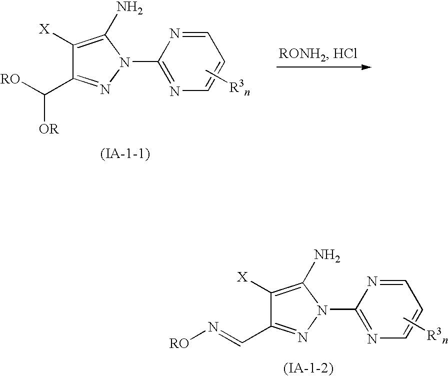

where X, R³ and n have the meanings described above and R represents alkyl.

In the process (E) according to the invention for preparing 3-oxime pyrazoles of the formula (IA-1-2), 3-acetalpyrazoles of the formula (IA-1-1) are reacted with O-alkyl hydroxylamine in organic solvents in the presence of acids.

Process (F)

Compounds of the formula (IA-1) according to the invention in which R¹ represents cyano, which compounds are referred to hereinbelow as compounds of the formula (IA-1-3), are synthesized, for example, according to process (F):

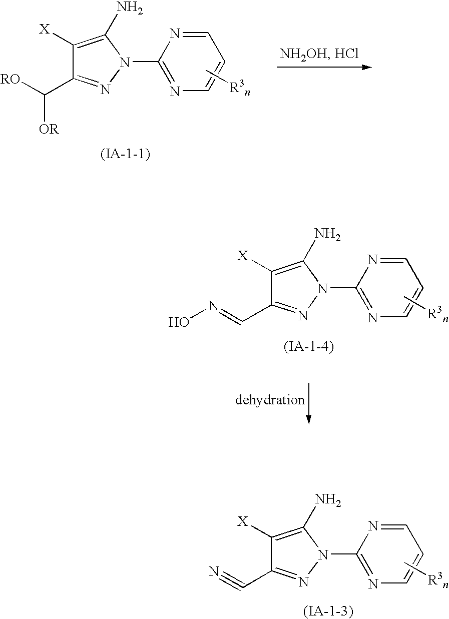

where X, R, R³ and n have the meanings described above and where the dehydrating agents used are acid chlorides, for example phosphorus oxychloride, or anhydrides.

Process (G)

Alternatively, compounds of the formula (IA-1) can also be prepared according to process (G) below:

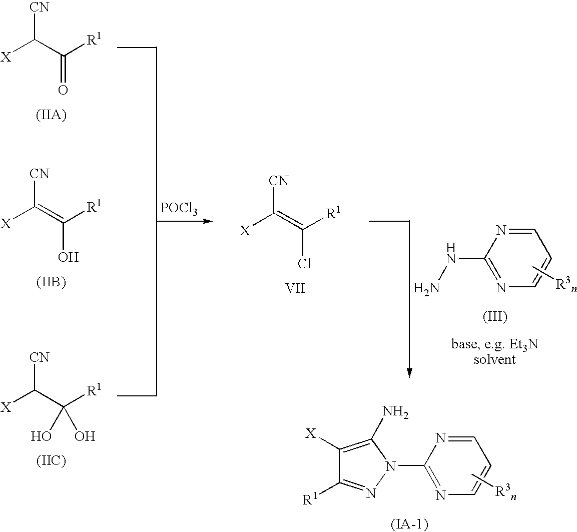

where X, R¹, R³ and n have the meanings given above.

In the process (G) according to the invention for preparing the compounds of the formula (IA-1), ketonitriles, their tautomers or hydrates of the formulae (IIA), (IIB) and (IIC), respectively, are reacted with chlorinating agents, for example phosphoryl chloride, thionyl chloride, phosgene, chlorine or oxalyl chloride, if appropriate diluted in an inert organic solvent, to give chloroacrylonitriles (VII), where the reaction may be carried out in a temperature range of from −20° C. to 120° C.

In a subsequent step, the product is condensed with pyrimidinylhydrazines (III) in a suitable organic solvent in the presence of basic auxiliaries, for example alkoxides or nitrogen bases, where the reaction may be carried out in a temperature range of from −20° C. to 120° C.

The ketonitriles can be present in the tautomeric forms (IIA) and (IIB) and as hydrate (IIC). Starting materials can also be employed in the form of their salts; for example, the ketonitriles can be used in the form of their alkali metal salts and the pyrimidinylhydrazines can be used in the form of their hydrochlorides.

The ketonitriles of the formula (II) can be prepared by known methods:
W. R. Nes, Alfred Burger, J. Amer. Chem. Soc. 72 (1950), 5409-5413

Some of the pyrimidinylhydrazines of the formula (III) are commercially available. The preparation of pyrimidinylhydrazines of the formula (III) is carried out according to the methods described in:
Methoden der Organischen Chemie [Methods of Organic Chemistry] (Houben-Weyl), Organische Stickstoff-Verbindungen [Organic Nitrogen Compounds], Volume E 16a, Part 1, pp. 678-775, Georg Thieme Verlag Stuttgart-New York, 1990.

The chloroacrylonitriles of the formula (VII) can also be prepared according to known methods:
JP08208620 (X=4-CF₃C₆H₆ and R¹═CF₃)

Process (H)

Alternatively, compounds of the formula (IA-1) can also be synthesized according to process (H) below:

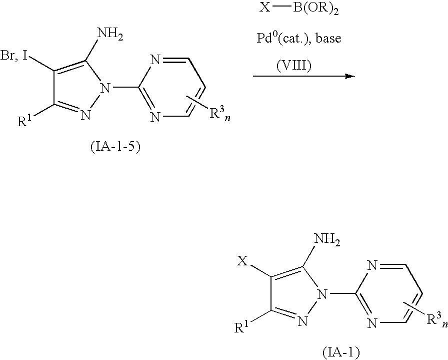

where X, R¹, R³ and n have the meanings given above.

In the process (H) according to the invention for preparing the compounds of the formula (IA-1), bromides or iodides of the formula (I-A-1-5) are reacted with boronic acids or boronic esters of the formula (VIII) in the presence of suitable palladium catalysts and bases (Suzuki reaction) in a temperature range of from −20° C. to 120° C. in suitable solvents.

The bromides or iodides of the formula (I-A-1-5) can be prepared according to known methods described, for example, in:
WO2005/11292
Chemistry of Heterocyclic Compounds (New York, N.Y., United States), 41(1), 105-110; 2005
Bioorganic & Medicinal Chemistry, 12(12), 3345-3355; 2004
Journal of Medicinal Chemistry, 20(12), 1562-9; 1977

Some of the boronic acids or boronic esters of the formula (VIII) are commercially available, or they can be prepared easily by known methods. This is described, for example, in:
WO 99/64428.

The active compounds of the invention, in combination with good plant tolerance and favourable toxicity to warm-blooded animals and being tolerated well by the environment, are suitable for protecting plants and plant organs, for increasing the harvest yields, for improving the quality of the harvested material and for controlling animal pests, in particular insects, arachnids, helminths, nematodes and molluscs, which are encountered in agriculture, in horticulture, in animal husbandry, in forests, in gardens and leisure facilities, in the protection of stored products and of materials, and in the hygiene sector. They may be preferably employed as plant protection agents. They are active against normally sensitive and resistant species and against all or some stages of development. The abovementioned pests include:

From the order of the Anoplura (Phthiraptera), for example, Damalinia spp., Haematopinus spp., Linognathus spp., Pediculus spp., Trichodectes spp.

From the class of the Arachnida, for example, Acarus siro, Aceria sheldoni, Aculops spp., Aculus spp., Amblyomma spp., Argas spp., Boophilus spp., Brevipalpus spp., Bryobia praetiosa, Chorioptes spp., Dermanyssus gallinae, Eotetranychus spp., Epitrimerus pyri, Eutetranychus spp., Eriophyes spp., Hemitarsonemus spp., Hyalomma spp., Ixodes spp., Latrodectus mactans, Metatetranychus spp., Oligonychus spp., Ornithodoros spp., Panonychus spp., Phyllocoptruta oleivora, Polyphagotarsonemus latus, Psoroptes spp., Rhipicephalus spp., Rhizoglyphus spp., Sarcoptes spp., Scorpio maurus, Stenotarsonemus spp., Tarsonemus spp., Tetranychus spp., Vasates lycopersici.

From the class of the Bivalva, for example, Dreissena spp.

From the order of the Chilopoda, for example, Geophilus spp., Scutigera spp.

From the order of the Coleoptera, for example, Acanthoscelides obtectus, Adoretus spp., Agelastica alni, Agriotes spp., Amphimallon solstitialis, Anobium punctatum, Anoplophora spp., Anthonomus spp., Anthrenus spp., Apogonia spp., Atomaria spp., Attagenus spp., Bruchidius obtectus, Bruchus spp., Ceuthorhynchus spp., Cleonus mendicus, Conoderus spp., Cosmopolites spp., Costelytra zealandica, Curculio spp., Cryptorhynchus lapathi, Dermestes spp., Diabrotica spp., Epilachna spp., Faustinus cubae, Gibbium psylloides, Heteronychus arator, Hylamorpha elegans, Hylotrupes bajulus, Hypera postica, Hypothenemus spp., Lachnosterna consanguinea, Leptinotarsa decemlineata, Lissorhoptrus oryzophilus, Lixus spp., Lyctus spp., Meligethes aeneus, Melolontha melolontha, Migdolus spp., Monochamus spp., Naupactus xanthographus, Niptus hololeucus, Oryctes rhinoceros, Oryzaephilus surinamensis, Otiorrhynchus sulcatus, Oxycetonia jucunda, Phaedon cochleariae, Phyllophaga spp., Popillia japonica, Premnotrypes spp., Psylliodes chrysocephala, Ptinus spp., Rhizobius ventralis, Rhizopertha dominica, Sitophilus spp., Sphenophorus spp., Sternechus spp., Symphyletes spp., Tenebrio molitor, Tribolium spp., Trogoderma spp., Tychius spp., Xylotrechus spp., Zabrus spp.

From the order of the Collembola, for example, Onychiurus armatus.

From the order of the Dermaptera, for example, Forficula auricularia.

From the order of the Diplopoda, for example, Blaniulus guttulatus.

From the order of the Diptera, for example, Aedes spp., Anopheles spp., Bibio hortulanus, Calliphora erythrocephala, Ceratitis capitata, Chrysomyia spp., Cochliomyia spp., Cordylobia anthropophaga, Culex spp., Cuterebra spp., Dacus oleae, Dermatobia hominis, Drosophila spp., Fannia spp., Gastrophilus spp., Hylemyia spp., Hyppobosca spp., Hypoderma spp., Liriomyza spp., Lucilia spp., Musca spp., Nezara spp., Oestrus spp., Oscinella frit, Pegomyia hyoscyami, Phorbia spp., Stomoxys spp., Tabanus spp., Tannia spp., Tipula paludosa, Wohlfahrtia spp.

From the class of the Gastropoda, for example, Arion spp., Biomphalaria spp., Bulinus spp., Deroceras spp., Galba spp., Lymnaea spp., Oncomelania spp., Succinea spp.

From the class of the helminths, for example, Ancylostoma duodenale, Ancylostoma ceylanicum, Acylostoma braziliensis, Ancylostoma spp., Ascaris lubricoides, Ascaris spp., Brugia malayi, Brugia timori, Bunostomum spp., Chabertia spp., Clonorchis spp., Cooperia spp., Dicrocoelium spp, Dictyocaulus filaria, Diphyllobothrium latum, Dracunculus medinensis, Echinococcus granulosus, Echinococcus multilocularis, Enterobius vermicularis, Faciola spp., Haemonchus spp., Heterakis spp., Hymenolepis nana, Hyostrongulus spp., Loa Loa, Nematodirus spp., Oesophagostomum spp., Opisthorchis spp., Onchocerca volvulus, Ostertagia spp., Paragonimus spp., Schistosomen spp, Strongyloides fuelleborni, Strongyloides stercoralis, Stronyloides spp., Taenia saginata, Taenia solium, Trichinella spiralis, Trichinella nativa, Trichinella britovi, Trichinella nelsoni, Trichinella pseudopsiralis, Trichostrongulus spp., Trichuris trichuria, Wuchereria bancrofti.

It is furthermore possible to control Protozoa, such as Eimeria.

From the order of the Heteroptera, for example, Anasa tristis, Antestiopsis spp., Blissus spp., Calocoris spp., Campylomma livida, Cavelerius spp., Cimex spp., Creontiades dilutus, Dasynus piperis, Dichelops furcatus, Diconocoris hewetti, Dysdercus spp., Euschistus spp., Eurygaster spp., Heliopeltis spp., Horcias nobilellus, Leptocorisa spp., Leptoglossus phyllopus, Lygus spp., Macropes excavatus, Miridae, Nezara spp., Oebalus spp., Pentomidae, Piesma quadrata, Piezodorus spp., Psallus seriatus, Pseudacysta persea, Rhodnius spp., Sahlbergella singularis, Scotinophora spp., Stephanitis nashi, Tibraca spp., Triatoma spp.

From the order of the Homoptera, for example, Acyrthosipon spp., Aeneolamia spp., Agonoscena spp., Aleurodes spp., Aleurolobus barodensis, Aleurothrixus spp., Amrasca spp., Anuraphis cardui, Aonidiella spp., Aphanostigma piri, Aphis spp., Arboridia apicalis, Aspidiella spp., Aspidiotus spp., Atanus spp., Aulacorthum solani, Bemisia spp., Brachycaudus helichrysii, Brachycolus spp., Brevicoryne brassicae, Calligypona marginata, Carneocephala fulgida, Ceratovacuna lanigera, Cercopidae, Ceroplastes spp., Chaetosiphon fragaefolii, Chionaspis tegalensis, Chlorita onukii, Chromaphis juglandicola, Chrysomphalus ficus, Cicadulina mbila, Coccomytilus halli, Coccus spp., Cryptomyzus ribis, Dalbulus spp., Dialeurodes spp., Diaphorina spp., Diaspis spp., Doralis spp., Drosicha spp., Dysaphis spp., Dysmicoccus spp., Empoasca spp., Eriosoma spp., Erythroneura spp., Euscelis bilobatus, Geococcus coffeae, Homalodisca coagulata, Hyalopterus arundinis, Icerya spp., Idiocerus spp., Idioscopus spp., Laodelphax striatellus, Lecanium spp., Lepidosaphes spp., Lipaphis erysimi, Macrosiphum spp., Mahanarva fimbriolata, Melanaphis sacchari, Metcalfiella spp., Metopolophium dirhodum, Monellia costalis, Monelliopsis pecanis, Myzus spp., Nasonovia ribisnigri, Nephotettix spp., Nilaparvata lugens, Oncometopia spp., Orthezia praelonga, Parabemisia myricae, Paratrioza spp., Parlatoria spp., Pemphigus spp., Peregrinus maidis, Phenacoccus spp., Phloeomyzus passerinii, Phorodon humuli, Phylloxera spp., Pinnaspis aspidistrae, Planococcus spp., Proto-pulvinaria pyriformis, Pseudaulacaspis pentagona, Pseudococcus spp., Psylla spp., Pteromalus spp., Pyrilla spp., Quadraspidiotus spp., Quesada gigas, Rastrococcus spp., Rhopalosiphum spp., Saissetia spp., Scaphoides titanus, Schizaphis graminum, Selenaspidus articulatus, Sogata spp., Sogatella furcifera, Sogatodes spp., Stictocephala festina, Tenalaphara malayensis, Tinocallis caryaefoliae, Tomaspis spp., Toxoptera spp., Trialeurodes vaporariorum, Trioza spp., Typhlocyba spp., Unaspis spp., Viteus vitifolii.

From the order of the Hymenoptera, for example, Diprion spp., Hoplocampa spp., Lasius spp., Monomorium pharaonis, Vespa spp.

From the order of the Isopoda, for example, Armadillidium vulgare, Oniscus asellus, Porcellio scaber.

From the order of the Isoptera, for example, Reticulitermes spp., Odontotermes spp.

From the order of the Lepidoptera, for example, Acronicta major, Aedia leucomelas, Agrotis spp., Alabama argillacea, Anticarsia spp., Barathra brassicae, Bucculatrix thurberiella, Bupalus piniarius, Cacoecia podana, Capua reticulana, Carpocapsa pomonella, Cheimatobia brumata, Chilo spp., Choristoneura fumiferana, Clysia ambiguella, Cnaphalocerus spp., Earias insulana, Ephestia kuehniella, Euproctis chrysorrhoea, Euxoa spp., Feltia spp., Galleria mellonella, Helicoverpa spp., Heliothis spp., Hofmannophila pseudospretella, Homona magnanima, Hyponomeuta padella, Laphygma spp., Lithocolletis blancardella, Lithophane antennata, Loxagrotis albicosta, Lymantria spp., Malacosoma neustria, Mamestra brassicae, Mocis repanda, Mythimna separata, Oria spp., Oulema oryzae, Panolis flammea, Pectinophora gossy-piella, Phyllocnistis citrella, Pieris spp., Plutella xylostella, Prodenia spp., Pseudaletia spp., Pseudoplusia includens, Pyrausta nubilalis, Spodoptera spp., Thermesia gemmatalis, Tinea pellionella, Tineola bisselliella, Tortrix viridana, Trichoplusia spp.

From the order of the Orthoptera, for example, Acheta domesticus, Blatta orientalis, Blattella germanica, Gryllotalpa spp., Leucophaea maderae, Locusta spp., Melanoplus spp., Periplaneta americana, Schistocerca gregaria.

From the order of the Siphonaptera, for example, Ceratophyllus spp., Xenopsylla cheopis.

From the order of the Symphyla, for example, Scutigerella immaculata.

From the order of the Thysanoptera, for example, Baliothrips biformis, Enneothrips flavens, Frankliniella spp., Heliothrips spp., Hercinothrips femoralis, Kakothrips spp., Rhipiphorothrips cruentatus, Scirtothrips spp., Taeniothrips cardamoni, Thrips spp.

From the order of the Thysanura, for example, Lepisma saccharina.

The phytoparasitic nematodes include, for example, Anguina spp., Aphelenchoides spp., Belonoaimus spp., Bursaphelenchus spp., Ditylenchus dipsaci, Globodera spp., Heliocotylenchus spp., Heterodera spp., Longidorus spp., Meloidogyne spp., Pratylenchus spp., Radopholus similis, Rotylenchus spp., Trichodorus spp., Tylenchorhynchus spp., Tylenchulus spp., Tylenchulus semipenetrans, Xiphinema spp.

If appropriate, the compounds according to the invention can, at certain concentrations or application rates, also be used as herbicides, safeners, growth regulators or agents to improve plant properties, or as microbicides, for example as fungicides, antimycotics, bactericides, viricides (including agents against viroids) or as agents against MLO (Mycoplasma-like organisms) and RLO (Rickettsia-like organisms). If appropriate, they can also be employed as intermediates or precursors for the synthesis of other active compounds.

The active compounds can be converted to the customary formulations, such as solutions, emulsions, wettable powders, water- and oil-based suspensions, powders, dusts, pastes, soluble powders, soluble granules, granules for broadcasting, suspension-emulsion concentrates, natural materials impregnated with active compound, synthetic materials impregnated with active compound, fertilizers and microencapsulations in polymeric substances.

These formulations are produced in a known manner, for example by mixing the active compounds with extenders, that is liquid solvents and/or solid carriers, optionally with the use of surfactants, that is emulsifiers and/or dispersants and/or foam-formers. The formulations are prepared either in suitable plants or else before or during the application.

Suitable for use as auxiliaries are substances which are suitable for imparting to the composition itself and/or to preparations derived therefrom (for example spray liquors, seed dressings) particular properties such as certain technical properties and/or also particular biological properties. Typical suitable auxiliaries are: extenders, solvents and carriers.

Suitable extenders are, for example, water, polar and nonpolar organic chemical liquids, for example from the classes of the aromatic and non-aromatic hydrocarbons (such as paraffins, alkylbenzenes, alkylnaphthalenes, chlorobenzenes), the alcohols and polyols (which, if appropriate, may also be substituted, etherified and/or esterified), the ketones (such as acetone, cyclohexanone), esters (including fats and oils) and (poly)ethers, the unsubstituted and substituted amines, amides, lactams (such as N-alkylpyrrolidones) and lactones, the sulphones and sulphoxides (such as dimethyl sulphoxide).

If the extender used is water, it is also possible to employ, for example, organic solvents as auxiliary solvents. Essentially, suitable liquid solvents are: aromatics such as xylene, toluene or alkylnaphthalenes, chlorinated aromatics and chlorinated aliphatic hydrocarbons such as chlorobenzenes, chloroethylenes or methylene chloride, aliphatic hydrocarbons such as cyclohexane or paraffins, for example petroleum fractions, mineral and vegetable oils, alcohols such as butanol or glycol and also their ethers and esters, ketones such as acetone, methyl ethyl ketone, methyl isobutyl ketone or cyclohexanone, strongly polar solvents such as dimethyl sulphoxide, and also water.

Suitable solid carriers are:

for example, ammonium salts and ground natural minerals such as kaolins, clays, talc, chalk, quartz, attapulgite, montmorillonite or diatomaceous earth, and ground synthetic minerals, such as finely divided silica, alumina and silicates; suitable solid carriers for granules are: for example, crushed and fractionated natural rocks such as calcite, marble, pumice, sepiolite and dolomite, and also synthetic granules of inorganic and organic meals, and granules of organic material such as paper, sawdust, coconut shells, maize cobs and tobacco stalks; suitable emulsifiers and/or foam-formers are: for example, nonionic and anionic emulsifiers, such as polyoxyethylene fatty acid esters, polyoxyethylene fatty alcohol ethers, for example alkylaryl polyglycol ethers, alkylsulphonates, alkyl sulphates, arylsulphonates and also protein hydrolysates; suitable dispersants are nonionic and/or ionic substances, for example from the classes of the alcohol-POE- and/or -POP-ethers, acid and/or POP-POE esters, alkyl aryl and/or POP-POE ethers, fat- and/or POP-POE adducts, POE- and/or POP-polyol derivatives, POE- and/or POP-sorbitan- or -sugar adducts, alkyl or aryl sulphates, alkyl- or arylsulphonates and alkyl or arylphosphates or the corresponding PO-ether adducts. Furthermore, suitable oligo- or polymers, for example those derived from vinylic monomers, from acrylic acid, from EO and/or PO alone or in combination with, for example, (poly)alcohols or (poly)amines. It is also possible to employ lignin and its sulphonic acid derivatives, unmodified and modified celluloses, aromatic and/or aliphatic sulphonic acids and their adducts with formaldehyde.

Tackifiers such as carboxymethylcellulose and natural and synthetic polymers in the form of powders, granules or latices, such as gum arabic, polyvinyl alcohol and polyvinyl acetate, as well as natural phospholipids such as cephalins and lecithins, and synthetic phospholipids, can be used in the formulations.

It is possible to use colorants such as inorganic pigments, for example iron oxide, titanium oxide and Prussian Blue, and organic dyestuffs, such as alizarin dyestuffs, azo dyestuffs and metal phthalocyanine dyestuffs, and trace nutrients such as salts of iron, manganese, boron, copper, cobalt, molybdenum and zinc.

Other possible additives are perfumes, mineral or vegetable, optionally modified oils, waxes and nutrients (including trace nutrients), such as salts of iron, manganese, boron, copper, cobalt, molybdenum and zinc.

Stabilizers, such as low-temperature stabilizers, preservatives, antioxidants, light stabilizers or other agents which improve chemical and/or physical stability may also be present.

The formulations generally comprise between 0.01 and 98% by weight of active compound, preferably between 0.5 and 90%.

The active compound according to the invention can be used in its commercially available formulations and in the use forms, prepared from these formulations, as a mixture with other active compounds, such as insecticides, attractants, sterilizing agents, bactericides, acaricides, nematicides, fungicides, growth-regulating substances, herbicides, safeners, fertilisers or semiochemicals.

Particularly favourable mixing components are, for example, the following compounds:

Fungicides:

Inhibitors of nucleic acid synthesis
benalaxyl, benalaxyl-M, bupirimate, chiralaxyl, clozylacon, dimethirimol, ethirimol, furalaxyl, hymexazol, mefenoxam, metalaxyl, metalaxyl-M, ofurace, oxadixyl, oxolinic acid

Inhibitors of mitosis and cell division
benomyl, carbendazim, diethofencarb, ethaboxam, fuberidazole, pencycuron, thiabendazole, thiophanate-methyl, zoxamide

Inhibitors of respiratory chain complex I
diflumetorim

Inhibitors of respiratory chain complex II
boscalid, carboxin, fenfuram, flutolanil, furametpyr, furmecyclox, mepronil, oxycarboxin, penthiopyrad, thifluzamide

Inhibitors of respiratory chain complex III
azoxystrobin, cyazofamid, dimoxystrobin, enestrobin, famoxadone, fenamidone, fluoxastrobin, kresoxim-methyl, metominostrobin, orysastrobin, pyraclostrobin, picoxystrobin, trifloxystrobin

Decouplers
dinocap, fluazinam

Inhibitors of ATP production
fentin acetate, fentin chloride, fentin hydroxide, silthiofam

Inhibitors of amino acid biosynthesis and protein biosynthesis
andoprim, blasticidin-S, cyprodinil, kasugamycin, kasugamycin hydrochloride hydrate, mepanipyrim, pyrimethanil

Inhibitors of signal transduction
fenpiclonil, fludioxonil, quinoxyfen

Inhibitors of lipid and membrane synthesis
chlozolinate, iprodione, procymidone, vinclozolin
ampropylfos, potassium-ampropylfos, edifenphos, etridiazole, iprobenfos (IBP), isoprothiolane, pyrazophos
tolclofos-methyl, biphenyl
iodocarb, propamocarb, propamocarb hydrochloride, propamocarb-fosetylate

Inhibitors of ergosterol biosynthesis
fenhexamid,
azaconazole, bitertanol, bromuconazole, cyproconazole, diclobutrazole, difenoconazole, diniconazole, diniconazole-M, epoxiconazole, etaconazole, fenarimol, fenbuconazole, fluquinconazole, flurprimidole, flusilazole, flutriafol, furconazole, furconazole-cis, hexaconazole, imazalil, imazalil sulphate, imibenconazole, ipconazole, metconazole, myclobutanil, nuarimol, oxpoconazole, paclobutrazole, penconazole, pefurazoate, prochloraz, propiconazole, prothioconazole, pyrifenox, simeconazole, tebuconazole, tetraconazole, triadimefon, triadimenol, triflumizole, triforine, triticonazole, uniconazole, voriconazole, viniconazole,
aldimorph, dodemorph, dodemorph acetate, fenpropidin, fenpropimorph, spiroxamine, tridemorph,
naftifine, pyributicarb, terbinafine

Inhibitors of cell wall synthesis
benthiavalicarb, bialaphos, dimethomorph, flumorph, iprovalicarb, mandipropamid, polyoxins, polyoxorim, validamycin A

Inhibitors of melanin biosynthesis
capropamid, diclocymet, fenoxanil, phthalid, pyroquilon, tricyclazole

Resistance inductors
acibenzolar-S-methyl, probenazole, tiadinil

Multisite
captafol, captan, chlorothalonil, copper salts such as: copper hydroxide, copper naphthenate, copper oxychloride, copper sulphate, copper oxide, oxine-copper and Bordeaux mixture, dichlofluanid, dithianon, dodine, dodine free base, ferbam, folpet, fluorofolpet, guazatine, guazatine acetate, iminoctadine, iminoctadine albesilate, iminoctadine triacetate, mancopper, mancozeb, maneb, metiram, metiram zinc, propineb, sulphur and sulphur preparations containing calcium polysulphide, thiram, tolylfluanid, zineb, ziram

Further fungicides
amibromdol, benthiazole, bethoxazin, capsimycin, carvone, quinomethionate, chloropicrin, cufraneb, cyflufenamid, cymoxanil, dazomet, debacarb, diclomezine, dichlorophen, dicloran, difenzoquat, difenzoquat metilsulphate, diphenylamine, ferimzone, flumetover, flusulphamide, fluopicolide, fluoroimide, fosetyl-aluminium, fosetyl-calcium, fosetyl-sodium, hexachlorobenzene, 8-hydroxyquinoline sulphate, irumamycin, isotianil, methasulphocarb, metrafenone, methyl isothiocyanate, mildiomycin, natamycin, nickel dimethyl dithiocarbamate, nitrothal-isopropyl, octhilinone, oxamocarb, oxyfenthiin, pentachlorophenol and salts, 2-phenylphenol and salts, piperalin, propanosine-sodium, proquinazid, pyribencarb, pyrrolnitrin, quintozene, tecloftalam, tecnazene, triazoxide, trichlamide, valiphenal, zarilamid,
2-(2-{[6-(3-chloro-2-methylphenoxy)-5-fluoropyrimidin-4-yl]oxy}phenyl)-2-(methoxyimino)-N-methylacetamide,
2-[[[[1-[3-(1-fluoro-2-phenylethyl)oxy]phenyl]ethylidene]amino]oxy]methyl]-alpha-(methoxyimino)-N-methyl-alpha-benzacetamide,
cis-1-(4-chlorophenyl)-2-(1H-1,2,4-triazol-1-yl)cycloheptanol,
1-[(4-methoxyphenoxy)methyl]-2,2-dimethylpropyl-1H-imidazole-1-carboxylic acid,
2,3,5,6-tetrachloro-4-(methylsulphonyl)pyridine,
2-butoxy-6-iodo-3-propylbenzopyranon-4-one,
2-chloro-N-(2,3-dihydro-1,1,3-trimethyl-1H-inden-4-yl)-3-pyridinecarboxamide,
3,4,5-trichloro-2,6-pyridinedicarbonitrile,
3-[5-(4-chlorophenyl)-2,3-dimethylisoxazolidin-3-yl]pyridine,
5-chloro-6-(2,4,6-trifluorophenyl)-N-[(1R)-1,2,2-trimethylpropyl][1,2,4]triazolo[1,5-a]pyrimidine-7-amine,
5-chloro-7-(4-methylpiperidin-1-yl)-6-(2,4,6-trifluorophenyl)[1,2,4]triazolo[1,5-a]pyrimidine,
5-chloro-N-[(1R)-1,2-dimethylpropyl]-6-(2,4,6-trifluorophenyl)[1,2,4]triazolo[1,5-a]pyrimidine-7-amine,
methyl 2-Mcyclopropyl[(4-methoxyphenyl)imino]methyl]thio]methyl]alpha-(methoxymethylene)benzacetate,
methyl 1-(2,3-dihydro-2,2-dimethyl-1H-inden-1-yl)-1H-imidazole-5-carboxylate,
N-(3′,4′-dichloro-5-fluorobiphenyl-2-yl)-3-(difluoromethyl)-1-methyl-1H-pyrazole-4-carboxamide,
N-(3-ethyl-3,5,5-trimethylcyclohexyl)-3-formylamino-2-hydroxy benzamide,
N-(4-chloro-2-nitrophenyl)-N-ethyl-4-methylbenzenesulphonamide,
N-(4-chlorobenzyl)-3-[3-methoxy-4-(prop-2-yn-1-yloxy)phenyl]propanamide,
N-[(4-chlorophenyl)(cyano)methyl]-3-[3-methoxy-4-(prop-2-yn-1-yloxy)phenyl]propanamide,
N-(5-bromo-3-chloropyridin-2-yl)methyl-2,4-dichloronicotinamide,
N-[1-(5-bromo-3-chloropyridin-2-yl]ethyl]-2,4-dichloronicotinamide,
(2S)-N-[2-[4-[[3-(4-chlorophenyl)-2-propynyl]oxy]-3-methoxyphenyl]ethyl]-3-methyl-2-[(methylsulphonyl)amino]butanamide,

N-{(Z)-[(cyclopropylmethoxy)imino][6-(difluoromethoxy)-2,3-difluorophenyl]methyl}-2-benzacetamide,
N-{2-[1,1′-bi(cyclopropyl)-2-yl]phenyl}-3-(difluoromethyl)-1-methyl-1H-pyrazole-4-carboxamide,
N-{2-[3-chloro-5-(trifluoromethyl)pyridin-2-yl]ethyl}-2-(trifluoromethyl)benzamide,
N-ethyl-N-methyl-N′-{2-methyl-5-(trifluoromethyl)-4-[3-(trimethylsilyl)propoxy]phenyl}imidoformamide,
N-ethyl-N-methyl-N′-{2-methyl-5-(difluoromethyl)-4-[3-(trimethylsilyl)propxy]phenyl}imidoformamide,
O-[1-[(4-methoxyphenoxy)methyl]-2,2-dimethylpropyl]-1H-imidazole-1-carbothioic acid,
2-amino-4-methyl-N-phenyl-5-thiazolecarboxamide,
2,4-dihydro-5-methoxy-2-methyl-4-[[[[1-[3-(trifluoromethyl)phenyl]ethylidene]amino]oxy]methyl]phenyl]-3H-1,2,4-triazol-3-one (CAS No. 185336-79-2),
N-(6-methoxy-3-pyridinyl)cyclopropane carboxamide,

Bactericides:

bronopol, dichlorophen, nitrapyrin, nickel dimethyldithiocarbamate, kasugamycin, octhilinone, furancarboxylic acid, oxytetracycline, probenazole, streptomycin, tecloftalam, copper sulphate and other copper preparations.

Insecticides/Acaricides/Nematicides:

Acetylcholine esterase (AChE) inhibitors
Carbamates,
   for example alanycarb, aldicarb, aldoxycarb, allyxycarb, aminocarb, bendiocarb, benfuracarb, bufencarb, butacarb, butocarboxim, butoxycarboxim, carbaryl, carbofuran, carbosulphan, cloethocarb, dimetilan, ethiofencarb, fenobucarb, fenothiocarb, formetanate, furathiocarb, isoprocarb, metam-sodium, methiocarb, methomyl, metolcarb, oxamyl, pirimicarb, promecarb, propoxur, thiodicarb, thiofanox, trimethacarb, XMC, xylylcarb, triazamate
Organophosphates,
   for example acephate, azamethiphos, azinphos (-methyl, -ethyl), bromophos-ethyl, bromfenvinfos (-methyl), butathiofos, cadusafos, carbophenothion, chlorethoxyfos, chlorfenvinphos, chlormephos, chlorpyrifos (-methyl/-ethyl), coumaphos, cyanofenphos, cyanophos, chlorfenvinphos, demeton-S -methyl, demeton-S-methylsulphone, dialifos, diazinon, dichlofenthion, dichlorvos/DDVP, dicrotophos, dimethoate, dimethylvinphos, dioxabenzofos, disulphoton, EPN, ethion, ethoprophos, etrimfos, famphur, fenamiphos, fenitrothion, fensulphothion, fenthion, flupyrazofos, fonofos, formothion, fosmethilan, fosthiazate, heptenophos, iodofenphos, iprobenfos, isazofos, isofenphos, isopropyl O-salicylate, isoxathion, malathion, mecarbam, methacrifos, methamidophos, methidathion, mevinphos, monocrotophos, naled, omethoate, oxydemeton-methyl, parathion (-methyl/-ethyl), phenthoate, phorate, phosalone, phosmet, phosphamidon, phosphocarb, phoxim, pirimiphos (-methyl/-ethyl), profenofos, propaphos, propetamphos, prothiofos, prothoate, pyraclofos, pyridaphenthion, pyridathion, quinalphos, sebufos, sulphotep, sulprofos, tebupirimfos, temephos, terbufos, tetrachlorvinphos, thiometon, triazophos, triclorfon, vamidothion

Sodium channel modulators/voltage-dependent sodium channel blockers
Pyrethroids,
   for example acrinathrin, allethrin (d-cis-trans, d-trans), beta-cyfluthrin, bifenthrin, bioallethrin, bioallethrin-S-cyclopentyl isomer, bioethanomethrin, biopermethrin, bioresmethrin, chlovaporthrin, cis-cypermethrin, cis-resmethrin, cis-permethrin, clocythrin, cycloprothrin, cyfluthrin, cyhalothrin, cypermethrin (alpha-, beta-, theta-, zeta-), cyphenothrin, deltamethrin, empenthrin (1R-isomer), esfenvalerate, etofenprox, fenfluthrin, fenpropathrin, fenpyrithrin, fenvalerate, flubrocythrinate, flucythrinate, flufenprox, flumethrin, fluvalinate, fubfenprox, gamma-cyhalothrin, imiprothrin, kadethrin, lambda-cyhalothrin, metofluthrin, permethrin (cis-, trans-), phenothrin (1R-trans-isomer), prallethrin, profluthrin, protrifenbute, pyresmethrin, resmethrin, RU 15525, silafluofen, tau-fluvalinate, tefluthrin, terallethrin, tetramethrin (1R isomer), tralomethrin, transfluthrin, ZXI 8901, pyrethrins (pyrethrum)
DDT
Oxadiazines,
for example indoxacarb
Semicarbazone,
for example metaflumizon (BAS3201)

Acetylcholine receptor agonists/antagonists
Chloronicotinyls,
for example acetamiprid, clothianidin, dinotefuran, imidacloprid, nitenpyram, nithiazine, thiacloprid, thiamethoxam
Nicotine, bensultap, cartap

Acetylcholine receptor modulators
Spinosyns,
for example spinosad, spinetoram

GABA-controlled chloride channel antagonists
Organochlorines,
for example camphechlor, chlordane, endosulphan, gamma-HCH, HCH, heptachlor, lindane, methoxychlor
Fiprols,
for example acetoprole, ethiprole, fipronil, pyrafluprole, pyriprole, vaniliprole

Chloride channel activators
Mectins,
for example abamectin, emamectin, emamectin-benzoate, ivermectin, lepimectin, milbemycin

Juvenile hormone mimetics,
for example diofenolan, epofenonane, fenoxycarb, hydroprene, kinoprene, methoprene, pyriproxifen, triprene

Ecdysone agonists/disruptors
Diacylhydrazines,
for example chromafenozide, halofenozide, methoxyfenozide, tebufenozide

Chitin biosynthesis inhibitors
Benzoylureas,
for example bistrifluron, chlofluazuron, diflubenzuron, fluazuron, flucycloxuron, flufenoxuron, hexaflumuron, lufenuron, novaluron, noviflumuron, penfluron, teflubenzuron, triflumuron
Buprofezin
Cyromazine

Oxidative phosphorylation inhibitors, ATP disruptors
Diafenthiuron
Organotin compounds,
for example azocyclotin, cyhexatin, fenbutatin-oxide

Oxidative phosphorylation decouplers acting by interrupting the H-proton gradient
Pyrroles,
for example chlorfenapyr
Dinitrophenols,
for example binapacryl, dinobuton, dinocap, DNOC, meptyldinocap

Site-I electron transport inhibitors
METI's,
for example fenazaquin, fenpyroximate, pyrimidifen, pyridaben, tebufenpyrad, tolfenpyrad
Hydramethylnon
Dicofol

Site-II electron transport inhibitors
Rotenone

Site-III electron transport inhibitors
Acequinocyl, fluacrypyrim

Microbial disruptors of the insect gut membrane
Bacillus thuringiensis strains

Lipid synthesis inhibitors
Tetronic acids,
for example spirodiclofen, spiromesifen

Tetramic acids,
for example spirotetramat, cis-3-(2,5-dimethylphenyl)-4-hydroxy-8-methoxy-1-azaspiro[4.5]dec-3-en-2-one
Carboxamides,
for example flonicamid
Octopaminergic agonists,
for example amitraz

Inhibitors of magnesium-stimulated ATPase,
Propargite
Nereistoxin analogues,
for example thiocyclam hydrogen oxalate, thiosultap-sodium

Ryanodine receptor agonists,
Benzodicarboxamides,
for example flubendiamide
Anthranilamides,
for example Rynaxypyr (3-bromo-N-{4-chloro-2-methyl-6-[(methylamino)carbonyl]-phenyl}-1-(3-chloropyridin-2-yl)-1H-pyrazole-5-carboxamide)

Biologicals, hormones or pheromones
azadirachtin, Bacillus spec., Beauveria spec., codlemone, Metarrhizium spec., Paecilomyces spec., thuringiensin, Verticillium spec.

Active compounds with unknown or unspecific mechanisms of action
Fumigants,
for example aluminium phosphide, methyl bromide, sulphuryl fluoride
Antifeedants,
for example cryolite, flonicamid, pymetrozine
Mite growth inhibitors,
for example clofentezine, etoxazole, hexythiazox
Amidoflumet, benclothiaz, benzoximate, bifenazate, bromopropylate, buprofezin, chinomethionat, chlordimeform, chlorobenzilate, chloropicrin, clothiazoben, cycloprene, cyflumetofen, dicyclanil, fenoxacrim, fentrifanil, flubenzimine, flufenerim, flutenzin, gossyplure, hydramethylnone, japonilure, metoxadiazone, petroleum, piperonyl butoxide, potassium oleate, pyridalyl, sulphluramid, tetradifon, tetrasul, triarathene, verbutin

A mixture with other known active compounds, such as herbicides, fertilizers, growth regulators, safeners, semiochemicals, or else with agents for improving the plant properties, is also possible.

When used as insecticides, the active compounds according to the invention can furthermore be present in their commercially available formulations and in the use forms, prepared from these formulations, as a mixture with synergistic agents. Synergistic agents are compounds which increase the action of the active compounds, without it being necessary for the synergistic agent added to be active itself.

When used as insecticides, the active compounds according to the invention can furthermore be present in their commercially available formulations and in the use forms, prepared from these formulations, as a mixture with inhibitors which reduce degradation of the active compound after use in the environment of the plant, on the surface of parts of plants or in plant tissues.

The active compound content of the use forms prepared from the commercially available formulations can vary within wide limits. The active compound concentration of the use forms can be from 0.00000001 to 95% by weight of active compound, preferably between 0.00001 and 1% by weight.

The compounds are employed in a customary manner appropriate for the use forms.

All plants and plant parts can be treated in accordance with the invention. Plants are to be understood as meaning in the present context all plants and plant populations such as desired and undesired wild plants or crop plants (including naturally occurring crop plants). Crop plants can be plants which can be obtained by conventional plant breeding and optimization methods or by biotechnological and genetic engineering methods or by combinations of these methods, including the transgenic plants and including the plant cultivars protectable or not protectable by plant breeders' rights. Plant parts are to be understood as meaning all parts and organs of plants above and below the ground, such as shoot, leaf, flower and root, examples which may be mentioned being leaves, needles, stalks, stems, flowers, fruit bodies, fruits, seeds, roots, tubers and rhizomes. The plant parts also include harvested material, and vegetative and generative propagation material, for example cuttings, tubers, rhizomes, offshoots and seeds.

Treatment according to the invention of the plants and plant parts with the active compounds is carried out directly or by allowing the compounds to act on the surroundings, habitat or storage space by the customary treatment methods, for example by immersion, spraying, evaporation, fogging, scattering, painting on, injection and, in the case of propagation material, in particular in the case of seeds, also by applying one or more coats.

As already mentioned above, it is possible to treat all plants and their parts according to the invention. In a preferred embodiment, wild plant species and plant cultivars, or those obtained by conventional biological breeding methods, such as crossing or protoplast fusion, and parts thereof, are treated. In a further preferred embodiment, transgenic plants and plant cultivars obtained by genetic engineering methods, if appropriate in combination with conventional methods (Genetically Modified Organisms), and parts thereof are treated. The terms “parts”, “parts of plants” and “plant parts” have been explained above.

Particularly preferably, plants of the plant cultivars which are in each case commercially available or in use are treated according to the invention. Plant cultivars are to be understood as meaning plants having novel properties (“traits”) which have been obtained by conventional breeding, by mutagenesis or by recombinant DNA techniques. These can be cultivars, bio- or genotypes.

Depending on the plant species or plant cultivars, their location and growth conditions (soils, climate, vegetation period, diet), the treatment according to the invention may also result in superadditive (“synergistic”) effects. Thus, for example, reduced application rates and/or a widening of the activity spectrum and/or an increase in the activity of the substances and compositions which can be used according to the invention, better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products are possible, which exceed the effects which were actually to be expected.

The transgenic plants or plant cultivars (obtained by genetic engineering) which are preferably to be treated according to the invention include all plants which, by virtue of the genetic modification, received genetic material which imparted particularly advantageous, useful traits to these plants. Examples of such traits are better plant growth, increased tolerance to high or low temperatures, increased tolerance to drought or to water or soil salt content, increased flowering performance, easier harvesting, accelerated maturation, higher harvest yields, higher quality and/or a higher nutritional value of the harvested products, better storage stability and/or processability of the harvested products. Further and particularly emphasized examples of such traits are a better defence of the plants against animal and microbial pests, such as against insects, mites, phytopathogenic fungi, bacteria and/or viruses, and also increased tolerance of the plants to certain herbicidally active compounds. Examples of transgenic plants which may be mentioned are the important crop plants, such as cereals (wheat, rice), maize, soya beans, potatoes, sugar beet, tomatoes, peas and other vegetable varieties, cotton, tobacco, oilseed rape and also fruit plants (with the fruits apples, pears, citrus fruits and grapes), and particular emphasis is given to maize, soya beans, potatoes, cotton, tobacco and oilseed rape. Traits that are emphasized are in particular increased defence of the plants against insects, arachnids, nematodes and worms by virtue of toxins formed in the plants, in particular those formed in the plants by the genetic material from Bacillus thuringiensis (for example by the genes CryIA(a), CryIA(b), CryIA(c), CryIIA, CryIIIA, CryIIIB2, Cry9c, Cry2Ab, Cry3Bb and CryIF and also combinations thereof) (referred to hereinbelow as “Bt plants”). Traits that are also particularly emphasized are the increased defence of the plants against fungi, bacteria and viruses by systemic acquired resistance (SAR), systemin, phytoalexins, elicitors and resistance genes and correspondingly expressed proteins and toxins. Traits that are furthermore particularly emphasized are the increased tolerance of the plants to certain herbicidally active compounds, for example imidazolinones, sulphonylureas, glyphosate or phosphinotricin (for example the “PAT” gene). The genes which impart the desired traits in question can also be present in combination with one another in the transgenic plants. Examples of “Bt plants” which may be mentioned are maize varieties, cotton varieties, soya bean varieties and potato varieties which are sold under the trade names YIELD GARD® (for example maize, cotton, soya beans), KnockOut® (for example maize), StarLink® (for example maize), Bollgard® (cotton), Nucotn® (cotton) and NewLeaf® (potato). Examples of herbicide-tolerant plants which may be mentioned are maize varieties, cotton varieties and soya bean varieties which are sold under the trade names Roundup Ready® (tolerance to glyphosate, for example maize, cotton, soya bean), Liberty Link® (tolerance to phosphinotricin, for example oilseed rape), IMF® (tolerance to imidazolinones) and STS® (tolerance to sulphonylureas, for example maize). Herbicide-resistant plants (plants bred in a conventional manner for herbicide tolerance) which may be mentioned include the varieties sold under the name Clearfield® (for example maize). Of course, these statements also apply to plant cultivars having these genetic traits or genetic traits still to be developed, which plants will be developed and/or marketed in the future.

The plants listed can be treated according to the invention in a particularly advantageous manner with the compounds of the general formula I and/or the active compound mixtures according to the invention. The preferred ranges stated above for the active compounds or mixtures also apply to the treatment of these plants. Particular emphasis is given to the treatment of plants with the compounds or mixtures specifically mentioned in the present text.

The active compounds according to the invention act not only against plant, hygiene and stored product pests, but also in the veterinary medicine sector against animal parasites (ecto- and endoparasites), such as hard ticks, soft ticks, mange mites, leaf mites, flies (biting and licking), parasitic fly larvae, lice, hair lice, feather lice and fleas. These parasites include:

From the order of the Anoplurida, for example, Haematopinus spp., Linognathus spp., Pediculus spp., Phtirus spp., Solenopotes spp.

From the order of the Mallophagida and the suborders Amblycerina and Ischnocerina, for example, Trimenopon spp., Menopon spp., Trinoton spp., Bovicola spp., Werneckiella spp., Lepikentron spp., Damalina spp., Trichodectes spp., Felicola spp.

From the order of the Diptera and the suborders Nematocerina and Brachycerina, for example, Aedes spp., Anopheles spp., Culex spp., Simulium spp., Eusimulium spp., Phlebotomus spp., Lutzomyia spp., Culicoides spp., Chrysops spp., Hybomitra spp., Atylotus spp., Tabanus spp., Haematopota spp., Philipomyia spp., Braula spp., Musca spp., Hydrotaea spp., Stomoxys spp., Haematobia spp., Morellia spp., Fannia spp., Glossina spp., Calliphora spp., Lucilia spp., Chrysomyia spp., Wohlfahrtia spp., Sarcophaga spp., Oestrus spp., Hypoderma spp., Gasterophilus spp., Hippobosca spp., Lipoptena spp., Melophagus spp.

From the order of the Siphonapterida, for example, Pulex spp., Ctenocephalides spp., Xenopsylla spp., Ceratophyllus spp.

From the order of the Heteropterida, for example, Cimex spp., Triatoma spp., Rhodnius spp., Panstrongylus spp.

From the order of the Blattarida, for example, Blatta orientalis, Periplaneta americana, Blattela germanica, Supella spp.

From the subclass of the Acari (Acarina) and the orders of the Meta- and Mesostigmata, for example, Argas spp., Ornithodorus spp., Otobius spp., Ixodes spp., Amblyomma spp., Boophilus spp., Dermacentor spp., Haemophysalis spp., Hyalomma spp., Rhipicephalus spp., Dermanyssus spp., Raillietia spp., Pneumonyssus spp., Sternostoma spp., Varroa spp.

From the order of the Actinedida (Prostigmata) and Acaridida (Astigmata), for example, Acarapis spp., Cheyletiella spp., Ornithocheyletia spp., Myobia spp., Psorergates spp., Demodex spp., Trombicula spp., Listrophorus spp., Acarus spp., Tyrophagus spp., Caloglyphus spp., Hypodectes spp., Pterolichus spp., Psoroptes spp., Chorioptes spp., Otodectes spp., Sarcoptes spp., Notoedres spp., Knemidocoptes spp., Cytodites spp., Laminosioptes spp.

The active compounds of the formula (I) according to the invention are also suitable for controlling arthropods which infest agricultural productive livestock, such as, for example, cattle, sheep, goats, horses, pigs, donkeys, camels, buffalo, rabbits, chickens, turkeys, ducks, geese and bees, other pets, such as, for example, dogs, cats, caged birds and aquarium fish, and also so-called test animals, such as, for example, hamsters, guinea pigs, rats and mice. By controlling these arthropods, cases of death and reduction in productivity (for meat, milk, wool, hides, eggs, honey etc.) should be diminished, so that more economic and easier animal husbandry is possible by use of the active compounds according to the invention.

The active compounds according to the invention are used in the veterinary sector and in animal husbandry in a known manner by enteral administration in the form of, for example, tablets, capsules, potions, drenches, granules, pastes, boluses, the feed-through process and suppositories, by parenteral administration, such as, for example, by injection (intramuscular, subcutaneous, intravenous, intraperitoneal and the like), implants by nasal administration, by dermal use in the form, for example, of dipping or bathing, spraying, pouring on and spotting on, washing and powdering, and also with the aid of moulded articles containing the active compound, such as collars, ear marks, tail marks, limb bands, halters, marking devices and the like.

When used for cattle, poultry, pets and the like, the active compounds of the formula (I) can be used as formulations (for example powders, emulsions, free-flowing compositions), which comprise the active compounds in an amount of Ito 80% by weight, directly or after 100 to 10 000-fold dilution, or they can be used as a chemical bath.

It has furthermore been found that the compounds according to the invention also have a strong insecticidal action against insects which destroy industrial materials.

The following insects may be mentioned as examples and as preferred—but without any limitation:

Beetles, such as Hylotrupes bajulus, Chlorophorus pilosis, Anobium punctatum, Xestobium rufovillosum, Ptilinus pecticornis, Dendrobium pertinex, Ernobius mollis, Priobium carpini, Lyctus brunneus, Lyctus africanus, Lyctus planicollis, Lyctus linearis, Lyctus pubescens, Trogoxylon aequale, Minthes rugicollis, Xyleborus spec. Tryptodendron spec. Apate monachus, Bostrychus capucins, Heterobostrychus brunneus, Sinoxylon spec. Dinoderus minutus;

Hymenopterons, such as Sirex juvencus, Urocerus gigas, Urocerus gigas taignus, Urocerus augur;

Termites, such as Kalotermes flavicollis, Cryptotermes brevis, Heterotermes indicola, Reticulitermes flavipes, Reticulitermes santonensis, Reticulitermes lucifugus, Mastotermes darwiniensis, Zootermopsis nevadensis, Coptotermes formosanus;

Bristletails, such as Lepisma saccharina.

Industrial materials in the present connection are to be understood as meaning non-living materials, such as, preferably, plastics, adhesives, sizes, papers and cardboards, leather, wood and processed wood products and coating compositions.

The ready-to-use compositions may, if appropriate, comprise further insecticides and, if appropriate, one or more fungicides.

With respect to possible additional additives, reference may be made to the insecticides and fungicides mentioned above.

The compounds according to the invention can likewise be employed for protecting objects which come into contact with saltwater or brackish water, such as hulls, screens, nets, buildings, moorings and signalling systems, against fouling.

Furthermore, the compounds according to the invention, alone or in combination with other active compounds, may be employed as antifouling agents.

In domestic, hygiene and stored-product protection, the active compounds are also suitable for controlling animal pests, in particular insects, arachnids and mites, which are found in enclosed spaces such as, for example, dwellings, factory halls, offices, vehicle cabins and the like. They can be employed alone or in combination with other active compounds and auxiliaries in domestic insecticide products for controlling these pests. They are active against sensitive and resistant species and against all developmental stages. These pests include:

From the order of the Scorpionidea, for example, Buthus occitanus.

From the order of the Acarina, for example, Argas persicus, Argas reflexus, Bryobia ssp., Dermanyssus gallinae, Glyciphagus domesticus, Ornithodorus moubat, Rhipicephalus sanguineus, Trombicula alfreddugesi, Neutrombicula autumnalis, Dermatophagoides pteronissimus, Dermatophagoides forinae.

From the order of the Araneae, for example, Aviculariidae, Araneidae.

From the order of the Opiliones, for example, Pseudoscorpiones chelifer, Pseudoscorpiones cheiridium, Opiliones phalangium.

From the order of the Isopoda, for example, Oniscus asellus, Porcellio scaber.

From the order of the Diplopoda, for example, Blaniulus guttulatus, Polydesmus spp.

From the order of the Chilopoda, for example, Geophilus spp.

From the order of the Zygentoma, for example, Ctenolepisma spp., Lepisma saccharina, Lepismodes inquilinus.

From the order of the Blattaria, for example, Blatta orientalies, Blattella germanica, Blattella asahinai, Leucophaea maderae, Panchlora spp., Parcoblatta spp., Periplaneta australasiae, Periplaneta americana, Periplaneta brunnea, Periplaneta fuliginosa, Supella longipalpa.

From the order of the Saltatoria, for example, Acheta domesticus.

From the order of the Dermaptera, for example, Forficula auricularia.

From the order of the Isoptera, for example, Kalotermes spp., Reticulitermes spp.

From the order of the Psocoptera, for example, Lepinatus spp., Liposcelis spp.

From the order of the Coleoptera, for example, Anthrenus spp., Attagenus spp., Dermestes spp., Latheticus oryzae, Necrobia spp., Ptinus spp., Rhizopertha dominica, Sitophilus granarius, Sitophilus oryzae, Sitophilus zeamais, Stegobium paniceum.

From the order of the Diptera, for example, Aedes aegypti, Aedes albopictus, Aedes taeniorhynchus, Anopheles spp., Calliphora erythrocephala, Chrysozona pluvialis, Culex quinquefasciatus, Culex pipiens, Culex tarsalis, Drosophila spp., Fannia canicularis, Musca domestica, Phlebotomus spp., Sarcophaga carnaria, Simulium spp., Stomoxys calcitrans, Tipula paludosa.

From the order of the Lepidoptera, for example, Achroia grisella, Galleria mellonella, Plodia interpunctella, Tinea cloacella, Tinea pellionella, Tineola bisselliella.

From the order of the Siphonaptera, for example, Ctenocephalides canis, Ctenocephalides felis, Pulex irritans, Tunga penetrans, Xenopsylla cheopis.

From the order of the Hymenoptera, for example, Camponotus herculeanus, Lasius fuliginosus, Lasius niger, Lasius umbratus, Monomorium pharaonis, Paravespula spp., Tetramorium caespitum.

From the order of the Anoplura, for example, Pediculus humanus capitis, Pediculus humanus corporis, Pemphigus spp., Phylloera vastatrix, Phthirus pubis.

From the order of the Heteroptera, for example, Cimex hemipterus, Cimex lectularius, Rhodinus prolixus, Triatoma infestans.

In the field of household insecticides, they are used alone or in combination with other suitable active compounds, such as phosphoric esters, carbamates, pyrethroids, neonicotinoides, growth regulators or active compounds from other known classes of insecticides.

They are used in aerosols, pressure-free spray products, for example pump and atomizer sprays, automatic fogging systems, foggers, foams, gels, evaporator products with evaporator tablets made of cellulose or polymer, liquid evaporators, gel and membrane evaporators, propeller-driven evaporators, energy-free, or passive, evaporation systems, moth papers, moth bags and moth gels, as granules or dusts, in baits for spreading or in bait stations.

The invention is illustrated by the following examples, without being limited thereby in any way.

In the tables, the following abbreviation are used:

Me CH₃

Et C₂H₅

nPr n-propyl

iPr i-propyl

nBu n-butyl

secBu s-butyl

iBu i-butyl

tBu t-butyl

Ph phenyl

cPr cyclopropyl

cPent cyclopentyl

### SYNTHESIS EXAMPLES

The compounds listed in the tables below can be prepared according to the general methods given above.

TABLE 1

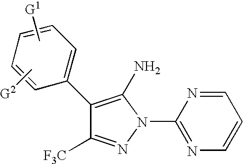

| Example No. | G¹ | G² | Physical data |
|---|---|---|---|
| 1- 01 | 2-F | H | NMR see below |
| 1- 02 | 3-F | H | NMR see below |
| 1- 03 | 4-F | H | NMR see below |
| 1- 04 | 2-Cl | H | logP = 2.59 |
| 1- 05 | 3-Cl | H | logP = 2.96 |
| 1- 06 | 4-Cl | H | NMR see below |
| 1- 07 | 2-Br | H | NMR see below |
| 1- 08 | 3-Br | H | NMR see below |
| 1- 09 | 4-Br | H | NMR see below |
| 1- 10 | 2-I | H | NMR see below |
| 1- 11 | 3-I | H | NMR see below |
| 1- 12 | 4-I | H | NMR see below |
| 1- 13 | 2-F | 3-F | NMR see below |
| 1- 14 | 2-F | 4-F | NMR see below |
| 1- 15 | 2-F | 5-F | NMR see below |
| 1- 16 | 2-F | 6-F | |
| 1- 17 | 2-F | 3-Cl | NMR see below |
| 1- 18 | 2-F | 4-Cl | NMR see below |
| 1- 19 | 2-F | 5-Cl | |
| 1- 20 | 2-F | 6-Cl | NMR see below |
| 1- 21 | 2-F | 3-Br | |
| 1- 22 | 2-F | 4-Br | NMR see below |
| 1- 23 | 2-F | 5-Br | NMR see below |
| 1- 24 | 2-F | 6-Br | |
| 1- 25 | 2-F | 3-I | |
| 1- 26 | 2-F | 4-I | |
| 1- 27 | 2-F | 5-I | |
| 1- 28 | 2-F | 6-I | |
| 1- 29 | 2-Cl | 3-F | |
| 1- 30 | 2-Cl | 4-F | NMR see below |
| 1- 31 | 2-Cl | 5-F | |
| 1- 32 | 2-Cl | 3-Cl | NMR see below |
| 1- 33 | 2-Cl | 4-Cl | NMR see below |
| 1- 34 | 2-Cl | 5-Cl | NMR see below |
| 1- 35 | 2-Cl | 6-Cl | NMR see below |
| 1- 36 | 2-Cl | 3-Br | |
| 1- 37 | 2-Cl | 4-Br | ¹H-NMR (d₆-DMSO): |
| | | | 8.92, d, 2H, 7.62, t, 1H |
| 1- 38 | 2-Cl | 5-Br | NMR see below |
| 1- 39 | 2-Cl | 6-Br | |
| 1- 40 | 2-Cl | 3-I | |
| 1- 41 | 2-Cl | 4-I | |
| 1- 42 | 2-Cl | 5-I | |
| 1- 43 | 2-Cl | 6-I | |
| 1- 44 | 2-Br | 3-F | |
| 1- 45 | 2-Br | 4-F | |
| 1- 46 | 2-Br | 5-F | |
| 1- 47 | 2-Br | 3-Cl | |
| 1- 48 | 2-Br | 4-Cl | |
| 1- 49 | 2-Br | 5-Cl | |
| 1- 50 | 2-Br | 3-Br | |
| 1- 51 | 2-Br | 4-Br | |
| 1- 52 | 2-Br | 5-Br | |
| 1- 53 | 2-Br | 6-Br | |
| 1- 54 | 2-Br | 3-I | |
| 1- 55 | 2-Br | 4-I | |
| 1- 56 | 2-Br | 5-I | |
| 1- 57 | 2-Br | 6-I | |
| 1- 58 | 2-I | 3-F | |
| 1- 59 | 2-I | 4-F | |
| 1- 60 | 2-I | 5-F | |
| 1- 61 | 2-I | 3-Cl | |
| 1- 62 | 2-I | 4-Cl | |
| 1- 63 | 2-I | 5-Cl | |
| 1- 64 | 2-I | 3-Br | |
| 1- 65 | 2-I | 4-Br | |
| 1- 66 | 2-I | 5-Br | |
| 1- 67 | 2-I | 3-I | |
| 1- 68 | 2-I | 4-I | |
| 1- 69 | 2-I | 5-I | |
| 1- 70 | 2-I | 6-I | |
| 1- 71 | 3-F | 4-F | NMR see below |
| 1- 72 | 3-F | 5-F | |
| 1- 73 | 3-F | 4-Cl | NMR see below |
| 1- 74 | 3-F | 5-Cl | NMR see below |
| 1- 75 | 3-F | 4-Br | NMR see below |
| 1- 76 | 3-F | 5-Br | |
| 1- 77 | 3-F | 4-I | |
| 1- 78 | 3-F | 5-I | |
| 1- 79 | 3-Cl | 4-F | Preparation Ex. |
| 1- 80 | 3-Cl | 4-Cl | NMR see below |
| 1- 81 | 3-Cl | 5-Cl | NMR see below |
| 1- 82 | 3-Cl | 4-Br | NMR see below |
| 1- 83 | 3-Cl | 5-Br | NMR see below |
| 1- 84 | 3-Cl | 4-I | ¹H-NMR (d₆-DMSO): |
| | | | 8.93, d, 2H, 7.07, dd, 1H |
| 1- 85 | 3-Cl | 5-I | |
| 1- 86 | 3-Br | 4-F | |
| 1- 87 | 3-Br | 4-Cl | NMR see below |
| 1- 88 | 3-Br | 4-Br | NMR see below |
| 1- 89 | 3-Br | 5-Br | NMR see below |
| 1- 90 | 3-Br | 4-I | |
| 1- 91 | 3-Br | 5-I | |
| 1- 92 | 3-I | 4-F | |
| 1- 93 | 3-I | 4-Cl | |
| 1- 94 | 3-I | 4-Br | |
| 1- 95 | 3-I | 4-I | |
| 1- 96 | 3-I | 4-I | |

TABLE 2

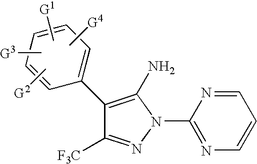

| Example No. | G¹ | G² | G³ | G⁴ | Physical data |
|---|---|---|---|---|---|
| 2- 01 | 2-F | 3-F | 4-F | H | |
| 2- 02 | 2-F | 3-F | 5-F | H | NMR see below |
| 2- 03 | 2-F | 3-F | 6-F | H | |
| 2- 04 | 2-F | 4-F | 5-F | H | NMR see below |
| 2- 05 | 2-F | 4-F | 6-F | H | NMR see below |
| 2- 06 | 3-F | 4-F | 5-F | H | NMR see below |
| 2- 07 | 2-Cl | 3-Cl | 4-Cl | H | |
| 2- 08 | 2-Cl | 3-Cl | 5-Cl | H | |
| 2- 09 | 2-Cl | 3-Cl | 6-Cl | H | |
| 2- 10 | 2-Cl | 4-Cl | 5-Cl | H | |
| 2- 11 | 2-Cl | 4-Cl | 6-Cl | H | |
| 2- 12 | 3-Cl | 4-Cl | 5-Cl | H | NMR see below |
| 2- 13 | 2-F | 4-Cl | 6-F | H | NMR see below |
| 2- 14 | 2-Cl | 4-Cl | 5-F | H | NMR see below |
| 2- 15 | 2-Cl | 4-F | 5-F | H | NMR see below |
| 2- 16 | 2-F | 4-F | 5-F | 3-Cl | NMR see below |
| 2- 17 | 2-F | 4-F | 3-Cl | H | NMR see below |
| 2- 18 | 2-Cl | 3-Cl | 6-F | H | |
| 2- 19 | 3-Cl | 4-Cl | 5-CF₃ | H | ¹H-NMR (d₆-DMSO): |
| | | | | | 8.94, d, 2H, 7.53, t, 1H |
| 2- 20 | 3-Cl | 4-CF₃ | 5-Cl | H | |
| 2- 21 | 3-Cl | 4-F | 5-Cl | H | |

TABLE 3

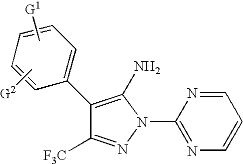

| Example No. | G¹ | G² | Physical data |
|---|---|---|---|
| 3- 01 | 2-CF₃ | H | |
| 3- 02 | 3-CF₃ | H | NMR see below |
| 3- 03 | 4-CF₃ | H | NMR see below |
| 3- 04 | 4-C₂F₅ | H | |
| 3- 05 | 4-nC₃F₇ | H | |
| 3- 06 | 4-iC₃F₇ | H | |
| 3- 07 | 4-CH₂CH₂CF₃ | H | |
| 3- 08 | 4-CH(CH₃)CF₃ | H | |
| 3- 09 | 2-CF₃ | 3-CF₃ | |
| 3- 10 | 2-CF₃ | 4-CF₃ | |
| 3- 11 | 2-CF₃ | 5-CF₃ | NMR see below |
| 3- 12 | 2-CF₃ | 6-CF₃ | |
| 3- 13 | 3-CF₃ | 4-CF₃ | |
| 3- 14 | 3-CF₃ | 5-CF₃ | NMR see below |
| 3- 15 | 4-CF₃ | 2-F | NMR see below |
| 3- 16 | 4-CF₃ | 2-Cl | |
| 3- 17 | 4-CF₃ | 2-Br | |
| 3- 18 | 4-CF₃ | 2-I | |
| 3- 19 | 4-CF₃ | 3-F | |
| 3- 20 | 4-CF₃ | 3-Cl | NMR see below |
| 3- 21 | 4-CF₃ | 3-Br | |
| 3- 22 | 4-CF₃ | 3-I | |
| 3- 23 | 3-CF₃ | 2-Cl | NMR see below |
| 3- 24 | 3-CF₃ | 4-Cl | NMR see below |
| 3- 25 | 3-CF₃ | 5-Cl | NMR see below, see also |
| | | | detailled Synthesis |
| | | | Examples |
| 3- 26 | 5-CF₃ | 2-Cl | NMR see below |
| 3- 27 | 2-CF₃ | 3-Cl | |
| 3- 28 | 2-CF₃ | 4-Cl | |
| 3- 29 | 2-CF₃ | 5-Cl | |
| 3- 30 | 2-CF₃ | 6-Cl | |
| 3- 31 | 3-CF₃ | 4-F | NMR see below |
| 3- 32 | 3-CF₃ | 5-F | NMR see below |
| 3- 33 | 5-CF₃ | 2-Br | NMR see below |
| 3- 34 | 3-CF₃ | 2-F | NMR see below |
| 3- 35 | 3-CF₃ | 4-Br | |

TABLE 4

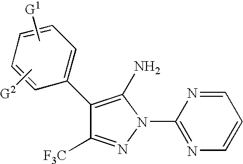

| Example No. | G¹ | G² | Physical data |
|---|---|---|---|
| 4- 01 | 2-OCHF₂ | H | |
| 4- 02 | 3-OCHF₂ | H | NMR see below |
| 4- 03 | 4-OCHF₂ | H | NMR see below |
| 4- 04 | 2-OCF₃ | H | |
| 4- 05 | 3-OCF₃ | H | NMR see below |
| 4- 06 | 4-OCF₃ | H | NMR see below |
| 4- 07 | 4-OCF₂Cl | H | NMR see below |
| 4- 08 | 4-OCF₂Br | H | |
| 4- 09 | 4-OCF₂CF₂H | H | |
| 4- 10 | 4-OCF₂CFClH | H | NMR see below |
| 4- 11 | 4-OCF₂CF₃ | H | |
| 4- 12 | 4-OCH₂CF₂H | H | |
| 4- 13 | 4-OCH₂CF₃ | H | |
| 4- 14 | 4-OCH₂CF₂CF₂H | H | |
| 4- 15 | 4-OCH₂CF₂CF₃ | H | |
| 4- 16 | 3-OCF₂CHFCF₃ | H | NMR see below |
| 4- 17 | 2-OCF₃ | 4-F | |
| 4- 18 | 3-OCF₃ | 4-F | NMR see below |
| 4- 19 | 4-OCF₃ | 3-F | NMR see below |
| 4- 20 | 2-OCF₃ | 4-Cl | |
| 4- 21 | 3-OCF₃ | 4-Cl | NMR see below |
| 4- 22 | 4-OCF₃ | 3-Cl | |
| 4- 23 | 3-OCF₃ | 5-Cl | NMR see below |
| 4- 24 | 2-OCF₃ | 4-Br | |
| 4- 25 | 3-OCF₃ | 4-Br | |
| 4- 26 | 4-OCF₃ | 3-Br | |
| 4- 27 | 2-OCF₃ | 4-I | |
| 4- 28 | 3-OCF₃ | 4-I | |
| 4- 29 | 4-OCF₃ | 3-I | |
| 4- 30 | 4-OCHF₂ | 3-F | |
| 4- 31 | 4-OCHF₂ | 3-Cl | |
| 4- 32 | 4-OCHF₂ | 3-Br | |
| 4- 33 | 4-OCHF₂ | 3-I | |
| 4- 34 | 3-OCF₃ | 4-OCF₃ | NMR see below |
| 4- 35 | 4-OCH₂CF₃ | 3-F | |
| 4- 36 | 4-OCH₂CF₃ | 3-Cl | 19F: −61.7 s, −74.4 tr, CF₃ |
| 4- 37 | 4-OCH₂CF₃ | 3-Br | |
| 4- 38 | 4-OCH₂CF₃ | 3-I | |
| 4- 39 | 4-OCH₂CF₃ | 2-F | |
| 4- 40 | 4-OCH₂CF₃ | 2-Cl | |
| 4- 41 | 4-OCH₂CF₃ | 2-Br | |
| 4- 42 | 4-OCH₂CF₃ | 2-I | |
| 4- 43 | 5-OCF₃ | 2-F | NMR see below |
| 4- 44 | 5-OCF₃ | 2-Cl | |
| 4- 45 | 5-OCF₃ | 2-Br | |
| 4- 46 | 5-OCF₃ | 2-I | |
| 4- 47 | 5-OCF₃ | 3-F | |
| 4- 48 | 5-OCF₃ | 3-Cl | |
| 4- 49 | 5-OCF₃ | 3-Br | |
| 4- 50 | 5-OCF₃ | 3-I | |
| 4- 51 | 4-OCF₃ | 2-F | |
| 4- 52 | 4-OCF₃ | 2-Cl | |
| 4- 53 | 4-OCF₃ | 2-Br | |
| 4- 54 | 4-OCF₃ | 2-I | |
| 4- 55 | 3-OCF₂Cl | H | NMR see below |

TABLE 5

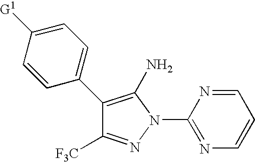

| Example No. | G¹ | Physical data |
|---|---|---|
| 5- 01 | H | NMR see below |
| 5- 02 | Me | NMR see below |
| 5- 03 | Et | |
| 5- 04 | nPr | |
| 5- 05 | iPr | 1H (d6DMSO): |
| | | 2.93, 1H; 1.24, 6H; iPr |
| 5- 06 | nBu | |
| 5- 07 | secBu | |
| 5- 08 | iBu | |
| 5- 09 | tBu | 1H (d6DMSO): 1.32; tBu |
| 5- 10 | n-C₅H₁₁ | |
| 5- 11 | n-C₆H₁₃ | |
| 5- 18 | CH₂OMe | |
| 5- 19 | CH₂OEt | |
| 5- 20 | CH₂OH | |
| 5- 21 | OH | |
| 5- 22 | OMe | NMR see below |
| 5- 23 | OEt | 1H: 4.07; OCH₂ |
| 5- 24 | OnPr | |
| 5- 25 | OiPr | 1H: 4.59, 1.37; OiPr |
| 5- 26 | OnBu | |
| 5- 27 | OsecBu | |
| 5- 28 | OiBu | |
| 5- 29 | OtBu | |
| 5- 30 | OCH₂OMe | |
| 5- 31 | OCH₂CH₂OMe | |
| 5- 32 | OCH₂cPr | |
| 5- 33 | OCH₂cpentyl | |
| 5- 34 | OCH₂chexyl | |
| 5- 35 | Ocpentyl | |
| 5- 36 | Ochexyl | |
| 5- 37 | OCH₂CH═CH₂ | |
| 5- 38 | OCH₂CCH | |
| 5- 39 | OCH₂phenyl | |
| 5- 40 | Ophenyl | logP 3.22 |
| 5- 41 | O-(4-F-phenyl) | |
| 5- 42 | O-(4-Cl-phenyl) | 1H: 7.04, 7.34, phenoxy |
| 5- 43 | O-(4-CF₃-phenyl) | |
| 5- 44 | O-(2-Cl-4-CF₃-phenyl) | 19F: −61.4, −62.5, CF₃ |
| 5- 45 | O-(4-Me-phenyl) | |
| 5- 46 | O-(2,4-Cl₂-phenyl) | |
| 5- 47 | O-(5-Cl-2-pyridyl) | |
| 5- 48 | O-(3,5-Cl₂-2-pyridyl) | |
| 5- 49 | O-(5-CF₃-2-pyridyl) | |
| 5- 50 | O-(3-Cl-5-CF₃-2-pyridyl) | 19F: −61.5, −62.1, CF₃ |
| 5- 51 | phenyl | |
| 5- 52 | 4-F-phenyl | |
| 5- 53 | 4-Cl-phenyl | 1H: 7.65, 7.56, 7.49, 7.43, 8H, |
| | | PhH |
| 5- 54 | 4-Br-phenyl | |
| 5- 55 | 2-pyridyl | |
| 5- 56 | 3-pyridyl | |
| 5- 57 | 4-pyridyl | |
| 5- 58 | SMe | NMR see below |
| 5- 59 | SOMe | NMR see below |
| 5- 60 | SO₂Me | NMR see below |
| 5- 61 | SO₂NMe₂ | |
| 5- 62 | SCF₃ | NMR see below |
| 5- 63 | SOCF₃ | |
| 5- 64 | SO₂CF₃ | |
| 5- 65 | CHO | |
| 5- 70 | COCH₃ | |
| 5- 71 | CN | NMR see below |
| 5- 72 | COOH | |
| 5- 73 | COOMe | |
| 5- 74 | COOEt | |
| 5- 75 | CONH₂ | |
| 5- 76 | CONMe₂ | |
| 5- 77 | SiMe₃ | |
| 5- 88 | benzyl | 1H (d6DMSO): 3.97 ppm, CH₂ |
| 5- 89 | NMe₂ | NMR see below |

TABLE 6

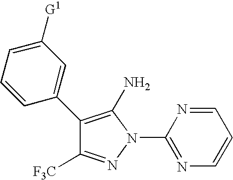

| Example No. | G¹ | Physical data |
|---|---|---|
| 6- 01 | Me | NMR see below |
| 6- 02 | Et | |
| 6- 03 | nPr | |
| 6- 04 | iPr | |
| 6- 05 | nBu | |
| 6- 06 | secBu | |
| 6- 07 | iBu | |
| 6- 08 | tBu | |
| 6- 09 | n-C₅H₁₁ | |
| 6- 10 | n-C₆H₁₃ | |
| 6- 17 | CH₂OMe | |
| 6- 18 | CH₂OEt | |
| 6- 19 | CH₂OH | |
| 6- 20 | OH | |
| 6- 21 | OMe | |
| 6- 22 | OEt | |
| 6- 23 | OnPr | |
| 6- 24 | OiPr | |
| 6- 25 | OnBu | |
| 6- 26 | OsecBu | |
| 6- 27 | OiBu | |
| 6- 28 | OtBu | |
| 6- 29 | OCH₂OMe | |
| 6- 30 | OCH₂CH₂OMe | |
| 6- 31 | OCH₂cPr | |
| 6- 32 | OCH₂cpentyl | |
| 6- 33 | OCH₂chexyl | |
| 6- 34 | Ocpentyl | |
| 6- 35 | Ochexyl | |
| 6- 36 | OCH₂CH═CH₂ | |
| 6- 37 | OCH₂CCH | |
| 6- 38 | OCH₂phenyl | |
| 6- 39 | Ophenyl | |
| 6- 40 | O-(4-F-phenyl) | |
| 6- 41 | O-(4-Cl-phenyl) | |
| 6- 42 | O-(4-CF₃-phenyl) | |
| 6- 43 | O-(2-Cl-4-CF₃-phenyl) | |
| 6- 44 | O-(4-Me-phenyl) | |
| 6- 45 | O-(2,4-Cl₂-phenyl) | |
| 6- 46 | O-(5-Cl-2-pyridyl) | |
| 6- 47 | O-(3,5-Cl₂-2-pyridyl) | |
| 6- 48 | O-(5-CF₃-2-pyridyl) | |
| 6- 49 | O-(3-Cl-5-CF₃-2-pyridyl) | |
| 6- 50 | phenyl | |
| 6- 51 | 4-F-phenyl | |
| 6- 52 | 4-Cl-phenyl | |
| 6- 53 | 4-Br-phenyl | |
| 6- 54 | 2-pyridyl | |
| 6- 55 | 3-pyridyl | |
| 6- 56 | 4-pyridyl | |
| 6- 57 | SMe | |
| 6- 58 | SOMe | |
| 6- 59 | SO₂Me | |
| 6- 60 | SO₂NMe₂ | |
| 6- 61 | SCF₃ | NMR see below |
| 6- 62 | SOCF₃ | |
| 6- 63 | SO₂CF₃ | |
| 6- 64 | CHO | |
| 6- 69 | COCH₃ | |
| 6- 70 | CN | NMR see below |
| 6- 71 | COOH | |
| 6- 72 | COOMe | NMR see below |
| 6- 73 | COOEt | |
| 6- 74 | CONH₂ | |
| 6- 75 | CONMe₂ | |
| 6- 76 | SiMe₃ | |
| 6- 77 | NMe₂ | NMR see below |
| 6- 78 | NO₂ | NMR see below |

TABLE 7

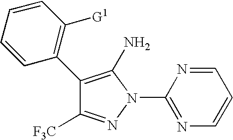

| Example No. | G¹ | Physical data |
|---|---|---|
| 7- 01 | Me | NMR see below |
| 7- 02 | Et | NMR see below |
| 7- 03 | nPr | |
| 7- 04 | iPr | |
| 7- 05 | nBu | |
| 7- 06 | secBu | |
| 7- 07 | iBu | |
| 7- 08 | tBu | |
| 7- 09 | n-C₅H₁₁ | |
| 7- 10 | n-C₆H₁₃ | |
| 7- 17 | CH₂OMe | |
| 7- 18 | CH₂OEt | |
| 7- 19 | CH₂OH | |
| 7- 20 | OH | |
| 7- 21 | OMe | |
| 7- 22 | OEt | |
| 7- 23 | OnPr | |
| 7- 24 | OiPr | |
| 7- 25 | OnBu | |
| 7- 26 | OsecBu | |
| 7- 27 | OiBu | |
| 7- 28 | OtBu | |
| 7- 29 | OCH₂OMe | |
| 7- 30 | OCH₂CH₂OMe | |
| 7- 31 | OCH₂cPr | |
| 7- 32 | OCH₂cpentyl | |
| 7- 33 | OCH₂chexyl | |
| 7- 34 | Ocpentyl | |
| 7- 35 | Ochexyl | |
| 7- 36 | OCH₂CH═CH₂ | |
| 7- 37 | OCH₂CCH | |
| 7- 38 | OCH₂phenyl | |
| 7- 39 | Ophenyl | |
| 7- 40 | O-(4-F-phenyl) | |
| 7- 41 | O-(4-Cl-phenyl) | |
| 7- 42 | O-(4-CF₃-phenyl) | |
| 7- 43 | O-(2-Cl-4-CF₃-phenyl) | |
| 7- 44 | O-(4-Me-phenyl) | |
| 7- 45 | O-(2,4-Cl₂-phenyl) | |
| 7- 46 | O-(5-Cl-2-pyridyl) | |
| 7- 47 | O-(3,5-Cl₂-2-pyridyl) | |
| 7- 48 | O-(5-CF₃-2-pyridyl) | |
| 7- 49 | O-(3-Cl-5-CF₃-2-pyridyl) | |
| 7- 50 | phenyl | |
| 7- 51 | 4-F-phenyl | |
| 7- 52 | 4-Cl-phenyl | |
| 7- 53 | 4-Br-phenyl | |
| 7- 54 | 2-pyridyl | |
| 7- 55 | 3-pyridyl | |
| 7- 56 | 4-pyridyl | |
| 7- 57 | SMe | |
| 7- 58 | SOMe | |
| 7- 59 | SO₂Me | |
| 7- 60 | SO₂NMe₂ | |
| 7- 61 | SCF₃ | |
| 7- 62 | SOCF₃ | |
| 7- 63 | SO₂CF₃ | |
| 7- 64 | CHO | |
| 7- 69 | COCH₃ | |
| 7- 70 | CN | |
| 7- 71 | COOH | |
| 7- 72 | COOMe | |
| 7- 73 | COOEt | |
| 7- 74 | CONH₂ | |
| 7- 75 | CONMe₂ | |
| 7- 76 | SiMe₃ | |

TABLE 8

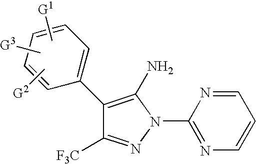

| Example No. | G¹ | G² | G³ | Physical data |
|---|---|---|---|---|
| 8- 01 | 2-Me | 3-Me | H | |
| 8- 02 | 2-Me | 4-Me | H | |
| 8- 03 | 2-Me | 5-Me | H | |
| 8- 04 | 2-Me | 6-Me | H | |
| 8- 05 | 3-Me | 4-Me | H | NMR see below |
| 8- 06 | 3-Me | 5-Me | H | NMR see below |
| 8- 07 | 2-OMe | 3-OMe | H | |
| 8- 08 | 2-OMe | 4-OMe | H | |
| 8- 09 | 2-OMe | 5-OMe | H | |
| 8- 10 | 2-OMe | 6-OMe | H | |
| 8- 11 | 3-OMe | 4-OMe | H | 1H: 3.89, 3.92 ppm, |
| | | | | OMe |
| 8- 12 | 3-OMe | 5-OMe | H | |
| 8- 13 | 2-Me | 3-Me | 4-Me | |
| 8- 14 | 2-Me | 3-Me | 5-Me | |
| 8- 15 | 2-Me | 3-Me | 6-Me | |
| 8- 16 | 2-Me | 4-Me | 5-Me | |
| 8- 17 | 2-Me | 4-Me | 6-Me | |
| 8- 18 | 3-Me | 4-Me | 5-Me | |
| 8- 19 | 2-OMe | 3-OMe | 4-OMe | |
| 8- 20 | 2-OMe | 3-OMe | 5-OMe | |
| 8- 21 | 2-OMe | 3-OMe | 6-OMe | |
| 8- 22 | 2-OMe | 4-OMe | 5-OMe | |
| 8- 23 | 2-OMe | 4-OMe | 6-OMe | |
| 8- 24 | 3-OMe | 4-OMe | 5-OMe | 1H: 6.63 ppm, PhH |
| 8- 25 | 3-F | 4-OMe | H | NMR see below |
| 8- 26 | 3-Cl | 4-OMe | H | NMR see below |
| 8- 27 | 3-Br | 4-OMe | H | NMR see below |
| 8- 28 | 3-I | 4-OMe | H | |
| 8- 29 | 3-F | 4-OEt | H | |
| 8- 30 | 3-Cl | 4-OEt | H | 1H: 7.00, 7.24, 7.42 |
| | | | | ppm, PhH |
| 8- 31 | 3-Br | 4-OEt | H | 1H: 4.16 ppm, OCH₂ |
| 8- 32 | 3-I | 4-OEt | H | |
| 8- 33 | 3-F | 4-OMe | 5-F | NMR see below |
| 8- 34 | 3-Cl | 4-OMe | 5-Cl | |
| 8- 35 | 3-Br | 4-OMe | 5-Br | |
| 8- 36 | 3-I | 4-OMe | 5-I | |
| 8- 37 | 3-F | 4-OEt | 5-F | |
| 8- 38 | 3-Cl | 4-OEt | 5-Cl | |
| 8- 39 | 3-Br | 4-OEt | 5-Br | |
| 8- 40 | 3-I | 4-OEt | 5-I | |
| 8- 41 | 4-F | 3-OPh | H | NMR see below |
| 8- 42 | 3-F | 5-Me | H | NMR see below |
| 8- 43 | 3-F | 4-Me | H | NMR see below |
| 8- 44 | 2-OMe | 5-Br | H | NMR see below |
| 8- 45 | 3-OMe | 4-Me | H | NMR see below |
| 8- 46 | 3-OMe | 5-Me | H | |
| 8- 47 | 2-OMe | 5-Me | H | |
| 8- 48 | 2-OMe | 5-Cl | H | NMR see below |
| 8- 49 | 2-OMe | 5-F | H | |
| 8- 50 | 2-F | 5-OMe | H | |
| 8- 51 | 2-Cl | 5-OMe | H | |
| 8- 52 | 2-Br | 5-OMe | H | |
| 8- 53 | 2-I | 5-OMe | H | |
| 8- 54 | 2-Cl | 4-Me | 5-Cl | NMR see below |
| 8- 55 | 2-Br | 4-Me | 5-Br | |
| 8- 56 | 2-Cl | 4-OMe | 5-Cl | |
| 8- 57 | 2-Br | 4-OMe | 5-Cl | |
| 8- 58 | 2-Cl | 4-Me | 6-Cl | |
| 8- 59 | 2-Br | 4-Me | 6-Br | |
| 8- 60 | 2-Cl | 4-OMe | 6-Cl | |
| 8- 61 | 2-Br | 4-OMe | 6-Br | |
| 8- 62 | 2-Cl | 3-OMe | 4-OMe | NMR see below |
| 8- 63 | 2-Br | 3-OMe | 4-OMe | |
| 8- 64 | 2-F | 3-OMe | 4-OMe | |
| 8- 65 | 2-F | 3-F | 4-OMe | NMR see below |
| 8- 66 | 4-OCHF₂ | 3-OMe | H | NMR see below |
| 8- 67 | 2-F | 4-OMe | 5-OMe | |
| 8- 68 | 2-Cl | 4-OMe | 5-OMe | 1H: 6.85, 6.99 ppm, |
| | | | | PhH |
| 8- 69 | 2-Br | 4-OMe | 5-OMe | 1H: 6.85, 7.14 ppm, |
| | | | | PhH |
| 8- 70 | 2-I | 4-OMe | 5-OMe | |
| 8- 71 | 2-OMe | 4-F | H | |
| 8- 72 | 2-OMe | 4-Cl | H | |
| 8- 73 | 2-OMe | 4-Br | H | 19F: −62.2 ppm, CF₃ |
| 8- 74 | 2-OMe | 4-I | H | |
| 8- 75 | 2-OEt | 4-F | H | |
| 8- 76 | 2-OEt | 4-Cl | H | |
| 8- 77 | 2-OEt | 4-Br | H | |
| 8- 78 | 2-OEt | 4-I | H | |
| 8- 79 | 3-OMe | 4-F | H | |
| 8- 80 | 3-OMe | 4-Cl | H | |
| 8- 81 | 3-OMe | 4-Br | H | |
| 8- 82 | 3-OMe | 4-I | H | NMR see below |
| 8- 83 | 3-OEt | 4-F | H | |
| 8- 84 | 3-OEt | 4-Cl | H | |
| 8- 85 | 3-OEt | 4-Br | H | |
| 8- 86 | 3-OEt | 4-I | H | |
| 8- 87 | 3-F | 4-OnPr | H | |
| 8- 88 | 3-Cl | 4-OnPr | H | NMR see below |
| 8- 89 | 3-Br | 4-OnPr | H | 1H: 4.03, 1.86, 1.10 |
| | | | | ppm, nPrO |
| 8- 90 | 3-I | 4-OnPr | H | |
| 8- 91 | 3-F | 4-OiPr | H | |
| 8- 92 | 3-Cl | 4-OiPr | H | NMR see detailled |
| | | | | description of |
| | | | | synthesis |
| 8- 93 | 3-Br | 4-OiPr | H | |
| 8- 94 | 3-I | 4-OiPr | H | |
| 8- 95 | 3-F | 4-OnBu | H | |
| 8- 96 | 3-Cl | 4-OnBu | H | NMR see below |
| 8- 97 | 3-Br | 4-OnBu | H | |
| 8- 98 | 3-I | 4-OnBu | H | |
| 8- 99 | 3-F | 4-OsecBu | H | |
| 8- 100 | 3-Cl | 4-OsecBu | H | |
| 8- 101 | 3-Br | 4-OsecBu | H | |
| 8- 102 | 3-I | 4-OsecBu | H | |
| 8- 103 | 3-F | 4-OiBu | H | |
| 8- 104 | 3-Cl | 4-OiBu | H | |
| 8- 105 | 3-Br | 4-OiBu | H | |
| 8- 106 | 3-I | 4-OiBu | H | |
| 8- 107 | 3,4-(O—CH₂—CH₂—O) | | 5-Cl | NMR see below |
| 8- 108 | 2-OMe | 5-OCF₃ | H | NMR see below |
| 8- 109 | 3-OMe | 4-OnPr | H | ¹H-NMR (d₆-DMSO): |
| | | | | 0.99, 1.74, 3.95 ppm, |
| | | | | OnPr |
| 8- 110 | 3-OMe | 4-OEt | H | ¹H-NMR (d₆-DMSO): |
| | | | | 1.35, 4.05 ppm, OEt |
| 8- 111 | 3-OMe | 5-CF₃ | H | NMR see below |
| 8- 112 | 3-OEt | 5-CF₃ | H | NMR see below |
| 8- 113 | 3-OEt | 5-F | H | NMR see below |

TABLE 9

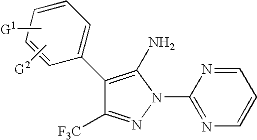

| Example No. | G¹ + G² | Physical data |
|---|---|---|
| 9- 01 | 2,3-C₃H₆ | |
| 9- 02 | 3,4-C₃H₆ | |
| 9- 03 | 2,3-C₄H₈ | |
| 9- 04 | 3,4-C₄H₈ | 1H (DMSO): 7.11, 7.25 ppm, PhH |
| 9- 05 | 2,3-OCH₂O | |
| 9- 06 | 3,4-OCH₂O | NMR see below |
| 9- 07 | 2,3-OC₂H₄O | |
| 9- 09 | 3,4-OC₂H₄O | NMR see below |
| 9- 09 | 2,3-OC₂H₄ | |
| 9- 10 | 3,4-OC₂H₄ | |
| 9- 11 | 2,3-C₂H₄O | |
| 9- 12 | 3,4-C₂H₄O | |
| 9- 13 | 2,3-OCF₂O | |
| 9- 14 | 3,4-OCF₂O | |

TABLE 10

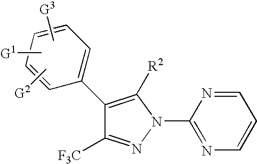

| Example No. | R² | G¹ | G² | G³ | Physical data |
|---|---|---|---|---|---|
| 10- 01 | N(CH₂Ph)₂ | H | H | H | |
| 10- 02 | N(CH₂Ph)₂ | 2-F | H | H | |
| 10- 03 | N(CH₂Ph)₂ | 3-F | H | H | |
| 10- 04 | N(CH₂Ph)₂ | 4-F | H | H | |
| 10- 05 | N(CH₂Ph)₂ | 2-Cl | H | H | |
| 10- 06 | N(CH₂Ph)₂ | 3-Cl | H | H | |
| 10- 07 | N(CH₂Ph)₂ | 4-Cl | H | H | |
| 10- 08 | N(CH₂Ph)₂ | 2-Br | H | H | |
| 10- 09 | N(CH₂Ph)₂ | 3-Br | H | H | |
| 10- 10 | N(CH₂Ph)₂ | 4-Br | H | H | |
| 10- 11 | N(CH₂Ph)₂ | 2-I | H | H | |
| 10- 12 | N(CH₂Ph)₂ | 3-I | H | H | |
| 10- 13 | N(CH₂Ph)₂ | 4-I | H | H | NMR see below |
| 10- 14 | N(CH₂Ph)₂ | 2-Cl | 3-Cl | H | |
| 10- 15 | N(CH₂Ph)₂ | 2-Cl | 4-Cl | H | |
| 10- 16 | N(CH₂Ph)₂ | 2-Cl | 5-Cl | H | |
| 10- 17 | N(CH₂Ph)₂ | 2-Cl | 6-Cl | H | |
| 10- 18 | N(CH₂Ph)₂ | 2-Cl | 4-F | H | |
| 10- 19 | N(CH₂Ph)₂ | 3-Cl | 4-Cl | H | |
| 10- 20 | N(CH₂Ph)₂ | 3-Cl | 5-Cl | H | |
| 10- 21 | N(CH₂Ph)₂ | 3-CF₃ | 4-Cl | H | NMR see below |
| 10- 22 | N(CH₂Ph)₂ | 3-F | 4-OMe | 5-F | NMR see below |
| 10- 23 | N(CH₂Ph)₂ | 3-OCHF₂ | H | H | NMR see below |
| 10- 24 | N(CH₂Ph)₂ | 3-F | 4-OMe | H | NMR see below |
| 10- 25 | N(CH₂Ph)₂ | 4-SMe | H | H | NMR see below |
| 10- 26 | N(CH₂Ph)₂ | 3-OCF₃ | 4-Cl | H | NMR see below |
| 10- 27 | N(CH₂Ph)₂ | 2-F | 3-Cl | H | NMR see below |
| 10- 28 | N(CH₂Ph)₂ | 4-OCHF₂ | H | H | NMR see below |
| 10- 29 | N(CH₂Ph)₂ | 3-Cl | 4-F | H | NMR see below |

TABLE 11

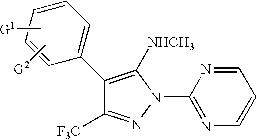

| Example | | | |
|---|---|---|---|
| No. | G¹ | G² | Physical data |
| 11-01 | H | H | |
| 11-02 | 2-F | H | |
| 11-03 | 3-F | H | |
| 11-04 | 4-F | H | |
| 11-05 | 2-Cl | H | |
| 11-06 | 3-Cl | H | |
| 11-07 | 4-Cl | H | NMR see below |
| 11-08 | 2-Br | H | |
| 11-09 | 3-Br | H | |
| 11-10 | 4-Br | H | 19F: −61.85 |
| | | | ppm |
| 11-11 | 2-I | H | |
| 11-12 | 3-I | H | |
| 11-13 | 4-I | H | |
| 11-14 | 2-F | 3-F | |
| 11-15 | 2-F | 4-F | |
| 11-16 | 2-F | 5-F | |
| 11-17 | 2-F | 6-F | |
| 11-18 | 2-F | 3-Cl | |
| 11-19 | 2-F | 4-Cl | |
| 11-20 | 2-F | 5-Cl | |
| 11-21 | 2-F | 6-Cl | |
| 11-22 | 2-F | 3-Br | |
| 11-23 | 2-F | 4-Br | |
| 11-24 | 2-F | 5-Br | |
| 11-25 | 2-F | 6-Br | |
| 11-26 | 2-F | 3-I | |
| 11-27 | 2-F | 4-I | |
| 11-28 | 2-F | 5-I | |
| 11-29 | 2-F | 6-I | |
| 11-30 | 2-Cl | 3-F | |
| 11-31 | 2-Cl | 4-F | |
| 11-32 | 2-Cl | 5-F | |
| 11-33 | 2-Cl | 3-Cl | |
| 11-34 | 2-Cl | 4-Cl | |
| 11-35 | 2-Cl | 5-Cl | |
| 11-36 | 2-Cl | 6-Cl | |
| 11-37 | 2-Cl | 3-Br | |
| 11-38 | 2-Cl | 4-Br | |
| 11-39 | 2-Cl | 5-Br | |
| 11-40 | 2-Cl | 6-Br | |
| 11-41 | 2-Cl | 3-I | |
| 11-42 | 2-Cl | 4-I | |
| 11-43 | 2-Cl | 5-I | |
| 11-44 | 2-Cl | 6-I | |
| 11-45 | 2-Br | 3-F | |
| 11-46 | 2-Br | 4-F | |
| 11-47 | 2-Br | 5-F | |
| 11-48 | 2-Br | 3-Cl | |
| 11-49 | 2-Br | 4-Cl | |
| 11-50 | 2-Br | 5-Cl | |
| 11-51 | 2-Br | 3-Br | |
| 11-52 | 2-Br | 4-Br | |
| 11-53 | 2-Br | 5-Br | |
| 11-54 | 2-Br | 6-Br | |
| 11-55 | 2-Br | 3-I | |
| 11-56 | 2-Br | 44 | |
| 11-57 | 2-Br | 5-I | |
| 11-58 | 2-Br | 6-I | |
| 11-59 | 2-I | 3-F | |
| 11-60 | 24 | 4-F | |
| 11-61 | 2-I | 5-F | |
| 11-62 | 2-I | 3-Cl | |
| 11-63 | 2-I | 4-Cl | |
| 11-64 | 2-I | 5-Cl | |
| 11-65 | 2-I | 3-Br | |
| 11-66 | 2-I | 4-Br | |
| 11-67 | 2-I | 5-Br | |
| 11-68 | 2-I | 3-I | |
| 11-69 | 2-I | 4-I | |
| 11-70 | 2-I | 5-I | |
| 11-71 | 2-I | 6-I | |
| 11-72 | 3-F | 4-F | |
| 11-73 | 3-F | 5-F | |
| 11-74 | 3-F | 4-Cl | |
| 11-75 | 3-F | 5-Cl | |
| 11-76 | 3-F | 4-Br | |
| 11-77 | 3-F | 5-Br | |
| 11-78 | 3-F | 4-I | |
| 11-79 | 3-F | 5-I | |
| 11-80 | 3-Cl | 4-F | |
| 11-81 | 3-Cl | 4-Cl | |
| 11-82 | 3-Cl | 5-Cl | NMR see below |
| 11-83 | 3-Cl | 4-Br | |
| 11-84 | 3-Cl | 5-Br | |
| 11-85 | 3-Cl | 4-I | |
| 11-86 | 3-Cl | 5-I | |
| 11-87 | 3-Br | 4-F | |
| 11-88 | 3-Br | 4-Cl | |
| 11-89 | 3-Br | 4-Br | |
| 11-90 | 3-Br | 5-Br | |
| 11-91 | 3-Br | 4-I | |
| 11-92 | 3-Br | 5-I | |
| 11-93 | 3-I | 4-F | |
| 11-94 | 3-1 | 4-Cl | |
| 11-95 | 3-I | 4-Br | |
| 11-96 | 3-I | 4-I | |
| 11-97 | 3-I | 4-I | |

TABLE 12

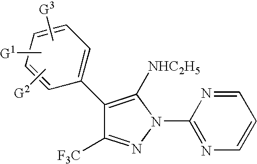

| Example | | | | |
|---|---|---|---|---|
| No. | G¹ | G² | G³ | Physical data |
| 12-01 | H | H | H | |
| 12-02 | 2-F | H | H | |
| 12-03 | 3-F | H | H | |
| 12-04 | 4-F | H | H | |
| 12-05 | 2-Cl | H | H | |
| 12-06 | 3-Cl | H | H | |
| 12-07 | 4-Cl | H | H | NMR see below |
| 12-08 | 2-Br | H | H | |
| 12-09 | 3-Br | H | H | |
| 12-10 | 4-Br | H | H | |
| 12-11 | 2-I | H | H | |
| 12-12 | 3-I | H | H | NMR see below |
| 12-13 | 4-I | H | H | |
| 12-14 | 2-F | 3-F | H | |
| 12-15 | 2-F | 4-F | H | |
| 12-16 | 2-F | 5-F | H | |
| 12-17 | 2-F | 6-F | H | |
| 12-18 | 2-F | 3-Cl | H | NMR see below |
| 12-19 | 2-F | 4-Cl | H | |
| 12-20 | 2-F | 5-Cl | H | |
| 12-21 | 2-F | 6-Cl | H | |
| 12-22 | 2-F | 3-Br | H | |
| 12-23 | 2-F | 4-Br | H | |
| 12-24 | 2-F | 5-Br | H | |
| 12-25 | 2-F | 6-Br | H | |
| 12-26 | 2-F | 3-I | H | |
| 12-27 | 2-F | 4-I | H | |
| 12-28 | 2-F | 5-I | H | |
| 12-29 | 2-F | 6-I | H | |
| 12-30 | 2-Cl | 3-F | H | |
| 12-31 | 2-Cl | 4-F | H | |
| 12-32 | 2-Cl | 5-F | H | |
| 12-33 | 2-Cl | 3-Cl | H | |
| 12-34 | 2-Cl | 4-Cl | H | |
| 12-35 | 2-Cl | 5-Cl | H | |
| 12-36 | 2-Cl | 6-Cl | H | |
| 12-37 | 2-Cl | 3-Br | H | |
| 12-38 | 2-Cl | 4-Br | H | |
| 12-39 | 2-Cl | 5-Br | H | |
| 12-40 | 2-Cl | 6-Br | H | |
| 12-41 | 2-Cl | 3-I | H | |
| 12-42 | 2-Cl | 4-I | H | |
| 12-43 | 2-Cl | 5-I | H | |
| 12-44 | 2-Cl | 6-I | H | |
| 12-45 | 2-Br | 3-F | H | |
| 12-46 | 2-Br | 4-F | H | |
| 12-47 | 2-Br | 5-F | H | |
| 12-48 | 2-Br | 3-Cl | H | |
| 12-49 | 2-Br | 4-Cl | H | |
| 12-50 | 2-Br | 5-Cl | H | |
| 12-51 | 2-Br | 3-Br | H | |
| 12-52 | 2-Br | 4-Br | H | |
| 12-53 | 2-Br | 5-Br | H | |
| 12-54 | 2-Br | 6-Br | H | |
| 12-55 | 2-Br | 3-I | H | |
| 12-56 | 2-Br | 4-I | H | |
| 12-57 | 2-Br | 5-I | H | |
| 12-58 | 2-Br | 6-I | H | |
| 12-59 | 2-I | 3-F | H | |
| 12-60 | 2-I | 4-F | H | |
| 12-61 | 2-I | 5-F | H | |
| 12-62 | 2-I | 3-Cl | H | |
| 12-63 | 2-I | 4-Cl | H | |
| 12-64 | 2-I | 5-Cl | H | |
| 12-65 | 2-I | 3-Br | H | |
| 12-66 | 2-I | 4-Br | H | |
| 12-67 | 2-I | 5-Br | H | |
| 12-68 | 2-I | 3-I | H | |
| 12-69 | 2-I | 4-I | H | |
| 12-70 | 2-I | 5-I | H | |
| 12-71 | 2-I | 6-I | H | |
| 12-72 | 3-F | 4-F | H | |
| 12-73 | 3-F | 5-F | H | |
| 12-74 | 3-F | 4-Cl | H | |
| 12-75 | 3-F | 5-Cl | H | |
| 12-76 | 3-F | 4-Br | H | |
| 12-77 | 3-F | 5-Br | H | |
| 12-78 | 3-F | 4-I | H | |
| 12-79 | 3-F | 5-I | H | |
| 12-80 | 3-Cl | 4-F | H | NMR see below |
| 12-81 | 3-Cl | 4-Cl | H | |
| 12-82 | 3-Cl | 5-Cl | H | NMR see below |
| 12-83 | 3-Cl | 4-Br | H | |
| 12-84 | 3-Cl | 5-Br | H | |
| 12-85 | 3-Cl | 4-I | H | |
| 12-86 | 3-Cl | 5-I | H | |
| 12-87 | 3-Br | 4-F | H | |
| 12-88 | 3-Br | 4-Cl | H | |
| 12-89 | 3-Br | 4-Br | H | |
| 12-90 | 3-Br | 5-Br | H | |
| 12-91 | 3-Br | 4-I | H | |
| 12-92 | 3-Br | 5-I | H | |
| 12-93 | 3-I | 4-F | H | |
| 12-94 | 3-I | 4-Cl | H | |
| 12-95 | 3-I | 4-Br | H | |
| 12-96 | 3-I | 4-I | H | |
| 12-97 | 3-I | 4-I | H | |
| 12-98 | 3-F | 4-OMe | 5-F | NMR see below |
| 12-99 | 3-CF₃ | 4-Cl | H | NMR see below |
| 12-100 | 4-SMe | H | H | NMR see below |
| 12-101 | 3-F | 4-OMe | H | NMR see below |
| 12-102 | 3-OCHF₂ | H | H | NMR see below |
| 12-103 | 4-OCHF₂ | H | H | NMR see below |
| 12-104 | 3-OMe | 4-Me | H | NMR see below |

TABLE 13

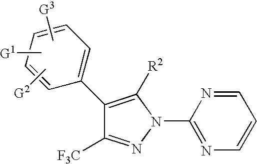

| Ex- | | | | | |
|---|---|---|---|---|---|
| am- | | | | | |
| ple | | | | | |
| No. | G¹ | G² | G³ | R² | Physical data |
| 13-01 | H | H | H | NMe₂ | |
| 13-02 | 2-F | H | H | NMe₂ | |
| 13-03 | 3-F | H | H | NMe₂ | |
| 13-04 | 4-F | H | H | NMe₂ | |
| 13-05 | 2-Cl | H | H | NMe₂ | |
| 13-06 | 3-Cl | H | H | NMe₂ | |
| 13-07 | 4-Cl | H | H | NMe₂ | |
| 13-08 | 2-Br | H | H | NMe₂ | |
| 13-09 | 3-Br | H | H | NMe₂ | |
| 13-10 | 4-Br | H | H | NMe₂ | |
| 13-11 | 2-I | H | H | NMe₂ | |
| 13-12 | 3-I | H | H | NMe₂ | |
| 13-13 | 4-I | H | H | NMe₂ | |
| 13-14 | 2-Cl | 3-Cl | H | NMe₂ | |
| 13-15 | 2-Cl | 4-Cl | H | NMe₂ | |
| 13-16 | 2-Cl | 5-Cl | H | NMe₂ | |
| 13-17 | 2-Cl | 6-Cl | H | NMe₂ | |
| 13-18 | 3-Cl | 4-Cl | H | NMe₂ | |
| 13-19 | 3-Cl | 5-Cl | H | NMe₂ | NMR see below |
| 13-20 | H | H | H | NEt₂ | |
| 13-21 | 2-F | H | H | NEt₂ | |
| 13-22 | 3-F | H | H | NEt₂ | |
| 13-23 | 4-F | H | H | NEt₂ | |
| 13-24 | 2-Cl | H | H | NEt₂ | |
| 13-25 | 3-Cl | H | H | NEt₂ | |
| 13-26 | 4-Cl | H | H | NEt₂ | |
| 13-27 | 2-Br | H | H | NEt₂ | |
| 13-28 | 3-Br | H | H | NEt₂ | |
| 13-29 | 4-Br | H | H | NEt₂ | |
| 13-30 | 2-I | H | H | NEt₂ | |
| 13-31 | 3-I | H | H | NEt₂ | NMR see below |
| 13-32 | 4-I | H | H | NEt₂ | NMR see below |
| 13-33 | 2-Cl | 3-Cl | H | NEt₂ | |
| 13-34 | 2-Cl | 4-Cl | H | NEt₂ | |
| 13-35 | 2-Cl | 5-Cl | H | NEt₂ | |
| 13-36 | 2-Cl | 6-Cl | H | NEt₂ | |
| 13-37 | 3-Cl | 4-Cl | H | NEt₂ | |
| 13-38 | 3-Cl | 5-Cl | H | NEt₂ | |
| 13-39 | H | H | H | NMeEt | |
| 13-40 | 2-F | H | H | NMeEt | |
| 13-41 | 3-F | H | H | NMeEt | |
| 13-42 | 4-F | H | H | NMeEt | |
| 13-43 | 2-Cl | H | H | NMeEt | |
| 13-44 | 3-Cl | H | H | NMeEt | |
| 13-45 | 4-Cl | H | H | NMeEt | |
| 13-46 | 2-Br | H | H | NMeEt | |
| 13-47 | 3-Br | H | H | NMeEt | |
| 13-48 | 4-Br | H | H | NMeEt | |
| 13-49 | 2-I | H | H | NMeEt | |
| 13-50 | 3-I | H | H | NMeEt | |
| 13-51 | 4-I | H | H | NMeEt | |
| 13-52 | 2-Cl | 3-Cl | H | NMeEt | |
| 13-53 | 2-Cl | 4-Cl | H | NMeEt | |
| 13-54 | 2-Cl | 5-Cl | H | NMeEt | |
| 13-55 | 2-Cl | 6-Cl | H | NMeEt | |
| 13-56 | 3-Cl | 4-Cl | H | NMeEt | |
| 13-57 | 3-Cl | 5-Cl | H | NMeEt | |
| 13-58 | H | H | H | N(C₄H₈) | |
| 13-59 | 2-F | H | H | N(C₄H₈) | |
| 13-60 | 3-F | H | H | N(C₄H₈) | |
| 13-61 | 4-F | H | H | N(C₄H₈) | |
| 13-62 | 2-Cl | H | H | N(C₄H₈) | |
| 13-63 | 3-Cl | H | H | N(C₄H₈) | |
| 13-64 | 4-Cl | H | H | N(C₄H₈) | |
| 13-65 | 2-Br | H | H | N(C₄H₈) | |
| 13-66 | 3-Br | H | H | N(C₄H₈) | |
| 13-67 | 4-Br | H | H | N(C₄H₈) | |
| 13-68 | 2-I | H | H | N(C₄H₈) | |
| 13-69 | 3-I | H | H | N(C₄H₈) | |
| 13-70 | 4-I | H | H | N(C₄H₈) | |
| 13-71 | 2-Cl | 3-Cl | H | N(C₄H₈) | |
| 13-72 | 2-Cl | 4-Cl | H | N(C₄H₈) | |
| 13-73 | 2-Cl | 5-Cl | H | N(C₄H₈) | |
| 13-74 | 2-Cl | 6-Cl | H | N(C₄H₈) | |
| 13-75 | 3-Cl | 4-Cl | H | N(C₄H₈) | |
| 13-76 | 3-Cl | 5-Cl | H | N(C₄H₈) | |
| 13-77 | H | H | H | N(C₅H10) | |
| 13-78 | 2-F | H | H | N(C₅H10) | |
| 13-79 | 3-F | H | H | N(C₅H10) | |
| 13-80 | 4-F | H | H | N(C₅H10) | |
| 13-81 | 2-Cl | H | H | N(C₅H10) | |
| 13-82 | 3-Cl | H | H | N(C₅H10) | |
| 13-83 | 4-Cl | H | H | N(C₅H10) | |
| 13-84 | 2-Br | H | H | N(C₅H10) | |
| 13-85 | 3-Br | H | H | N(C₅H10) | |
| 13-86 | 4-Br | H | H | N(C₅H10) | |
| 13-87 | 2-I | H | H | N(C₅H10) | |
| 13-88 | 3-I | H | H | N(C₅H10) | |
| 13-89 | 4-I | H | H | N(C₅H10) | |
| 13-90 | 2-Cl | 3-Cl | H | N(C₅H10) | |
| 13-91 | 2-Cl | 4-Cl | H | N(C₅H10) | |
| 13-92 | 2-Cl | 5-Cl | H | N(C₅H10) | |
| 13-93 | 2-Cl | 6-Cl | H | N(C₅H10) | |
| 13-94 | 3-Cl | 4-Cl | H | N(C₅H10) | |
| 13-95 | 3-Cl | 5-Cl | H | N(C₅H10) | |
| 13-96 | H | H | H | N(C₂H₄OC₂H₄) | |
| 13-97 | 2-F | H | H | N(C₂H₄OC₂H₄) | |
| 13-98 | 3-F | H | H | N(C₂H₄OC₂H₄) | |
| 13-99 | 4-F | H | H | N(C₂H₄OC₂H₄) | |
| 13-100 | 2-Cl | H | H | N(C₂H₄OC₂H₄) | |
| 13-101 | 3-Cl | H | H | N(C₂H₄OC₂H₄) | |
| 13-102 | 4-Cl | H | H | N(C₂H₄OC₂H₄) | |
| 13-103 | 2-Br | H | H | N(C₂H₄OC₂H₄) | |
| 13-104 | 3-Br | H | H | N(C₂H₄OC₂H₄) | |
| 13-105 | 4-Br | H | H | N(C₂H₄OC₂H₄) | |
| 13-106 | 2-I | H | H | N(C₂H₄OC₂H₄) | |
| 13-107 | 3-I | H | H | N(C₂H₄OC₂H₄) | |
| 13-108 | 4-I | H | H | N(C₂H₄OC₂H₄) | |
| 13-109 | 2-Cl | 3-Cl | H | N(C₂H₄OC₂H₄) | |
| 13-110 | 2-Cl | 4-Cl | H | N(C₂H₄OC₂H₄) | |
| 13-111 | 2-Cl | 5-Cl | H | N(C₂H₄OC₂H₄) | |
| 13-112 | 2-Cl | 6-Cl | H | N(C₂H₄OC₂H₄) | |
| 13-113 | 3-Cl | 4-Cl | H | N(C₂H₄OC₂H₄) | |
| 13-114 | 3-Cl | 5-Cl | H | N(C₂H₄OC₂H₄) | |
| 13-115 | 3-Cl | 4-F | H | NEt₂ | NMR see below |
| 13-116 | 3- | 4-Cl | H | NEt₂ | NMR see below |
| | OCF₃ | | | | |
| 13-117 | 3-F | 4- | 5-F | NEt₂ | NMR see below |
| | | OMe | | | |
| 13-118 | 3-Cl | 5-Cl | H | N(C₂H₄CH(CH₃)C₂H₄) | NMR see below |

TABLE 14

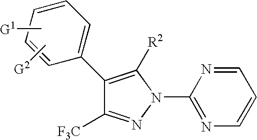

| Example | | | | |
|---|---|---|---|---|
| No. | G¹ | G² | R² | Physical data |
| 14-01 | H | H | Cl | |
| 14-02 | 2-F | H | Cl | |
| 14-03 | 3-F | H | Cl | |
| 14-04 | 4-F | H | Cl | |
| 14-05 | 2-Cl | H | Cl | |
| 14-06 | 3-Cl | H | Cl | lgP = 3.88 |
| 14-07 | 4-Cl | H | Cl | see Preparation Ex. |
| 14-08 | 2-Br | H | Cl | |
| 14-09 | 3-Br | H | Cl | |
| 14-10 | 4-Br | H | Cl | |
| 14-11 | 2-I | H | Cl | |
| 14-12 | 3-I | H | Cl | |
| 14-13 | 4-I | H | Cl | |
| 14-14 | 2-Cl | 3-Cl | Cl | |
| 14-15 | 2-Cl | 4-Cl | Cl | |
| 14-16 | 2-Cl | 5-Cl | Cl | |
| 14-17 | 2-Cl | 6-Cl | Cl | |
| 14-18 | 3-Cl | 4-Cl | Cl | NMR see below |
| 14-19 | 3-Cl | 5-Cl | Cl | |
| 14-20 | H | H | Br | |
| 14-21 | 2-F | H | Br | |
| 14-22 | 3-F | H | Br | |
| 14-23 | 4-F | H | Br | |
| 14-24 | 2-Cl | H | Br | |
| 14-25 | 3-Cl | H | Br | lgP = 3.89 |
| 14-26 | 4-Cl | H | Br | |
| 14-27 | 2-Br | H | Br | |
| 14-28 | 3-Br | H | Br | |
| 14-29 | 4-Br | H | Br | logP 3.75 |
| 14-30 | 2-I | H | Br | |
| 14-31 | 3-I | H | Br | |
| 14-32 | 4-I | H | Br | |
| 14-33 | 2-Cl | 3-Cl | Br | |
| 14-34 | 2-Cl | 4-Cl | Br | |
| 14-35 | 2-Cl | 5-Cl | Br | |
| 14-36 | 2-Cl | 6-Cl | Br | |
| 14-37 | 3-Cl | 4-Cl | Br | NMR see below |
| 14-38 | 3-Cl | 5-Cl | Br | lgP = 4.62 |
| 14-39 | H | H | I | |
| 14-40 | 2-F | H | I | |
| 14-41 | 3-F | H | I | |
| 14-42 | 4-F | H | I | |
| 14-43 | 2-Cl | H | I | |
| 14-44 | 3-Cl | H | I | lgP = 3.78 |
| 14-45 | 4-Cl | H | I | |
| 14-46 | 2-Br | H | I | |
| 14-47 | 3-Br | H | I | |
| 14-48 | 4-Br | H | I | |
| 14-49 | 2-I | H | I | |
| 14-50 | 3-I | H | I | |
| 14-51 | 4-I | H | I | |
| 14-52 | 2-Cl | 3-Cl | I | |
| 14-53 | 2-Cl | 4-Cl | I | |
| 14-54 | 2-Cl | 5-Cl | I | |
| 14-55 | 2-Cl | 6-Cl | I | |
| 14-56 | 3-Cl | 4-Cl | I | lgP = 4.39 |
| 14-57 | 3-Cl | 5-Cl | I | lgP = 4.51 |
| 14-58 | 3-Cl | 5-CF₃ | Br | lgP = 4.64 |
| 14-59 | 3-Cl | 5-CF₃ | I | lgP = 4.39 |

TABLE 15

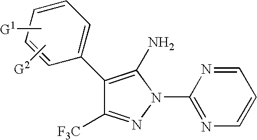

| Example | | | | |
|---|---|---|---|---|
| No. | G¹ | G² | R¹ | Physical data |
| 15-01 | H | H | CH₂F | |
| 15-02 | 2-F | H | CH₂F | |
| 15-03 | 3-F | H | CH₂F | |
| 15-04 | 4-F | H | CH₂F | |
| 15-05 | 2-Cl | H | CH₂F | |
| 15-06 | 3-Cl | H | CH₂F | |
| 15-07 | 4-Cl | H | CH₂F | NMR see below |
| 15-08 | 2-Br | H | CH₂F | |
| 15-09 | 3-Br | H | CH₂F | |
| 15-10 | 4-Br | H | CH₂F | |
| 15-11 | 2-I | H | CH₂F | |
| 15-12 | 3-I | H | CH₂F | |
| 15-13 | 4-I | H | CH₂F | |
| 15-14 | 2-Cl | 3-Cl | CH₂F | |
| 15-15 | 2-Cl | 4-Cl | CH₂F | |
| 15-16 | 2-Cl | 5-Cl | CH₂F | |
| 15-17 | 2-Cl | 6-Cl | CH₂F | |
| 15-18 | 3-Cl | 4-Cl | CH₂F | |
| 15-19 | 3-Cl | 5-Cl | CH₂F | NMR see below |
| 15-20 | H | H | CHF₂ | |
| 15-21 | 2-F | H | CHF₂ | |
| 15-22 | 3-F | H | CHF₂ | |
| 15-23 | 4-F | H | CHF₂ | |
| 15-24 | 2-Cl | H | CHF₂ | |
| 15-25 | 3-Cl | H | CHF₂ | |
| 15-26 | 4-Cl | H | CHF₂ | |
| 15-27 | 2-Br | H | CHF₂ | |
| 15-28 | 3-Br | H | CHF₂ | |
| 15-29 | 4-Br | H | CHF₂ | |
| 15-30 | 2-I | H | CHF₂ | |
| 15-31 | 3-I | H | CHF₂ | |
| 15-32 | 4-I | H | CHF₂ | |
| 15-33 | 2-Cl | 3-Cl | CHF₂ | |
| 15-34 | 2-Cl | 4-Cl | CHF₂ | |
| 15-35 | 2-Cl | 5-Cl | CHF₂ | |
| 15-36 | 2-Cl | 6-Cl | CHF₂ | |
| 15-37 | 3-Cl | 4-Cl | CHF₂ | |
| 15-38 | 3-Cl | 5-Cl | CHF₂ | NMR see below |
| 15-39 | H | H | CClF₂ | |
| 15-40 | 2-F | H | CClF₂ | |
| 15-41 | 3-F | H | CClF₂ | |
| 15-42 | 4-F | H | CClF₂ | |
| 15-43 | 2-Cl | H | CClF₂ | |
| 15-44 | 3-Cl | H | CClF₂ | |
| 15-45 | 4-Cl | H | CClF₂ | NMR see below |
| 15-46 | 2-Br | H | CClF₂ | |
| 15-47 | 3-Br | H | CClF₂ | |
| 15-48 | 4-Br | H | CClF₂ | NMR see below |
| 15-49 | 2-I | H | CClF₂ | |
| 15-50 | 3-I | H | CClF₂ | |
| 15-51 | 4-I | H | CClF₂ | NMR see below |
| 15-52 | 2-Cl | 3-Cl | CClF₂ | |
| 15-53 | 2-Cl | 4-Cl | CClF₂ | |
| 15-54 | 2-Cl | 5-Cl | CClF₂ | |
| 15-55 | 2-Cl | 6-Cl | CClF₂ | |
| 15-56 | 3-Cl | 4-Cl | CClF₂ | |
| 15-57 | 3-Cl | 5-Cl | CClF₂ | NMR see below |
| 15-58 | H | H | CCl₂F | |
| 15-59 | 2-F | H | CCl₂F | |
| 15-60 | 3-F | H | CCl₂F | |
| 15-61 | 4-F | H | CCl₂F | |
| 15-62 | 2-Cl | H | CCl₂F | |
| 15-63 | 3-Cl | H | CCl₂F | |
| 15-64 | 4-Cl | H | CCl₂F | |
| 15-65 | 2-Br | H | CCl₂F | |
| 15-66 | 3-Br | H | CCl₂F | |
| 15-67 | 4-Br | H | CCl₂F | |
| 15-68 | 2-I | H | CCl₂F | |
| 15-69 | 3-I | H | CCl₂F | |
| 15-70 | 4-I | H | CCl₂F | |
| 15-71 | 2-Cl | 3-Cl | CCl₂F | |
| 15-72 | 2-Cl | 4-Cl | CCl₂F | |
| 15-73 | 2-Cl | 5-Cl | CCl₂F | |
| 15-74 | 2-Cl | 6-Cl | CCl₂F | |
| 15-75 | 3-Cl | 4-Cl | CCl₂F | |
| 15-76 | 3-Cl | 5-Cl | CCl₂F | |
| 15-77 | H | H | CHClF | |
| 15-78 | 2-F | H | CHClF | |
| 15-79 | 3-F | H | CHClF | |
| 15-80 | 4-F | H | CHClF | |
| 15-81 | 2-Cl | H | CHClF | |
| 15-82 | 3-Cl | H | CHClF | |
| 15-83 | 4-Cl | H | CHClF | |
| 15-84 | 2-Br | H | CHClF | |
| 15-85 | 3-Br | H | CHClF | |
| 15-86 | 4-Br | H | CHClF | |
| 15-87 | 2-I | H | CHClF | |
| 15-88 | 3-I | H | CHClF | |
| 15-89 | 4-I | H | CHClF | |
| 15-90 | 2-Cl | 3-Cl | CHClF | |
| 15-91 | 2-Cl | 4-Cl | CHClF | |
| 15-92 | 2-Cl | 5-Cl | CHClF | |
| 15-93 | 2-Cl | 6-Cl | CHClF | |
| 15-94 | 3-Cl | 4-Cl | CHClF | |
| 15-95 | 3-Cl | 5-Cl | CHClF | NMR see below |
| 15-96 | H | H | CBrF₂ | |
| 15-97 | 2-F | H | CBrF₂ | |
| 15-98 | 3-F | H | CBrF₂ | |
| 15-99 | 4-F | H | CBrF₂ | |
| 15-100 | 2-Cl | H | CBrF₂ | |
| 15-101 | 3-Cl | H | CBrF₂ | |
| 15-102 | 4-Cl | H | CBrF₂ | |
| 15-103 | 2-Br | H | CBrF₂ | |
| 15-104 | 3-Br | H | CBrF₂ | |
| 15-105 | 4-Br | H | CBrF₂ | NMR see below |
| 15-106 | 2-I | H | CBrF₂ | |
| 15-107 | 3-I | H | CBrF₂ | |
| 15-108 | 4-I | H | CBrF₂ | NMR see below |
| 15-109 | 2-Cl | 3-Cl | CBrF₂ | |
| 15-110 | 2-Cl | 4-Cl | CBrF₂ | |
| 15-111 | 2-Cl | 5-Cl | CBrF₂ | |
| 15-112 | 2-Cl | 6-Cl | CBrF₂ | |
| 15-113 | 3-Cl | 4-Cl | CBrF₂ | |
| 15-114 | 3-Cl | 5-Cl | CBrF₂ | NMR see below |
| 15-115 | H | H | CBr₂F | |
| 15-116 | 2-F | H | CBr₂F | |
| 15-117 | 3-F | H | CBr₂F | |
| 15-118 | 4-F | H | CBr₂F | |
| 15-119 | 2-Cl | H | CBr₂F | |
| 15-120 | 3-Cl | H | CBr₂F | |
| 15-121 | 4-Cl | H | CBr₂F | |
| 15-122 | 2-Br | H | CBr₂F | |
| 15-123 | 3-Br | H | CBr₂F | |
| 15-124 | 4-Br | H | CBr₂F | |
| 15-125 | 2-I | H | CBr₂F | |
| 15-126 | 3-I | H | CBr₂F | |
| 15-127 | 4-I | H | CBr₂F | |
| 15-128 | 2-Cl | 3-Cl | CBr₂F | |
| 15-129 | 2-Cl | 4-Cl | CBr₂F | |
| 15-130 | 2-Cl | 5-Cl | CBr₂F | |
| 15-131 | 2-Cl | 6-Cl | CBr₂F | |
| 15-132 | 3-Cl | 4-Cl | CBr₂F | |
| 15-133 | 3-Cl | 5-Cl | CBr₂F | |
| 15-134 | H | H | CHBrF | |
| 15-135 | 2-F | H | CHBrF | |
| 15-136 | 3-F | H | CHBrF | |
| 15-137 | 4-F | H | CHBrF | |
| 15-138 | 2-Cl | H | CHBrF | |
| 15-139 | 3-Cl | H | CHBrF | |
| 15-140 | 4-Cl | H | CHBrF | |
| 15-141 | 2-Br | H | CHBrF | |
| 15-142 | 3-Br | H | CHBrF | |
| 15-143 | 4-Br | H | CHBrF | |
| 15-144 | 2-I | H | CHBrF | |
| 15-145 | 3-I | H | CHBrF | |
| 15-146 | 4-I | H | CHBrF | |
| 15-147 | 2-Cl | 3-Cl | CHBrF | |
| 15-148 | 2-Cl | 4-Cl | CHBrF | |
| 15-149 | 2-Cl | 5-Cl | CHBrF | |
| 15-150 | 2-Cl | 6-Cl | CHBrF | |
| 15-151 | 3-Cl | 4-Cl | CHBrF | |
| 15-152 | 3-Cl | 5-Cl | CHBrF | |
| 15-153 | 3-OCF₃ | 4-Cl | CClF₂ | NMR see below |
| 15-154 | 3-OCF₃ | 4-Cl | CBrF₂ | NMR see below |
| 15-155 | 3-CF₃ | 4-Cl | CClF₂ | NMR see below |
| 15-156 | 3-CF₃ | 4-Cl | CBrF₂ | NMR see below |
| 15-157 | 3-Br | 5-Br | CClF₂ | NMR see below |
| 15-158 | 3-Br | 5-Br | CBrF₂ | NMR see below |
| 15-159 | 3-Cl | 5-OCF₃ | CClF₂ | NMR see below |
| 15-160 | 3-Cl | 4-Br | CClF₂ | NMR see below |
| 15-161 | 3-Cl | 5-CF₃ | CClF₂ | NMR see below |
| 15-162 | 3-Cl | 5-CF₃ | CBrF₂ | NMR see below |
| 15-163 | 3-CF₃ | 5-CF₃ | CClF₂ | NMR see below |
| 15-164 | 3-CF₃ | 5-CF₃ | CBrF₂ | NMR see below |
| 15-165 | 3-F | 5-CF₃ | CClF₂ | NMR see below |
| 15-166 | 3-F | 5-CF₃ | CBrF₂ | NMR see below |
| 15-167 | 3-Cl | 5-CF₃ | CH₂F | NMR see below |
| 15-168 | 3-Cl | 4-Br | CF₂Br | NMR see below |
| 15-169 | 3-Cl | 5-OCF₃ | CF₂Br | NMR see below |
| 15-170 | 3-Cl | 5-CF₃ | CCl₂F | |
| 15-171 | 3-Cl | 4-CF₃ | CCl₂F | |
| 15-172 | 3-CF₃ | 5-CF₃ | CCl₂F | |
| 15-173 | 2-F | 4-Br | CClF₂ | |
| 15-174 | 2-F | 4-Br | CBrF₂ | |
| 15-175 | 2-F | 4-Br | CCl₂F | |
| 15-176 | 2-Cl | 4-Br | CClF₂ | |
| 15-177 | 2-Cl | 4-Br | CBrF₂ | |
| 15-178 | 2-Cl | 4-Br | CCl₂F | |
| 15-179 | 3-Cl | 4-Br | CClF₂ | |
| 15-180 | 3-Cl | 4-Br | CBrF₂ | |
| 15-181 | 3-Cl | 4-Br | CCl₂F | |
| 15-182 | 3-Cl | 5-Br | CClF₂ | |
| 15-183 | 3-Cl | 5-Br | CBrF₂ | |
| 15-184 | 3-Cl | 5-Br | CCl₂F | |
| 15-185 | 3-CF₃ | 4-Br | CClF₂ | |
| 15-186 | 3-CF₃ | 4-Br | CBrF₂ | |
| 15-187 | 3-CF₃ | 4-Br | CCl₂F | |
| 15-188 | 3-Cl | 4-I | CClF₂ | |
| 15-189 | 3-Cl | 4-I | CBrF₂ | |
| 15-190 | 3-Cl | 4-I | CCl₂F | |

TABLE 16

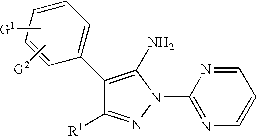

| Example | | | | |
|---|---|---|---|---|
| No. | G¹ | G² | R¹ | Physical data |
| 16-01 | H | H | Me | |
| 16-02 | 2-F | H | Me | |
| 16-03 | 3-F | H | Me | |
| 16-04 | 4-F | H | Me | NMR see below |
| 16-05 | 2-Cl | H | Me | NMR see below |
| 16-06 | 3-Cl | H | Me | NMR see below |
| 16-07 | 4-Cl | H | Me | NMR see below |
| 16-08 | 2-Br | H | Me | |
| 16-09 | 3-Br | H | Me | |
| 16-10 | 4-Br | H | Me | |
| 16-11 | 2-I | H | Me | |
| 16-12 | 3-I | H | Me | |
| 16-13 | 4-I | H | Me | |
| 16-14 | 2-Cl | 3-Cl | Me | |
| 16-15 | 2-Cl | 4-Cl | Me | NMR see below |
| 16-16 | 2-Cl | 5-Cl | Me | |
| 16-17 | 2-Cl | 6-Cl | Me | |
| 16-18 | 2-Cl | 4-F | Me | logP = 1.71 |
| 16-19 | 3-Cl | 4-Cl | Me | |
| 16-20 | 3-Cl | 5-Cl | Me | |
| 16-21 | H | H | Et | |
| 16-22 | 2-F | H | Et | |
| 16-23 | 3-F | H | Et | |
| 16-24 | 4-F | H | Et | |
| 16-25 | 2-Cl | H | Et | |
| 16-26 | 3-Cl | H | Et | |
| 16-27 | 4-Cl | H | Et | |
| 16-28 | 2-Br | H | Et | |
| 16-29 | 3-Br | H | Et | |
| 16-30 | 4-Br | H | Et | 1H (DMSO): 1.08, 2.56 ppm, Et |
| 16-31 | 2-I | H | Et | |
| 16-32 | 3-I | H | Et | |
| 16-33 | 4-I | H | Et | |
| 16-34 | 2-Cl | 3-Cl | Et | |
| 16-35 | 2-Cl | 4-Cl | Et | |
| 16-36 | 2-Cl | 5-Cl | Et | |
| 16-37 | 2-Cl | 6-Cl | Et | |
| 16-38 | 3-Cl | 4-Cl | Et | 1H: 1.17, 2.70 ppm, Et |
| 16-39 | 3-Cl | 5-Cl | Et | NMR see below |
| 16-40 | H | H | nPr | |
| 16-41 | 2-F | H | nPr | |
| 16-42 | 3-F | H | nPr | |
| 16-43 | 4-F | H | nPr | |
| 16-44 | 2-Cl | H | nPr | |
| 16-45 | 3-Cl | H | nPr | |
| 16-46 | 4-Cl | H | nPr | |
| 16-47 | 2-Br | H | nPr | |
| 16-48 | 3-Br | H | nPr | |
| 16-49 | 4-Br | H | nPr | 1H: 2.65, 1.60, 0.87 ppm, nPr |
| 16-50 | 2-I | H | nPr | |
| 16-51 | 3-I | H | nPr | |
| 16-52 | 4-I | H | nPr | |
| 16-53 | 2-Cl | 3-Cl | nPr | |
| 16-54 | 2-Cl | 4-Cl | nPr | |
| 16-55 | 2-Cl | 5-Cl | nPr | |
| 16-56 | 2-Cl | 6-Cl | nPr | |
| 16-57 | 3-Cl | 4-Cl | nPr | |
| 16-58 | 3-Cl | 5-Cl | nPr | |
| 16-59 | H | H | iPr | |
| 16-60 | 2-F | H | iPr | |
| 16-61 | 3-F | H | iPr | |
| 16-62 | 4-F | H | iPr | |
| 16-63 | 2-Cl | H | iPr | |
| 16-64 | 3-Cl | H | iPr | |
| 16-65 | 4-Cl | H | iPr | |
| 16-66 | 2-Br | H | iPr | |
| 16-67 | 3-Br | H | iPr | |
| 16-68 | 4-Br | H | iPr | 1H (d6DMSO): 2.96, |
| | | | | 1.14 ppm, iPr |
| 16-69 | 2-I | H | iPr | |
| 16-70 | 3-I | H | iPr | |
| 16-71 | 4-I | H | iPr | |
| 16-72 | 2-Cl | 3-Cl | iPr | |
| 16-73 | 2-Cl | 4-Cl | iPr | |
| 16-74 | 2-Cl | 5-Cl | iPr | |
| 16-75 | 2-Cl | 6-Cl | iPr | |
| 16-76 | 3-Cl | 4-Cl | iPr | |
| 16-77 | 3-Cl | 5-Cl | iPr | |
| 16-78 | H | H | nBu | |
| 16-79 | 2-F | H | nBu | |
| 16-80 | 3-F | H | nBu | |
| 16-81 | 4-F | H | nBu | |
| 16-82 | 2-Cl | H | nBu | |
| 16-83 | 3-Cl | H | nBu | |
| 16-84 | 4-Cl | H | nBu | |
| 16-85 | 2-Br | H | nBu | |
| 16-86 | 3-Br | H | nBu | |
| 16-87 | 4-Br | H | nBu | 1H (d6DMSO): 0.82, 1.27, |
| | | | | 1.48, 2.53, nBu |
| 16-88 | 2-I | H | nBu | |
| 16-89 | 3-I | H | nBu | |
| 16-90 | 4-I | H | nBu | |
| 16-91 | 2-Cl | 3-Cl | nBu | |
| 16-92 | 2-Cl | 4-Cl | nBu | |
| 16-93 | 2-Cl | 5-Cl | nBu | |
| 16-94 | 2-Cl | 6-Cl | nBu | |
| 16-95 | 3-Cl | 4-Cl | nBu | |
| 16-96 | 3-Cl | 5-Cl | nBu | |
| 16-97 | H | H | secBu | |
| 16-98 | 2-F | H | secBu | |
| 16-99 | 3-F | H | secBu | |
| 16-100 | 4-F | H | secBu | |
| 16-101 | 2-Cl | H | secBu | |
| 16-102 | 3-Cl | H | secBu | |
| 16-103 | 4-Cl | H | secBu | |
| 16-104 | 2-Br | H | secBu | |
| 16-105 | 3-Br | H | secBu | |
| 16-106 | 4-Br | H | secBu | |
| 16-107 | 2-I | H | secBu | |
| 16-108 | 3-I | H | secBu | |
| 16-109 | 4-I | H | secBu | |
| 16-110 | 2-Cl | 3-Cl | secBu | |
| 16-111 | 2-Cl | 4-Cl | secBu | |
| 16-112 | 2-Cl | 5-Cl | secBu | |
| 16-113 | 2-Cl | 6-Cl | secBu | |
| 16-114 | 3-Cl | 4-Cl | secBu | |
| 16-115 | 3-Cl | 5-Cl | secBu | |
| 16-116 | H | H | iBu | |
| 16-117 | 2-F | H | iBu | |
| 16-118 | 3-F | H | iBu | |
| 16-119 | 4-F | H | iBu | |
| 16-120 | 2-Cl | H | iBu | |
| 16-121 | 3-Cl | H | iBu | |
| 16-122 | 4-Cl | H | iBu | |
| 16-123 | 2-Br | H | iBu | |
| 16-124 | 3-Br | H | iBu | |
| 16-125 | 4-Br | H | iBu | |
| 16-126 | 2-I | H | iBu | |
| 16-127 | 3-I | H | iBu | |
| 16-128 | 4-I | H | iBu | |
| 16-129 | 2-Cl | 3-Cl | iBu | |
| 16-130 | 2-Cl | 4-Cl | iBu | |
| 16-131 | 2-Cl | 5-Cl | iBu | |
| 16-132 | 2-Cl | 6-Cl | iBu | |
| 16-133 | 3-Cl | 4-Cl | iBu | |
| 16-134 | 3-Cl | 5-Cl | iBu | NMR see below |
| 16-135 | H | H | tBu | |
| 16-136 | 2-F | H | tBu | |
| 16-137 | 3-F | H | tBu | |
| 16-138 | 4-F | H | tBu | |
| 16-139 | 2-Cl | H | tBu | |
| 16-140 | 3-Cl | H | tBu | |
| 16-141 | 4-Cl | H | tBu | |
| 16-142 | 2-Br | H | tBu | |
| 16-143 | 3-Br | H | tBu | |
| 16-144 | 4-Br | H | tBu | |
| 16-145 | 2-I | H | tBu | |
| 16-146 | 3-I | H | tBu | |
| 16-147 | 4-I | H | tBu | |
| 16-148 | 2-Cl | 3-Cl | tBu | |
| 16-149 | 2-Cl | 4-Cl | tBu | |
| 16-150 | 2-Cl | 5-Cl | tBu | |
| 16-151 | 2-Cl | 6-Cl | tBu | |
| 16-152 | 3-Cl | 4-Cl | tBu | |
| 16-153 | 3-Cl | 5-Cl | tBu | |
| 16-153 | H | H | tBuCH₂ | |
| 16-154 | 2-F | H | tBuCH₂ | |
| 16-155 | 3-F | H | tBuCH₂ | |
| 16-156 | 4-F | H | tBuCH₂ | |
| 16-157 | 2-Cl | H | tBuCH₂ | |
| 16-158 | 3-Cl | H | tBuCH₂ | |
| 16-159 | 4-Cl | H | tBuCH₂ | |
| 16-160 | 2-Br | H | tBuCH₂ | |
| 16-161 | 3-Br | H | tBuCH₂ | 1H-NMR (CDCl₃: 0.79, |
| | | | | (s, 9H), 2.64 ppm |
| | | | | (s, 2H), tBuCH₂ |
| 16-162 | 4-Br | H | tBuCH₂ | |
| 16-163 | 2-I | H | tBuCH₂ | |
| 16-164 | 3-I | H | tBuCH₂ | |
| 16-165 | 4-I | H | tBuCH₂ | |
| 16-166 | 2-Cl | 3-Cl | tBuCH₂ | |
| 16-167 | 2-Cl | 4-Cl | tBuCH₂ | |
| 16-168 | 2-Cl | 5-Cl | tBuCH₂ | |
| 16-169 | 2-Cl | 6-Cl | tBuCH₂ | |
| 16-170 | 3-Cl | 4-Cl | tBuCH₂ | |
| 16-171 | 3-Cl | 5-Cl | tBuCH₂ | |
| 16-172 | 3-Cl | 4-CF₃ | Me | 1H-NMR (d6-DMSO): |
| | | | | 8.83, d, 2H, 7.36, t, 1H, |
| | | | | 6.82, br s, 2H |
| 16-173 | 3-Cl | 5-CF₃ | Me | |
| 16-174 | 5-CF₃ | 5-CF₃ | Me | |
| 16-175 | 3-Cl | 4-CF₃ | Et | |
| 16-176 | 3-Cl | 5-CF₃ | Et | |
| 16-177 | 5-CF₃ | 5-CF₃ | Et | |
| 16-178 | 3-Cl | 4-CF₃ | iPr | |
| 16-179 | 3-Cl | 5-CF₃ | iPr | |
| 16-180 | 5-CF₃ | 5-CF₃ | iPr | |
| 16-181 | 3-Cl | 4-CF₃ | nPr | |
| 16-182 | 3-Cl | 5-CF₃ | nPr | |
| 16-183 | 5-CF₃ | 5-CF₃ | nPr | |
| 16-184 | 3-Cl | 4-CF₃ | tBu | |
| 16-185 | 3-Cl | 5-CF₃ | tBu | |
| 16-186 | 5-CF₃ | 5-CF₃ | tBu | |

TABLE 17

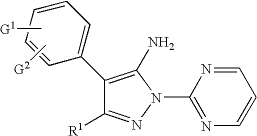

| Example | | | | |
|---|---|---|---|---|
| No. | G¹ | G² | R¹ | Physical data |
| 17-01 | H | H | CH₂OH | |
| 17-02 | 2-F | H | CH₂OH | |
| 17-03 | 3-F | H | CH₂OH | |
| 17-04 | 4-F | H | CH₂OH | |
| 17-05 | 2-Cl | H | CH₂OH | |
| 17-06 | 3-Cl | H | CH₂OH | |
| 17-07 | 4-Cl | H | CH₂OH | |
| 17-08 | 2-Br | H | CH₂OH | |
| 17-09 | 3-Br | H | CH₂OH | |
| 17-10 | 4-Br | H | CH₂OH | 1H: 4.67 ppm, CH₂O |
| 17-11 | 2-I | H | CH₂OH | |
| 17-12 | 3-I | H | CH₂OH | |
| 17-13 | 4-I | H | CH₂OH | |
| 17-14 | 2-Cl | 3-Cl | CH₂OH | |
| 17-15 | 2-Cl | 4-Cl | CH₂OH | |
| 17-16 | 2-Cl | 5-Cl | CH₂OH | |
| 17-17 | 2-Cl | 6-Cl | CH₂OH | |
| 17-18 | 3-Cl | 4-Cl | CH₂OH | |
| 17-19 | 3-Cl | 5-Cl | CH₂OH | |
| 17-20 | H | H | CH₂OMe | |
| 17-21 | 2-F | H | CH₂OMe | |
| 17-22 | 3-F | H | CH₂OMe | |
| 17-23 | 4-F | H | CH₂OMe | |
| 17-24 | 2-Cl | H | CH₂OMe | |
| 17-25 | 3-Cl | H | CH₂OMe | |
| 17-26 | 4-Cl | H | CH₂OMe | |
| 17-27 | 2-Br | H | CH₂OMe | |
| 17-28 | 3-Br | H | CH₂OMe | |
| 17-29 | 4-Br | H | CH₂OMe | |
| 17-30 | 2-I | H | CH₂OMe | |
| 17-31 | 3-I | H | CH₂OMe | |
| 17-32 | 4-I | H | CH₂OMe | |
| 17-33 | 2-Cl | 3-Cl | CH₂OMe | |
| 17-34 | 2-Cl | 4-Cl | CH₂OMe | |
| 17-35 | 2-Cl | 5-Cl | CH₂OMe | |
| 17-36 | 2-Cl | 6-Cl | CH₂OMe | |
| 17-37 | 3-Cl | 4-Cl | CH₂OMe | 1H: 4.30 ppm, CH₂O |
| 17-38 | 3-Cl | 5-Cl | CH₂OMe | |
| 17-39 | H | H | CH₂OEt | |
| 17-40 | 2-F | H | CH₂OEt | |
| 17-41 | 3-F | H | CH₂OEt | |
| 17-42 | 4-F | H | CH₂OEt | |
| 17-43 | 2-Cl | H | CH₂OEt | |
| 17-44 | 3-Cl | H | CH₂OEt | |
| 17-45 | 4-Cl | H | CH₂OEt | |
| 17-46 | 2-Br | H | CH₂OEt | |
| 17-47 | 3-Br | H | CH₂OEt | |
| 17-48 | 4-Br | H | CH₂OEt | 1H: 4.49 ppm, OCH₂ |
| 17-49 | 2-I | H | CH₂OEt | |
| 17-50 | 3-I | H | CH₂OEt | |
| 17-51 | 4-I | H | CH₂OEt | |
| 17-52 | 2-Cl | 3-Cl | CH₂OEt | |
| 17-53 | 2-Cl | 4-Cl | CH₂OEt | |
| 17-54 | 2-Cl | 5-Cl | CH₂OEt | |
| 17-55 | 2-Cl | 6-Cl | CH₂OEt | |
| 17-56 | 3-Cl | 4-Cl | CH₂OEt | |
| 17-57 | 3-Cl | 5-Cl | CH₂OEt | |
| 17-58 | H | H | CH₂SMe | |
| 17-59 | 2-F | H | CH₂SMe | |
| 17-60 | 3-F | H | CH₂SMe | |
| 17-61 | 4-F | H | CH₂SMe | |
| 17-62 | 2-Cl | H | CH₂SMe | |
| 17-63 | 3-Cl | H | CH₂SMe | |
| 17-64 | 4-Cl | H | CH₂SMe | |
| 17-65 | 2-Br | H | CH₂SMe | |
| 17-66 | 3-Br | H | CH₂SMe | |
| 17-67 | 4-Br | H | CH₂SMe | 1H: 3.68 ppm, CH₂S |
| 17-68 | 2-I | H | CH₂SMe | |
| 17-69 | 3-I | H | CH₂SMe | |
| 17-70 | 4-I | H | CH₂SMe | |
| 17-71 | 2-Cl | 3-Cl | CH₂SMe | |
| 17-72 | 2-Cl | 4-Cl | CH₂SMe | |
| 17-73 | 2-Cl | 5-Cl | CH₂SMe | |
| 17-74 | 2-Cl | 6-Cl | CH₂SMe | |
| 17-75 | 3-Cl | 4-Cl | CH₂SMe | |
| 17-76 | 3-Cl | 5-Cl | CH₂SMe | |
| 17-77 | H | H | CH₂SOMe | |
| 17-78 | 2-F | H | CH₂SOMe | |
| 17-79 | 3-F | H | CH₂SOMe | |
| 17-80 | 4-F | H | CH₂SOMe | |
| 17-81 | 2-Cl | H | CH₂SOMe | |
| 17-82 | 3-Cl | H | CH₂SOMe | |
| 17-83 | 4-Cl | H | CH₂SOMe | |
| 17-84 | 2-Br | H | CH₂SOMe | |
| 17-85 | 3-Br | H | CH₂SOMe | |
| 17-86 | 4-Br | H | CH₂SOMe | |
| 17-87 | 2-I | H | CH₂SOMe | |
| 17-88 | 3-I | H | CH₂SOMe | |
| 17-89 | 4-I | H | CH₂SOMe | |
| 17-90 | 2-Cl | 3-Cl | CH₂SOMe | |
| 17-91 | 2-Cl | 4-Cl | CH₂SOMe | |
| 17-92 | 2-Cl | 5-Cl | CH₂SOMe | |
| 17-93 | 2-Cl | 6-Cl | CH₂SOMe | |
| 17-94 | 3-Cl | 4-Cl | CH₂SOMe | |
| 17-95 | 3-Cl | 5-Cl | CH₂SOMe | |
| 17-96 | H | H | CH₂SO₂Me | |
| 17-97 | 2-F | H | CH₂SO₂Me | |
| 17-98 | 3-F | H | CH₂SO₂Me | |
| 17-99 | 4-F | H | CH₂SO₂Me | |
| 17-100 | 2-Cl | H | CH₂SO₂Me | |
| 17-101 | 3-Cl | H | CH₂SO₂Me | |
| 17-102 | 4-Cl | H | CH₂SO₂Me | |
| 17-103 | 2-Br | H | CH₂SO₂Me | |
| 17-104 | 3-Br | H | CH₂SO₂Me | |
| 17-105 | 4-Br | H | CH₂SO₂Me | |
| 17-106 | 2-I | H | CH₂SO₂Me | |
| 17-107 | 3-I | H | CH₂SO₂Me | |
| 17-108 | 4-I | H | CH₂SO₂Me | |
| 17-109 | 2-Cl | 3-Cl | CH₂SO₂Me | |
| 17-110 | 2-Cl | 4-Cl | CH₂SO₂Me | |
| 17-111 | 2-Cl | 5-Cl | CH₂SO₂Me | |
| 17-112 | 2-Cl | 6-Cl | CH₂SO₂Me | |
| 17-113 | 3-Cl | 4-Cl | CH₂SO₂Me | |
| 17-114 | 3-Cl | 5-Cl | CH₂SO₂Me | |
| 17-115 | H | H | CH(OMe)Me | |
| 17-116 | 2-F | H | CH(OMe)Me | |
| 17-117 | 3-F | H | CH(OMe)Me | |
| 17-118 | 4-F | H | CH(OMe)Me | |
| 17-119 | 2-Cl | H | CH(OMe)Me | |
| 17-120 | 3-Cl | H | CH(OMe)Me | |
| 17-121 | 4-Cl | H | CH(OMe)Me | |
| 17-122 | 2-Br | H | CH(OMe)Me | |
| 17-123 | 3-Br | H | CH(OMe)Me | |
| 17-124 | 4-Br | H | CH(OMe)Me | 1H (DMSO): 4.36 ppm, |
| | | | | CHOMe |
| 17-125 | 2-I | H | CH(OMe)Me | |
| 17-126 | 3-I | H | CH(OMe)Me | |
| 17-127 | 4-I | H | CH(OMe)Me | |
| 17-128 | 2-Cl | 3-Cl | CH(OMe)Me | |
| 17-129 | 2-Cl | 4-Cl | CH(OMe)Me | |
| 17-130 | 2-Cl | 5-Cl | CH(OMe)Me | |
| 17-131 | 2-Cl | 6-Cl | CH(OMe)Me | |
| 17-132 | 3-Cl | 4-Cl | CH(OMe)Me | |
| 17-133 | 3-Cl | 5-Cl | CH(OMe)Me | |
| 17-153 | H | H | CH₂CH₂COOMe | |
| 17-154 | 2-F | H | CH₂CH₂COOMe | |
| 17-155 | 3-F | H | CH₂CH₂COOMe | |
| 17-156 | 4-F | H | CH₂CH₂COOMe | |
| 17-157 | 2-Cl | H | CH₂CH₂COOMe | |
| 17-158 | 3-Cl | H | CH₂CH₂COOMe | |
| 17-159 | 4-Cl | H | CH₂CH₂COOMe | |
| 17-160 | 2-Br | H | CH₂CH₂COOMe | |
| 17-161 | 3-Br | H | CH₂CH₂COOMe | |
| 17-162 | 4-Br | H | CH₂CH₂COOMe | 1H: 2.69, 3.00 ppm, |
| | | | | CH₂CH₂ |
| 17-163 | 2-I | H | CH₂CH₂COOMe | |
| 17-164 | 3-I | H | CH₂CH₂COOMe | |
| 17-165 | 4-I | H | CH₂CH₂COOMe | |
| 17-166 | 2-Cl | 3-Cl | CH₂CH₂COOMe | |
| 17-167 | 2-Cl | 4-Cl | CH₂CH₂COOMe | |
| 17-168 | 2-Cl | 5-Cl | CH₂CH₂COOMe | |
| 17-169 | 2-Cl | 6-Cl | CH₂CH₂COOMe | |
| 17-170 | 3-Cl | 4-Cl | CH₂CH₂COOMe | |
| 17-171 | 3-Cl | 5-Cl | CH₂CH₂COOMe | |
| 17-172 | 3-Cl | 4-CF₃ | CH₂OMe | ¹H-NMR (d6-DMSO): |
| | | | | 8.87, d, 2H, 7.41, t, |
| | | | | 1H, 4.31, s, (MeO) |

TABLE 18

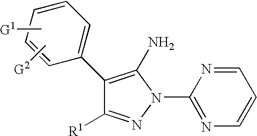

| Example | | | | |
|---|---|---|---|---|
| No. | G¹ | G² | R¹ | Physical data |
| 18-01 | H | H | CHFCH₃ | |
| 18-02 | 2-F | H | CHFCH₃ | |
| 18-03 | 3-F | H | CHFCH₃ | |
| 18-04 | 4-F | H | CHFCH₃ | |
| 18-05 | 2-Cl | H | CHFCH₃ | |
| 18-06 | 3-Cl | H | CHFCH₃ | |
| 18-07 | 4-Cl | H | CHFCH₃ | NMR see below |
| 18-08 | 2-Br | H | CHFCH₃ | |
| 18-09 | 3-Br | H | CHFCH₃ | |
| 18-10 | 4-Br | H | CHFCH₃ | 19F: −164.0 |
| | | | | ppm, CHF |
| 18-11 | 2-I | H | CHFCH₃ | |
| 18-12 | 3-I | H | CHFCH₃ | |
| 18-13 | 4-I | H | CHFCH₃ | |
| 18-14 | 2-Cl | 3-Cl | CHFCH₃ | |
| 18-15 | 2-Cl | 4-Cl | CHFCH₃ | |
| 18-16 | 2-Cl | 5-Cl | CHFCH₃ | |
| 18-17 | 2-Cl | 6-Cl | CHFCH₃ | |
| 18-18 | 3-Cl | 4-Cl | CHFCH₃ | |
| 18-19 | 3-Cl | 5-Cl | CHFCH₃ | NMR see below |
| 18-20 | H | H | CF₂CH₃ | |
| 18-21 | 2-F | H | CF₂CH₃ | |
| 18-22 | 3-F | H | CF₂CH₃ | |
| 18-23 | 4-F | H | CF₂CH₃ | |
| 18-24 | 2-Cl | H | CF₂CH₃ | |
| 18-25 | 3-Cl | H | CF₂CH₃ | |
| 18-26 | 4-Cl | H | CF₂CH₃ | NMR see below |
| 18-27 | 2-Br | H | CF₂CH₃ | |
| 18-28 | 3-Br | H | CF₂CH₃ | |
| 18-29 | 4-Br | H | CF₂CH₃ | |
| 18-30 | 2-I | H | CF₂CH₃ | |
| 18-31 | 3-I | H | CF₂CH₃ | |
| 18-32 | 4-I | H | CF₂CH₃ | NMR see below |
| 18-33 | 2-Cl | 3-Cl | CF₂CH₃ | |
| 18-34 | 2-Cl | 4-Cl | CF₂CH₃ | NMR see below |
| 18-35 | 2-Cl | 5-Cl | CF₂CH₃ | |
| 18-36 | 2-Cl | 6-Cl | CF₂CH₃ | |
| 18-37 | 3-Cl | 4-Cl | CF₂CH₃ | |
| 18-38 | 3-Cl | 5-Cl | CF₂CH₃ | NMR see below |
| 18-39 | H | H | CF₂CF₃ | |
| 18-40 | 2-F | H | CF₂CF₃ | |
| 18-41 | 3-F | H | CF₂CF₃ | |
| 18-42 | 4-F | H | CF₂CF₃ | |
| 18-43 | 2-Cl | H | CF₂CF₃ | |
| 18-44 | 3-Cl | H | CF₂CF₃ | |
| 18-45 | 4-Cl | H | CF₂CF₃ | NMR see below |
| 18-46 | 2-Br | H | CF₂CF₃ | |
| 18-47 | 3-Br | H | CF₂CF₃ | |
| 18-48 | 4-Br | H | CF₂CF₃ | NMR see below |
| 18-49 | 2-I | H | CF₂CF₃ | |
| 18-50 | 3-I | H | CF₂CF₃ | |
| 18-51 | 4-I | H | CF₂CF₃ | NMR see below |
| 18-52 | 2-Cl | 3-Cl | CF₂CF₃ | |
| 18-53 | 2-Cl | 4-Cl | CF₂CF₃ | |
| 18-54 | 2-Cl | 5-Cl | CF₂CF₃ | |
| 18-55 | 2-Cl | 6-Cl | CF₂CF₃ | |
| 18-56 | 3-Cl | 4-Cl | CF₂CF₃ | |
| 18-57 | 3-Cl | 5-Cl | CF₂CF₃ | NMR see below |
| 18-58 | H | H | CHFCF₃ | |
| 18-59 | 2-F | H | CHFCF₃ | |
| 18-60 | 3-F | H | CHFCF₃ | |
| 18-61 | 4-F | H | CHFCF₃ | |
| 18-62 | 2-Cl | H | CHFCF₃ | |
| 18-63 | 3-Cl | H | CHFCF₃ | |
| 18-64 | 4-Cl | H | CHFCF₃ | |
| 18-65 | 2-Br | H | CHFCF₃ | |
| 18-66 | 3-Br | H | CHFCF₃ | |
| 18-67 | 4-Br | H | CHFCF₃ | |
| 18-68 | 2-I | H | CHFCF₃ | |
| 18-69 | 3-I | H | CHFCF₃ | |
| 18-70 | 4-I | H | CHFCF₃ | |
| 18-71 | 2-Cl | 3-Cl | CHFCF₃ | |
| 18-72 | 2-Cl | 4-Cl | CHFCF₃ | |
| 18-73 | 2-Cl | 5-Cl | CHFCF₃ | |
| 18-74 | 2-Cl | 6-Cl | CHFCF₃ | |
| 18-75 | 3-Cl | 4-Cl | CHFCF₃ | |
| 18-76 | 3-Cl | 5-Cl | CHFCF₃ | |
| 18-77 | H | H | CF₂CF₂H | |
| 18-78 | 2-F | H | CF₂CF₂H | |
| 18-79 | 3-F | H | CF₂CF₂H | |
| 18-80 | 4-F | H | CF₂CF₂H | |
| 18-81 | 2-Cl | H | CF₂CF₂H | |
| 18-82 | 3-Cl | H | CF₂CF₂H | |
| 18-83 | 4-Cl | H | CF₂CF₂H | |
| 18-84 | 2-Br | H | CF₂CF₂H | |
| 18-85 | 3-Br | H | CF₂CF₂H | |
| 18-86 | 4-Br | H | CF₂CF₂H | |
| 18-87 | 2-I | H | CF₂CF₂H | |
| 18-88 | 3-I | H | CF₂CF₂H | |
| 18-89 | 4-I | H | CF₂CF₂H | |
| 18-90 | 2-Cl | 3-Cl | CF₂CF₂H | |
| 18-91 | 2-Cl | 4-Cl | CF₂CF₂H | |
| 18-92 | 2-Cl | 5-Cl | CF₂CF₂H | |
| 18-93 | 2-Cl | 6-Cl | CF₂CF₂H | |
| 18-94 | 3-Cl | 4-Cl | CF₂CF₂H | |
| 18-95 | 3-Cl | 5-Cl | CF₂CF₂H | |
| 18-96 | H | H | CF₂CF₂CF₃ | |
| 18-97 | 2-F | H | CF₂CF₂CF₃ | |
| 18-98 | 3-F | H | CF₂CF₂CF₃ | |
| 18-99 | 4-F | H | CF₂CF₂CF₃ | |
| 18-100 | 2-Cl | H | CF₂CF₂CF₃ | |
| 18-101 | 3-Cl | H | CF₂CF₂CF₃ | |
| 18-102 | 4-Cl | H | CF₂CF₂CF₃ | |
| 18-103 | 2-Br | H | CF₂CF₂CF₃ | |
| 18-104 | 3-Br | H | CF₂CF₂CF₃ | |
| 18-105 | 4-Br | H | CF₂CF₂CF₃ | |
| 18-106 | 2-I | H | CF₂CF₂CF₃ | |
| 18-107 | 3-I | H | CF₂CF₂CF₃ | |
| 18-108 | 4-I | H | CF₂CF₂CF₃ | |
| 18-109 | 2-Cl | 3-Cl | CF₂CF₂CF₃ | |
| 18-110 | 2-Cl | 4-Cl | CF₂CF₂CF₃ | |
| 18-111 | 2-Cl | 5-Cl | CF₂CF₂CF₃ | |
| 18-112 | 2-Cl | 6-Cl | CF₂CF₂CF₃ | |
| 18-113 | 3-Cl | 4-Cl | CF₂CF₂CF₃ | |
| 18-114 | 3-Cl | 5-Cl | CF₂CF₂CF₃ | |
| 18-115 | H | H | CF(CH₃)₂ | |
| 18-116 | 2-F | H | CF(CH₃)₂ | |
| 18-117 | 3-F | H | CF(CH₃)₂ | |
| 18-118 | 4-F | H | CF(CH₃)₂ | |
| 18-119 | 2-Cl | H | CF(CH₃)₂ | |
| 18-120 | 3-Cl | H | CF(CH₃)₂ | |
| 18-121 | 4-Cl | H | CF(CH₃)₂ | |
| 18-122 | 2-Br | H | CF(CH₃)₂ | |
| 18-123 | 3-Br | H | CF(CH₃)₂ | |
| 18-124 | 4-Br | H | CF(CH₃)₂ | 1H: 1.61 ppm, CH₃ |
| 18-125 | 2-I | H | CF(CH₃)₂ | |
| 18-126 | 3-I | H | CF(CH₃)₂ | |
| 18-127 | 4-I | H | CF(CH₃)₂ | |
| 18-128 | 2-Cl | 3-Cl | CF(CH₃)₂ | |
| 18-129 | 2-Cl | 4-Cl | CF(CH₃)₂ | |
| 18-130 | 2-Cl | 5-Cl | CF(CH₃)₂ | |
| 18-131 | 2-Cl | 6-Cl | CF(CH₃)₂ | |
| 18-132 | 3-Cl | 4-Cl | CF(CH₃)₂ | |
| 18-133 | 3-Cl | 5-Cl | CF(CH₃)₂ | |
| 18-134 | H | H | CH₂CH₂CF₃ | |
| 18-135 | 2-F | H | CH₂CH₂CF₃ | |
| 18-136 | 3-F | H | CH₂CH₂CF₃ | |
| 18-137 | 4-F | H | CH₂CH₂CF₃ | |
| 18-138 | 2-Cl | H | CH₂CH₂CF₃ | |
| 18-139 | 3-Cl | H | CH₂CH₂CF₃ | |
| 18-140 | 4-Cl | H | CH₂CH₂CF₃ | |
| 18-141 | 2-Br | H | CH₂CH₂CF₃ | |
| 18-142 | 3-Br | H | CH₂CH₂CF₃ | |
| 18-143 | 4-Br | H | CH₂CH₂CF₃ | 1H (d6DMSO): 2.79, |
| | | | | 2.59; CH₂CH₂ |
| 18-144 | 2-I | H | CH₂CH₂CF₃ | |
| 18-145 | 3-I | H | CH₂CH₂CF₃ | |
| 18-146 | 4-I | H | CH₂CH₂CF₃ | |
| 18-147 | 2-Cl | 3-Cl | CH₂CH₂CF₃ | |
| 18-148 | 2-Cl | 4-Cl | CH₂CH₂CF₃ | |
| 18-149 | 2-Cl | 5-Cl | CH₂CH₂CF₃ | |
| 18-150 | 2-Cl | 6-Cl | CH₂CH₂CF₃ | |
| 18-151 | 3-Cl | 4-Cl | CH₂CH₂CF₃ | |
| 18-152 | 3-Cl | 5-Cl | CH₂CH₂CF₃ | |
| 18-153 | H | H | CH(CH₃)CF₃ | |
| 18-154 | 2-F | H | CH(CH₃)CF₃ | |
| 18-155 | 3-F | H | CH(CH₃)CF₃ | |
| 18-156 | 4-F | H | CH(CH₃)CF₃ | |
| 18-157 | 2-Cl | H | CH(CH₃)CF₃ | |
| 18-158 | 3-Cl | H | CH(CH₃)CF₃ | |
| 18-159 | 4-Cl | H | CH(CH₃)CF₃ | |
| 18-160 | 2-Br | H | CH(CH₃)CF₃ | |
| 18-161 | 3-Br | H | CH(CH₃)CF₃ | |
| 18-162 | 4-Br | H | CH(CH₃)CF₃ | |
| 18-163 | 2-I | H | CH(CH₃)CF₃ | |
| 18-164 | 3-I | H | CH(CH₃)CF₃ | |
| 18-165 | 4-I | H | CH(CH₃)CF₃ | |
| 18-166 | 2-Cl | 3-Cl | CH(CH₃)CF₃ | |
| 18-167 | 2-Cl | 4-Cl | CH(CH₃)CF₃ | |
| 18-168 | 2-Cl | 5-Cl | CH(CH₃)CF₃ | |
| 18-169 | 2-Cl | 6-Cl | CH(CH₃)CF₃ | |
| 18-170 | 3-Cl | 4-Cl | CH(CH₃)CF₃ | |
| 18-171 | 3-Cl | 5-Cl | CH(CH₃)CF₃ | |
| 18-172 | H | H | CHFC₂H₅ | |
| 18-173 | 2-F | H | CHFC₂H₅ | |
| 18-174 | 3-F | H | CHFC₂H₅ | |
| 18-175 | 4-F | H | CHFC₂H₅ | |
| 18-176 | 2-Cl | H | CHFC₂H₅ | |
| 18-177 | 3-Cl | H | CHFC₂H₅ | |
| 18-178 | 4-Cl | H | CHFC₂H₅ | |
| 18-179 | 2-Br | H | CHFC₂H₅ | |
| 18-180 | 3-Br | H | CHFC₂H₅ | |
| 18-181 | 4-Br | H | CHFC₂H₅ | 19F: −171.6 |
| | | | | ppm, CHF |
| 18-182 | 2-I | H | CHFC₂H₅ | |
| 18-183 | 3-I | H | CHFC₂H₅ | |
| 18-184 | 4-I | H | CHFC₂H₅ | |
| 18-185 | 2-Cl | 3-Cl | CHFC₂H₅ | |
| 18-186 | 2-Cl | 4-Cl | CHFC₂H₅ | |
| 18-187 | 2-Cl | 5-Cl | CHFC₂H₅ | |
| 18-188 | 2-Cl | 6-Cl | CHFC₂H₅ | |
| 18-189 | 3-Cl | 4-Cl | CHFC₂H₅ | |
| 18-190 | 3-Cl | 5-Cl | CHFC₂H₅ | NMR see below |
| 18-191 | H | H | CHFnPr | |
| 18-192 | 2-F | H | CHFnPr | |
| 18-193 | 3-F | H | CHFnPr | |
| 18-194 | 4-F | H | CHFnPr | |
| 18-195 | 2-Cl | H | CHFnPr | |
| 18-196 | 3-Cl | H | CHFnPr | |
| 18-197 | 4-Cl | H | CHFnPr | |
| 18-198 | 2-Br | H | CHFnPr | |
| 18-199 | 3-Br | H | CHFnPr | |
| 18-200 | 4-Br | H | CHFnPr | 1H: 5.54 ppm, CHF |
| 18-201 | 2-I | H | CHFnPr | |
| 18-202 | 3-I | H | CHFnPr | |
| 18-203 | 4-I | H | CHFnPr | |
| 18-204 | 2-Cl | 3-Cl | CHFnPr | |
| 18-205 | 2-Cl | 4-Cl | CHFnPr | |
| 18-206 | 2-Cl | 5-Cl | CHFnPr | |
| 18-207 | 2-Cl | 6-Cl | CHFnPr | |
| 18-208 | 3-Cl | 4-Cl | CHFnPr | |
| 18-209 | 3-Cl | 5-Cl | CHFnPr | NMR see below |
| 18-210 | H | H | CH₂CH(CF₃)CH₃ | |
| 18-211 | 2-F | H | CH₂CH(CF₃)CH₃ | |
| 18-212 | 3-F | H | CH₂CH(CF₃)CH₃ | |
| 18-213 | 4-F | H | CH₂CH(CF₃)CH₃ | |
| 18-214 | 2-Cl | H | CH₂CH(CF₃)CH₃ | |
| 18-215 | 3-Cl | H | CH₂CH(CF₃)CH₃ | |
| 18-216 | 4-Cl | H | CH₂CH(CF₃)CH₃ | |
| 18-217 | 2-Br | H | CH₂CH(CF₃)CH₃ | |
| 18-218 | 3-Br | H | CH₂CH(CF₃)CH₃ | |
| 18-219 | 4-Br | H | CH₂CH(CF₃)CH₃ | 1H: 1.01 ppm, d, CH₃ |
| 18-220 | 2-I | H | CH₂CH(CF₃)CH₃ | |
| 18-221 | 3-I | H | CH₂CH(CF₃)CH₃ | |
| 18-222 | 4-I | H | CH₂CH(CF₃)CH₃ | |
| 18-223 | 2-Cl | 3-Cl | CH₂CH(CF₃)CH₃ | |
| 18-224 | 2-Cl | 4-Cl | CH₂CH(CF₃)CH₃ | |
| 18-225 | 2-Cl | 5-Cl | CH₂CH(CF₃)CH₃ | |
| 18-226 | 2-Cl | 6-Cl | CH₂CH(CF₃)CH₃ | |
| 18-227 | 3-Cl | 4-Cl | CH₂CH(CF₃)CH₃ | |
| 18-228 | 3-Cl | 5-Cl | CH₂CH(CF₃)CH₃ | |
| 18-229 | 3-Cl | 5-Cl | CF₂CF₂OMe | NMR see below |
| 18-230 | 3-Br | 5-Br | CF₂CH₃ | NMR see below |
| 18-231 | 3-CF₃ | 5-CF₃ | CHFCH₃ | NMR see below |
| 18-232 | 3-Cl | 5-CF₃ | CHFCH₃ | NMR see below |
| 18-233 | 3-CF₃ | 5-CF₃ | CF₂CH₃ | NMR see below |
| 18-234 | 3-CF₃ | 5-CF₃ | CF₂CF₃ | NMR see below |
| 18-235 | 3-Cl | 5-CF₃ | CF₂CH₃ | NMR see below |
| 18-236 | 3-Cl | 5-CF₃ | CF₂CF₃ | NMR see below |
| 18-237 | 3-F | 5-CF₃ | CF₂CF₃ | NMR see below |
| 18-238 | 3-CF₃ | 4-Cl | CF₂CH₂Br | ¹H-NMR (CD₃CN): |
| | | | | 8.82 (d, |
| | | | | 2H), 7.36 (t, 1H), |
| | | | | 4.08 ppm (t, 2H) |
| 18-239 | 3-Cl | 5-CF₃ | (S)-CHFCH₃ | ¹H-NMR |
| | | | | (d₆-DMSO): |
| | | | | 8.89 (d, 2H), |
| | | | | 7.44 ppm (t, 1H) |
| 18-240 | 3-F | 5-CF₃ | CF₂CH₃ | NMR see below |
| 18-241 | 3-Cl | 5-OCF₃ | CF₂CF₃ | NMR see below |
| 18-242 | 3-Br | 5-Br | CF₂CF₃ | NMR see below |
| 18-243 | 3-Br | 5-Br | CHFCH₃ | NMR see below |
| 18-244 | 3-CF₃ | 4-Cl | CF₂CF₃ | NMR see below |
| 18-245 | 3-CF₃ | 4-Cl | CHFCH₃ | NMR see below |
| 18-246 | 3-Cl | 4-Br | CF₂CF₃ | NMR see below |
| 18-247 | 3-CF₃ | 4-Cl | CF₂CH₃ | |
| 18-248 | 2-F | 4-Br | CHFCH₃ | |
| 18-249 | 2-F | 4-Br | CF₂CF₃ | |
| 18-250 | 2-F | 4-Br | CF₂CH₃ | |
| 18-251 | 2-Cl | 4-Br | CHFCH₃ | |
| 18-252 | 2-Cl | 4-Br | CF₂CF₃ | |
| 18-253 | 2-Cl | 4-Br | CF₂CH₃ | |
| 18-254 | 3-Cl | 5-Br | CHFCH₃ | |
| 18-255 | 3-Cl | 5-Br | CF₂CF₃ | |
| 18-256 | 3-Cl | 5-Br | CF₂CH₃ | |
| 18-257 | 3-Cl | 4-Br | CHFCH₃ | |
| 18-258 | 3-Cl | 4-Br | CF₂CF₃ | |
| 18-259 | 2-Cl | 4-Br | CF₂CH₃ | |
| 18-260 | 3-CF₃ | 4-Br | CHFCH₃ | |
| 18-261 | 3-CF₃ | 4-Br | CF₂CF₃ | |
| 18-262 | 3-CF₃ | 4-Br | CF₂CH₃ | |
| 18-263 | 3-Cl | 4-I | CHFCH₃ | |
| 18-264 | 3-Cl | 4-I | CF₂CF₃ | |
| 18-265 | 3-Cl | 4-I | CF₂CH₃ | |

TABLE 19

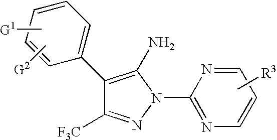

| Example | | | | |
|---|---|---|---|---|
| No. | R³ | G¹ | G² | Physical data |
| 19-01 | 4-Me | H | H | |
| 19-02 | 4-Me | 2-F | H | |
| 19-03 | 4-Me | 3-F | H | |
| 19-04 | 4-Me | 4-F | H | |
| 19-05 | 4-Me | 2-Cl | H | |
| 19-06 | 4-Me | 3-Cl | H | |
| 19-07 | 4-Me | 4-Cl | H | ¹H-NMR (d₆ DMSO): |
| | | | | 8.75 ppm, (d, 1H) |
| 19-08 | 4-Me | 2-Br | H | |
| 19-09 | 4-Me | 3-Br | H | lgP = 3.84 |
| 19-10 | 4-Me | 4-Br | H | lgP = 3.37 |
| 19-11 | 4-Me | 2-I | H | |
| 19-12 | 4-Me | 3-I | H | |
| 19-13 | 4-Me | 4-I | H | |
| 19-14 | 4-Me | 2-Cl | 3-Cl | |
| 19-15 | 4-Me | 2-Cl | 4-Cl | |
| 19-16 | 4-Me | 2-Cl | 5-Cl | |
| 19-17 | 4-Me | 2-Cl | 6-Cl | |
| 19-18 | 4-Me | 3-Cl | 4-Cl | lgP = 3.73 |
| 19-19 | 4-Me | 3-Cl | 5-Cl | |
| 19-20 | 5-Me | H | H | |
| 19-21 | 5-Me | 2-F | H | |
| 19-22 | 5-Me | 3-F | H | |
| 19-23 | 5-Me | 4-F | H | |
| 19-24 | 5-Me | 2-Cl | H | |
| 19-25 | 5-Me | 3-Cl | H | |
| 19-26 | 5-Me | 4-Cl | H | NMR see below |
| 19-27 | 5-Me | 2-Br | H | |
| 19-28 | 5-Me | 3-Br | H | |
| 19-29 | 5-Me | 4-Br | H | |
| 19-30 | 5-Me | 2-I | H | |
| 19-31 | 5-Me | 3-I | H | |
| 19-32 | 5-Me | 4-I | H | |
| 19-33 | 5-Me | 2-Cl | 3-Cl | |
| 19-34 | 5-Me | 2-Cl | 4-Cl | |
| 19-35 | 5-Me | 2-Cl | 5-Cl | |
| 19-36 | 5-Me | 2-Cl | 6-Cl | |
| 19-37 | 5-Me | 3-Cl | 4-Cl | NMR see below |
| 19-38 | 5-Me | 3-Cl | 5-Cl | |
| 19-39 | 5-F | H | H | |
| 19-40 | 5-F | 2-F | H | |
| 19-41 | 5-F | 3-F | H | |
| 19-42 | 5-F | 4-F | H | |
| 19-43 | 5-F | 2-Cl | H | |
| 19-44 | 5-F | 3-Cl | H | |
| 19-45 | 5-F | 4-Cl | H | NMR see below |
| 19-46 | 5-F | 2-Br | H | |
| 19-47 | 5-F | 3-Br | H | |
| 19-48 | 5-F | 4-Br | H | lgP = 3.19 |
| 19-49 | 5-F | 2-I | H | |
| 19-50 | 5-F | 3-I | H | |
| 19-51 | 5-F | 4-I | H | |
| 19-52 | 5-F | 2-Cl | 3-Cl | |
| 19-53 | 5-F | 2-Cl | 4-Cl | |
| 19-54 | 5-F | 2-Cl | 5-Cl | |
| 19-55 | 5-F | 2-Cl | 6-Cl | |
| 19-56 | 5-F | 3-Cl | 4-Cl | NMR see below |
| 19-57 | 5-F | 3-Cl | 5-Cl | lgP = 3.63 |
| 19-58 | 5-Cl | H | H | |
| 19-59 | 5-Cl | 2-F | H | |
| 19-60 | 5-Cl | 3-F | H | |
| 19-61 | 5-Cl | 4-F | H | |
| 19-62 | 5-Cl | 2-Cl | H | |
| 19-63 | 5-Cl | 3-Cl | H | |
| 19-64 | 5-Cl | 4-Cl | H | lgP = 3.53 |
| 19-65 | 5-Cl | 2-Br | H | |
| 19-66 | 5-Cl | 3-Br | H | |
| 19-67 | 5-Cl | 4-Br | H | |
| 19-68 | 5-Cl | 2-I | H | |
| 19-69 | 5-Cl | 3-I | H | |
| 19-70 | 5-Cl | 4-I | H | |
| 19-71 | 5-Cl | 2-Cl | 3-Cl | |
| 19-72 | 5-Cl | 2-Cl | 4-Cl | |
| 19-73 | 5-Cl | 2-Cl | 5-Cl | |
| 19-74 | 5-Cl | 2-Cl | 6-Cl | |
| 19-75 | 5-Cl | 3-Cl | 4-Cl | NMR see below |
| 19-76 | 5-Cl | 3-Cl | 5-Cl | lgP = 4.10 |
| 19-77 | 5-Br | H | H | |
| 19-78 | 5-Br | 2-F | H | |
| 19-79 | 5-Br | 3-F | H | |
| 19-80 | 5-Br | 4-F | H | |
| 19-81 | 5-Br | 2-Cl | H | |
| 19-82 | 5-Br | 3-Cl | H | |
| 19-83 | 5-Br | 4-Cl | H | |
| 19-84 | 5-Br | 2-Br | H | |
| 19-85 | 5-Br | 3-Br | H | |
| 19-86 | 5-Br | 4-Br | H | |
| 19-87 | 5-Br | 2-I | H | |
| 19-88 | 5-Br | 3-I | H | |
| 19-89 | 5-Br | 4-I | H | |
| 19-90 | 5-Br | 2-Cl | 3-Cl | |
| 19-91 | 5-Br | 2-Cl | 4-Cl | |
| 19-92 | 5-Br | 2-Cl | 5-Cl | |
| 19-93 | 5-Br | 2-Cl | 6-Cl | |
| 19-94 | 5-Br | 3-Cl | 4-Cl | |
| 19-95 | 5-Br | 3-Cl | 5-Cl | lgP = 4.15 |
| 19-96 | 5-I | H | H | |
| 19-97 | 5-I | 2-F | H | |
| 19-98 | 5-I | 3-F | H | |
| 19-99 | 5-I | 4-F | H | |
| 19-100 | 5-I | 2-Cl | H | |
| 19-101 | 5-I | 3-Cl | H | |
| 19-102 | 5-I | 4-Cl | H | |
| 19-103 | 5-I | 2-Br | H | |
| 19-104 | 5-I | 3-Br | H | |
| 19-105 | 5-I | 4-Br | H | |
| 19-106 | 5-I | 2-I | H | |
| 19-107 | 5-I | 3-I | H | |
| 19-108 | 5-I | 4-I | H | |
| 19-109 | 5-I | 2-Cl | 3-Cl | |
| 19-110 | 5-I | 2-Cl | 4-Cl | |
| 19-111 | 5-I | 2-Cl | 5-Cl | |
| 19-112 | 5-I | 2-Cl | 6-Cl | |
| 19-113 | 5-I | 3-Cl | 4-Cl | |
| 19-114 | 5-I | 3-Cl | 5-Cl | |
| 19-115 | 5-CF₃ | H | H | |
| 19-116 | 5-CF₃ | 2-F | H | |
| 19-117 | 5-CF₃ | 3-F | H | |
| 19-118 | 5-CF₃ | 4-F | H | |
| 19-119 | 5-CF₃ | 2-Cl | H | |
| 19-120 | 5-CF₃ | 3-Cl | H | |
| 19-121 | 5-CF₃ | 4-Cl | H | NMR see below |
| 19-122 | 5-CF₃ | 2-Br | H | |
| 19-123 | 5-CF₃ | 3-Br | H | |
| 19-124 | 5-CF₃ | 4-Br | H | |
| 19-125 | 5-CF₃ | 2-I | H | |
| 19-126 | 5-CF₃ | 3-I | H | |
| 19-127 | 5-CF₃ | 4-I | H | |
| 19-128 | 5-CF₃ | 2-Cl | 3-Cl | |
| 19-129 | 5-CF₃ | 2-Cl | 4-Cl | |
| 19-130 | 5-CF₃ | 2-Cl | 5-Cl | |
| 19-131 | 5-CF₃ | 2-Cl | 6-Cl | |
| 19-132 | 5-CF₃ | 3-Cl | 4-Cl | |
| 19-133 | 5-CF₃ | 3-Cl | 5-Cl | |
| 19-134 | 4-CF₃ | H | H | |
| 19-135 | 4-CF₃ | 2-F | H | |
| 19-136 | 4-CF₃ | 3-F | H | |
| 19-137 | 4-CF₃ | 4-F | H | |
| 19-138 | 4-CF₃ | 2-Cl | H | |
| 19-139 | 4-CF₃ | 3-Cl | H | |
| 19-140 | 4-CF₃ | 4-Cl | H | |
| 19-141 | 4-CF₃ | 2-Br | H | |
| 19-142 | 4-CF₃ | 3-Br | H | |
| 19-143 | 4-CF₃ | 4-Br | H | 19F: −61.9, −70.4 ppm |
| 19-144 | 4-CF₃ | 2-I | H | |
| 19-145 | 4-CF₃ | 3-I | H | |
| 19-146 | 4-CF₃ | 4-I | H | |
| 19-147 | 4-CF₃ | 2-Cl | 3-Cl | |
| 19-148 | 4-CF₃ | 2-Cl | 4-Cl | |
| 19-149 | 4-CF₃ | 2-Cl | 5-Cl | |
| 19-150 | 4-CF₃ | 2-Cl | 6-Cl | |
| 19-151 | 4-CF₃ | 3-Cl | 4-Cl | |
| 19-152 | 4-CF₃ | 3-Cl | 5-Cl | lgP = 4.25 |
| 19-153 | 4-OMe | H | H | |
| 19-154 | 4-OMe | 2-F | H | |
| 19-155 | 4-OMe | 3-F | H | |
| 19-156 | 4-OMe | 4-F | H | |
| 19-157 | 4-OMe | 2-Cl | H | |
| 19-158 | 4-OMe | 3-Cl | H | |
| 19-159 | 4-OMe | 4-Cl | H | |
| 19-160 | 4-OMe | 2-Br | H | |
| 19-161 | 4-OMe | 3-Br | H | |
| 19-162 | 4-OMe | 4-Br | H | 1H: 4.10 ppm, OCH₃ |
| 19-163 | 4-OMe | 2-I | H | |
| 19-164 | 4-OMe | 3-I | H | |
| 19-165 | 4-OMe | 4-I | H | |
| 19-166 | 4-OMe | 2-Cl | 3-Cl | |
| 19-167 | 4-OMe | 2-Cl | 4-Cl | |
| 19-168 | 4-OMe | 2-Cl | 5-Cl | |
| 19-169 | 4-OMe | 2-Cl | 6-Cl | |
| 19-170 | 4-OMe | 3-Cl | 4-Cl | |
| 19-171 | 4-OMe | 3-Cl | 5-Cl | lgP = 2.93 |
| 19-172 | 4-NMe₂ | H | H | |
| 19-173 | 4-NMe₂ | 2-F | H | |
| 19-174 | 4-NMe₂ | 3-F | H | |
| 19-175 | 4-NMe₂ | 4-F | H | |
| 19-176 | 4-NMe₂ | 2-Cl | H | |
| 19-177 | 4-NMe₂ | 3-Cl | H | |
| 19-178 | 4-NMe₂ | 4-Cl | H | |
| 19-179 | 4-NMe₂ | 2-Br | H | |
| 19-180 | 4-NMe₂ | 3-Br | H | |
| 19-181 | 4-NMe₂ | 4-Br | H | 1H: 6.41, 8.23 ppm; |
| | | | | pyrimidine-H |
| 19-182 | 4-NMe₂ | 2-I | H | |
| 19-183 | 4-NMe₂ | 3-I | H | |
| 19-184 | 4-NMe₂ | 4-I | H | |
| 19-185 | 4-NMe₂ | 2-Cl | 3-Cl | |
| 19-186 | 4-NMe₂ | 2-Cl | 4-Cl | |
| 19-187 | 4-NMe₂ | 2-Cl | 5-Cl | |
| 19-188 | 4-NMe₂ | 2-Cl | 6-Cl | |
| 19-189 | 4-NMe₂ | 3-Cl | 4-Cl | |
| 19-190 | 4-NMe₂ | 3-Cl | 5-Cl | |
| 19-191 | 5-OCH₃ | 4-Cl | H | ¹H-NMR (d₆-DMSO): |
| | | | | 8.66 ppm, (s, 2H) |
| 19-192 | 5-OCH₃ | 3-Cl | 5-Cl | ¹H-NMR (d₆-DMSO): |
| | | | | 8.66 ppm, (s, 2H) |
| 19-193 | 5-OCH₃ | 3-Cl | 4-Cl | ¹H-NMR (d₆-DMSO): |
| | | | | 8.66 ppm, (s, 2H) |
| 19-194 | 5-CN | 3-Cl | 5-Cl | ¹H-NMR (d₆-DMSO): |
| | | | | 9.36 ppm, (s, 2H) |
| 19-195 | 4-CF₃ | 3-Br | 4-Cl | NMR see below |
| 19-196 | 4-CF₃ | 3-Cl | 4-Br | NMR see below |
| 19-197 | 4-CF₃ | 3-CF₃ | 5-CF₃ | NMR see below |
| 19-198 | 4-CH₃ | 3-Cl | 5-F | lgP = 3.42 |
| 19-199 | 4-CH₃ | 3-F | 5-F | lgP = 3.06 |
| 19-200 | 4-CH₃ | 2-F | 4-Br | lgP = 3.28 |
| 19-201 | 4-CH₃ | 3-F | 4-F | lgP = 3.02 |
| 19-202 | 4-CH₃ | 3-CH₃ | 5-CH₃ | lgP = 3.42 |
| 19-203 | 5-CH₃ | 3-Cl | 5-F | lgP = 3.42 |
| 19-204 | 5-CH₃ | 3-F | 5-F | lgP = 3.06 |
| 19-205 | 5-CH₃ | 2-F | 4-Br | lgP = 3.28 |
| 19-206 | 5-CH₃ | 3-F | 4-F | lgP = 3.02 |
| 19-207 | 5-CH₃ | 3-Br | 5-Br | lgP = 4.05 |
| 19-208 | 5-CH₃ | 3-CH₃ | 5-CH₃ | lgP = 3.47 |
| 19-209 | 4-CN | 3-Cl | 5-Cl | lgP = 3.84 |

TABLE 24

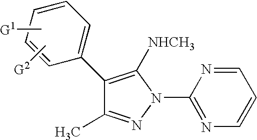

| Example | | | |
|---|---|---|---|
| No. | G¹ | G² | Physical data |
| 24-01 | H | H | |
| 24-02 | 2-F | H | |
| 24-03 | 3-F | H | |
| 24-04 | 4-F | H | |
| 24-05 | 2-Cl | H | |
| 24-06 | 3-Cl | H | NMR see below |
| 24-07 | 4-Cl | H | |
| 24-08 | 2-Br | H | |
| 24-09 | 3-Br | H | |
| 24-10 | 4-Br | H | |
| 24-11 | 2-I | H | |
| 24-12 | 3-I | H | |
| 24-13 | 4-I | H | |
| 24-14 | 2-F | 3-F | |
| 24-15 | 2-F | 4-F | |
| 24-16 | 2-F | 5-F | |
| 24-17 | 2-F | 6-F | |
| 24-18 | 2-F | 3-Cl | |
| 24-19 | 2-F | 4-Cl | |
| 24-20 | 2-F | 5-Cl | |
| 24-21 | 2-F | 6-Cl | |
| 24-22 | 2-F | 3-Br | |
| 24-23 | 2-F | 4-Br | |
| 24-24 | 2-F | 5-Br | |
| 24-25 | 2-F | 6-Br | |
| 24-26 | 2-F | 3-I | |
| 24-27 | 2-F | 4-I | |
| 24-28 | 2-F | 5-I | |
| 24-29 | 2-F | 6-I | |
| 24-30 | 2-Cl | 3-F | |
| 24-31 | 2-Cl | 4-F | |
| 24-32 | 2-Cl | 5-F | |
| 24-33 | 2-Cl | 3-Cl | |
| 24-34 | 2-Cl | 4-Cl | |
| 24-35 | 2-Cl | 5-Cl | |
| 24-36 | 2-Cl | 6-Cl | |
| 24-37 | 2-Cl | 3-Br | |
| 24-38 | 2-Cl | 4-Br | |
| 24-39 | 2-Cl | 5-Br | |
| 24-40 | 2-Cl | 6-Br | |
| 24-41 | 2-Cl | 3-I | |
| 24-42 | 2-Cl | 4-I | |
| 24-43 | 2-Cl | 5-I | |
| 24-44 | 2-Cl | 6-I | |
| 24-45 | 2-Br | 3-F | |
| 24-46 | 2-Br | 4-F | |
| 24-47 | 2-Br | 5-F | |
| 24-48 | 2-Br | 3-Cl | |
| 24-49 | 2-Br | 4-Cl | |
| 24-50 | 2-Br | 5-Cl | |
| 24-51 | 2-Br | 3-Br | |
| 24-52 | 2-Br | 4-Br | |
| 24-53 | 2-Br | 5-Br | |
| 24-54 | 2-Br | 6-Br | |
| 24-55 | 2-Br | 3-I | |
| 24-56 | 2-Br | 4-I | |
| 24-57 | 2-Br | 5-I | |
| 24-58 | 2-Br | 6-I | |
| 24-59 | 2-I | 3-F | |
| 24-60 | 2-I | 4-F | |
| 24-61 | 2-I | 5-F | |
| 24-62 | 2-I | 3-Cl | |
| 24-63 | 2-I | 4-Cl | |
| 24-64 | 2-I | 5-Cl | |
| 24-65 | 2-I | 3-Br | |
| 24-66 | 2-I | 4-Br | |
| 24-67 | 2-I | 5-Br | |
| 24-68 | 2-I | 3-I | |
| 24-69 | 2-I | 4-I | |
| 24-70 | 2-I | 5-I | |
| 24-71 | 2-I | 6-I | |
| 24-72 | 3-F | 4-F | |
| 24-73 | 3-F | 5-F | |
| 24-74 | 3-F | 4-Cl | |
| 24-75 | 3-F | 5-Cl | |
| 24-76 | 3-F | 4-Br | |
| 24-77 | 3-F | 5-Br | |
| 24-78 | 3-F | 4-I | |
| 24-79 | 3-F | 5-I | |
| 24-80 | 3-Cl | 4-F | |
| 24-81 | 3-Cl | 4-Cl | |
| 24-82 | 3-Cl | 5-Cl | |
| 24-83 | 3-Cl | 4-Br | |
| 24-84 | 3-Cl | 5-Br | |
| 24-85 | 3-Cl | 4-I | |
| 24-86 | 3-Cl | 5-I | |
| 24-87 | 3-Br | 4-F | |
| 24-88 | 3-Br | 4-Cl | |
| 24-89 | 3-Br | 4-Br | |
| 24-90 | 3-Br | 5-Br | |
| 24-91 | 3-Br | 4-I | |
| 24-92 | 3-Br | 5-I | |
| 24-93 | 3-I | 4-F | |
| 24-94 | 3-I | 4-Cl | |
| 24-95 | 3-I | 4-Br | |
| 24-96 | 3-I | 4-I | |
| 24-97 | 3-I | 4-I | |

TABLE 25

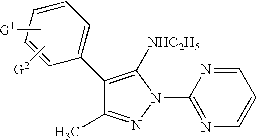

| Example | | | |
|---|---|---|---|
| No. | G¹ | G² | Physical data |
| 25-01 | H | H | |
| 25-02 | 2-F | H | |
| 25-03 | 3-F | H | |
| 25-04 | 4-F | H | logP = 2.59 |
| 25-05 | 2-Cl | H | logP = 2.69 |
| 25-06 | 3-Cl | H | logP = 3.02 |
| 25-07 | 4-Cl | H | NMR see below |
| 25-08 | 2-Br | H | |
| 25-09 | 3-Br | H | |
| 25-10 | 4-Br | H | |
| 25-11 | 2-I | H | |
| 25-12 | 3-I | H | |
| 25-13 | 4-I | H | |
| 25-14 | 2-F | 3-F | |
| 25-15 | 2-F | 4-F | |
| 25-16 | 2-F | 5-F | |
| 25-17 | 2-F | 6-F | |
| 25-18 | 2-F | 3-Cl | |
| 25-19 | 2-F | 4-Cl | |
| 25-20 | 2-F | 5-Cl | |
| 25-21 | 2-F | 6-Cl | |
| 25-22 | 2-F | 3-Br | |
| 25-23 | 2-F | 4-Br | |
| 25-24 | 2-F | 5-Br | |
| 25-25 | 2-F | 6-Br | |
| 25-26 | 2-F | 3-I | |
| 25-27 | 2-F | 4-I | |
| 25-28 | 2-F | 5-I | |
| 25-29 | 2-F | 6-I | |
| 25-30 | 2-Cl | 3-F | |
| 25-31 | 2-Cl | 4-F | logP = 2.79 |
| 25-32 | 2-Cl | 5-F | |
| 25-33 | 2-Cl | 3-Cl | |
| 25-34 | 2-Cl | 4-Cl | NMR see below |
| 25-35 | 2-Cl | 5-Cl | |
| 25-36 | 2-Cl | 6-Cl | |
| 25-37 | 2-Cl | 3-Br | |
| 25-38 | 2-Cl | 4-Br | |
| 25-39 | 2-Cl | 5-Br | |
| 25-40 | 2-Cl | 6-Br | |
| 25-41 | 2-Cl | 3-I | |
| 25-42 | 2-Cl | 4-I | |
| 25-43 | 2-Cl | 5-I | |
| 25-44 | 2-Cl | 6-I | |
| 25-45 | 2-Br | 3-F | |
| 25-46 | 2-Br | 4-F | |
| 25-47 | 2-Br | 5-F | |
| 25-48 | 2-Br | 3-Cl | |
| 25-49 | 2-Br | 4-Cl | |
| 25-50 | 2-Br | 5-Cl | |
| 25-51 | 2-Br | 3-Br | |
| 25-52 | 2-Br | 4-Br | |
| 25-53 | 2-Br | 5-Br | |
| 25-54 | 2-Br | 6-Br | |
| 25-55 | 2-Br | 3-I | |
| 25-56 | 2-Br | 4-I | |
| 25-57 | 2-Br | 5-I | |
| 25-58 | 2-Br | 6-I | |
| 25-59 | 2-I | 3-F | |
| 25-60 | 2-I | 4-F | |
| 25-61 | 2-I | 5-F | |
| 25-62 | 2-I | 3-Cl | |
| 25-63 | 2-I | 4-Cl | |
| 25-64 | 2-I | 5-Cl | |
| 25-65 | 2-I | 3-Br | |
| 25-66 | 2-I | 4-Br | |
| 25-67 | 2-I | 5-Br | |
| 25-68 | 2-I | 3-I | |
| 25-69 | 2-I | 4-I | |
| 25-70 | 2-I | 5-I | |
| 25-71 | 2-I | 6-I | |
| 25-72 | 3-F | 4-F | |
| 25-73 | 3-F | 5-F | |
| 25-74 | 3-F | 4-Cl | |
| 25-75 | 3-F | 5-Cl | |
| 25-76 | 3-F | 4-Br | |
| 25-77 | 3-F | 5-Br | |
| 25-78 | 3-F | 4-I | |
| 25-79 | 3-F | 5-I | |
| 25-80 | 3-Cl | 4-F | |
| 25-81 | 3-Cl | 4-Cl | |
| 25-82 | 3-Cl | 5-Cl | |
| 25-83 | 3-Cl | 4-Br | |
| 25-84 | 3-Cl | 5-Br | |
| 25-85 | 3-Cl | 4-I | |
| 25-86 | 3-Cl | 5-I | |
| 25-87 | 3-Br | 4-F | |
| 25-88 | 3-Br | 4-Cl | |
| 25-89 | 3-Br | 4-Br | |
| 25-90 | 3-Br | 5-Br | |
| 25-91 | 3-Br | 4-I | |
| 25-92 | 3-Br | 5-I | |
| 25-93 | 3-I | 4-F | |
| 25-94 | 3-I | 4-Cl | |
| 25-95 | 3-I | 4-Br | |
| 25-96 | 3-I | 4-I | |
| 25-97 | 3-I | 4-I | |

TABLE 26

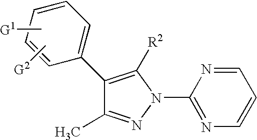

| Example | | | | |
|---|---|---|---|---|
| No. | G¹ | G² | R² | Physical data |
| 26-01 | H | H | NHnPr | |
| 26-02 | 2-F | H | NHnPr | |
| 26-03 | 3-F | H | NHnPr | |
| 26-04 | 4-F | H | NHnPr | |
| 26-05 | 2-Cl | H | NHnPr | |
| 26-06 | 3-Cl | H | NHnPr | logP = 3.37 |
| 26-07 | 4-Cl | H | NHnPr | logP = 3.42 |
| 26-08 | 2-Br | H | NHnPr | |
| 26-09 | 3-Br | H | NHnPr | |
| 26-10 | 4-Br | H | NHnPr | |
| 26-11 | 2-I | H | NHnPr | |
| 26-12 | 3-I | H | NHnPr | |
| 26-13 | 4-I | H | NHnPr | |
| 26-14 | 2-Cl | 3-Cl | NHnPr | |
| 26-15 | 2-Cl | 4-Cl | NHnPr | |
| 26-16 | 2-Cl | 5-Cl | NHnPr | |
| 26-17 | 2-Cl | 6-Cl | NHnPr | |
| 26-18 | 3-Cl | 4-Cl | NHnPr | |
| 26-19 | 3-Cl | 5-Cl | NHnPr | |
| 26-20 | H | H | NHiPr | |
| 26-21 | 2-F | H | NHiPr | |
| 26-22 | 3-F | H | NHiPr | |
| 26-23 | 4-F | H | NHiPr | NMR see below |
| 26-24 | 2-Cl | H | NHiPr | NMR see below |
| 26-25 | 3-Cl | H | NHiPr | |
| 26-26 | 4-Cl | H | NHiPr | NMR see below |
| 26-27 | 2-Br | H | NHiPr | |
| 26-28 | 3-Br | H | NHiPr | |
| 26-29 | 4-Br | H | NHiPr | |
| 26-30 | 2-I | H | NHiPr | |
| 26-31 | 3-I | H | NHiPr | |
| 26-32 | 4-I | H | NHiPr | |
| 26-33 | 2-Cl | 3-Cl | NHiPr | |
| 26-34 | 2-Cl | 4-Cl | NHiPr | |
| 26-35 | 2-Cl | 5-Cl | NHiPr | |
| 26-36 | 2-Cl | 6-Cl | NHiPr | |
| 26-37 | 2-Cl | 4-F | NHiPr | logP = 2.91 |
| 26-38 | 3-Cl | 4-Cl | NHiPr | |
| 26-39 | 3-Cl | 5-Cl | NHiPr | |
| 26-40 | H | H | NHCH₂phenyl | |
| 26-41 | 2-F | H | NHCH₂phenyl | |
| 26-42 | 3-F | H | NHCH₂phenyl | |
| 26-43 | 4-F | H | NHCH₂phenyl | |
| 26-44 | 2-Cl | H | NHCH₂phenyl | logP = 3.37 |
| 26-45 | 3-Cl | H | NHCH₂phenyl | |
| 26-46 | 4-Cl | H | NHCH₂phenyl | NMR see below |
| 26-47 | 2-Br | H | NHCH₂phenyl | |
| 26-48 | 3-Br | H | NHCH₂phenyl | |
| 26-49 | 4-Br | H | NHCH₂phenyl | |
| 26-50 | 2-I | H | NHCH₂phenyl | |
| 26-51 | 3-I | H | NHCH₂phenyl | |
| 26-52 | 4-I | H | NHCH₂phenyl | |
| 26-53 | 2-Cl | 3-Cl | NHCH₂phenyl | |
| 26-54 | 2-Cl | 4-Cl | NHCH₂phenyl | logP = 4.23 |
| 26-55 | 2-Cl | 5-Cl | NHCH₂phenyl | |
| 26-56 | 2-Cl | 6-Cl | NHCH₂phenyl | |
| 26-57 | 3-Cl | 4-Cl | NHCH₂phenyl | |
| 26-58 | 3-Cl | 5-Cl | NHCH₂phenyl | |
| 26-59 | H | H | NHCH₂cPr | |
| 26-60 | 2-F | H | NHCH₂cPr | |
| 26-61 | 3-F | H | NHCH₂cPr | |
| 26-62 | 4-F | H | NHCH₂cPr | logP = 3.15 |
| 26-63 | 2-Cl | H | NHCH₂cPr | |
| 26-64 | 3-Cl | H | NHCH₂cPr | |
| 26-65 | 4-Cl | H | NHCH₂cPr | NMR see below |
| 26-66 | 2-Br | H | NHCH₂cPr | |
| 26-67 | 3-Br | H | NHCH₂cPr | |
| 26-68 | 4-Br | H | NHCH₂cPr | |
| 26-69 | 2-I | H | NHCH₂cPr | |
| 26-70 | 3-I | H | NHCH₂cPr | |
| 26-71 | 4-I | H | NHCH₂cPr | |
| 26-72 | 2-Cl | 3-Cl | NHCH₂cPr | |
| 26-73 | 2-Cl | 4-Cl | NHCH₂cPr | |
| 26-74 | 2-Cl | 5-Cl | NHCH₂cPr | |
| 26-75 | 2-Cl | 6-Cl | NHCH₂cPr | |
| 26-76 | 3-Cl | 4-Cl | NHCH₂cPr | |
| 26-77 | 3-Cl | 5-Cl | NHCH₂cPr | |
| 26-78 | H | H | NHCH₂C═CMe₂ | |
| 26-79 | 2-F | H | NHCH₂C═CMe₂ | |
| 26-80 | 3-F | H | NHCH₂C═CMe₂ | |
| 26-81 | 4-F | H | NHCH₂C═CMe₂ | |
| 26-82 | 2-Cl | H | NHCH₂C═CMe₂ | |
| 26-83 | 3-Cl | H | NHCH₂C═CMe₂ | |
| 26-84 | 4-Cl | H | NHCH₂C═CMe₂ | logP = 3.84 |
| 26-85 | 2-Br | H | NHCH₂C═CMe₂ | |
| 26-86 | 3-Br | H | NHCH₂C═CMe₂ | |
| 26-87 | 4-Br | H | NHCH₂C═CMe₂ | |
| 26-88 | 2-I | H | NHCH₂C═CMe₂ | |
| 26-89 | 3-I | H | NHCH₂C═CMe₂ | |
| 26-90 | 4-I | H | NHCH₂C═CMe₂ | |
| 26-91 | 2-Cl | 3-Cl | NHCH₂C═CMe₂ | |
| 26-92 | 2-Cl | 4-Cl | NHCH₂C═CMe₂ | |
| 26-93 | 2-Cl | 5-Cl | NHCH₂C═CMe₂ | |
| 26-94 | 2-Cl | 6-Cl | NHCH₂C═CMe₂ | |
| 26-95 | 3-Cl | 4-Cl | NHCH₂C═CMe₂ | |
| 26-96 | 3-Cl | 5-Cl | NHCH₂C═CMe₂ | |
| 26-97 | H | H | NH(2′-i-pentyl) | |
| 26-98 | 2-F | H | NH(2′-i-pentyl) | |
| 26-99 | 3-F | H | NH(2′-i-pentyl) | |
| 26-100 | 4-F | H | NH(2′-i-pentyl) | NMR see below |
| 26-101 | 2-Cl | H | NH(2′-i-pentyl) | NMR see below |
| 26-102 | 3-Cl | H | NH(2′-i-pentyl) | NMR see below |
| 26-103 | 4-Cl | H | NH(2′-i-pentyl) | NMR see below |
| 26-104 | 2-Br | H | NH(2′-i-pentyl) | |
| 26-105 | 3-Br | H | NH(2′-i-pentyl) | |
| 26-106 | 4-Br | H | NH(2′-i-pentyl) | |
| 26-107 | 2-I | H | NH(2′-i-pentyl) | |
| 26-108 | 3-I | H | NH(2′-i-pentyl) | |
| 26-109 | 4-I | H | NH(2′-i-pentyl) | |
| 26-110 | 2-Cl | 3-Cl | NH(2′-i-pentyl) | |
| 26-111 | 2-Cl | 4-Cl | NH(2′-i-pentyl) | NMR see below |
| 26-112 | 2-Cl | 5-Cl | NH(2′-i-pentyl) | |
| 26-113 | 2-Cl | 6-Cl | NH(2′-i-pentyl) | |
| 26-114 | 3-Cl | 4-Cl | NH(2′-i-pentyl) | |
| 26-115 | 3-Cl | 5-Cl | NH(2′-i-pentyl) | |
| 26-116 | 4-Cl | H | NHCH₂CO₂Et | NMR see below |
| 26-117 | 2-Cl | 4-F | NH(2′-i-pentyl) | NMR see below |
| 26-118 | 2-Cl | 4-F | NH(2′-i-pentyl) | NMR see below |
| 26-119 | H | H | NHnBu | |
| 26-120 | 2-F | H | NHnBu | |
| 26-121 | 3-F | H | NHnBu | |
| 26-122 | 4-F | H | NHnBu | |
| 26-123 | 2-Cl | H | NHnBu | NMR see below |
| 26-124 | 3-Cl | H | NHnBu | NMR see below |
| 26-125 | 4-Cl | H | NHnBu | NMR see below |
| 26-126 | 2-Br | H | NHnBu | |
| 26-127 | 3-Br | H | NHnBu | |
| 26-128 | 4-Br | H | NHnBu | |
| 26-129 | 2-I | H | NHnBu | |
| 26-130 | 3-I | H | NHnBu | |
| 26-131 | 4-I | H | NHnBu | |
| 26-132 | 2-Cl | 3-Cl | NHnBu | |
| 26-133 | 2-Cl | 4-Cl | NHnBu | |
| 26-134 | 2-Cl | 5-Cl | NHnBu | |
| 26-135 | 2-Cl | 6-Cl | NHnBu | |
| 26-136 | 3-Cl | 4-Cl | NHnBu | |
| 26-137 | 3-Cl | 5-Cl | NHnBu | |
| 26-138 | 2-Cl | 4-F | NHnBu | NMR see below |
| 26-139 | 3-Cl | H | NHiBu | |
| 26-140 | 4-Cl | H | NHiBu | |
| 26-141 | 3-Cl | H | NHCH₂CO₂Et | NMR see below |
| 26-142 | 2-Cl | 4-F | NHnPr | |

TABLE 27

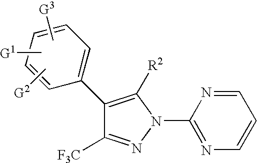

| Ex- | | | | | |
|---|---|---|---|---|---|
| ample | | | | | |
| No. | G¹ | G² | G³ | R² | Physical data |
| 27-01 | H | H | H | NHnPr | |
| 27-02 | H | 2-F | H | NHnPr | |
| 27-03 | H | 3-F | H | NHnPr | |
| 27-04 | H | 4-F | H | NHnPr | |
| 27-05 | H | 2-Cl | H | NHnPr | |
| 27-06 | H | 3-Cl | H | NHnPr | |
| 27-07 | H | 4-Cl | H | NHnPr | |
| 27-08 | H | 2-Br | H | NHnPr | |
| 27-09 | H | 3-Br | H | NHnPr | |
| 27-10 | H | 4-Br | H | NHnPr | |
| 27-11 | H | 2-I | H | NHnPr | |
| 27-12 | H | 3-I | H | NHnPr | |
| 27-13 | H | 4-I | H | NHnPr | |
| 27-14 | 3-Cl | 2-Cl | H | NHnPr | |
| 27-15 | 4-Cl | 2-Cl | H | NHnPr | |
| 27-16 | 5-Cl | 2-Cl | H | NHnPr | |
| 27-17 | 6-Cl | 2-Cl | H | NHnPr | |
| 27-18 | 4-Cl | 3-Cl | H | NHnPr | |
| 27-19 | 5-Cl | 3-Cl | H | NHnPr | NMR see below |
| 27-20 | H | H | H | NHiPr | |
| 27-21 | H | 2-F | H | NHiPr | |
| 27-22 | H | 3-F | H | NHiPr | |
| 27-23 | H | 4-F | H | NHiPr | |
| 27-24 | H | 2-Cl | H | NHiPr | |
| 27-25 | H | 3-Cl | H | NHiPr | |
| 27-26 | H | 4-Cl | H | NHiPr | NMR see below |
| 27-27 | H | 2-Br | H | NHiPr | |
| 27-28 | H | 3-Br | H | NHiPr | |
| 27-29 | H | 4-Br | H | NHiPr | |
| 27-30 | H | 2-I | H | NHiPr | |
| 27-31 | H | 3-I | H | NHiPr | |
| 27-32 | H | 4-I | H | NHiPr | |
| 27-33 | 3-Cl | 2-Cl | H | NHiPr | |
| 27-34 | 4-Cl | 2-Cl | H | NHiPr | |
| 27-35 | 5-Cl | 2-Cl | H | NHiPr | |
| 27-36 | 6-Cl | 2-Cl | H | NHiPr | |
| 27-37 | 4-Cl | 3-Cl | H | NHiPr | |
| 27-38 | 5-Cl | 3-Cl | H | NHiPr | NMR see below |
| 27-39 | H | H | H | NHCH₂phenyl | |
| 27-40 | H | 2-F | H | NHCH₂phenyl | |
| 27-41 | H | 3-F | H | NHCH₂phenyl | |
| 27-42 | H | 4-F | H | NHCH₂phenyl | |
| 27-43 | H | 2-Cl | H | NHCH₂phenyl | |
| 27-44 | H | 3-Cl | H | NHCH₂phenyl | |
| 27-45 | H | 4-Cl | H | NHCH₂phenyl | NMR see below |
| 27-46 | H | 2-Br | H | NHCH₂phenyl | |
| 27-47 | H | 3-Br | H | NHCH₂phenyl | |
| 27-48 | H | 4-Br | H | NHCH₂phenyl | |
| 27-49 | H | 2-I | H | NHCH₂phenyl | |
| 27-50 | H | 3-I | H | NHCH₂phenyl | NMR see below |
| 27-51 | H | 4-I | H | NHCH₂phenyl | NMR see below |
| 27-52 | 3-Cl | 2-Cl | H | NHCH₂phenyl | |
| 27-53 | 4-Cl | 2-Cl | H | NHCH₂phenyl | |
| 27-54 | 5-Cl | 2-Cl | H | NHCH₂phenyl | |
| 27-55 | 6-Cl | 2-Cl | H | NHCH₂phenyl | |
| 27-56 | 4-Cl | 3-Cl | H | NHCH₂phenyl | |
| 27-57 | 5-Cl | 3-Cl | H | NHCH₂phenyl | |
| 27-58 | H | H | H | NHCH₂cPr | |
| 27-59 | H | 2-F | H | NHCH₂cPr | |
| 27-60 | H | 3-F | H | NHCH₂cPr | |
| 27-61 | H | 4-F | H | NHCH₂cPr | |
| 27-62 | H | 2-Cl | H | NHCH₂cPr | |
| 27-63 | H | 3-Cl | H | NHCH₂cPr | |
| 27-64 | H | 4-Cl | H | NHCH₂cPr | |
| 27-65 | H | 2-Br | H | NHCH₂cPr | |
| 27-66 | H | 3-Br | H | NHCH₂cPr | |
| 27-67 | H | 4-Br | H | NHCH₂cPr | |
| 27-68 | H | 2-I | H | NHCH₂cPr | |
| 27-69 | H | 3-I | H | NHCH₂cPr | |
| 27-70 | H | 4-I | H | NHCH₂cPr | |
| 27-71 | 3-Cl | 2-Cl | H | NHCH₂cPr | |
| 27-72 | 4-Cl | 2-Cl | H | NHCH₂cPr | |
| 27-73 | 5-Cl | 2-Cl | H | NHCH₂cPr | |
| 27-74 | 6-Cl | 2-Cl | H | NHCH₂cPr | |
| 27-75 | 4-Cl | 3-Cl | H | NHCH₂cPr | |
| 27-76 | 5-Cl | 3-Cl | H | NHCH₂cPr | NMR see below |
| 27-77 | H | H | H | NHCH₂C═CMe₂ | |
| 27-78 | H | 2-F | H | NHCH₂C═CMe₂ | |
| 27-79 | H | 3-F | H | NHCH₂C═CMe₂ | |
| 27-80 | H | 4-F | H | NHCH₂C═CMe₂ | |
| 27-81 | H | 2-Cl | H | NHCH₂C═CMe₂ | |
| 27-82 | H | 3-Cl | H | NHCH₂C═CMe₂ | |
| 27-83 | H | 4-Cl | H | NHCH₂C═CMe₂ | |
| 27-84 | H | 2-Br | H | NHCH₂C═CMe₂ | |
| 27-85 | H | 3-Br | H | NHCH₂C═CMe₂ | |
| 27-86 | H | 4-Br | H | NHCH₂C═CMe₂ | |
| 27-87 | H | 2-I | H | NHCH₂C═CMe₂ | |
| 27-88 | H | 3-I | H | NHCH₂C═CMe₂ | |
| 27-89 | H | 4-I | H | NHCH₂C═CMe₂ | |
| 27-90 | 3-Cl | 2-Cl | H | NHCH₂C═CMe₂ | |
| 27-91 | 4-Cl | 2-Cl | H | NHCH₂C═CMe₂ | |
| 27-92 | 5-Cl | 2-Cl | H | NHCH₂C═CMe₂ | |
| 27-93 | 6-Cl | 2-Cl | H | NHCH₂C═CMe₂ | |
| 27-94 | 4-Cl | 3-Cl | H | NHCH₂C═CMe₂ | |
| 27-95 | 5-Cl | 3-Cl | H | NHCH₂C═CMe₂ | |
| 27-96 | H | H | H | NHCH₂CH═CH₂ | |
| 27-97 | H | 2-F | H | NHCH₂CH═CH₂ | |
| 27-98 | H | 3-F | H | NHCH₂CH═CH₂ | |
| 27-99 | H | 4-F | H | NHCH₂CH═CH₂ | |
| 27-100 | H | 2-Cl | H | NHCH₂CH═CH₂ | |
| 27-101 | H | 3-Cl | H | NHCH₂CH═CH₂ | |
| 27-102 | H | 4-Cl | H | NHCH₂CH═CH₂ | |
| 27-103 | H | 2-Br | H | NHCH₂CH═CH₂ | |
| 27-104 | H | 3-Br | H | NHCH₂CH═CH₂ | |
| 27-105 | H | 4-Br | H | NHCH₂CH═CH₂ | |
| 27-106 | H | 2-I | H | NHCH₂CH═CH₂ | |
| 27-107 | H | 3-I | H | NHCH₂CH═CH₂ | |
| 27-108 | H | 4-I | H | NHCH₂CH═CH₂ | |
| 27-109 | 3-Cl | 2-Cl | H | NHCH₂CH═CH₂ | |
| 27-110 | 4-Cl | 2-Cl | H | NHCH₂CH═CH₂ | |
| 27-111 | 5-Cl | 2-Cl | H | NHCH₂CH═CH₂ | |
| 27-112 | 6-Cl | 2-Cl | H | NHCH₂CH═CH₂ | |
| 27-113 | 4-Cl | 3-Cl | H | NHCH₂CH═CH₂ | |
| 27-114 | 5-Cl | 3-Cl | H | NHCH₂CH═CH₂ | NMR see below |
| 27-115 | H | H | H | NHiBu | |
| 27-116 | H | 2-F | H | NHiBu | |
| 27-117 | H | 3-F | H | NHiBu | |
| 27-118 | H | 4-F | H | NHiBu | |
| 27-119 | H | 2-Cl | H | NHiBu | |
| 27-120 | H | 3-Cl | H | NHiBu | |
| 27-121 | H | 4-Cl | H | NHiBu | |
| 27-122 | H | 2-Br | H | NHiBu | |
| 27-123 | H | 3-Br | H | NHiBu | |
| 27-124 | H | 4-Br | H | NHiBu | |
| 27-125 | H | 2-I | H | NHiBu | |
| 27-126 | H | 3-I | H | NHiBu | |
| 27-127 | H | 4-I | H | NHiBu | |
| 27-128 | 3-Cl | 2-Cl | H | NHiBu | |
| 27-129 | 4-Cl | 2-Cl | H | NHiBu | |
| 27-130 | 5-Cl | 2-Cl | H | NHiBu | |
| 27-131 | 6-Cl | 2-Cl | H | NHiBu | |
| 27-132 | 4-Cl | 3-Cl | H | NHiBu | |
| 27-133 | 5-Cl | 3-Cl | H | NHiBu | NMR see below |
| 27-134 | H | H | H | NHCH₂-4-Cl-Ph | |
| 27-135 | H | 2-F | H | NHCH₂-4-Cl-Ph | |
| 27-136 | H | 3-F | H | NHCH₂-4-Cl-Ph | |
| 27-137 | H | 4-F | H | NHCH₂-4-Cl-Ph | |
| 27-138 | H | 2-Cl | H | NHCH₂-4-Cl-Ph | |
| 27-139 | H | 3-Cl | H | NHCH₂-4-Cl-Ph | |
| 27-140 | H | 4-Cl | H | NHCH₂-4-Cl-Ph | NMR see below |
| 27-141 | H | 2-Br | H | NHCH₂-4-Cl-Ph | |
| 27-142 | H | 3-Br | H | NHCH₂-4-Cl-Ph | |
| 27-143 | H | 4-Br | H | NHCH₂-4-Cl-Ph | |
| 27-144 | H | 2-I | H | NHCH₂-4-Cl-Ph | |
| 27-145 | H | 3-I | H | NHCH₂-4-Cl-Ph | |
| 27-146 | H | 4-I | H | NHCH₂-4-Cl-Ph | |
| 27-147 | 3-Cl | 2-Cl | H | NHCH₂-4-Cl-Ph | |
| 27-148 | 4-Cl | 2-Cl | H | NHCH₂-4-Cl-Ph | |
| 27-149 | 5-Cl | 2-Cl | H | NHCH₂-4-Cl-Ph | |
| 27-150 | 6-Cl | 2-Cl | H | NHCH₂-4-Cl-Ph | |
| 27-151 | 4-Cl | 3-Cl | H | NHCH₂-4-Cl-Ph | |
| 27-152 | 5-Cl | 3-Cl | H | NHCH₂-4-Cl-Ph | |
| 27-153 | 5-Cl | 3-Cl | H | NHCH₂-3-Br- | NMR see below |
| | | | | 5-Br-Ph | |
| 27-154 | 5-Cl | 3-Cl | H | NH-cpent | NMR see below |
| 27-155 | 4-F | 3-Cl | H | NHCH₂phenyl | NMR see below |
| 27-156 | H | 4-OCHF₂ | H | NHCH₂phenyl | NMR see below |
| 27-157 | 3-Cl | 2-F | H | NHCH₂phenyl | NMR see below |
| 27-158 | 4-Cl | 3-CF₃ | H | NHCH₂phenyl | NMR see below |
| 27-159 | 4-Me | 3-OMe | H | NHCH₂phenyl | NMR see below |
| 27-160 | 4-Cl | 3-OCF₃ | H | NHCH₂phenyl | NMR see below |
| 27-161 | H | 4-SMe | H | NHCH₂phenyl | NMR see below |
| 27-162 | H | 3-OCHF₂ | H | NHCH₂phenyl | NMR see below |
| 27-163 | 4-OMe | 3-F | 5-F | NHCH₂phenyl | NMR see below |
| 27-164 | 3-Cl | 5-CF₃ | H | NHCH₂phenyl | |
| 27-165 | 3-CF₃ | 4-Cl | H | NHCH₂phenyl | |
| 27-166 | 3-CF₃ | 5-CF₃ | H | NHCH₂phenyl | |
| 27-167 | 3-Cl | 5-CF₃ | H | NHCH₂-4-Cl-Ph | |
| 27-168 | 3-CF₃ | 4-Cl | H | NHCH₂-4-Cl-Ph | |
| 27-169 | 3-CF₃ | 5-CF₃ | H | NHCH₂-4-Cl-Ph | |
| 27-170 | 3-Cl | 5-CF₃ | H | NHCH₂CH═CH₂ | |
| 27-171 | 3-CF₃ | 4-Cl | H | NHCH₂CH═CH₂ | |
| 27-172 | 3-CF₃ | 5-CF₃ | H | NHCH₂CH═CH₂ | |

TABLE 28

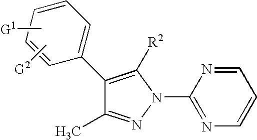

| Example | | | | |
|---|---|---|---|---|
| No. | G¹ | G² | R² | Physical data |
| 28-01 | H | H | NHCOCH₃ | |
| 28-02 | 2-F | H | NHCOCH₃ | |
| 28-03 | 3-F | H | NHCOCH₃ | |
| 28-04 | 4-F | H | NHCOCH₃ | |
| 28-05 | 2-Cl | H | NHCOCH₃ | |
| 28-06 | 3-Cl | H | NHCOCH₃ | |
| 28-07 | 4-Cl | H | NHCOCH₃ | NMR see below |
| 28-08 | 2-Br | H | NHCOCH₃ | |
| 28-09 | 3-Br | H | NHCOCH₃ | |
| 28-10 | 4-Br | H | NHCOCH₃ | |
| 28-11 | 2-I | H | NHCOCH₃ | |
| 28-12 | 3-I | H | NHCOCH₃ | |
| 28-13 | 4-I | H | NHCOCH₃ | |
| 28-14 | 2-Cl | 3-Cl | NHCOCH₃ | |
| 28-15 | 2-Cl | 4-Cl | NHCOCH₃ | |
| 28-16 | 2-Cl | 5-Cl | NHCOCH₃ | |
| 28-17 | 2-Cl | 6-Cl | NHCOCH₃ | |
| 28-18 | 3-Cl | 4-Cl | NHCOCH₃ | |
| 28-19 | 3-Cl | 5-Cl | NHCOCH₃ | |
| 28-20 | H | H | N(COCH₃)₂ | |
| 28-21 | 2-F | H | N(COCH₃)₂ | |
| 28-22 | 3-F | H | N(COCH₃)₂ | |
| 28-23 | 4-F | H | N(COCH₃)₂ | |
| 28-24 | 2-Cl | H | N(COCH₃)₂ | |
| 28-25 | 3-Cl | H | N(COCH₃)₂ | |
| 28-26 | 4-Cl | H | N(COCH₃)₂ | |
| 28-27 | 2-Br | H | N(COCH₃)₂ | |
| 28-28 | 3-Br | H | N(COCH₃)₂ | |
| 28-29 | 4-Br | H | N(COCH₃)₂ | |
| 28-30 | 2-I | H | N(COCH₃)₂ | |
| 28-31 | 3-I | H | N(COCH₃)₂ | |
| 28-32 | 4-I | H | N(COCH₃)₂ | |
| 28-33 | 2-Cl | 3-Cl | N(COCH₃)₂ | |
| 28-34 | 2-Cl | 4-Cl | N(COCH₃)₂ | |
| 28-35 | 2-Cl | 5-Cl | N(COCH₃)₂ | |
| 28-36 | 2-Cl | 6-Cl | N(COCH₃)₂ | |
| 28-37 | 3-Cl | 4-Cl | N(COCH₃)₂ | |
| 28-38 | 3-Cl | 5-Cl | N(COCH₃)₂ | |
| 28-39 | H | H | NHCOOEt | |
| 28-40 | 2-F | H | NHCOOEt | |
| 28-41 | 3-F | H | NHCOOEt | |
| 28-42 | 4-F | H | NHCOOEt | |
| 28-43 | 2-Cl | H | NHCOOEt | |
| 28-44 | 3-Cl | H | NHCOOEt | |
| 28-45 | 4-Cl | H | NHCOOEt | |
| 28-46 | 2-Br | H | NHCOOEt | |
| 28-47 | 3-Br | H | NHCOOEt | |
| 28-48 | 4-Br | H | NHCOOEt | |
| 28-49 | 2-I | H | NHCOOEt | |
| 28-50 | 3-I | H | NHCOOEt | |
| 28-51 | 4-I | H | NHCOOEt | |
| 28-52 | 2-Cl | 3-Cl | NHCOOEt | |
| 28-53 | 2-Cl | 4-Cl | NHCOOEt | |
| 28-54 | 2-Cl | 5-Cl | NHCOOEt | |
| 28-55 | 2-Cl | 6-Cl | NHCOOEt | |
| 28-56 | 3-Cl | 4-Cl | NHCOOEt | |
| 28-57 | 3-Cl | 5-Cl | NHCOOEt | |

TABLE 29

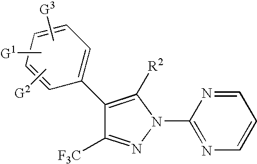

| Ex- | | | | | |
|---|---|---|---|---|---|
| ample | | | | | |
| No. | G¹ | G² | G³ | R² | Physical data |
| 29-01 | H | H | H | NHCOCH₃ | |
| 29-02 | H | 2-F | H | NHCOCH₃ | |
| 29-03 | H | 3-F | H | NHCOCH₃ | |
| 29-04 | H | 4-F | H | NHCOCH₃ | |
| 29-05 | H | 2-Cl | H | NHCOCH₃ | |
| 29-06 | H | 3-Cl | H | NHCOCH₃ | |
| 29-07 | H | 4-Cl | H | NHCOCH₃ | NMR see below |
| 29-08 | H | 2-Br | H | NHCOCH₃ | |
| 29-09 | H | 3-Br | H | NHCOCH₃ | |
| 29-10 | H | 4-Br | H | NHCOCH₃ | |
| 29-11 | H | 2-I | H | NHCOCH₃ | |
| 29-12 | H | 3-I | H | NHCOCH₃ | NMR see below |
| 29-13 | H | 4-I | H | NHCOCH₃ | |
| 29-14 | 3-Cl | 2-Cl | H | NHCOCH₃ | |
| 29-15 | 4-Cl | 2-Cl | H | NHCOCH₃ | |
| 29-16 | 5-Cl | 2-Cl | H | NHCOCH₃ | |
| 29-17 | 6-Cl | 2-Cl | H | NHCOCH₃ | |
| 29-18 | 4-Cl | 3-Cl | H | NHCOCH₃ | |
| 29-19 | 5-Cl | 3-Cl | H | NHCOCH₃ | NMR see below |
| 29-20 | H | H | H | N(COCH₃)₂ | |
| 29-21 | H | 2-F | H | N(COCH₃)₂ | |
| 29-22 | H | 3-F | H | N(COCH₃)₂ | |
| 29-23 | H | 4-F | H | N(COCH₃)₂ | |
| 29-24 | H | 2-Cl | H | N(COCH₃)₂ | |
| 29-25 | H | 3-Cl | H | N(COCH₃)₂ | |
| 29-26 | H | 4-Cl | H | N(COCH₃)₂ | NMR see below |
| 29-27 | H | 2-Br | H | N(COCH₃)₂ | |
| 29-28 | H | 3-Br | H | N(COCH₃)₂ | |
| 29-29 | H | 4-Br | H | N(COCH₃)₂ | 1H: 2.25 ppm, |
| | | | | | diacetyl |
| 29-30 | H | 2-I | H | N(COCH₃)₂ | |
| 29-31 | H | 3-I | H | N(COCH₃)₂ | |
| 29-32 | H | 4-I | H | N(COCH₃)₂ | |
| 29-33 | 3-Cl | 2-Cl | H | N(COCH₃)₂ | |
| 29-34 | 4-Cl | 2-Cl | H | N(COCH₃)₂ | |
| 29-35 | 5-Cl | 2-Cl | H | N(COCH₃)₂ | |
| 29-36 | 6-Cl | 2-Cl | H | N(COCH₃)₂ | |
| 29-37 | 4-Cl | 3-Cl | H | N(COCH₃)₂ | 1H: 2.27 ppm, |
| | | | | | diacetyl |
| 29-38 | 5-Cl | 3-Cl | H | N(COCH₃)₂ | |
| 29-39 | H | H | H | NHCOOMe | |
| 29-40 | H | 2-F | H | NHCOOMe | |
| 29-41 | H | 3-F | H | NHCOOMe | |
| 29-42 | H | 4-F | H | NHCOOMe | |
| 29-43 | H | 2-Cl | H | NHCOOMe | |
| 29-44 | H | 3-Cl | H | NHCOOMe | |
| 29-45 | H | 4-Cl | H | NHCOOMe | |
| 29-46 | H | 2-Br | H | NHCOOMe | |
| 29-47 | H | 3-Br | H | NHCOOMe | |
| 29-48 | H | 4-Br | H | NHCOOMe | |
| 29-49 | H | 2-I | H | NHCOOMe | |
| 29-50 | H | 3-I | H | NHCOOMe | |
| 29-51 | H | 4-I | H | NHCOOMe | |
| 29-52 | 3-Cl | 2-Cl | H | NHCOOMe | |
| 29-53 | 4-Cl | 2-Cl | H | NHCOOMe | |
| 29-54 | 5-Cl | 2-Cl | H | NHCOOMe | |
| 29-55 | 6-Cl | 2-Cl | H | NHCOOMe | |
| 29-56 | 4-Cl | 3-Cl | H | NHCOOMe | |
| 29-57 | 5-Cl | 3-Cl | H | NHCOOMe | |
| 29-58 | H | H | H | NHCOOEt | |
| 29-59 | H | 2-F | H | NHCOOEt | |
| 29-60 | H | 3-F | H | NHCOOEt | |
| 29-61 | H | 4-F | H | NHCOOEt | |
| 29-62 | H | 2-Cl | H | NHCOOEt | |
| 29-63 | H | 3-Cl | H | NHCOOEt | |
| 29-64 | H | 4-Cl | H | NHCOOEt | NMR see below |
| 29-65 | H | 2-Br | H | NHCOOEt | |
| 29-66 | H | 3-Br | H | NHCOOEt | |
| 29-67 | H | 4-Br | H | NHCOOEt | |
| 29-68 | H | 2-I | H | NHCOOEt | |
| 29-69 | H | 3-I | H | NHCOOEt | |
| 29-70 | H | 4-I | H | NHCOOEt | |
| 29-71 | 3-Cl | 2-Cl | H | NHCOOEt | |
| 29-72 | 4-Cl | 2-Cl | H | NHCOOEt | |
| 29-73 | 5-Cl | 2-Cl | H | NHCOOEt | |
| 29-74 | 6-Cl | 2-Cl | H | NHCOOEt | |
| 29-75 | 4-Cl | 3-Cl | H | NHCOOEt | |
| 29-76 | 5-Cl | 3-Cl | H | NHCOOEt | |
| 29-77 | H | H | H | N(COOMe)₂ | |
| 29-78 | H | 2-F | H | N(COOMe)₂ | |
| 29-79 | H | 3-F | H | N(COOMe)₂ | |
| 29-80 | H | 4-F | H | N(COOMe)₂ | |
| 29-81 | H | 2-Cl | H | N(COOMe)₂ | |
| 29-82 | H | 3-Cl | H | N(COOMe)₂ | |
| 29-83 | H | 4-Cl | H | N(COOMe)₂ | |
| 29-84 | H | 2-Br | H | N(COOMe)₂ | |
| 29-85 | H | 3-Br | H | N(COOMe)₂ | |
| 29-86 | H | 4-Br | H | N(COOMe)₂ | |
| 29-87 | H | 2-I | H | N(COOMe)₂ | |
| 29-88 | H | 3-I | H | N(COOMe)₂ | |
| 29-89 | H | 4-I | H | N(COOMe)₂ | |
| 29-90 | 3-Cl | 2-Cl | H | N(COOMe)₂ | |
| 29-91 | 4-Cl | 2-Cl | H | N(COOMe)₂ | |
| 29-92 | 5-Cl | 2-Cl | H | N(COOMe)₂ | |
| 29-93 | 6-Cl | 2-Cl | H | N(COOMe)₂ | |
| 29-94 | 4-Cl | 3-Cl | H | N(COOMe)₂ | |
| 29-95 | 5-Cl | 3-Cl | H | N(COOMe)₂ | |
| 29-96 | H | H | H | N(COOEt)₂ | |
| 29-97 | H | 2-F | H | N(COOEt)₂ | |
| 29-98 | H | 3-F | H | N(COOEt)₂ | |
| 29-99 | H | 4-F | H | N(COOEt)₂ | |
| 29-100 | H | 2-Cl | H | N(COOEt)₂ | |
| 29-101 | H | 3-Cl | H | N(COOEt)₂ | |
| 29-102 | H | 4-Cl | H | N(COOEt)₂ | |
| 29-103 | H | 2-Br | H | N(COOEt)₂ | |
| 29-104 | H | 3-Br | H | N(COOEt)₂ | |
| 29-105 | H | 4-Br | H | N(COOEt)₂ | |
| 29-106 | H | 2-I | H | N(COOEt)₂ | |
| 29-107 | H | 3-I | H | N(COOEt)₂ | |
| 29-108 | H | 4-I | H | N(COOEt)₂ | |
| 29-109 | 3-Cl | 2-Cl | H | N(COOEt)₂ | |
| 29-110 | 4-Cl | 2-Cl | H | N(COOEt)₂ | |
| 29-111 | 5-Cl | 2-Cl | H | N(COOEt)₂ | |
| 29-112 | 6-Cl | 2-Cl | H | N(COOEt)₂ | |
| 29-113 | 4-Cl | 3-Cl | H | N(COOEt)₂ | |
| 29-114 | 5-Cl | 3-Cl | H | N(COOEt)₂ | |
| 29-115 | H | H | H | NHCOC₂H₅ | |
| 29-116 | H | 2-F | H | NHCOC₂H₅ | |
| 29-117 | H | 3-F | H | NHCOC₂H₅ | |
| 29-118 | H | 4-F | H | NHCOC₂H₅ | |
| 29-119 | H | 2-Cl | H | NHCOC₂H₅ | |
| 29-120 | H | 3-Cl | H | NHCOC₂H₅ | |
| 29-121 | H | 4-Cl | H | NHCOC₂H₅ | |
| 29-122 | H | 2-Br | H | NHCOC₂H₅ | |
| 29-123 | H | 3-Br | H | NHCOC₂H₅ | |
| 29-124 | H | 4-Br | H | NHCOC₂H₅ | |
| 29-125 | H | 2-I | H | NHCOC₂H₅ | |
| 29-126 | H | 3-I | H | NHCOC₂H₅ | |
| 29-127 | H | 4-I | H | NHCOC₂H₅ | |
| 29-128 | 3-Cl | 2-Cl | H | NHCOC₂H₅ | |
| 29-129 | 4-Cl | 2-Cl | H | NHCOC₂H₅ | |
| 29-130 | 5-Cl | 2-Cl | H | NHCOC₂H₅ | |
| 29-131 | 6-Cl | 2-Cl | H | NHCOC₂H₅ | |
| 29-132 | 4-Cl | 3-Cl | H | NHCOC₂H₅ | |
| 29-133 | 5-Cl | 3-Cl | H | NHCOC₂H₅ | |
| 29-134 | H | H | H | N(COC₂H₅)₂ | |
| 29-135 | H | 2-F | H | N(COC₂H₅)₂ | |
| 29-136 | H | 3-F | H | N(COC₂H₅)₂ | |
| 29-137 | H | 4-F | H | N(COC₂H₅)₂ | |
| 29-138 | H | 2-Cl | H | N(COC₂H₅)₂ | |
| 29-139 | H | 3-Cl | H | N(COC₂H₅)₂ | |
| 29-140 | H | 4-Cl | H | N(COC₂H₅)₂ | |
| 29-141 | H | 2-Br | H | N(COC₂H₅)₂ | |
| 29-142 | H | 3-Br | H | N(COC₂H₅)₂ | |
| 29-143 | H | 4-Br | H | N(COC₂H₅)₂ | 1H: 1.02, 2.53 |
| | | | | | ppm, COCH₂CH₃ |
| 29-144 | H | 2-I | H | N(COC₂H₅)₂ | |
| 29-145 | H | 3-I | H | N(COC₂H₅)₂ | |
| 29-146 | H | 4-I | H | N(COC₂H₅)₂ | |
| 29-147 | 3-Cl | 2-Cl | H | N(COC₂H₅)₂ | |
| 29-148 | 4-Cl | 2-Cl | H | N(COC₂H₅)₂ | |
| 29-149 | 5-Cl | 2-Cl | H | N(COC₂H₅)₂ | |
| 29-150 | 6-Cl | 2-Cl | H | N(COC₂H₅)₂ | |
| 29-151 | 4-Cl | 3-Cl | H | N(COC₂H₅)₂ | |
| 29-152 | 5-Cl | 3-Cl | H | N(COC₂H₅)₂ | |
| 29-153 | 4-OMe | 3-F | 5-F | NHCOCH₃ | NMR see below |
| 29-154 | 4-Me | 3-OMe | H | NHCOCH₃ | NMR see below |
| 29-155 | 4-Cl | 3-OCF₃ | H | NHCOCH₃ | NMR see below |
| 29-156 | H | 4-OCHF₂ | H | NHCOCH₃ | NMR see below |
| 29-157 | 4-F | 3-Cl | H | NHCOCH₃ | NMR see below |
| 29-158 | 4-OMe | 3-F | 5-F | N(COCH₃)₂ | NMR see below |
| 29-159 | 4-Me | 3-OMe | H | N(COCH₃)₂ | NMR see below |
| 29-160 | 4-Cl | 3-OCF₃ | H | N(COCH₃)₂ | NMR see below |
| 29-161 | H | 4-OCHF₂ | H | N(COCH₃)₂ | NMR see below |
| 29-162 | 4-F | 3-Cl | H | N(COCH₃)₂ | NMR see below |
| 29-163 | 3-CF₃ | 5-CF₃ | | NHCOCH₃ | NMR see below |
| 29-164 | 3-Cl | 5-CF₃ | | NHCOCH₃ | NMR see below |
| 29-165 | 3-Cl | 5-CF₃ | | NHCO₂CH₂CCH | NMR see below |
| 29-166 | 3-Cl | 5-CF₃ | | N(CO₂CH₂CCH)₂ | NMR see below |
| 29-167 | 3-CF₃ | 5-CF₃ | | N(CO₂CH₂CCH)₂ | NMR see below |

TABLE 30

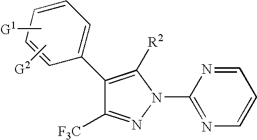

| Example | | | | |
|---|---|---|---|---|
| No. | G¹ | G² | R² | Physical data |
| 30-01 | H | H | NH—CO—COOMe | |
| 30-02 | 2-F | H | NH—CO—COOMe | |
| 30-03 | 3-F | H | NH—CO—COOMe | |
| 30-04 | 4-F | H | NH—CO—COOMe | |
| 30-05 | 2-Cl | H | NH—CO—COOMe | |
| 30-06 | 3-Cl | H | NH—CO—COOMe | |
| 30-07 | 4-Cl | H | NH—CO—COOMe | |
| 30-08 | 2-Br | H | NH—CO—COOMe | |
| 30-09 | 3-Br | H | NH—CO—COOMe | |
| 30-10 | 4-Br | H | NH—CO—COOMe | 1H: 3.90 ppm, COOMe |
| 30-11 | 2-I | H | NH—CO—COOMe | |
| 30-12 | 3-I | H | NH—CO—COOMe | |
| 30-13 | 4-I | H | NH—CO—COOMe | |
| 30-14 | 2-Cl | 3-Cl | NH—CO—COOMe | |
| 30-15 | 2-Cl | 4-Cl | NH—CO—COOMe | |
| 30-16 | 2-Cl | 5-Cl | NH—CO—COOMe | |
| 30-17 | 2-Cl | 6-Cl | NH—CO—COOMe | |
| 30-18 | 3-Cl | 4-Cl | NH—CO—COOMe | |
| 30-19 | 3-Cl | 5-Cl | NH—CO—COOMe | |
| 30-20 | H | H | NH—CO—COOEt | |
| 30-21 | 2-F | H | NH—CO—COOEt | |
| 30-22 | 3-F | H | NH—CO—COOEt | |
| 30-23 | 4-F | H | NH—CO—COOEt | |
| 30-24 | 2-Cl | H | NH—CO—COOEt | |
| 30-25 | 3-Cl | H | NH—CO—COOEt | |
| 30-26 | 4-Cl | H | NH—CO—COOEt | |
| 30-27 | 2-Br | H | NH—CO—COOEt | |
| 30-28 | 3-Br | H | NH—CO—COOEt | |
| 30-29 | 4-Br | H | NH—CO—COOEt | |
| 30-30 | 2-I | H | NH—CO—COOEt | |
| 30-31 | 3-I | H | NH—CO—COOEt | |
| 30-32 | 4-I | H | NH—CO—COOEt | |
| 30-33 | 2-Cl | 3-Cl | NH—CO—COOEt | |
| 30-34 | 2-Cl | 4-Cl | NH—CO—COOEt | |
| 30-35 | 2-Cl | 5-Cl | NH—CO—COOEt | |
| 30-36 | 2-Cl | 6-Cl | NH—CO—COOEt | |
| 30-37 | 3-Cl | 4-Cl | NH—CO—COOEt | |
| 30-38 | 3-Cl | 5-Cl | NH—CO—COOEt | |

TABLE 31

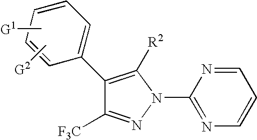

| Example | | | | |
|---|---|---|---|---|
| No. | G¹ | G² | R² | Physical data |
| 31-01 | H | H | NHCO-Ph | |
| 31-02 | 2-F | H | NHCO-Ph | |
| 31-03 | 3-F | H | NHCO-Ph | |
| 31-04 | 4-F | H | NHCO-Ph | |
| 31-05 | 2-Cl | H | NHCO-Ph | |
| 31-06 | 3-Cl | H | NHCO-Ph | |
| 31-07 | 4-Cl | H | NHCO-Ph | |
| 31-08 | 2-Br | H | NHCO-Ph | |
| 31-09 | 3-Br | H | NHCO-Ph | |
| 31-10 | 4-Br | H | NHCO-Ph | |
| 31-11 | 2-I | H | NHCO-Ph | |
| 31-12 | 3-I | H | NHCO-Ph | |
| 31-13 | 4-I | H | NHCO-Ph | |
| 31-14 | 2-Cl | 3-Cl | NHCO-Ph | |
| 31-15 | 2-Cl | 4-Cl | NHCO-Ph | |
| 31-16 | 2-Cl | 5-Cl | NHCO-Ph | |
| 31-17 | 2-Cl | 6-Cl | NHCO-Ph | |
| 31-18 | 3-Cl | 4-Cl | NHCO-Ph | |
| 31-19 | 3-Cl | 5-Cl | NHCO-Ph | |
| 31-20 | H | H | NHCO-2-Cl—C₆H₄ | |
| 31-21 | 2-F | H | NHCO-2-Cl—C₆H₄ | |
| 31-22 | 3-F | H | NHCO-2-Cl—C₆H₄ | |
| 31-23 | 4-F | H | NHCO-2-Cl—C₆H₄ | |
| 31-24 | 2-Cl | H | NHCO-2-Cl—C₆H₄ | |
| 31-25 | 3-Cl | H | NHCO-2-Cl—C₆H₄ | |
| 31-26 | 4-Cl | H | NHCO-2-Cl—C₆H₄ | |
| 31-27 | 2-Br | H | NHCO-2-Cl—C₆H₄ | |
| 31-28 | 3-Br | H | NHCO-2-Cl—C₆H₄ | |
| 31-29 | 4-Br | H | NHCO-2-Cl—C₆H₄ | 19F: −61.25 ppm, CF₃. |
| 31-30 | 2-I | H | NHCO-2-Cl—C₆H₄ | |
| 31-31 | 3-I | H | NHCO-2-Cl—C₆H₄ | |
| 31-32 | 4-I | H | NHCO-2-Cl—C₆H₄ | |
| 31-33 | 2-Cl | 3-Cl | NHCO-2-Cl—C₆H₄ | |
| 31-34 | 2-Cl | 4-Cl | NHCO-2-Cl—C₆H₄ | |
| 31-35 | 2-Cl | 5-Cl | NHCO-2-Cl—C₆H₄ | |
| 31-36 | 2-Cl | 6-Cl | NHCO-2-Cl—C₆H₄ | |
| 31-37 | 3-Cl | 4-Cl | NHCO-2-Cl—C₆H₄ | |
| 31-38 | 3-Cl | 5-Cl | NHCO-2-Cl—C₆H₄ | |
| 31-39 | H | H | NHCO-3-Cl—C₆H₄ | |
| 31-40 | 2-F | H | NHCO-3-Cl—C₆H₄ | |
| 31-41 | 3-F | H | NHCO-3-Cl—C₆H₄ | |
| 31-42 | 4-F | H | NHCO-3-Cl—C₆H₄ | |
| 31-43 | 2-Cl | H | NHCO-3-Cl—C₆H₄ | |
| 31-44 | 3-Cl | H | NHCO-3-Cl—C₆H₄ | |
| 31-45 | 4-Cl | H | NHCO-3-Cl—C₆H₄ | |
| 31-46 | 2-Br | H | NHCO-3-Cl—C₆H₄ | |
| 31-47 | 3-Br | H | NHCO-3-Cl—C₆H₄ | |
| 31-48 | 4-Br | H | NHCO-3-Cl—C₆H₄ | |
| 31-49 | 2-I | H | NHCO-3-Cl—C₆H₄ | |
| 31-50 | 3-I | H | NHCO-3-Cl—C₆H₄ | |
| 31-51 | 4-I | H | NHCO-3-Cl—C₆H₄ | |
| 31-52 | 2-Cl | 3-Cl | NHCO-3-Cl—C₆H₄ | |
| 31-53 | 2-Cl | 4-Cl | NHCO-3-Cl—C₆H₄ | |
| 31-54 | 2-Cl | 5-Cl | NHCO-3-Cl—C₆H₄ | |
| 31-55 | 2-Cl | 6-Cl | NHCO-3-Cl—C₆H₄ | |
| 31-56 | 3-Cl | 4-Cl | NHCO-3-Cl—C₆H₄ | |
| 31-57 | 3-Cl | 5-Cl | NHCO-3-Cl—C₆H₄ | |
| 31-58 | H | H | NHCO-4-Cl—C₆H₄ | |
| 31-59 | 2-F | H | NHCO-2-F—C₆H₄ | |
| 31-60 | 3-F | H | NHCO-2-F—C₆H₄ | |
| 31-61 | 4-F | H | NHCO-2-F—C₆H₄ | |
| 31-62 | 2-Cl | H | NHCO-2-F—C₆H₄ | |
| 31-63 | 3-Cl | H | NHCO-2-F—C₆H₄ | |
| 31-64 | 4-Cl | H | NHCO-2-F—C₆H₄ | |
| 31-65 | 2-Br | H | NHCO-2-F—C₆H₄ | |
| 31-66 | 3-Br | H | NHCO-2-F—C₆H₄ | |
| 31-67 | 4-Br | H | NHCO-2-F—C₆H₄ | |
| 31-68 | 2-I | H | NHCO-2-F—C₆H₄ | |
| 31-69 | 3-I | H | NHCO-2-F—C₆H₄ | |
| 31-70 | 4-I | H | NHCO-2-F—C₆H₄ | |
| 31-71 | 2-Cl | 3-Cl | NHCO-2-F—C₆H₄ | |
| 31-72 | 2-Cl | 4-Cl | NHCO-2-F—C₆H₄ | |
| 31-73 | 2-Cl | 5-Cl | NHCO-2-F—C₆H₄ | |
| 31-74 | 2-Cl | 6-Cl | NHCO-2-F—C₆H₄ | |
| 31-75 | 3-Cl | 4-Cl | NHCO-2-F—C₆H₄ | |
| 31-76 | 3-Cl | 5-Cl | NHCO-2-F—C₆H₄ | |
| 31-77 | H | H | NHCO-3-F—C₆H₄ | |
| 31-78 | 2-F | H | NHCO-3-F—C₆H₄ | |
| 31-79 | 3-F | H | NHCO-3-F—C₆H₄ | |
| 31-80 | 4-F | H | NHCO-3-F—C₆H₄ | |
| 31-81 | 2-Cl | H | NHCO-3-F—C₆H₄ | |
| 31-82 | 3-Cl | H | NHCO-3-F—C₆H₄ | |
| 31-83 | 4-Cl | H | NHCO-3-F—C₆H₄ | |
| 31-84 | 2-Br | H | NHCO-3-F—C₆H₄ | |
| 31-85 | 3-Br | H | NHCO-3-F—C₆H₄ | |
| 31-86 | 4-Br | H | NHCO-3-F—C₆H₄ | |
| 31-87 | 2-I | H | NHCO-3-F—C₆H₄ | |
| 31-88 | 3-I | H | NHCO-3-F—C₆H₄ | |
| 31-89 | 4-I | H | NHCO-3-F—C₆H₄ | |
| 31-90 | 2-Cl | 3-Cl | NHCO-3-F—C₆H₄ | |
| 31-91 | 2-Cl | 4-Cl | NHCO-3-F—C₆H₄ | |
| 31-92 | 2-Cl | 5-Cl | NHCO-3-F—C₆H₄ | |
| 31-93 | 2-Cl | 6-Cl | NHCO-3-F—C₆H₄ | |
| 31-94 | 3-Cl | 4-Cl | NHCO-3-F—C₆H₄ | |
| 31-95 | 3-Cl | 5-Cl | NHCO-3-F—C₆H₄ | |
| 31-96 | H | H | NHCO-4-F—C₆H₄ | |
| 31-97 | 2-F | H | NHCO-4-F—C₆H₄ | |
| 31-98 | 3-F | H | NHCO-4-F—C₆H₄ | |
| 31-99 | 4-F | H | NHCO-4-F—C₆H₄ | |
| 31-100 | 2-Cl | H | NHCO-4-F—C₆H₄ | |
| 31-101 | 3-Cl | H | NHCO-4-F—C₆H₄ | |
| 31-102 | 4-Cl | H | NHCO-4-F—C₆H₄ | |
| 31-103 | 2-Br | H | NHCO-4-F—C₆H₄ | |
| 31-104 | 3-Br | H | NHCO-4-F—C₆H₄ | |
| 31-105 | 4-Br | H | NHCO-4-F—C₆H₄ | |
| 31-106 | 2-I | H | NHCO-4-F—C₆H₄ | |
| 31-107 | 3-I | H | NHCO-4-F—C₆H₄ | |
| 31-108 | 4-I | H | NHCO-4-F—C₆H₄ | |
| 31-109 | 2-Cl | 3-Cl | NHCO-4-F—C₆H₄ | |
| 31-110 | 2-Cl | 4-Cl | NHCO-4-F—C₆H₄ | |
| 31-111 | 2-Cl | 5-Cl | NHCO-4-F—C₆H₄ | |
| 31-112 | 2-Cl | 6-Cl | NHCO-4-F—C₆H₄ | |
| 31-113 | 3-Cl | 4-Cl | NHCO-4-F—C₆H₄ | |
| 31-114 | 3-Cl | 5-Cl | NHCO-4-F—C₆H₄ | |

TABLE 32

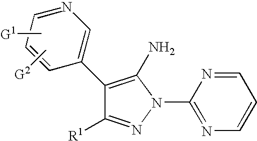

| Example | | | |
|---|---|---|---|
| No. | R¹ | G¹ | Physical data |
| 32-01 | CF₃ | H | |
| 32-02 | CF₃ | 2-Cl | |
| 32-03 | CF₃ | 6-Cl | NMR see below |
| 32-04 | CF₃ | 5-Cl | |
| 32-05 | CF₃ | 4-Cl | |
| 32-06 | CF₃ | 5-F | |
| 32-07 | CF₃ | 5-Br | |
| 32-08 | CF₃ | 5-I | |
| 32-09 | CF₃ | 2-OMe | |
| 32-10 | CF₃ | 4-OMe | |
| 32-11 | CF₃ | 5-OMe | |
| 32-12 | CF₃ | 6-OMe | NMR see below |
| 32-13 | CF₃ | 2-OEt | |
| 32-14 | CF₃ | 4-OEt | |
| 32-15 | CF₃ | 5-OEt | |
| 32-16 | CF₃ | 6-OEt | |
| 32-17 | CF₃ | 6-CF₃ | NMR see below |
| 32-18 | Me | H | |
| 32-19 | Me | 2-Cl | |
| 32-20 | Me | 6-Cl | |
| 32-21 | Me | 5-Cl | |
| 32-22 | Me | 4-Cl | |
| 32-23 | Me | 5-F | |
| 32-24 | Me | 5-Br | |
| 32-25 | Me | 5-I | |
| 32-26 | Me | 2-OMe | |
| 32-27 | Me | 4-OMe | |
| 32-28 | Me | 5-OMe | |
| 32-29 | Me | 6-OMe | |
| 32-30 | Me | 2-OEt | |
| 32-31 | Me | 4-OEt | |
| 32-32 | Me | 5-OEt | |
| 32-33 | Me | 6-OEt | |
| 32-34 | Me | 6-CF₃ | |
| 32-35 | Et | H | |
| 32-36 | Et | 2-Cl | |
| 32-37 | Et | 6-Cl | lgP = 1.76 |
| 32-38 | Et | 5-Cl | |
| 32-39 | Et | 4-Cl | |
| 32-40 | Et | 5-F | |
| 32-41 | Et | 5-Br | |
| 32-42 | Et | 5-I | |
| 32-43 | Et | 2-OMe | |
| 32-44 | Et | 4-OMe | |
| 32-45 | Et | 5-OMe | |
| 32-46 | Et | 6-OMe | |
| 32-47 | Et | 2-OEt | |
| 32-48 | Et | 4-OEt | |
| 32-49 | Et | 5-OEt | |
| 32-50 | Et | 6-OEt | |
| 32-51 | Et | 6-CF₃ | |
| 32-52 | CF₃ | 6-NMe₂ | NMR see below |
| 32 53 | CF₃ | 6-OiPr | NMR see below |
| 32-54 | CF₃ | G1 = 6-OMe | NMR see below |
| | | G2 = 5-Cl | |

TABLE 33

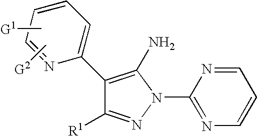

| Example | | | | |
|---|---|---|---|---|
| No. | R¹ | G¹ | G² | Physical data |
| 33-01 | CF₃ | H | H | |
| 33-02 | CF₃ | 3-Cl | H | |
| 33-03 | CF₃ | 4-Cl | H | |
| 33-04 | CF₃ | 5-Cl | H | |
| 33-05 | CF₃ | 6-Cl | H | |
| 33-06 | CF₃ | 5-F | H | |
| 33-07 | CF₃ | 5-Br | H | |
| 33-08 | CF₃ | 5-I | H | |
| 33-09 | CF₃ | 3-OMe | H | |
| 33-10 | CF₃ | 4-OMe | H | |
| 33-11 | CF₃ | 5-OMe | H | |
| 33-12 | CF₃ | 6-OMe | H | |
| 33-13 | CF₃ | 3-OEt | H | |
| 33-14 | CF₃ | 4-OEt | H | |
| 33-15 | CF₃ | 5-OEt | H | |
| 33-16 | CF₃ | 6-OEt | H | |
| 33-17 | CF₃ | 3-CF₃ | H | |
| 33-18 | CF₃ | 4-CF₃ | H | |
| 33-19 | CF₃ | 5-CF₃ | H | |
| 33-20 | CF₃ | 6-CF₃ | H | |
| 33-21 | CF₃ | 3-Cl | 5-Cl | |
| 33-22 | CH₃ | H | H | |
| 33-23 | CH₃ | 3-Cl | H | |
| 33-24 | CH₃ | 4-Cl | H | |
| 33-25 | CH₃ | 5-Cl | H | |
| 33-26 | CH₃ | 6-Cl | H | |
| 33-27 | CH₃ | 5-F | H | |
| 33-28 | CH₃ | 5-Br | H | |
| 33-29 | CH₃ | 5-I | H | |
| 33-30 | CH₃ | 3-OMe | H | |
| 33-31 | CH₃ | 4-OMe | H | |
| 33-32 | CH₃ | 5-OMe | H | |
| 33-33 | CH₃ | 6-OMe | H | |
| 33-34 | CH₃ | 3-OEt | H | |
| 33-35 | CH₃ | 4-OEt | H | |
| 33-36 | CH₃ | 5-OEt | H | |
| 33-37 | CH₃ | 6-OEt | H | |
| 33-38 | CH₃ | 3-CF₃ | H | |
| 33-39 | CH₃ | 4-CF₃ | H | |
| 33-40 | CH₃ | 5-CF₃ | H | |
| 33-41 | CH₃ | 6-CF₃ | H | |
| 33-42 | CH₃ | 3-Cl | 5-Cl | |
| 33-43 | C₂H₅ | H | H | |
| 33-44 | C₂H₅ | 3-Cl | H | |
| 33-45 | C₂H₅ | 4-Cl | H | |
| 33-46 | C₂H₅ | 5-Cl | H | |
| 33-47 | C₂H₅ | 6-Cl | H | |
| 33-48 | C₂H₅ | 5-F | H | |
| 33-49 | C₂H₅ | 5-Br | H | |
| 33-50 | C₂H₅ | 5-I | H | |
| 33-51 | C₂H₅ | 3-OMe | H | |
| 33-52 | C₂H₅ | 4-OMe | H | |
| 33-53 | C₂H₅ | 5-OMe | H | |
| 33-54 | C₂H₅ | 6-OMe | H | |
| 33-55 | C₂H₅ | 3-OEt | H | |
| 33-56 | C₂H₅ | 4-OEt | H | |
| 33-57 | C₂H₅ | 5-OEt | H | |
| 33-58 | C₂H₅ | 6-OEt | H | |
| 33-59 | C₂H₅ | 3-CF₃ | H | |
| 33-60 | C₂H₅ | 4-CF₃ | H | |
| 33-61 | C₂H₅ | 5-CF₃ | H | |
| 33-62 | C₂H₅ | 6-CF₃ | H | |
| 33-63 | C₂H₅ | 3-Cl | 5-Cl | |

TABLE 34

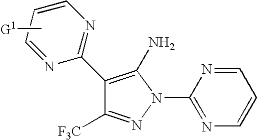

| Example | | |
|---|---|---|
| No. | G¹ | Physical data |
| 34-01 | H | |
| 34-02 | 5-F | |
| 34-03 | 5-Cl | |
| 34-04 | 5-Br | |
| 34-05 | 5-I | |
| 34-06 | 5-CF₃ | |

TABLE 35

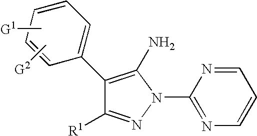

| Example | | | | |
|---|---|---|---|---|
| No. | G¹ | G² | R¹ | Physical data |
| 35-01 | H | H | cyclopropyl | |
| 35-02 | 2-F | H | cyclopropyl | |
| 35-03 | 3-F | H | cyclopropyl | |
| 35-04 | 4-F | H | cyclopropyl | |
| 35-05 | 2-Cl | H | cyclopropyl | |
| 35-06 | 3-Cl | H | cyclopropyl | |
| 35-07 | 4-Cl | H | cyclopropyl | logP 2.66; ¹H-NMR |
| | | | | (CD₃CN): 8.74 |
| | | | | (d, 2H), 7.22 (t, 1H) |
| 35-08 | 2-Br | H | cyclopropyl | |
| 35-09 | 3-Br | H | cyclopropyl | |
| 35-10 | 4-Br | H | cyclopropyl | |
| 35-11 | 2-I | H | cyclopropyl | |
| 35-12 | 3-I | H | cyclopropyl | |
| 35-13 | 4-I | H | cyclopropyl | |
| 35-14 | 2-Cl | 3-Cl | cyclopropyl | |
| 35-15 | 2-Cl | 4-Cl | cyclopropyl | |
| 35-16 | 2-Cl | 5-Cl | cyclopropyl | |
| 35-17 | 2-Cl | 6-Cl | cyclopropyl | |
| 35-18 | 2-Cl | 4-F | cyclopropyl | |
| 35-19 | 3-Cl | 4-Cl | cyclopropyl | |
| 35-20 | 3-Cl | 5-Cl | cyclopropyl | NMR see below |
| 35-21 | H | H | 1-Cl-cyclopropyl | |
| 35-22 | 2-F | H | 1-Cl-cyclopropyl | |
| 35-23 | 3-F | H | 1-Cl-cyclopropyl | |
| 35-24 | 4-F | H | 1-Cl-cyclopropyl | |
| 35-25 | 2-Cl | H | 1-Cl-cyclopropyl | |
| 35-26 | 3-Cl | H | 1-Cl-cyclopropyl | |
| 35-27 | 4-Cl | H | 1-Cl-cyclopropyl | |
| 35-28 | 2-Br | H | 1-Cl-cyclopropyl | |
| 35-29 | 3-Br | H | 1-Cl-cyclopropyl | |
| 35-30 | 4-Br | H | 1-Cl-cyclopropyl | |
| 35-31 | 2-I | H | 1-Cl-cyclopropyl | |
| 35-32 | 3-I | H | 1-Cl-cyclopropyl | |
| 35-33 | 4-I | H | 1-Cl-cyclopropyl | |
| 35-34 | 2-Cl | 3-Cl | 1-Cl-cyclopropyl | |
| 35-35 | 2-Cl | 4-Cl | 1-Cl-cyclopropyl | |
| 35-36 | 2-Cl | 5-Cl | 1-Cl-cyclopropyl | |
| 35-37 | 2-Cl | 6-Cl | 1-Cl-cyclopropyl | |
| 35-38 | 3-Cl | 4-Cl | 1-Cl-cyclopropyl | |
| 35-39 | 3-Cl | 5-Cl | 1-Cl-cyclopropyl | NMR see below |
| 35-40 | H | H | 1-CF₃-cyclopropyl | |
| 35-41 | 2-F | H | 1-CF₃-cyclopropyl | |
| 35-42 | 3-F | H | 1-CF₃-cyclopropyl | |
| 35-43 | 4-F | H | 1-CF₃-cyclopropyl | |
| 35-44 | 2-Cl | H | 1-CF₃-cyclopropyl | |
| 35-45 | 3-Cl | H | 1-CF₃-cyclopropyl | |
| 35-46 | 4-Cl | H | 1-CF₃-cyclopropyl | |
| 35-47 | 2-Br | H | 1-CF₃-cyclopropyl | |
| 35-48 | 3-Br | H | 1-CF₃-cyclopropyl | |
| 35-49 | 4-Br | H | 1-CF₃-cyclopropyl | |
| 35-50 | 2-1 | H | 1-CF₃-cyclopropyl | |
| 35-51 | 3-I | H | 1-CF₃-cyclopropyl | |
| 35-52 | 4-I | H | 1-CF₃-cyclopropyl | |
| 35-53 | 2-Cl | 3-Cl | 1-CF₃-cyclopropyl | |
| 35-54 | 2-Cl | 4-Cl | 1-CF₃-cyclopropyl | |
| 35-55 | 2-Cl | 5-Cl | 1-CF₃-cyclopropyl | |
| 35-56 | 2-Cl | 6-Cl | 1-CF₃-cyclopropyl | |
| 35-57 | 3-Cl | 4-Cl | 1-CF₃-cyclopropyl | |
| 35-58 | 3-Cl | 5-Cl | 1-CF₃-cyclopropyl | |
| 35-59 | H | H | cyclobutyl | |
| 35-60 | 2-F | H | cyclobutyl | |
| 35-61 | 3-F | H | cyclobutyl | |
| 35-62 | 4-F | H | cyclobutyl | |
| 35-63 | 2-Cl | H | cyclobutyl | |
| 35-64 | 3-Cl | H | cyclobutyl | |
| 35-65 | 4-Cl | H | cyclobutyl | |
| 35-66 | 2-Br | H | cyclobutyl | |
| 35-67 | 3-Br | H | cyclobutyl | |
| 35-68 | 4-Br | H | cyclobutyl | |
| 35-69 | 2-I | H | cyclobutyl | |
| 35-70 | 3-I | H | cyclobutyl | |
| 35-71 | 4-I | H | cyclobutyl | |
| 35-72 | 2-Cl | 3-Cl | cyclobutyl | |
| 35-73 | 2-Cl | 4-Cl | cyclobutyl | |
| 35-74 | 2-Cl | 5-Cl | cyclobutyl | |
| 35-75 | 2-Cl | 6-Cl | cyclobutyl | |
| 35-76 | 3-Cl | 4-Cl | cyclobutyl | |
| 35-77 | 3-Cl | 5-Cl | cyclobutyl | NMR see below |
| 35-78 | H | H | cyclopentyl | |
| 35-79 | 2-F | H | cyclopentyl | |
| 35-80 | 3-F | H | cyclopentyl | |
| 35-81 | 4-F | H | cyclopentyl | |
| 35-82 | 2-Cl | H | cyclopentyl | |
| 35-83 | 3-Cl | H | cyclopentyl | |
| 35-84 | 4-Cl | H | cyclopentyl | |
| 35-85 | 2-Br | H | cyclopentyl | |
| 35-86 | 3-Br | H | cyclopentyl | |
| 35-87 | 4-Br | H | cyclopentyl | logP 3.34 |
| 35-88 | 2-I | H | cyclopentyl | |
| 35-89 | 3-I | H | cyclopentyl | |
| 35-90 | 4-I | H | cyclopentyl | |
| 35-91 | 2-Cl | 3-Cl | cyclopentyl | |
| 35-92 | 2-Cl | 4-Cl | cyclopentyl | |
| 35-93 | 2-Cl | 5-Cl | cyclopentyl | |
| 35-94 | 2-Cl | 6-Cl | cyclopentyl | |
| 35-95 | 3-Cl | 4-Cl | cyclopentyl | |
| 35-96 | 3-Cl | 5-Cl | cyclopentyl | |
| 35-97 | H | H | cyclohexyl | |
| 35-98 | 2-F | H | cyclohexyl | |
| 35-99 | 3-F | H | cyclohexyl | |
| 35-100 | 4-F | H | cyclohexyl | |
| 35-101 | 2-Cl | H | cyclohexyl | |
| 35-102 | 3-Cl | H | cyclohexyl | |
| 35-103 | 4-Cl | H | cyclohexyl | |
| 35-104 | 2-Br | H | cyclohexyl | |
| 35-105 | 3-Br | H | cyclohexyl | |
| 35-106 | 4-Br | H | cyclohexyl | |
| 35-107 | 2-I | H | cyclohexyl | |
| 35-108 | 3-I | H | cyclohexyl | |
| 35-109 | 4-I | H | cyclohexyl | |
| 35-110 | 2-Cl | 3-Cl | cyclohexyl | |
| 35-111 | 2-Cl | 4-Cl | cyclohexyl | |
| 35-112 | 2-Cl | 5-Cl | cyclohexyl | |
| 35-113 | 2-Cl | 6-Cl | cyclohexyl | |
| 35-114 | 3-Cl | 4-Cl | cyclohexyl | |
| 35-115 | 3-Cl | 5-Cl | cyclohexyl | |
| 35-116 | H | H | 1-F-cyclopropyl | |
| 35-117 | 2-F | H | 1-F-cyclopropyl | |
| 35-118 | 3-F | H | 1-F-cyclopropyl | |
| 35-119 | 4-F | H | 1-F-cyclopropyl | |
| 35-120 | 2-Cl | H | 1-F-cyclopropyl | |
| 35-121 | 3-Cl | H | 1-F-cyclopropyl | |
| 35-122 | 4-Cl | H | 1-F-cyclopropyl | |
| 35-123 | 2-Br | H | 1-F-cyclopropyl | |
| 35-124 | 3-Br | H | 1-F-cyclopropyl | |
| 35-125 | 4-Br | H | 1-F-cyclopropyl | 19F: −175.31 ppm, |
| | | | | 1-FcPr |
| 35-126 | 2-I | H | 1-F-cyclopropyl | |
| 35-127 | 3-I | H | 1-F-cyclopropyl | |
| 35-128 | 4-I | H | 1-F-cyclopropyl | |
| 35-129 | 2-Cl | 3-Cl | 1-F-cyclopropyl | |
| 35-130 | 2-Cl | 4-Cl | 1-F-cyclopropyl | |
| 35-131 | 2-Cl | 5-Cl | 1-F-cyclopropyl | |
| 35-132 | 2-Cl | 6-Cl | 1-F-cyclopropyl | |
| 35-133 | 3-Cl | 4-Cl | 1-F-cyclopropyl | |
| 35-134 | 3-Cl | 5-Cl | 1-F-cyclopropyl | NMR see below |
| 35-135 | H | H | CH₂-cyclopropyl | |
| 35-136 | 2-F | H | CH₂-cyclopropyl | |
| 35-137 | 3-F | H | CH₂-cyclopropyl | |
| 35-138 | 4-F | H | CH₂-cyclopropyl | |
| 35-139 | 2-Cl | H | CH₂-cyclopropyl | |
| 35-140 | 3-Cl | H | CH₂-cyclopropyl | |
| 35-141 | 4-Cl | H | CH₂-cyclopropyl | |
| 35-142 | 2-Br | H | CH₂-cyclopropyl | |
| 35-143 | 3-Br | H | CH₂-cyclopropyl | |
| 35-144 | 4-Br | H | CH₂-cyclopropyl | 1H (d6DMSO): 0.84, |
| | | | | 1H; 0.30, 2H, |
| | | | | 0.00, 2H; cyclopropyl |
| 35-145 | 2-I | H | CH₂-cyclopropyl | |
| 35-146 | 3-1 | H | CH₂-cyclopropyl | |
| 35-147 | 4-I | H | CH₂-cyclopropyl | |
| 35-148 | 2-Cl | 3-Cl | CH₂-cyclopropyl | |
| 35-149 | 2-Cl | 4-Cl | CH₂-cyclopropyl | |
| 35-150 | 2-Cl | 5-Cl | CH₂-cyclopropyl | |
| 35-151 | 2-Cl | 6-Cl | CH₂-cyclopropyl | |
| 35-152 | 3-Cl | 4-Cl | CH₂-cyclopropyl | |
| 35-153 | 3-Cl | 5-Cl | CH₂-cyclopropyl | |
| 35-154 | H | H | CH₂cyclohexyl | |
| 35-155 | 2-F | H | CH₂cyclohexyl | |
| 35-156 | 3-F | H | CH₂cyclohexyl | |
| 35-157 | 4-F | H | CH₂cyclohexyl | |
| 35-158 | 2-Cl | H | CH₂cyclohexyl | |
| 35-159 | 3-Cl | H | CH₂cyclohexyl | |
| 35-160 | 4-Cl | H | CH₂cyclohexyl | |
| 35-161 | 2-Br | H | CH₂cyclohexyl | |
| 35-162 | 3-Br | H | CH₂cyclohexyl | |
| 35-163 | 4-Br | H | CH₂cyclohexyl | 1H (d6DMSO): 2.42 |
| | | | | ppm, CH₂ |
| 35-164 | 2-I | H | CH₂cyclohexyl | |
| 35-165 | 3-I | H | CH₂cyclohexyl | |
| 35-166 | 4-I | H | CH₂cyclohexyl | |
| 35-167 | 2-Cl | 3-Cl | CH₂cyclohexyl | |
| 35-168 | 2-Cl | 4-Cl | CH₂cyclohexyl | |
| 35-169 | 2-Cl | 5-Cl | CH₂cyclohexyl | |
| 35-170 | 2-Cl | 6-Cl | CH₂cyclohexyl | |
| 35-171 | 3-Cl | 4-Cl | CH₂cyclohexyl | |
| 35-172 | 3-Cl | 5-Cl | CH₂cyclohexyl | |
| 35-173 | H | H | CH₂CH₂cyclohexyl | |
| 35-174 | 2-F | H | CH₂CH₂cyclohexyl | |
| 35-175 | 3-F | H | CH₂CH₂cyclohexyl | |
| 35-176 | 4-F | H | CH₂CH₂cyclohexyl | |
| 35-177 | 2-Cl | H | CH₂CH₂cyclohexyl | |
| 35-178 | 3-Cl | H | CH₂CH₂cyclohexyl | |
| 35-179 | 4-Cl | H | CH₂CH₂cyclohexyl | |
| 35-180 | 2-Br | H | CH₂CH₂cyclohexyl | |
| 35-181 | 3-Br | H | CH₂CH₂cyclohexyl | |
| 35-182 | 4-Br | H | CH₂CH₂cyclohexyl | 1H (d6DMSO): 0.82, |
| | | | | 1.14, 1.59, |
| | | | | cyclohexyl |
| 35-183 | 2-I | H | CH₂CH₂cyclohexyl | |
| 35-184 | 3-I | H | CH₂CH₂cyclohexyl | |
| 35-185 | 4-I | H | CH₂CH₂cyclohexyl | |
| 35-186 | 2-Cl | 3-Cl | CH₂CH₂cyclohexyl | |
| 35-187 | 2-Cl | 4-Cl | CH₂CH₂cyclohexyl | |
| 35-188 | 2-Cl | 5-Cl | CH₂CH₂cyclohexyl | |
| 35-189 | 2-Cl | 6-Cl | CH₂CH₂cyclohexyl | |
| 35-190 | 3-Cl | 4-Cl | CH₂CH₂cyclohexyl | |
| 35-191 | 3-Cl | 5-Cl | CH₂CH₂cyclohexyl | |
| 35-192 | H | H | CH₂cyclopentyl | |
| 35-193 | 2-F | H | CH₂cyclopentyl | |
| 35-194 | 3-F | H | CH₂cyclopentyl | |
| 35-195 | 4-F | H | CH₂cyclopentyl | |
| 35-196 | 2-Cl | H | CH₂cyclopentyl | |
| 35-197 | 3-Cl | H | CH₂cyclopentyl | |
| 35-198 | 4-Cl | H | CH₂cyclopentyl | |
| 35-199 | 2-Br | H | CH₂cyclopentyl | |
| 35-200 | 3-Br | H | CH₂cyclopentyl | |
| 35-201 | 4-Br | H | CH₂cyclopentyl | |
| 35-202 | 2-I | H | CH₂cyclopentyl | |
| 35-203 | 3-I | H | CH₂cyclopentyl | |
| 35-204 | 4-I | H | CH₂cyclopentyl | |
| 35-205 | 2-Cl | 3-Cl | CH₂cyclopentyl | |
| 35-206 | 2-Cl | 4-Cl | CH₂cyclopentyl | |
| 35-207 | 2-Cl | 5-Cl | CH₂cyclopentyl | |
| 35-208 | 2-Cl | 6-Cl | CH₂cyclopentyl | |
| 35-209 | 3-Cl | 4-Cl | CH₂cyclopentyl | |
| 35-210 | 3-Cl | 5-Cl | CH₂cyclopentyl | |

TABLE 36

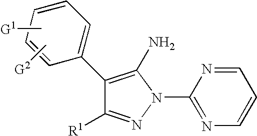

| Example | | | | |
|---|---|---|---|---|
| No. | G¹ | G² | R¹ | |
| 36-01 | H | H | CH(OMe)₂ | |
| 36-02 | 2-F | H | CH(OMe)₂ | |
| 36-03 | 3-F | H | CH(OMe)₂ | |
| 36-04 | 4-F | H | CH(OMe)₂ | |
| 36-05 | 2-Cl | H | CH(OMe)₂ | |
| 36-06 | 3-Cl | H | CH(OMe)₂ | |
| 36-07 | 4-Cl | H | CH(OMe)₂ | |
| 36-08 | 2-Br | H | CH(OMe)₂ | |
| 36-09 | 3-Br | H | CH(OMe)₂ | |
| 36-10 | 4-Br | H | CH(OMe)₂ | 1H: 5.22 ppm, |
| | | | | CH(OMe)₂ |
| 36-11 | 2-I | H | CH(OMe)₂ | |
| 36-12 | 3-I | H | CH(OMe)₂ | |
| 36-13 | 4-I | H | CH(OMe)₂ | |
| 36-14 | 2-Cl | 3-Cl | CH(OMe)₂ | |
| 36-15 | 2-Cl | 4-Cl | CH(OMe)₂ | |
| 36-16 | 2-Cl | 5-Cl | CH(OMe)₂ | |
| 36-17 | 2-Cl | 6-Cl | CH(OMe)₂ | |
| 36-18 | 3-Cl | 4-Cl | CH(OMe)₂ | |
| 36-19 | 3-Cl | 5-Cl | CH(OMe)₂ | |
| 36-20 | H | H | CH(OEt)₂ | |
| 36-21 | 2-F | H | CH(OEt)₂ | |
| 36-22 | 3-F | H | CH(OEt)₂ | |
| 36-23 | 4-F | H | CH(OEt)₂ | |
| 36-24 | 2-Cl | H | CH(OEt)₂ | |
| 36-25 | 3-Cl | H | CH(OEt)₂ | |
| 36-26 | 4-Cl | H | CH(OEt)₂ | |
| 36-27 | 2-Br | H | CH(OEt)₂ | |
| 36-28 | 3-Br | H | CH(OEt)₂ | |
| 36-29 | 4-Br | H | CH(OEt)₂ | |
| 36-30 | 2-I | H | CH(OEt)₂ | |
| 36-31 | 3-I | H | CH(OEt)₂ | |
| 36-32 | 4-I | H | CH(OEt)₂ | |
| 36-33 | 2-Cl | 3-Cl | CH(OEt)₂ | |
| 36-34 | 2-Cl | 4-Cl | CH(OEt)₂ | |
| 36-35 | 2-Cl | 5-Cl | CH(OEt)₂ | |
| 36-36 | 2-Cl | 6-Cl | CH(OEt)₂ | |
| 36-37 | 3-Cl | 4-Cl | CH(OEt)₂ | |
| 36-38 | 3-Cl | 5-Cl | CH(OEt)₂ | |
| 36-39 | H | H | CH(OnPr)₂ | |
| 36-40 | 2-F | H | CH(OnPr)₂ | |
| 36-41 | 3-F | H | CH(OnPr)₂ | |
| 36-42 | 4-F | H | CH(OnPr)₂ | |
| 36-43 | 2-Cl | H | CH(OnPr)₂ | |
| 36-44 | 3-Cl | H | CH(OnPr)₂ | |
| 36-45 | 4-Cl | H | CH(OnPr)₂ | |
| 36-46 | 2-Br | H | CH(OnPr)₂ | |
| 36-47 | 3-Br | H | CH(OnPr)₂ | |
| 36-48 | 4-Br | H | CH(OnPr)₂ | |
| 36-49 | 2-I | H | CH(OnPr)₂ | |
| 36-50 | 3-I | H | CH(OnPr)₂ | |
| 36-51 | 4-I | H | CH(OnPr)₂ | |
| 36-52 | 2-Cl | 3-Cl | CH(OnPr)₂ | |
| 36-53 | 2-Cl | 4-Cl | CH(OnPr)₂ | |
| 36-54 | 2-Cl | 5-Cl | CH(OnPr)₂ | |
| 36-55 | 2-Cl | 6-Cl | CH(OnPr)₂ | |
| 36-56 | 3-Cl | 4-Cl | CH(OnPr)₂ | |
| 36-57 | 3-Cl | 5-Cl | CH(OnPr)₂ | |
| 36-58 | H | H | CH(OnBu)₂ | |
| 36-59 | 2-F | H | CH(OnBu)₂ | |
| 36-60 | 3-F | H | CH(OnBu)₂ | |
| 36-61 | 4-F | H | CH(OnBu)₂ | |
| 36-62 | 2-Cl | H | CH(OnBu)₂ | |
| 36-63 | 3-Cl | H | CH(OnBu)₂ | |
| 36-64 | 4-Cl | H | CH(OnBu)₂ | |
| 36-65 | 2-Br | H | CH(OnBu)₂ | |
| 36-66 | 3-Br | H | CH(OnBu)₂ | |
| 36-67 | 4-Br | H | CH(OnBu)₂ | |
| 36-68 | 2-I | H | CH(OnBu)₂ | |
| 36-69 | 3-I | H | CH(OnBu)₂ | |
| 36-70 | 4-I | H | CH(OnBu)₂ | |
| 36-71 | 2-Cl | 3-Cl | CH(OnBu)₂ | |
| 36-72 | 2-Cl | 4-Cl | CH(OnBu)₂ | |
| 36-73 | 2-Cl | 5-Cl | CH(OnBu)₂ | |
| 36-74 | 2-Cl | 6-Cl | CH(OnBu)₂ | |
| 36-75 | 3-Cl | 4-Cl | CH(OnBu)₂ | |
| 36-76 | 3-Cl | 5-Cl | CH(OnBu)₂ | |
| 36-77 | 4-Cl | 3-CF₃ | CH(OMe)₂ | ¹H-NMR (d₆-DMSO): |
| | | | | 8.87, (d, 2H), |
| | | | | 7.42 (t, 1H), |
| | | | | 5.23 ppm (s, 1H) |
| 36-78 | 3-Cl | 5-CF₃ | CH(OMe)₂ | |
| 36-79 | 4-Cl | 3-CF₃ | CH(OEt)₂ | |
| 36-80 | 3-Cl | 5-CF₃ | CH(OEt)₂ | |

TABLE 37

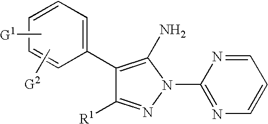

| Example | | | | |
|---|---|---|---|---|
| No. | G¹ | G² | R¹ | Physical data |
| 37-01 | H | H | CN | |
| 37-02 | 2-F | H | CN | |
| 37-03 | 3-F | H | CN | |
| 37-04 | 4-F | H | CN | |
| 37-05 | 2-Cl | H | CN | |
| 37-06 | 3-Cl | H | CN | |
| 37-07 | 4-Cl | H | CN | |
| 37-08 | 2-Br | H | CN | |
| 37-09 | 3-Br | H | CN | |
| 37-10 | 4-Br | H | CN | 1H(d6DMSO): 7.48, 7.71 ppm, 4-Br- |
| | | | | Ph. |
| 37-11 | 2-I | H | CN | |
| 37-12 | 3-I | H | CN | |
| 37-13 | 4-I | H | CN | |
| 37-14 | 2-Cl | 3-Cl | CN | |
| 37-15 | 2-Cl | 4-Cl | CN | |
| 37-16 | 2-Cl | 5-Cl | CN | |
| 37-17 | 2-Cl | 6-Cl | CN | |
| 37-18 | 3-Cl | 4-Cl | CN | |
| 37-19 | 3-Cl | 5-Cl | CN | |
| 37-20 | H | H | CHO | |
| 37-21 | 2-F | H | CHO | |
| 37-22 | 3-F | H | CHO | |
| 37-23 | 4-F | H | CHO | |
| 37-24 | 2-Cl | H | CHO | |
| 37-25 | 3-Cl | H | CHO | |
| 37-26 | 4-Cl | H | CHO | |
| 37-27 | 2-Br | H | CHO | |
| 37-28 | 3-Br | H | CHO | |
| 37-29 | 4-Br | H | CHO | |
| 37-30 | 2-I | H | CHO | |
| 37-31 | 3-I | H | CHO | |
| 37-32 | 4-I | H | CHO | |
| 37-33 | 2-Cl | 3-Cl | CHO | |
| 37-34 | 2-Cl | 4-Cl | CHO | |
| 37-35 | 2-Cl | 5-Cl | CHO | |
| 37-36 | 2-Cl | 6-Cl | CHO | |
| 37-37 | 3-Cl | 4-Cl | CHO | |
| 37-38 | 3-Cl | 5-Cl | CHO | |

TABLE 38

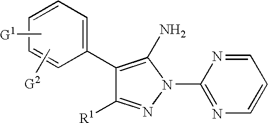

| Example | | | | |
|---|---|---|---|---|
| No. | G¹ | G² | R¹ | Physical data |
| 38-01 | H | H | CH═NOH | |
| 38-02 | 2-F | H | CH═NOH | |
| 38-03 | 3-F | H | CH═NOH | |
| 38-04 | 4-F | H | CH═NOH | |
| 38-05 | 2-Cl | H | CH═NOH | |
| 38-06 | 3-Cl | H | CH═NOH | |
| 38-07 | 4-Cl | H | CH═NOH | |
| 38-08 | 2-Br | H | CH═NOH | |
| 38-09 | 3-Br | H | CH═NOH | |
| 38-10 | 4-Br | H | CH═NOH | 1H(d6DMSO): 7.96, 1H, s; 11.13 ppm, 1H, s; CH═NOH |
| 38-11 | 2-I | H | CH═NOH | |
| 38-12 | 3-I | H | CH═NOH | |
| 38-13 | 4-I | H | CH═NOH | |
| 38-14 | 2-Cl | 3-Cl | CH═NOH | |
| 38-15 | 2-Cl | 4-Cl | CH═NOH | |
| 38-16 | 2-Cl | 5-Cl | CH═NOH | |
| 38-17 | 2-Cl | 6-Cl | CH═NOH | |
| 38-18 | 3-Cl | 4-Cl | CH═NOH | |
| 38-19 | 3-Cl | 5-Cl | CH═NOH | |
| 38-20 | H | H | CH═NOMe | |
| 38-21 | 2-F | H | CH═NOMe | |
| 38-22 | 3-F | H | CH═NOMe | |
| 38-23 | 4-F | H | CH═NOMe | |
| 38-24 | 2-Cl | H | CH═NOMe | |
| 38-25 | 3-Cl | H | CH═NOMe | |
| 38-26 | 4-Cl | H | CH═NOMe | |
| 38-27 | 2-Br | H | CH═NOMe | |
| 38-28 | 3-Br | H | CH═NOMe | |
| 38-29 | 4-Br | H | CH═NOMe | 1H(d6DMSO): 3.76 ppm; OMe. |
| 38-30 | 2-I | H | CH═NOMe | |
| 38-31 | 3-I | H | CH═NOMe | |
| 38-32 | 4-I | H | CH═NOMe | |
| 38-33 | 2-Cl | 3-Cl | CH═NOMe | |
| 38-34 | 2-Cl | 4-Cl | CH═NOMe | |
| 38-35 | 2-Cl | 5-Cl | CH═NOMe | |
| 38-36 | 2-Cl | 6-Cl | CH═NOMe | |
| 38-37 | 3-Cl | 4-Cl | CH═NOMe | |
| 38-38 | 3-Cl | 5-Cl | CH═NOMe | |
| 38-39 | H | H | CH═NOEt | |
| 38-40 | 2-F | H | CH═NOEt | |
| 38-41 | 3-F | H | CH═NOEt | |
| 38-42 | 4-F | H | CH═NOEt | |
| 38-43 | 2-Cl | H | CH═NOEt | |
| 38-44 | 3-Cl | H | CH═NOEt | |
| 38-45 | 4-Cl | H | CH═NOEt | |
| 38-46 | 2-Br | H | CH═NOEt | |
| 38-47 | 3-Br | H | CH═NOEt | |
| 38-48 | 4-Br | H | CH═NOEt | 1H(d6DMSO): 3.98, 1.14 ppm; OEt |
| 38-49 | 2-I | H | CH═NOEt | |
| 38-50 | 3-I | H | CH═NOEt | |
| 38-51 | 4-I | H | CH═NOEt | |
| 38-52 | 2-Cl | 3-Cl | CH═NOEt | |
| 38-53 | 2-Cl | 4-Cl | CH═NOEt | |
| 38-54 | 2-Cl | 5-Cl | CH═NOEt | |
| 38-55 | 2-Cl | 6-Cl | CH═NOEt | |
| 38-56 | 3-Cl | 4-Cl | CH═NOEt | |
| 38-57 | 3-Cl | 5-Cl | CH═NOEt | |
| 38-58 | H | H | CH═NOnPr | |
| 38-59 | 2-F | H | CH═NOnPr | |
| 38-60 | 3-F | H | CH═NOnPr | |
| 38-61 | 4-F | H | CH═NOnPr | |
| 38-62 | 2-Cl | H | CH═NOnPr | |
| 38-63 | 3-Cl | H | CH═NOnPr | |
| 38-64 | 4-Cl | H | CH═NOnPr | |
| 38-65 | 2-Br | H | CH═NOnPr | |
| 38-66 | 3-Br | H | CH═NOnPr | |
| 38-67 | 4-Br | H | CH═NOnPr | |
| 38-68 | 2-I | H | CH═NOnPr | |
| 38-69 | 3-I | H | CH═NOnPr | |
| 38-70 | 4-I | H | CH═NOnPr | |
| 38-71 | 2-Cl | 3-Cl | CH═NOnPr | |
| 38-72 | 2-Cl | 4-Cl | CH═NOnPr | |
| 38-73 | 2-Cl | 5-Cl | CH═NOnPr | |
| 38-74 | 2-Cl | 6-Cl | CH═NOnPr | |
| 38-75 | 3-Cl | 4-Cl | CH═NOnPr | |
| 38-76 | 3-Cl | 5-Cl | CH═NOnPr | |
| 38-77 | H | H | CH═NOiPr | |
| 38-78 | 2-F | H | CH═NOiPr | |
| 38-79 | 3-F | H | CH═NOiPr | |
| 38-80 | 4-F | H | CH═NOiPr | |
| 38-81 | 2-Cl | H | CH═NOiPr | |
| 38-82 | 3-Cl | H | CH═NOiPr | |
| 38-83 | 4-Cl | H | CH═NOiPr | |
| 38-84 | 2-Br | H | CH═NOiPr | |
| 38-85 | 3-Br | H | CH═NOiPr | |
| 38-86 | 4-Br | H | CH═NOiPr | |
| 38-87 | 2-I | H | CH═NOiPr | |
| 38-88 | 3-I | H | CH═NOiPr | |
| 38-89 | 4-I | H | CH═NOiPr | |
| 38-90 | 2-Cl | 3-Cl | CH═NOiPr | |
| 38-91 | 2-Cl | 4-Cl | CH═NOiPr | |
| 38-92 | 2-Cl | 5-Cl | CH═NOiPr | |
| 38-93 | 2-Cl | 6-Cl | CH═NOiPr | |
| 38-94 | 3-Cl | 4-Cl | CH═NOiPr | |
| 38-95 | 3-Cl | 5-Cl | CH═NOiPr | |

TABLE 39

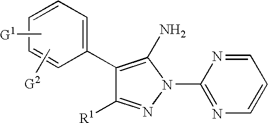

| Example No. | G¹ | G² | R¹ | Physical data |
|---|---|---|---|---|
| 39-01 | H | H | C₆H₅—CH₂ | |
| 39-02 | 2-F | H | C₆H₅—CH₂ | |
| 39-03 | 3-F | H | C₆H₅—CH₂ | |
| 39-04 | 4-F | H | C₆H₅—CH₂ | |
| 39-05 | 2-Cl | H | C₆H₅—CH₂ | |
| 39-06 | 3-Cl | H | C₆H₅—CH₂ | |
| 39-07 | 4-Cl | H | C₆H₅—CH₂ | |
| 39-08 | 2-Br | H | C₆H₅—CH₂ | |
| 39-09 | 3-Br | H | C₆H₅—CH₂ | |
| 39-10 | 4-Br | H | C₆H₅—CH₂ | 1H: 4.03 ppm, CH₂ |
| 39-11 | 2-I | H | C₆H₅—CH₂ | |
| 39-12 | 3-I | H | C₆H₅—CH₂ | |
| 39-13 | 4-I | H | C₆H₅—CH₂ | |
| 39-14 | 2-Cl | 3-Cl | C₆H₅—CH₂ | |
| 39-15 | 2-Cl | 4-Cl | C₆H₅—CH₂ | |
| 39-16 | 2-Cl | 5-Cl | C₆H₅—CH₂ | |
| 39-17 | 2-Cl | 6-Cl | C₆H₅—CH₂ | |
| 39-18 | 3-Cl | 4-Cl | C₆H₅—CH₂ | |
| 39-19 | 3-Cl | 5-Cl | C₆H₅—CH₂ | |
| 39-20 | H | H | 2-Cl—C₆H₄—CH₂ | |
| 39-21 | 2-F | H | 2-Cl—C₆H₄—CH₂ | |
| 39-22 | 3-F | H | 2-Cl—C₆H₄—CH₂ | |
| 39-23 | 4-F | H | 2-Cl—C₆H₄—CH₂ | |
| 39-24 | 2-Cl | H | 2-Cl—C₆H₄—CH₂ | |
| 39-25 | 3-Cl | H | 2-Cl—C₆H₄—CH₂ | |
| 39-26 | 4-Cl | H | 2-Cl—C₆H₄—CH₂ | |
| 39-27 | 2-Br | H | 2-Cl—C₆H₄—CH₂ | |
| 39-28 | 3-Br | H | 2-Cl—C₆H₄—CH₂ | |
| 39-29 | 4-Br | H | 2-Cl—C₆H₄—CH₂ | |
| 39-30 | 2-I | H | 2-Cl—C₆H₄—CH₂ | |
| 39-31 | 3-I | H | 2-Cl—C₆H₄—CH₂ | |
| 39-32 | 4-I | H | 2-Cl—C₆H₄—CH₂ | |
| 39-33 | 2-Cl | 3-Cl | 2-Cl—C₆H₄—CH₂ | |
| 39-34 | 2-Cl | 4-Cl | 2-Cl—C₆H₄—CH₂ | |
| 39-35 | 2-Cl | 5-Cl | 2-Cl—C₆H₄—CH₂ | |
| 39-36 | 2-Cl | 6-Cl | 2-Cl—C₆H₄—CH₂ | |
| 39-37 | 3-Cl | 4-Cl | 2-Cl—C₆H₄—CH₂ | |
| 39-38 | 3-Cl | 5-Cl | 2-Cl—C₆H₄—CH₂ | |
| 39-39 | H | H | 3-Cl—C₆H₄—CH₂ | |
| 39-40 | 2-F | H | 3-Cl—C₆H₄—CH₂ | |
| 39-41 | 3-F | H | 3-Cl—C₆H₄—CH₂ | |
| 39-42 | 4-F | H | 3-Cl—C₆H₄—CH₂ | |
| 39-43 | 2-Cl | H | 3-Cl—C₆H₄—CH₂ | |
| 39-44 | 3-Cl | H | 3-Cl—C₆H₄—CH₂ | |
| 39-45 | 4-Cl | H | 3-Cl—C₆H₄—CH₂ | |
| 39-46 | 2-Br | H | 3-Cl—C₆H₄—CH₂ | |
| 39-47 | 3-Br | H | 3-Cl—C₆H₄—CH₂ | |
| 39-48 | 4-Br | H | 3-Cl—C₆H₄—CH₂ | 1H: 4.00 ppm, CH₂ |
| 39-49 | 2-I | H | 3-Cl—C₆H₄—CH₂ | |
| 39-50 | 3-I | H | 3-Cl—C₆H₄—CH₂ | |
| 39-51 | 4-I | H | 3-Cl—C₆H₄—CH₂ | |
| 39-52 | 2-Cl | 3-Cl | 3-Cl—C₆H₄—CH₂ | |
| 39-53 | 2-Cl | 4-Cl | 3-Cl—C₆H₄—CH₂ | |
| 39-54 | 2-Cl | 5-Cl | 3-Cl—C₆H₄—CH₂ | |
| 39-55 | 2-Cl | 6-Cl | 3-Cl—C₆H₄—CH₂ | |
| 39-56 | 3-Cl | 4-Cl | 3-Cl—C₆H₄—CH₂ | |
| 39-57 | 3-Cl | 5-Cl | 3-Cl—C₆H₄—CH₂ | |
| 39-58 | H | H | 4-Cl—C₆H₄—CH₂ | |
| 39-59 | 2-F | H | 4-Cl—C₆H₄—CH₂ | |
| 39-60 | 3-F | H | 4-Cl—C₆H₄—CH₂ | |
| 39-61 | 4-F | H | 4-Cl—C₆H₄—CH₂ | |
| 39-62 | 2-Cl | H | 4-Cl—C₆H₄—CH₂ | |
| 39-63 | 3-Cl | H | 4-Cl—C₆H₄—CH₂ | |
| 39-64 | 4-Cl | H | 4-Cl—C₆H₄—CH₂ | |
| 39-65 | 2-Br | H | 4-Cl—C₆H₄—CH₂ | |
| 39-66 | 3-Br | H | 4-Cl—C₆H₄—CH₂ | |
| 39-67 | 4-Br | H | 4-Cl—C₆H₄—CH₂ | 1H: 3.98 ppm, CH₂ |
| 39-68 | 2-I | H | 4-Cl—C₆H₄—CH₂ | |
| 39-69 | 3-I | H | 4-Cl—C₆H₄—CH₂ | |
| 39-70 | 4-I | H | 4-Cl—C₆H₄—CH₂ | |
| 39-71 | 2-Cl | 3-Cl | 4-Cl—C₆H₄—CH₂ | |
| 39-72 | 2-Cl | 4-Cl | 4-Cl—C₆H₄—CH₂ | |
| 39-73 | 2-Cl | 5-Cl | 4-Cl—C₆H₄—CH₂ | |
| 39-74 | 2-Cl | 6-Cl | 4-Cl—C₆H₄—CH₂ | |
| 39-75 | 3-Cl | 4-Cl | 4-Cl—C₆H₄—CH₂ | |
| 39-76 | 3-Cl | 5-Cl | 4-Cl—C₆H₄—CH₂ | |
| 39-77 | H | H | 2-F—C₆H₄—CH₂ | |
| 39-78 | 2-F | H | 2-F—C₆H₄—CH₂ | |
| 39-79 | 3-F | H | 2-F—C₆H₄—CH₂ | |
| 39-80 | 4-F | H | 2-F—C₆H₄—CH₂ | |
| 39-81 | 2-Cl | H | 2-F—C₆H₄—CH₂ | |
| 39-82 | 3-Cl | H | 2-F—C₆H₄—CH₂ | |
| 39-83 | 4-Cl | H | 2-F—C₆H₄—CH₂ | |
| 39-84 | 2-Br | H | 2-F—C₆H₄—CH₂ | |
| 39-85 | 3-Br | H | 2-F—C₆H₄—CH₂ | |
| 39-86 | 4-Br | H | 2-F—C₆H₄—CH₂ | 19F: −118.45 ppm, PhF |
| 39-87 | 2-I | H | 2-F—C₆H₄—CH₂ | |
| 39-88 | 3-I | H | 2-F—C₆H₄—CH₂ | |
| 39-89 | 4-I | H | 2-F—C₆H₄—CH₂ | |
| 39-90 | 2-Cl | 3-Cl | 2-F—C₆H₄—CH₂ | |
| 39-91 | 2-Cl | 4-Cl | 2-F—C₆H₄—CH₂ | |
| 39-92 | 2-Cl | 5-Cl | 2-F—C₆H₄—CH₂ | |
| 39-93 | 2-Cl | 6-Cl | 2-F—C₆H₄—CH₂ | |
| 39-94 | 3-Cl | 4-Cl | 2-F—C₆H₄—CH₂ | |
| 39-95 | 3-Cl | 5-Cl | 2-F—C₆H₄—CH₂ | |
| 39-96 | H | H | 3-F—C₆H₄—CH₂ | |
| 39-97 | 2-F | H | 3-F—C₆H₄—CH₂ | |
| 39-98 | 3-F | H | 3-F—C₆H₄—CH₂ | |
| 39-99 | 4-F | H | 3-F—C₆H₄—CH₂ | |
| 39-100 | 2-Cl | H | 3-F—C₆H₄—CH₂ | |
| 39-101 | 3-Cl | H | 3-F—C₆H₄—CH₂ | |
| 39-102 | 4-Cl | H | 3-F—C₆H₄—CH₂ | |
| 39-103 | 2-Br | H | 3-F—C₆H₄—CH₂ | |
| 39-104 | 3-Br | H | 3-F—C₆H₄—CH₂ | |
| 39-105 | 4-Br | H | 3-F—C₆H₄—CH₂ | 1H(d6DMSO): 6.90, 6.96, 7.26 ppm, F- |
| | | | | phenylH |
| 39-106 | 2-I | H | 3-F—C₆H₄—CH₂ | |
| 39-107 | 3-I | H | 3-F—C₆H₄—CH₂ | |
| 39-108 | 4-I | H | 3-F—C₆H₄—CH₂ | |
| 39-109 | 2-Cl | 3-Cl | 3-F—C₆H₄—CH₂ | |
| 39-110 | 2-Cl | 4-Cl | 3-F—C₆H₄—CH₂ | |
| 39-111 | 2-Cl | 5-Cl | 3-F—C₆H₄—CH₂ | |
| 39-112 | 2-Cl | 6-Cl | 3-F—C₆H₄—CH₂ | |
| 39-113 | 3-Cl | 4-Cl | 3-F—C₆H₄—CH₂ | |
| 39-114 | 3-Cl | 5-Cl | 3-F—C₆H₄—CH₂ | |
| 39-115 | H | H | 4-F—C₆H₄—CH₂ | |
| 39-116 | 2-F | H | 4-F—C₆H₄—CH₂ | |
| 39-117 | 3-F | H | 4-F—C₆H₄—CH₂ | |
| 39-118 | 4-F | H | 4-F—C₆H₄—CH₂ | |
| 39-119 | 2-Cl | H | 4-F—C₆H₄—CH₂ | |
| 39-120 | 3-Cl | H | 4-F—C₆H₄—CH₂ | |
| 39-121 | 4-Cl | H | 4-F—C₆H₄—CH₂ | |
| 39-122 | 2-Br | H | 4-F—C₆H₄—CH₂ | |
| 39-123 | 3-Br | H | 4-F—C₆H₄—CH₂ | |
| 39-124 | 4-Br | H | 4-F—C₆H₄—CH₂ | 1H(d6DMSO): 7.04, 7.14, F-phenylH |
| 39-125 | 2-I | H | 4-F—C₆H₄—CH₂ | |
| 39-126 | 3-I | H | 4-F—C₆H₄—CH₂ | |
| 39-127 | 4-I | H | 4-F—C₆H₄—CH₂ | |
| 39-128 | 2-Cl | 3-Cl | 4-F—C₆H₄—CH₂ | |
| 39-129 | 2-Cl | 4-Cl | 4-F—C₆H₄—CH₂ | |
| 39-130 | 2-Cl | 5-Cl | 4-F—C₆H₄—CH₂ | |
| 39-131 | 2-Cl | 6-Cl | 4-F—C₆H₄—CH₂ | |
| 39-132 | 3-Cl | 4-Cl | 4-F—C₆H₄—CH₂ | |
| 39-133 | 3-Cl | 5-Cl | 4-F—C₆H₄—CH₂ | |
| 39-134 | H | H | 2-CF₃—C₆H₄—CH₂ | |
| 39-135 | 2-F | H | 2-CF₃—C₆H₄—CH₂ | |
| 39-136 | 3-F | H | 2-CF₃—C₆H₄—CH₂ | |
| 39-137 | 4-F | H | 2-CF₃—C₆H₄—CH₂ | |
| 39-138 | 2-Cl | H | 2-CF₃—C₆H₄—CH₂ | |
| 39-139 | 3-Cl | H | 2-CF₃—C₆H₄—CH₂ | |
| 39-140 | 4-Cl | H | 2-CF₃—C₆H₄—CH₂ | |
| 39-141 | 2-Br | H | 2-CF₃—C₆H₄—CH₂ | |
| 39-142 | 3-Br | H | 2-CF₃—C₆H₄—CH₂ | |
| 39-143 | 4-Br | H | 2-CF₃—C₆H₄—CH₂ | |
| 39-144 | 2-I | H | 2-CF₃—C₆H₄—CH₂ | |
| 39-145 | 3-I | H | 2-CF₃—C₆H₄—CH₂ | |
| 39-146 | 4-I | H | 2-CF₃—C₆H₄—CH₂ | |
| 39-147 | 2-Cl | 3-Cl | 2-CF₃—C₆H₄—CH₂ | |
| 39-148 | 2-Cl | 4-Cl | 2-CF₃—C₆H₄—CH₂ | |
| 39-149 | 2-Cl | 5-Cl | 2-CF₃—C₆H₄—CH₂ | |
| 39-150 | 2-Cl | 6-Cl | 2-CF₃—C₆H₄—CH₂ | |
| 39-151 | 3-Cl | 4-Cl | 2-CF₃—C₆H₄—CH₂ | |
| 39-152 | 3-Cl | 5-Cl | 2-CF₃—C₆H₄—CH₂ | |
| 39-153 | H | H | 3-CF₃—C₆H₄—CH₂ | |
| 39-154 | 2-F | H | 3-CF₃—C₆H₄—CH₂ | |
| 39-155 | 3-F | H | 3-CF₃—C₆H₄—CH₂ | |
| 39-156 | 4-F | H | 3-CF₃—C₆H₄—CH₂ | |
| 39-157 | 2-Cl | H | 3-CF₃—C₆H₄—CH₂ | |
| 39-158 | 3-Cl | H | 3-CF₃—C₆H₄—CH₂ | |
| 39-159 | 4-Cl | H | 3-CF₃—C₆H₄—CH₂ | |
| 39-160 | 2-Br | H | 3-CF₃—C₆H₄—CH₂ | |
| 39-161 | 3-Br | H | 3-CF₃—C₆H₄—CH₂ | |
| 39-162 | 4-Br | H | 3-CF₃—C₆H₄—CH₂ | 1H: 4.08 ppm, CH₂ |
| 39-163 | 2-I | H | 3-CF₃—C₆H₄—CH₂ | |
| 39-164 | 3-I | H | 3-CF₃—C₆H₄—CH₂ | |
| 39-165 | 4-I | H | 3-CF₃—C₆H₄—CH₂ | |
| 39-166 | 2-Cl | 3-Cl | 3-CF₃—C₆H₄—CH₂ | |
| 39-167 | 2-Cl | 4-Cl | 3-CF₃—C₆H₄—CH₂ | |
| 39-168 | 2-Cl | 5-Cl | 3-CF₃—C₆H₄—CH₂ | |
| 39-169 | 2-Cl | 6-Cl | 3-CF₃—C₆H₄—CH₂ | |
| 39-170 | 3-Cl | 4-Cl | 3-CF₃—C₆H₄—CH₂ | |
| 39-171 | 3-Cl | 5-Cl | 3-CF₃—C₆H₄—CH₂ | |
| 39-172 | H | H | 4-CF₃—C₆H₄—CH₂ | |
| 39-173 | 2-F | H | 4-CF₃—C₆H₄—CH₂ | |
| 39-174 | 3-F | H | 4-CF₃—C₆H₄—CH₂ | |
| 39-175 | 4-F | H | 4-CF₃—C₆H₄—CH₂ | |
| 39-176 | 2-Cl | H | 4-CF₃—C₆H₄—CH₂ | |
| 39-177 | 3-Cl | H | 4-CF₃—C₆H₄—CH₂ | |
| 39-178 | 4-Cl | H | 4-CF₃—C₆H₄—CH₂ | |
| 39-179 | 2-Br | H | 4-CF₃—C₆H₄—CH₂ | |
| 39-180 | 3-Br | H | 4-CF₃—C₆H₄—CH₂ | |
| 39-181 | 4-Br | H | 4-CF₃—C₆H₄—CH₂ | |
| 39-182 | 2-I | H | 4-CF₃—C₆H₄—CH₂ | |
| 39-183 | 3-I | H | 4-CF₃—C₆H₄—CH₂ | |
| 39-184 | 4-I | H | 4-CF₃—C₆H₄—CH₂ | |
| 39-185 | 2-Cl | 3-Cl | 4-CF₃—C₆H₄—CH₂ | |
| 39-186 | 2-Cl | 4-Cl | 4-CF₃—C₆H₄—CH₂ | |
| 39-187 | 2-Cl | 5-Cl | 4-CF₃—C₆H₄—CH₂ | |
| 39-188 | 2-Cl | 6-Cl | 4-CF₃—C₆H₄—CH₂ | |
| 39-189 | 3-Cl | 4-Cl | 4-CF₃—C₆H₄—CH₂ | |
| 39-190 | 3-Cl | 5-Cl | 4-CF₃—C₆H₄—CH₂ | |
| 39-191 | H | H | 2,4-Cl₂—C₆H₃—CH₂ | |
| 39-192 | 2-F | H | 2,4-Cl₂—C₆H₃—CH₂ | |
| 39-193 | 3-F | H | 2,4-Cl₂—C₆H₃—CH₂ | |
| 39-194 | 4-F | H | 2,4-Cl₂—C₆H₃—CH₂ | |
| 39-195 | 2-Cl | H | 2,4-Cl₂—C₆H₃—CH₂ | |
| 39-196 | 3-Cl | H | 2,4-Cl₂—C₆H₃—CH₂ | |
| 39-197 | 4-Cl | H | 2,4-Cl₂—C₆H₃—CH₂ | |
| 39-198 | 2-Br | H | 2,4-Cl₂—C₆H₃—CH₂ | |
| 39-199 | 3-Br | H | 2,4-Cl₂—C₆H₃—CH₂ | |
| 39-200 | 4-Br | H | 2,4-Cl₂—C₆H₃—CH₂ | 1H(d6DMSO): 7.30, 7.38, 7.62, PhH. |
| 39-201 | 2-I | H | 2,4-Cl₂—C₆H₃—CH₂ | |
| 39-202 | 3-I | H | 2,4-Cl₂—C₆H₃—CH₂ | |
| 39-203 | 4-I | H | 2,4-Cl₂—C₆H₃—CH₂ | |
| 39-204 | 2-Cl | 3-Cl | 2,4-Cl₂—C₆H₃—CH₂ | |
| 39-205 | 2-Cl | 4-Cl | 2,4-Cl₂—C₆H₃—CH₂ | |
| 39-206 | 2-Cl | 5-Cl | 2,4-Cl₂—C₆H₃—CH₂ | |
| 39-207 | 2-Cl | 6-Cl | 2,4-Cl₂—C₆H₃—CH₂ | |
| 39-208 | 3-Cl | 4-Cl | 2,4-Cl₂—C₆H₃—CH₂ | |
| 39-209 | 3-Cl | 5-Cl | 2,4-Cl₂—C₆H₃—CH₂ | |
| 39-210 | H | H | 2-Br—C₆H₄—CH₂ | |
| 39-211 | 2-F | H | 2-Br—C₆H₄—CH₂ | |
| 39-212 | 3-F | H | 2-Br—C₆H₄—CH₂ | |
| 39-213 | 4-F | H | 2-Br—C₆H₄—CH₂ | |
| 39-214 | 2-Cl | H | 2-Br—C₆H₄—CH₂ | |
| 39-215 | 3-Cl | H | 2-Br—C₆H₄—CH₂ | |
| 39-216 | 4-Cl | H | 2-Br—C₆H₄—CH₂ | |
| 39-217 | 2-Br | H | 2-Br—C₆H₄—CH₂ | |
| 39-218 | 3-Br | H | 2-Br—C₆H₄—CH₂ | |
| 39-219 | 4-Br | H | 2-Br—C₆H₄—CH₂ | 1H(d6DMSO): 3.97 ppm, CH₂ |
| 39-220 | 2-I | H | 2-Br—C₆H₄—CH₂ | |
| 39-221 | 3-I | H | 2-Br—C₆H₄—CH₂ | |
| 39-222 | 4-I | H | 2-Br—C₆H₄—CH₂ | |
| 39-223 | 2-Cl | 3-Cl | 2-Br—C₆H₄—CH₂ | |
| 39-224 | 2-Cl | 4-Cl | 2-Br—C₆H₄—CH₂ | |
| 39-225 | 2-Cl | 5-Cl | 2-Br—C₆H₄—CH₂ | |
| 39-226 | 2-Cl | 6-Cl | 2-Br—C₆H₄—CH₂ | |
| 39-227 | 3-Cl | 4-Cl | 2-Br—C₆H₄—CH₂ | |
| 39-228 | 3-Cl | 5-Cl | 2-Br—C₆H₄—CH₂ | |
| 39-229 | H | H | 3-Br—C₆H₄—CH₂ | |
| 39-230 | 2-F | H | 3-Br—C₆H₄—CH₂ | |
| 39-231 | 3-F | H | 3-Br—C₆H₄—CH₂ | |
| 39-232 | 4-F | H | 3-Br—C₆H₄—CH₂ | |
| 39-233 | 2-Cl | H | 3-Br—C₆H₄—CH₂ | |
| 39-234 | 3-Cl | H | 3-Br—C₆H₄—CH₂ | |
| 39-235 | 4-Cl | H | 3-Br—C₆H₄—CH₂ | |
| 39-236 | 2-Br | H | 3-Br—C₆H₄—CH₂ | |
| 39-237 | 3-Br | H | 3-Br—C₆H₄—CH₂ | |
| 39-238 | 4-Br | H | 3-Br—C₆H₄—CH₂ | |
| 39-239 | 2-I | H | 3-Br—C₆H₄—CH₂ | |
| 39-240 | 3-I | H | 3-Br—C₆H₄—CH₂ | |
| 39-241 | 4-I | H | 3-Br—C₆H₄—CH₂ | |
| 39-242 | 2-Cl | 3-Cl | 3-Br—C₆H₄—CH₂ | |
| 39-243 | 2-Cl | 4-Cl | 3-Br—C₆H₄—CH₂ | |
| 39-244 | 2-Cl | 5-Cl | 3-Br—C₆H₄—CH₂ | |
| 39-245 | 2-Cl | 6-Cl | 3-Br—C₆H₄—CH₂ | |
| 39-246 | 3-Cl | 4-Cl | 3-Br—C₆H₄—CH₂ | |
| 39-247 | 3-Cl | 5-Cl | 3-Br—C₆H₄—CH₂ | |
| 39-248 | H | H | 4-Br—C₆H₄—CH₂ | |
| 39-249 | 2-F | H | 4-Br—C₆H₄—CH₂ | |
| 39-250 | 3-F | H | 4-Br—C₆H₄—CH₂ | |
| 39-251 | 4-F | H | 4-Br—C₆H₄—CH₂ | |
| 39-252 | 2-Cl | H | 4-Br—C₆H₄—CH₂ | |
| 39-253 | 3-Cl | H | 4-Br—C₆H₄—CH₂ | |
| 39-254 | 4-Cl | H | 4-Br—C₆H₄—CH₂ | |
| 39-255 | 2-Br | H | 4-Br—C₆H₄—CH₂ | |
| 39-256 | 3-Br | H | 4-Br—C₆H₄—CH₂ | |
| 39-257 | 4-Br | H | 4-Br—C₆H₄—CH₂ | 1H: 3.97 ppm, CH₂ |
| 39-258 | 2-I | H | 4-Br—C₆H₄—CH₂ | |
| 39-259 | 3-I | H | 4-Br—C₆H₄—CH₂ | |
| 39-260 | 4-I | H | 4-Br—C₆H₄—CH₂ | |
| 39-261 | 2-Cl | 3-Cl | 4-Br—C₆H₄—CH₂ | |
| 39-262 | 2-Cl | 4-Cl | 4-Br—C₆H₄—CH₂ | |
| 39-263 | 2-Cl | 5-Cl | 4-Br—C₆H₄—CH₂ | |
| 39-264 | 2-Cl | 6-Cl | 4-Br—C₆H₄—CH₂ | |
| 39-265 | 3-Cl | 4-Cl | 4-Br—C₆H₄—CH₂ | |
| 39-266 | 3-Cl | 5-Cl | 4-Br—C₆H₄—CH₂ | |

TABLE 40

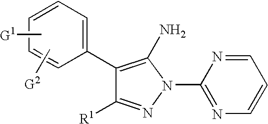

| Example No. | G¹ | G² | R¹ | Physical data |
|---|---|---|---|---|
| 40-01 | H | H | 2-CH₃—C₆H₄—CH₂ | |
| 40-02 | 2-F | H | 2-CH₃—C₆H₄—CH₂ | |
| 40-03 | 3-F | H | 2-CH₃—C₆H₄—CH₂ | |
| 40-04 | 4-F | H | 2-CH₃—C₆H₄—CH₂ | |
| 40-05 | 2-Cl | H | 2-CH₃—C₆H₄—CH₂ | |
| 40-06 | 3-Cl | H | 2-CH₃—C₆H₄—CH₂ | |
| 40-07 | 4-Cl | H | 2-CH₃—C₆H₄—CH₂ | |
| 40-08 | 2-Br | H | 2-CH₃—C₆H₄—CH₂ | |
| 40-09 | 3-Br | H | 2-CH₃—C₆H₄—CH₂ | |
| 40-10 | 4-Br | H | 2-CH₃—C₆H₄—CH₂ | 1H(d6DMSO): 2.20 ppm, Ph-CH₃ |
| 40-11 | 2-I | H | 2-CH₃—C₆H₄—CH₂ | |
| 40-12 | 3-I | H | 2-CH₃—C₆H₄—CH₂ | |
| 40-13 | 4-I | H | 2-CH₃—C₆H₄—CH₂ | |
| 40-14 | 2-Cl | 3-Cl | 2-CH₃—C₆H₄—CH₂ | |
| 40-15 | 2-Cl | 4-Cl | 2-CH₃—C₆H₄—CH₂ | |
| 40-16 | 2-Cl | 5-Cl | 2-CH₃—C₆H₄—CH₂ | |
| 40-17 | 2-Cl | 6-Cl | 2-CH₃—C₆H₄—CH₂ | |
| 40-18 | 3-Cl | 4-Cl | 2-CH₃—C₆H₄—CH₂ | |
| 40-19 | 3-Cl | 5-Cl | 2-CH₃—C₆H₄—CH₂ | |
| 40-20 | H | H | 3-CH₃—C₆H₄—CH₂ | |
| 40-21 | 2-F | H | 3-CH₃—C₆H₄—CH₂ | |
| 40-22 | 3-F | H | 3-CH₃—C₆H₄—CH₂ | |
| 40-23 | 4-F | H | 3-CH₃—C₆H₄—CH₂ | |
| 40-24 | 2-Cl | H | 3-CH₃—C₆H₄—CH₂ | |
| 40-25 | 3-Cl | H | 3-CH₃—C₆H₄—CH₂ | |
| 40-26 | 4-Cl | H | 3-CH₃—C₆H₄—CH₂ | |
| 40-27 | 2-Br | H | 3-CH₃—C₆H₄—CH₂ | |
| 40-28 | 3-Br | H | 3-CH₃—C₆H₄—CH₂ | |
| 40-29 | 4-Br | H | 3-CH₃—C₆H₄—CH₂ | 1H(d6DMSO): 2.20 ppm, Ph-CH₃ |
| 40-30 | 2-I | H | 3-CH₃—C₆H₄—CH₂ | |
| 40-31 | 3-I | H | 3-CH₃—C₆H₄—CH₂ | |
| 40-32 | 4-I | H | 3-CH₃—C₆H₄—CH₂ | |
| 40-33 | 2-Cl | 3-Cl | 3-CH₃—C₆H₄—CH₂ | |
| 40-34 | 2-Cl | 4-Cl | 3-CH₃—C₆H₄—CH₂ | |
| 40-35 | 2-Cl | 5-Cl | 3-CH₃—C₆H₄—CH₂ | |
| 40-36 | 2-Cl | 6-Cl | 3-CH₃—C₆H₄—CH₂ | |
| 40-37 | 3-Cl | 4-Cl | 3-CH₃—C₆H₄—CH₂ | |
| 40-38 | 3-Cl | 5-Cl | 3-CH₃—C₆H₄—CH₂ | |
| 40-39 | H | H | 4-CH₃—C₆H₄—CH₂ | |
| 40-40 | 2-F | H | 4-CH₃—C₆H₄—CH₂ | |
| 40-41 | 3-F | H | 4-CH₃—C₆H₄—CH₂ | |
| 40-42 | 4-F | H | 4-CH₃—C₆H₄—CH₂ | |
| 40-43 | 2-Cl | H | 4-CH₃—C₆H₄—CH₂ | |
| 40-44 | 3-Cl | H | 4-CH₃—C₆H₄—CH₂ | |
| 40-45 | 4-Cl | H | 4-CH₃—C₆H₄—CH₂ | |
| 40-46 | 2-Br | H | 4-CH₃—C₆H₄—CH₂ | |
| 40-47 | 3-Br | H | 4-CH₃—C₆H₄—CH₂ | |
| 40-48 | 4-Br | H | 4-CH₃—C₆H₄—CH₂ | 1H(d6DMSO): 2.22 ppm, Ph-CH₃ |
| 40-49 | 2-I | H | 4-CH₃—C₆H₄—CH₂ | |
| 40-50 | 3-I | H | 4-CH₃—C₆H₄—CH₂ | |
| 40-51 | 4-I | H | 4-CH₃—C₆H₄—CH₂ | |
| 40-52 | 2-Cl | 3-Cl | 4-CH₃—C₆H₄—CH₂ | |
| 40-53 | 2-Cl | 4-Cl | 4-CH₃—C₆H₄—CH₂ | |
| 40-54 | 2-Cl | 5-Cl | 4-CH₃—C₆H₄—CH₂ | |
| 40-55 | 2-Cl | 6-Cl | 4-CH₃—C₆H₄—CH₂ | |
| 40-56 | 3-Cl | 4-Cl | 4-CH₃—C₆H₄—CH₂ | |
| 40-57 | 3-Cl | 5-Cl | 4-CH₃—C₆H₄—CH₂ | |
| 40-58 | H | H | 2,4,6-Me₃-C₆H₂—CH₂ | |
| 40-59 | 2-F | H | 2,4,6-Me₃-C₆H₂—CH₂ | |
| 40-60 | 3-F | H | 2,4,6-Me₃-C₆H₂—CH₂ | |
| 40-61 | 4-F | H | 2,4,6-Me₃-C₆H₂—CH₂ | |
| 40-62 | 2-Cl | H | 2,4,6-Me₃-C₆H₂—CH₂ | |
| 40-63 | 3-Cl | H | 2,4,6-Me₃-C₆H₂—CH₂ | |
| 40-64 | 4-Cl | H | 2,4,6-Me₃-C₆H₂—CH₂ | |
| 40-65 | 2-Br | H | 2,4,6-Me₃-C₆H₂—CH₂ | |
| 40-66 | 3-Br | H | 2,4,6-Me₃-C₆H₂—CH₂ | |
| 40-67 | 4-Br | H | 2,4,6-Me₃-C₆H₂—CH₂ | |
| 40-68 | 2-I | H | 2,4,6-Me₃-C₆H₂—CH₂ | |
| 40-69 | 3-I | H | 2,4,6-Me₃-C₆H₂—CH₂ | |
| 40-70 | 4-I | H | 2,4,6-Me₃-C₆H₂—CH₂ | |
| 40-71 | 2-Cl | 3-Cl | 2,4,6-Me₃-C₆H₂—CH₂ | |
| 40-72 | 2-Cl | 4-Cl | 2,4,6-Me₃-C₆H₂—CH₂ | |
| 40-73 | 2-Cl | 5-Cl | 2,4,6-Me₃-C₆H₂—CH₂ | |
| 40-74 | 2-Cl | 6-Cl | 2,4,6-Me₃-C₆H₂—CH₂ | |
| 40-75 | 3-Cl | 4-Cl | 2,4,6-Me₃-C₆H₂—CH₂ | |
| 40-76 | 3-Cl | 5-Cl | 2,4,6-Me₃-C₆H₂—CH₂ | |
| 40-77 | H | H | 2-MeO—C₆H₄—CH₂ | |
| 40-78 | 2-F | H | 2-MeO—C₆H₄—CH₂ | |
| 40-79 | 3-F | H | 2-MeO—C₆H₄—CH₂ | |
| 40-80 | 4-F | H | 2-MeO—C₆H₄—CH₂ | |
| 40-81 | 2-Cl | H | 2-MeO—C₆H₄—CH₂ | |
| 40-82 | 3-Cl | H | 2-MeO—C₆H₄—CH₂ | |
| 40-83 | 4-Cl | H | 2-MeO—C₆H₄—CH₂ | |
| 40-84 | 2-Br | H | 2-MeO—C₆H₄—CH₂ | |
| 40-85 | 3-Br | H | 2-MeO—C₆H₄—CH₂ | |
| 40-86 | 4-Br | H | 2-MeO—C₆H₄—CH₂ | 1H(d6DMSO): 3.71, OMe |
| 40-87 | 2-I | H | 2-MeO—C₆H₄—CH₂ | |
| 40-88 | 3-I | H | 2-MeO—C₆H₄—CH₂ | |
| 40-89 | 4-I | H | 2-MeO—C₆H₄—CH₂ | |
| 40-90 | 2-Cl | 3-Cl | 2-MeO—C₆H₄—CH₂ | |
| 40-91 | 2-Cl | 4-Cl | 2-MeO—C₆H₄—CH₂ | |
| 40-92 | 2-Cl | 5-Cl | 2-MeO—C₆H₄—CH₂ | |
| 40-93 | 2-Cl | 6-Cl | 2-MeO—C₆H₄—CH₂ | |
| 40-94 | 3-Cl | 4-Cl | 2-MeO—C₆H₄—CH₂ | |
| 40-95 | 3-Cl | 5-Cl | 2-MeO—C₆H₄—CH₂ | |
| 40-96 | H | H | 3-MeO—C₆H₄—CH₂ | |
| 40-97 | 2-F | H | 3-MeO—C₆H₄—CH₂ | |
| 40-98 | 3-F | H | 3-MeO—C₆H₄—CH₂ | |
| 40-99 | 4-F | H | 3-MeO—C₆H₄—CH₂ | |
| 40-100 | 2-Cl | H | 3-MeO—C₆H₄—CH₂ | |
| 40-101 | 3-Cl | H | 3-MeO—C₆H₄—CH₂ | |
| 40-102 | 4-Cl | H | 3-MeO—C₆H₄—CH₂ | |
| 40-103 | 2-Br | H | 3-MeO—C₆H₄—CH₂ | |
| 40-104 | 3-Br | H | 3-MeO—C₆H₄—CH₂ | |
| 40-105 | 4-Br | H | 3-MeO—C₆H₄—CH₂ | |
| 40-106 | 2-I | H | 3-MeO—C₆H₄—CH₂ | |
| 40-107 | 3-I | H | 3-MeO—C₆H₄—CH₂ | |
| 40-108 | 4-I | H | 3-MeO—C₆H₄—CH₂ | |
| 40-109 | 2-Cl | 3-Cl | 3-MeO—C₆H₄—CH₂ | |
| 40-110 | 2-Cl | 4-Cl | 3-MeO—C₆H₄—CH₂ | |
| 40-111 | 2-Cl | 5-Cl | 3-MeO—C₆H₄—CH₂ | |
| 40-112 | 2-Cl | 6-Cl | 3-MeO—C₆H₄—CH₂ | |
| 40-113 | 3-Cl | 4-Cl | 3-MeO—C₆H₄—CH₂ | |
| 40-114 | 3-Cl | 5-Cl | 3-MeO—C₆H₄—CH₂ | |
| 40-115 | H | H | 4-MeO—C₆H₄—CH₂ | |
| 40-116 | 2-F | H | 4-MeO—C₆H₄—CH₂ | |
| 40-117 | 3-F | H | 4-MeO—C₆H₄—CH₂ | |
| 40-118 | 4-F | H | 4-MeO—C₆H₄—CH₂ | |
| 40-119 | 2-Cl | H | 4-MeO—C₆H₄—CH₂ | |
| 40-120 | 3-Cl | H | 4-MeO—C₆H₄—CH₂ | |
| 40-121 | 4-Cl | H | 4-MeO—C₆H₄—CH₂ | |
| 40-122 | 2-Br | H | 4-MeO—C₆H₄—CH₂ | |
| 40-123 | 3-Br | H | 4-MeO—C₆H₄—CH₂ | |
| 40-124 | 4-Br | H | 4-MeO—C₆H₄—CH₂ | ¹H (d₆-DMSO): 6.79, 7.05, MeOphenyl |
| 40-125 | 2-I | H | 4-MeO—C₆H₄—CH₂ | |
| 40-126 | 3-I | H | 4-MeO—C₆H₄—CH₂ | |
| 40-127 | 4-I | H | 4-MeO—C₆H₄—CH₂ | |
| 40-128 | 2-Cl | 3-Cl | 4-MeO—C₆H₄—CH₂ | |
| 40-129 | 2-Cl | 4-Cl | 4-MeO—C₆H₄—CH₂ | |
| 40-130 | 2-Cl | 5-Cl | 4-MeO—C₆H₄—CH₂ | |
| 40-131 | 2-Cl | 6-Cl | 4-MeO—C₆H₄—CH₂ | |
| 40-132 | 3-Cl | 4-Cl | 4-MeO—C₆H₄—CH₂ | |
| 40-133 | 3-Cl | 5-Cl | 4-MeO—C₆H₄—CH₂ | |
| 40-134 | 4-Br | H | 3,4-(MeO)₂—C₆H₃—CH₂ | ¹H (d₆-DMSO): 3.59 (s, 3H, OMe), 3.66 |
| | | | | ppm (s, 3H, OMe) |

TABLE 41

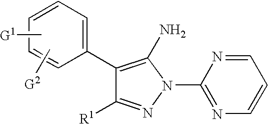

| Example | | | | |
|---|---|---|---|---|
| No. | G¹ | G² | R¹ | Physical data |
| 41-01 | H | H | C₆H₅—CH₂CH₂ | |
| 41-02 | 2-F | H | C₆H₅—CH₂CH₂ | |
| 41-03 | 3-F | H | C₆H₅—CH₂CH₂ | |
| 41-04 | 4-F | H | C₆H₅—CH₂CH₂ | |
| 41-05 | 2-Cl | H | C₆H₅—CH₂CH₂ | |
| 41-06 | 3-Cl | H | C₆H₅—CH₂CH₂ | |
| 41-07 | 4-Cl | H | C₆H₅—CH₂CH₂ | |
| 41-08 | 2-Br | H | C₆H₅—CH₂CH₂ | |
| 41-09 | 3-Br | H | C₆H₅—CH₂CH₂ | |
| 41-10 | 4-Br | H | C₆H₅—CH₂CH₂ | 1H(d6DMSO): 2.86, C₂H₄ |
| 41-11 | 2-I | H | C₆H₅—CH₂CH₂ | |
| 41-12 | 3-I | H | C₆H₅—CH₂CH₂ | |
| 41-13 | 4-I | H | C₆H₅—CH₂CH₂ | |
| 41-14 | 2-Cl | 3-Cl | C₆H₅—CH₂CH₂ | |
| 41-15 | 2-Cl | 4-Cl | C₆H₅—CH₂CH₂ | |
| 41-16 | 2-Cl | 5-Cl | C₆H₅—CH₂CH₂ | |
| 41-17 | 2-Cl | 6-Cl | C₆H₅—CH₂CH₂ | |
| 41-18 | 3-Cl | 4-Cl | C₆H₅—CH₂CH₂ | |
| 41-19 | 3-Cl | 5-Cl | C₆H₅—CH₂CH₂ | |
| 41-20 | H | H | 2-Cl—C₆H₄—CH₂CH₂ | |
| 41-21 | 2-F | H | 2-Cl—C₆H₄—CH₂CH₂ | |
| 41-22 | 3-F | H | 2-Cl—C₆H₄—CH₂CH₂ | |
| 41-23 | 4-F | H | 2-Cl—C₆H₄—CH₂CH₂ | |
| 41-24 | 2-Cl | H | 2-Cl—C₆H₄—CH₂CH₂ | |
| 41-25 | 3-Cl | H | 2-Cl—C₆H₄—CH₂CH₂ | |
| 41-26 | 4-Cl | H | 2-Cl—C₆H₄—CH₂CH₂ | |
| 41-27 | 2-Br | H | 2-Cl—C₆H₄—CH₂CH₂ | |
| 41-28 | 3-Br | H | 2-Cl—C₆H₄—CH₂CH₂ | |
| 41-29 | 4-Br | H | 2-Cl—C₆H₄—CH₂CH₂ | |
| 41-30 | 2-I | H | 2-Cl—C₆H₄—CH₂CH₂ | |
| 41-31 | 3-I | H | 2-Cl—C₆H₄—CH₂CH₂ | |
| 41-32 | 4-I | H | 2-Cl—C₆H₄—CH₂CH₂ | |
| 41-33 | 2-Cl | 3-Cl | 2-Cl—C₆H₄—CH₂CH₂ | |
| 41-34 | 2-Cl | 4-Cl | 2-Cl—C₆H₄—CH₂CH₂ | |
| 41-35 | 2-Cl | 5-Cl | 2-Cl—C₆H₄—CH₂CH₂ | |
| 41-36 | 2-Cl | 6-Cl | 2-Cl—C₆H₄—CH₂CH₂ | |
| 41-37 | 3-Cl | 4-Cl | 2-Cl—C₆H₄—CH₂CH₂ | |
| 41-38 | 3-Cl | 5-Cl | 2-Cl—C₆H₄—CH₂CH₂ | |
| 41-39 | H | H | 3-Cl—C₆H₄—CH₂CH₂ | |
| 41-40 | 2-F | H | 3-Cl—C₆H₄—CH₂CH₂ | |
| 41-41 | 3-F | H | 3-Cl—C₆H₄—CH₂CH₂ | |
| 41-42 | 4-F | H | 3-Cl—C₆H₄—CH₂CH₂ | |
| 41-43 | 2-Cl | H | 3-Cl—C₆H₄—CH₂CH₂ | |
| 41-44 | 3-Cl | H | 3-Cl—C₆H₄—CH₂CH₂ | |
| 41-45 | 4-Cl | H | 3-Cl—C₆H₄—CH₂CH₂ | |
| 41-46 | 2-Br | H | 3-Cl—C₆H₄—CH₂CH₂ | |
| 41-47 | 3-Br | H | 3-Cl—C₆H₄—CH₂CH₂ | |
| 41-48 | 4-Br | H | 3-Cl—C₆H₄—CH₂CH₂ | |
| 41-49 | 2-I | H | 3-Cl—C₆H₄—CH₂CH₂ | |
| 41-50 | 3-I | H | 3-Cl—C₆H₄—CH₂CH₂ | |
| 41-51 | 4-I | H | 3-Cl—C₆H₄—CH₂CH₂ | |
| 41-52 | 2-Cl | 3-Cl | 3-Cl—C₆H₄—CH₂CH₂ | |
| 41-53 | 2-Cl | 4-Cl | 3-Cl—C₆H₄—CH₂CH₂ | |
| 41-54 | 2-Cl | 5-Cl | 3-Cl—C₆H₄—CH₂CH₂ | |
| 41-55 | 2-Cl | 6-Cl | 3-Cl—C₆H₄—CH₂CH₂ | |
| 41-56 | 3-Cl | 4-Cl | 3-Cl—C₆H₄—CH₂CH₂ | |
| 41-57 | 3-Cl | 5-Cl | 3-Cl—C₆H₄—CH₂CH₂ | |
| 41-58 | H | H | 4-Cl—C₆H₄—CH₂CH₂ | |
| 41-59 | 2-F | H | 4-Cl—C₆H₄—CH₂CH₂ | |
| 41-60 | 3-F | H | 4-Cl—C₆H₄—CH₂CH₂ | |
| 41-61 | 4-F | H | 4-Cl—C₆H₄—CH₂CH₂ | |
| 41-62 | 2-Cl | H | 4-Cl—C₆H₄—CH₂CH₂ | |
| 41-63 | 3-Cl | H | 4-Cl—C₆H₄—CH₂CH₂ | |
| 41-64 | 4-Cl | H | 4-Cl—C₆H₄—CH₂CH₂ | |
| 41-65 | 2-Br | H | 4-Cl—C₆H₄—CH₂CH₂ | |
| 41-66 | 3-Br | H | 4-Cl—C₆H₄—CH₂CH₂ | |
| 41-67 | 4-Br | H | 4-Cl—C₆H₄—CH₂CH₂ | |
| 41-68 | 2-I | H | 4-Cl—C₆H₄—CH₂CH₂ | |
| 41-69 | 3-I | H | 4-Cl—C₆H₄—CH₂CH₂ | |
| 41-70 | 4-I | H | 4-Cl—C₆H₄—CH₂CH₂ | |
| 41-71 | 2-Cl | 3-Cl | 4-Cl—C₆H₄—CH₂CH₂ | |
| 41-72 | 2-Cl | 4-Cl | 4-Cl—C₆H₄—CH₂CH₂ | |
| 41-73 | 2-Cl | 5-Cl | 4-Cl—C₆H₄—CH₂CH₂ | |
| 41-74 | 2-Cl | 6-Cl | 4-Cl—C₆H₄—CH₂CH₂ | |
| 41-75 | 3-Cl | 4-Cl | 4-Cl—C₆H₄—CH₂CH₂ | |
| 41-76 | 3-Cl | 5-Cl | 4-Cl—C₆H₄—CH₂CH₂ | |
| 41-77 | H | H | C₆H₅—CH₂CH₂CH₂ | |
| 41-78 | 2-F | H | C₆H₅—CH₂CH₂CH₂ | |
| 41-79 | 3-F | H | C₆H₅—CH₂CH₂CH₂ | |
| 41-80 | 4-F | H | C₆H₅—CH₂CH₂CH₂ | |
| 41-81 | 2-Cl | H | C₆H₅—CH₂CH₂CH₂ | |
| 41-82 | 3-Cl | H | C₆H₅—CH₂CH₂CH₂ | |
| 41-83 | 4-Cl | H | C₆H₅—CH₂CH₂CH₂ | |
| 41-84 | 2-Br | H | C₆H₅—CH₂CH₂CH₂ | |
| 41-85 | 3-Br | H | C₆H₅—CH₂CH₂CH₂ | |
| 41-86 | 4-Br | H | C₆H₅—CH₂CH₂CH₂ | 1H(d6DMSO): 1.79, 2.55 ppm C₃H₆ |
| 41-87 | 2-I | H | C₆H₅—CH₂CH₂CH₂ | |
| 41-88 | 3-I | H | C₆H₅—CH₂CH₂CH₂ | |
| 41-89 | 4-I | H | C₆H₅—CH₂CH₂CH₂ | |
| 41-90 | 2-Cl | 3-Cl | C₆H₅—CH₂CH₂CH₂ | |
| 41-91 | 2-Cl | 4-Cl | C₆H₅—CH₂CH₂CH₂ | |
| 41-92 | 2-Cl | 5-Cl | C₆H₅—CH₂CH₂CH₂ | |
| 41-93 | 2-Cl | 6-Cl | C₆H₅—CH₂CH₂CH₂ | |
| 41-94 | 3-Cl | 4-Cl | C₆H₅—CH₂CH₂CH₂ | |
| 41-95 | 3-Cl | 5-Cl | C₆H₅—CH₂CH₂CH₂ | |

TABLE 42

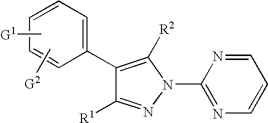

| Example | | | | | |
|---|---|---|---|---|---|
| No. | G¹ | G² | R¹ | R² | Physical data |
| 42-01 | H | H | CH₂F | NHEt | |
| 42-02 | 2-F | H | CH₂F | NHEt | |
| 42-03 | 3-F | H | CH₂F | NHEt | |
| 42-04 | 4-F | H | CH₂F | NHEt | |
| 42-05 | 2-Cl | H | CH₂F | NHEt | |
| 42-06 | 3-Cl | H | CH₂F | NHEt | |
| 42-07 | 4-Cl | H | CH₂F | NHEt | |
| 42-08 | 2-Br | H | CH₂F | NHEt | |
| 42-09 | 3-Br | H | CH₂F | NHEt | |
| 42-10 | 4-Br | H | CH₂F | NHEt | |
| 42-11 | 2-I | H | CH₂F | NHEt | |
| 42-12 | 3-I | H | CH₂F | NHEt | |
| 42-13 | 4-I | H | CH₂F | NHEt | |
| 42-14 | 2-Cl | 3-Cl | CH₂F | NHEt | |
| 42-15 | 2-Cl | 4-Cl | CH₂F | NHEt | |
| 42-16 | 2-Cl | 5-Cl | CH₂F | NHEt | |
| 42-17 | 2-Cl | 6-Cl | CH₂F | NHEt | |
| 42-18 | 2-Cl | 4-F | CH₂F | NHEt | |
| 42-19 | 3-Cl | 4-Cl | CH₂F | NHEt | |
| 42-20 | 3-Cl | 5-Cl | CH₂F | NHEt | NMR see below |
| 42-21 | H | H | CH₂F | NHCH₂Ph | |
| 42-22 | 2-F | H | CH₂F | NHCH₂Ph | |
| 42-23 | 3-F | H | CH₂F | NHCH₂Ph | |
| 42-24 | 4-F | H | CH₂F | NHCH₂Ph | |
| 42-25 | 2-Cl | H | CH₂F | NHCH₂Ph | |
| 42-26 | 3-Cl | H | CH₂F | NHCH₂Ph | |
| 42-27 | 4-Cl | H | CH₂F | NHCH₂Ph | |
| 42-28 | 2-Br | H | CH₂F | NHCH₂Ph | |
| 42-29 | 3-Br | H | CH₂F | NHCH₂Ph | |
| 42-30 | 4-Br | H | CH₂F | NHCH₂Ph | |
| 42-31 | 2-I | H | CH₂F | NHCH₂Ph | |
| 42-32 | 3-I | H | CH₂F | NHCH₂Ph | |
| 42-33 | 4-I | H | CH₂F | NHCH₂Ph | |
| 42-34 | 2-Cl | 3-Cl | CH₂F | NHCH₂Ph | |
| 42-35 | 2-Cl | 4-Cl | CH₂F | NHCH₂Ph | |
| 42-36 | 2-Cl | 5-Cl | CH₂F | NHCH₂Ph | |
| 42-37 | 2-Cl | 6-Cl | CH₂F | NHCH₂Ph | |
| 42-38 | 3-Cl | 4-Cl | CH₂F | NHCH₂Ph | |
| 42-39 | 3-Cl | 5-Cl | CH₂F | NHCH₂Ph | NMR see below |
| 42-40 | H | H | CH₂F | N(CH₂Ph)₂ | |
| 42-41 | 2-F | H | CH₂F | N(CH₂Ph)₂ | |
| 42-42 | 3-F | H | CH₂F | N(CH₂Ph)₂ | |
| 42-43 | 4-F | H | CH₂F | N(CH₂Ph)₂ | |
| 42-44 | 2-Cl | H | CH₂F | N(CH₂Ph)₂ | |
| 42-45 | 3-Cl | H | CH₂F | N(CH₂Ph)₂ | |
| 42-46 | 4-Cl | H | CH₂F | N(CH₂Ph)₂ | |
| 42-47 | 2-Br | H | CH₂F | N(CH₂Ph)₂ | |
| 42-48 | 3-Br | H | CH₂F | N(CH₂Ph)₂ | |
| 42-49 | 4-Br | H | CH₂F | N(CH₂Ph)₂ | |
| 42-50 | 2-I | H | CH₂F | N(CH₂Ph)₂ | |
| 42-51 | 3-I | H | CH₂F | N(CH₂Ph)₂ | |
| 42-52 | 4-I | H | CH₂F | N(CH₂Ph)₂ | |
| 42-53 | 2-Cl | 3-Cl | CH₂F | N(CH₂Ph)₂ | |
| 42-54 | 2-Cl | 4-Cl | CH₂F | N(CH₂Ph)₂ | |
| 42-55 | 2-Cl | 5-Cl | CH₂F | N(CH₂Ph)₂ | |
| 42-56 | 2-Cl | 6-Cl | CH₂F | N(CH₂Ph)₂ | |
| 42-57 | 3-Cl | 4-Cl | CH₂F | N(CH₂Ph)₂ | |
| 42-58 | 3-Cl | 5-Cl | CH₂F | N(CH₂Ph)₂ | NMR see below |
| 42-59 | H | H | CHF₂ | NEt₂ | |
| 42-60 | 2-F | H | CHF₂ | NEt₂ | |
| 42-61 | 3-F | H | CHF₂ | NEt₂ | |
| 42-62 | 4-F | H | CHF₂ | NEt₂ | |
| 42-63 | 2-Cl | H | CHF₂ | NEt₂ | |
| 42-64 | 3-Cl | H | CHF₂ | NEt₂ | |
| 42-65 | 4-Cl | H | CHF₂ | NEt₂ | |
| 42-66 | 2-Br | H | CHF₂ | NEt₂ | |
| 42-67 | 3-Br | H | CHF₂ | NEt₂ | |
| 42-68 | 4-Br | H | CHF₂ | NEt₂ | |
| 42-69 | 2-I | H | CHF₂ | NEt₂ | |
| 42-70 | 3-I | H | CHF₂ | NEt₂ | |
| 42-71 | 4-I | H | CHF₂ | NEt₂ | |
| 42-72 | 2-Cl | 3-Cl | CHF₂ | NEt₂ | |
| 42-73 | 2-Cl | 4-Cl | CHF₂ | NEt₂ | |
| 42-74 | 2-Cl | 5-Cl | CHF₂ | NEt₂ | |
| 42-75 | 2-Cl | 6-Cl | CHF₂ | NEt₂ | |
| 42-76 | 3-Cl | 4-Cl | CHF₂ | NEt₂ | |
| 42-77 | 3-Cl | 5-Cl | CHF₂ | NEt₂ | NMR see below |
| 42-78 | H | H | CHF₂ | NHCH₂Ph | |
| 42-79 | 2-F | H | CHF₂ | NHCH₂Ph | |
| 42-80 | 3-F | H | CHF₂ | NHCH₂Ph | |
| 42-81 | 4-F | H | CHF₂ | NHCH₂Ph | |
| 42-82 | 2-Cl | H | CHF₂ | NHCH₂Ph | |
| 42-83 | 3-Cl | H | CHF₂ | NHCH₂Ph | |
| 42-84 | 4-Cl | H | CHF₂ | NHCH₂Ph | |
| 42-85 | 2-Br | H | CHF₂ | NHCH₂Ph | |
| 42-86 | 3-Br | H | CHF₂ | NHCH₂Ph | |
| 42-87 | 4-Br | H | CHF₂ | NHCH₂Ph | |
| 42-88 | 2-I | H | CHF₂ | NHCH₂Ph | |
| 42-89 | 3-I | H | CHF₂ | NHCH₂Ph | |
| 42-90 | 4-I | H | CHF₂ | NHCH₂Ph | |
| 42-91 | 2-Cl | 3-Cl | CHF₂ | NHCH₂Ph | |
| 42-92 | 2-Cl | 4-Cl | CHF₂ | NHCH₂Ph | |
| 42-93 | 2-Cl | 5-Cl | CHF₂ | NHCH₂Ph | |
| 42-94 | 2-Cl | 6-Cl | CHF₂ | NHCH₂Ph | |
| 42-95 | 3-Cl | 4-Cl | CHF₂ | NHCH₂Ph | |
| 42-96 | 3-Cl | 5-Cl | CHF₂ | NHCH₂Ph | NMR see below |
| 42-97 | H | H | CHF₂ | N(CH₂Ph)₂ | |
| 42-98 | 2-F | H | CHF₂ | N(CH₂Ph)₂ | |
| 42-99 | 3-F | H | CHF₂ | N(CH₂Ph)₂ | |
| 42-100 | 4-F | H | CHF₂ | N(CH₂Ph)₂ | |
| 42-101 | 2-Cl | H | CHF₂ | N(CH₂Ph)₂ | |
| 42-102 | 3-Cl | H | CHF₂ | N(CH₂Ph)₂ | |
| 42-103 | 4-Cl | H | CHF₂ | N(CH₂Ph)₂ | |
| 42-104 | 2-Br | H | CHF₂ | N(CH₂Ph)₂ | |
| 42-105 | 3-Br | H | CHF₂ | N(CH₂Ph)₂ | |
| 42-106 | 4-Br | H | CHF₂ | N(CH₂Ph)₂ | |
| 42-107 | 2-I | H | CHF₂ | N(CH₂Ph)₂ | |
| 42-108 | 3-I | H | CHF₂ | N(CH₂Ph)₂ | |
| 42-109 | 4-I | H | CHF₂ | N(CH₂Ph)₂ | |
| 42-110 | 2-Cl | 3-Cl | CHF₂ | N(CH₂Ph)₂ | |
| 42-111 | 2-Cl | 4-Cl | CHF₂ | N(CH₂Ph)₂ | |
| 42-112 | 2-Cl | 5-Cl | CHF₂ | N(CH₂Ph)₂ | |
| 42-113 | 2-Cl | 6-Cl | CHF₂ | N(CH₂Ph)₂ | |
| 42-114 | 3-Cl | 4-Cl | CHF₂ | N(CH₂Ph)₂ | |
| 42-115 | 3-Cl | 5-Cl | CHF₂ | N(CH₂Ph)₂ | NMR see below |
| 42-116 | H | H | CHF₂ | NHCOCH₃ | |
| 42-117 | 2-F | H | CHF₂ | NHCOCH₃ | |
| 42-118 | 3-F | H | CHF₂ | NHCOCH₃ | |
| 42-119 | 4-F | H | CHF₂ | NHCOCH₃ | |
| 42-120 | 2-Cl | H | CHF₂ | NHCOCH₃ | |
| 42-121 | 3-Cl | H | CHF₂ | NHCOCH₃ | |
| 42-122 | 4-Cl | H | CHF₂ | NHCOCH₃ | |
| 42-123 | 2-Br | H | CHF₂ | NHCOCH₃ | |
| 42-124 | 3-Br | H | CHF₂ | NHCOCH₃ | |
| 42-125 | 4-Br | H | CHF₂ | NHCOCH₃ | |
| 42-126 | 2-I | H | CHF₂ | NHCOCH₃ | |
| 42-127 | 3-I | H | CHF₂ | NHCOCH₃ | |
| 42-128 | 4-I | H | CHF₂ | NHCOCH₃ | |
| 42-129 | 2-Cl | 3-Cl | CHF₂ | NHCOCH₃ | |
| 42-130 | 2-Cl | 4-Cl | CHF₂ | NHCOCH₃ | |
| 42-131 | 2-Cl | 5-Cl | CHF₂ | NHCOCH₃ | |
| 42-132 | 2-Cl | 6-Cl | CHF₂ | NHCOCH₃ | |
| 42-133 | 2-Cl | 4-F | CHF₂ | NHCOCH₃ | |
| 42-134 | 3-Cl | 4-Cl | CHF₂ | NHCOCH₃ | |
| 42-135 | 3-Cl | 5-Cl | CHF₂ | NHCOCH₃ | NMR see below |

TABLE 43

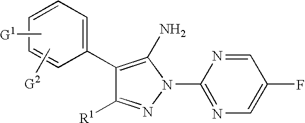

| Example | | | | |
|---|---|---|---|---|
| No. | G¹ | G² | R¹ | Physical data |
| 43-01 | 3-CF₃ | 5-Cl | (R)-CH(CH₃)F | ¹H-NMR (CD₃CN): 8.71 ppm (s, 1H) |
| 43-02 | 3-CF₃ | 4-Cl | CF₃ | NMR see below |
| 43-03 | 3-CF₃ | 5-Cl | CF₂Br | NMR see below |
| 43-04 | 3-CF₃ | 5-Cl | CF₂Cl | NMR see below |
| 43-05 | 3-CF₃ | 5-Cl | CF₃ | NMR see below |
| 43-06 | 3-CF₃ | 5-Cl | CF₂CH₃ | NMR see below |
| 43-07 | 3-Cl | 5-F | CF₃ | lgP = 3.24 |
| 43-08 | 3-F | 5-F | CF₃ | lgP = 2.93 |
| 43-09 | 2-F | 4-Br | CF₃ | lgP = 3.15 |
| 43-10 | 3-F | 4-F | CF₃ | lgP = 2.84 |
| 43-11 | 3-Br | 5-Br | CF₃ | lgP = 3.84 |
| 43-12 | 3-CH₃ | 3-CH₃ | CF₃ | lgP = 3.28 |
| 43-13 | 3-CF₃ | 5-Cl | CF₂CF₃ | |
| 43-14 | 3-CF₃ | 5-Cl | CCl₂F | |
| 43-15 | 3-CF₃ | 4-Cl | CH(CH₃)F | |
| 43-16 | 3-CF₃ | 4-Cl | CF₂Br | |
| 43-17 | 3-CF₃ | 4-Cl | CF₂Cl | |
| 43-18 | 3-CF₃ | 4-Cl | CCl₂F | |
| 43-19 | 3-CF₃ | 4-Cl | CF₂CH₃ | |
| 43-20 | 3-CF₃ | 4-Cl | CF₂CF₃ | |
| 43-21 | 3-Cl | 5-Cl | CF₂Br | |
| 43-22 | 3-Cl | 5-Cl | CF₂Cl | |
| 43-23 | 3-Cl | 5-Cl | CCl₂F | |
| 43-24 | 3-Cl | 5-Cl | CH(CH₃)F | |
| 43-25 | 3-Cl | 5-Cl | CF₂CH₃ | |
| 43-26 | 3-Cl | 5-Cl | CF₂CF₃ | |
| 43-27 | 3-CF₃ | 5-CF₃ | CF₃ | |
| 43-28 | 3-CF₃ | 5-CF₃ | CF₂Br | |
| 43-29 | 3-CF₃ | 5-CF₃ | CF₂Cl | |
| 43-30 | 3-CF₃ | 5-CF₃ | CCl₂F | |
| 43-31 | 3-CF₃ | 5-CF₃ | CH(CH₃)F | |
| 43-32 | 3-CF₃ | 5-CF₃ | CF₂CH₃ | |
| 43-33 | 3-CF₃ | 5-CF₃ | CF₂CF₃ | |
| 43-34 | 4-I | H | CF₂Br | |
| 43-35 | 4-I | H | CF₂Cl | |
| 43-36 | 4-I | H | CCl₂F | |
| 43-37 | 4-I | H | CH(CH₃)F | |
| 43-38 | 4-I | H | CF₂CH₃ | |
| 43-39 | 4-I | H | CF₂CF₃ | |
| 43-40 | 3-Cl | 4-Cl | CF₂Br | |
| 43-41 | 3-Cl | 4-Cl | CF₂Cl | |
| 43-42 | 3-Cl | 4-Cl | CCl₂F | |
| 43-43 | 3-Cl | 4-Cl | CH(CH₃)F | |
| 43-44 | 3-Cl | 4-Cl | CF₂CH₃ | |
| 43-45 | 3-Cl | 4-Cl | CF₂CF₃ | |

The logP values given in the tables were determined in accordance with EEC-Directive 79/831 Annex V.A8 by HPLC (gradient method, acetonitrile/0.1% strength aqueous phosphoric acid or formic acid).

NMR Data for the Synthesis Examples

### Ex. No. 1-01

¹H-NMR (400 MHz, CDCl₃), δ 8.83 (d, 2H), 7.37 (m, 2H), 7.21 (m, 2H), 7.16 (m, 1H), 6.03 (br. s, 2H, NH).

### Ex. No. 1-02

¹H-NMR (400 MHz, d₆-DMSO, δ, ppm): 8.93 (d, 2H), 7.53-7.46 (m, 2H), 7.21-7.14 (m, 3H), 6.83 (broad s, 2H, NH₂).

### Ex. No. 1-03

¹H-NMR (400 MHz, CD₃CN, δ, ppm): 8.83 (d, 2H), 7.43-7.35 (m, 3H), 7.22-7.16 (m, 2H), 6.17 (broad s, 2H, NH₂).

### Ex. No. 1-06

¹H-NMR (400 MHz, CD₃CN, δ, ppm): 8.83 (d, 2H), 7.48 (d, 2H), 7.39-7.35 (m, 3H), 6.23 (broad s, 2H, NH₂).

### Ex. No. 1-07

¹H-NMR (400 MHz, CDCl₃), δ 8.84 (d, 2H), 7.37 (m, 2H), 7.28 (t, 1H), 7.22 (m,1H), 7.12 (m, 1H), 6.01 (br. s, 2H, NH).

### Ex. No. 1-08

¹H-NMR (400 MHz, d₆-DMSO), δ 8.96 (d, 2H), 7.55 (m, 3H), 7.42 (t, 1H), 7.36 (d, 1H), 7.04 (s, 2H, NH).

### Ex. No. 1-09

¹H-NMR (400 MHz, CD₃CN, δ, ppm): 8.83 (d, 2H), 7.61 (d, 2H), 7.37 (t, 1H), 7.33 (d, 2H), 6.23 (broad s, 2H, NH₂).

### Ex. No. 1-10

¹H-NMR (400 MHz, CDCl₃), δ 8.82 (d, 2H), 7.96 (d, 1H), 7.42 (d, 1H), 7.37 (dd, 1H), 7.28 (t, 1H), 7.08 (dd, 1H), 5.84 (s, 2H, NH).

### Ex. No. 1-11

¹H-NMR (400 MHz, CDCl₃), δ 8.83 (d, 2H), 7.78 (s, 1H), 7.69 (d, 1H), 7.39 (d, 1H), 7.28 (t, 1H), 7.18 (t, 1H), 6.05 (s, 2H, NH).

### Ex. No. 1-12

¹H-NMR (400 MHz, CDCl₃), δ 8.82 (d, 2H), 7.78 (d, 2H), 7.27 (t, 1H), 7.14 (d,2H), 6.02 (s, 2H, NH).

### Ex. No. 1-13

¹H-NMR (400 MHz, d₆-DMSO), δ 8.96 (d, 2H), 7.56 (t, 1H), 7.44 (m, 1H), 7.26 (m, 1H), 7.16 (m, 1H), 7.11 (s, 2H, NH).

### Ex. No. 1-14

¹H-NMR (400 MHz, CDCl₃), δ 8.84 (d, 2H), 7.36 (m, 1H), 7.28 (t, 1H), 6.92 (m, 2H), 5.99 (s, 2H, NH).

### Ex. No. 1-15

¹H-NMR (400 MHz, CDCl₃), δ 8.83 (d, 2H), 7.25 (t, 1H), 7.04-7.17 (m, 3H), 6.09 (s, 2H, NH).

### Ex. No. 1-17

¹H-NMR (400 MHz, CDCl₃), δ 8.84 (d, 2H), 7.42 (dt, 1H), 7.28 (m, 2H), 7.17 (t, 1H), 6.06 (s, 2H, NH).

### Ex. No. 1-18

¹H-NMR (400 MHz, CDCl₃), δ 8.84 (d, 2H), 7.29-7.36 (m, 3H), 7.19 (m, 1H), 6.03 (s, 2H, NH).

### Ex. No. 1-20

¹H-NMR (400 MHz, CDCl₃), δ 8.85 (d, 2H), 7.57 (m, 1H), 7.34 (m, 2H), 7.12 (dt, 1H), 6.52 (br. s, 2H, NH).

### Ex. No. 1-22

¹H-NMR (400 MHz, CDCl₃), δ 8.82 (d, 2H), 7.39 (m, 2H), 7.32 (t, 1H), 7.25 (m, 1H), 6.41 (s, 2H, NH).

### Ex. No. 1-23

¹H-NMR (400 MHz, CDCl₃), δ 8.85 (d, 2H), 7.49 (m, 2H), 7.35 (dd, 1H), 7.09 (t, 1H), 6.37 (s, 2H, NH).

### Ex. No. 1-30

¹H-NMR (400 MHz, CDCl₃), δ 8.84 (d, 2H), 7.36 (dd, 1H), 7.29 (m, 1H), 7.27 (t, 1H), 7.06 (dt, 1H), 5.92 (br. s, 2H, NH).

### Ex. No. 1-32

¹H-NMR (400 MHz, d₆-DMSO), δ 8.94 (d, 2H), 7.69 (dd, 1H), 7.55 (t, 1H), 7.42 (dd, 1H), 7.37 (dd, 1H), 7.03 (s, 2H, NH).

### Ex. No. 1-33

¹H-NMR (400 MHz, CD₃CN, δ, ppm): 8.83 (d, 2H), 7.63 (s, 1H), 7.43-7.36 (m, 3H), 6.18 (broad s, 2H, NH₂).

### Ex. No. 1-34

¹H-NMR (400 MHz, d₆-DMSO), δ 8.96 (d, 2H), 7.62 (d, 1H), 7.50 (m, 2H), 7.47 (d, 1H), 7.05 (s, 2H, NH).

### Ex. No. 1-35

¹H-NMR (400 MHz, CDCl₃), δ 8.85 (d, 2H), 7.58 (m, 1H), 7.41 (m, 1H), 7.35 (t, 1H), 7.13 (m, 1H), 6.54 (br. s, 2H, NH).

### Ex. No. 1-38

¹H-NMR (400 MHz, CDCl₃), δ 8.83 (d, 2H), 7.49 (dd, 1H), 7.46 (d, 1H), 7.38 (d, 1H), 7.28 (t, 1H), 5.97 (br. s, 2H, NH).

### Ex. No. 1-71

¹H-NMR (400 MHz, CDCl₃), δ 8.84 (d, 2H), 7.22-7.31 (m, 3H), 7.16 (m, 1H), 6.04 (s, 2H, NH).

### Ex. No. 1-73

¹H-NMR (400 MHz, d₆-DMSO), δ 8.94 (d, 2H), 7.64 (t, 1H), 7.57 (t, 1H), 7.37 (dd, 1H), 7.19 (dd, 1H), 7.09 (s, 2H, NH).

### Ex. No. 1-74

¹H-NMR (400 MHz, CDCl₃), δ 8.82 (d, 2H), 7.28 (t, 1H), 7.20 (s, 1H), 7.03 (m, 2H), 6.18 (s, 2H, NH).

### Ex. No. 1-75

¹H-NMR (400 MHz, CDCl₃), δ 8.84 (d, 2H), 7.61 (t, 1H), 7.28 (t, 1H), 7.19 (dd, 1H), 7.09 (d, 1H), 6.09 (br. s, 2H, NH).

### Ex. No. 1-79

¹H-NMR (400 MHz, CDCl₃), δ 8.82 (d, 2H), 7.46 (dd, 1H), 7.21-7.30 (m, 3H), 6.03 (s, 2H, NH).

### Ex. No. 1-80

¹H-NMR (400 MHz, CD₃CN, δ, ppm): 8.84 (d, 2H), 7.60-7.55 (d+s, 2H), 7.37 (t, 1H), 7.33 (d, 1H), 6.33 (broad s, 2H, NH₂).

### Ex. No. 1-81

¹H-NMR (400 MHz, CD₃CN, δ, ppm): 8.84 (d, 2H), 7.46 (s, 1H), 7.39-7.36 (m, 3H), 6.38 (broad s, 2H, NH₂).

### Ex. No. 1-82

¹H-NMR (400 MHz, d₆-DMSO), δ 8.95 (d, 2H), 7.84 (d, 1H), 7.57 (d, 1H), 7.54 (t, 1H), 7.23 (dd, 1H), 7.12 (s, 2H, NH).

### Ex. No. 1-83

¹H-NMR (400 MHz, CDCl₃), δ 8.84 (d, 2H), 7.52 (m, 1H), 7.46 (m, 1H), 7.36 (m, 1H), 7.29 (t, 1H), 6.17 (br. s, 2H, NH).

### Ex. No. 1-87

¹H-NMR (400 MHz, d₆-DMSO), δ 8.97 (d, 2H), 7.72 (d, 1H), 7.69 (d, 1H), 7.56 (t, 1H), 7.37 (dd, 1H), 7.14 (br. s, 2H, NH).

### Ex. No. 1-88

¹H-NMR (400 MHz, CDCl₃), δ 8.83 (d, 2H), 7.70 (m, 1H), 7.68 (m, 1H), 7.28 (t, 1H), 7.20 (dd, 1H), 6.09 (s, 2H, NH).

### Ex. No. 1-89

¹H-NMR (400 MHz, CDCl₃), δ 8.84 (d, 2H), 7.67 (m, 1H), 7.52 (m, 2H), 7.30 (t, 1H), 6.12 (s, 2H, NH).

### Ex. No. 2-02

¹H-NMR (400 MHz, CDCl₃), δ 8.84 (d, 2H), 7.29 (t, 1H), 6.88-7.04 (m, 2H), 6.13 (s, 2H, NH).

### Ex. No. 2-04

¹H-NMR (400 MHz, d₆-DMSO), δ 8.96 (d, 2H), 7.64 (m, 1H), 7.56 (t, 1H), 7.50 (m, 1H), 7.13 (s, 2H, NH).

### Ex. No. 2-05

¹H-NMR (400 MHz, CDCl₃), δ 8.81 (d, 2H), 7.28 (t, 1H), 6.78 (d, 1H), 6.75 (d, 1H), 6.08 (s, 2H, NH).

### Ex. No. 2-06

¹H-NMR (400 MHz, d₆-DMSO, δ, ppm): 8.93 (d, 2H), 7.53 (t, 1H), 7.26-7.22 (dd, 2H), 6.96 (broad s, 2H, NH₂).

### Ex. No. 2-12

¹H-NMR (400 MHz, CDCl₃), δ]8.84 (d, 2H), 7.65 (s, 1H), 7.49 (s, 1H), 7.27 (t, 1H), 5.98 (s, 2H).

### Ex. No. 2-13

¹H-NMR (400 MHz, CDCl₃), δ 8.82 (d, 2H), 7.25 (t, 1H), 7.04 (d, 2H), 6.04 (s,2H, NH).

### Ex. No. 2-14

¹H-NMR (400 MHz, CDCl₃), δ 8.84 (d, 2H), 7.58 (m, 1H), 7.28 (t, 1H), 7.20 (m, 1H), 5.98 (s, 2H, NH).

### Ex. No. 2-15

¹H-NMR (400 MHz, CDCl₃), δ 8.85 (d, 2H), 7.38 (m, 1H), 7.33 (t, 1H), 7.22 (m, 1H), 6.32 (br. s, 2H, NH).

### Ex. No. 2-16

¹H-NMR (400 MHz, CDCl₃), δ 8.84 (d, 2H), 7.32 (t, 1H), 7.14 (m, 1H), 6.09 (br. d, 2H, NH).

### Ex. No. 2-17

¹H-NMR (400 MHz, CDCl₃), δ 8.84 (d, 2H), 7.33 (t, 1H), 7.24 (m, 1H), 7.06 (m, 1H), 6.03 (s, 2H, NH).

### Ex. No. 3-02

¹H-NMR (400 MHz, d₆-DMSO, δ, ppm): 8.94 (d, 2H), 7.71-7.64 (m, 3H), 7.53 (t, 1H), 6.90 (broad s, 2H, NH₂).

### Ex. No. 3-03

¹H-NMR (400 MHz, CD₃CN, δ, ppm): 8.94 (d, 2H), 7.79 (d, 2H), 7.60 (d, 2H), 7.53 (t, 1H), 6.92 (broad s, 2H, NH₂).

### Ex. No. 3-11

¹H-NMR (400 MHz, CDCl₃), δ 8.84 (d, 2H), 7.96 (d, 1H), 7.82 (d, 1H), 7.68 (s, 1H), 7.29 (t, 1H), 5.84 (br. s, 2H, NH).

### Ex. No. 3-14

¹H-NMR (400 MHz, CDCl₃), δ 8.85 (d, 2H), 7.88 (m, 3H), 7.31 (t, 1H),6.18 (s, 2H, NH).

### Ex. No. 3-15

¹H-NMR (400 MHz, CDCl₃), δ 8.83 (d, 2H), 7.52 (m, 2H), 7.45 (d, 1H), 7.29 (t, 1H), 6.12 (s, 2H, NH).

### Ex. No. 3-20

¹H-NMR (400 MHz, CDCl₃), δ 8.85 (d, 2H), 7.78 (d, 1H), 7.60 (s, 1H), 7.44 (d, 1H), 7.32 (t, 1H), 6.52 (s, 2H, NH).

### Ex. No. 3-23

¹H-NMR (400 MHz, CDCl₃), δ 8.83 (d, 2H), 7.63 (m, 2H), 7.31 (d, 1H), 7.22 (t, 1H), 6.05 (br. s, 2H, NH).

### Ex. No. 3-24

¹H-NMR (400 MHz, d₆-DMSO), δ 8.94 (d, 2H), 7.82 (d, 1H), 7.77 (d, 1H), 7.66 (dd, 1H), 7.55 (t, 1H), 7.19 (s, 2H, NH).

### Ex. No. 3-26

¹H-NMR (400 MHz, CDCl₃), δ 8.84 (d, 2H), 7.62 (m, 3H), 7.29 (t, 1H), 5.99 (br. s, 2H, NH).

### Ex. No. 3-31

¹H-NMR (400 MHz, CDCl₃), δ 8.84 (d, 2H), 7.66 (d, 1H), 7.57 (m, 1H), 7.29 (m, 2H), 6.04 (s, 2H, NH).

### Ex. No. 3-32

### ¹H-NMR (400 MHz, CDCl₃), δ 8.83 (d, 2H), 7.49 (s, 1H), 7.30 (m, 2H), 7.28 (t, 1H), 6.18 (s, 2H, NH).

### Ex. No. 3-33

¹H-NMR (400 MHz, CDCl₃), δ 8.82 (d, 2H), 7.60 (m, 3H), 7.31 (t, 1H), 6.02 (br. s. 2H, NH).

### Ex. No. 3-34

¹H-NMR (400 MHz, CDCl₃), δ 8.84 (d, 2H), 7.60 (m, 2H), 7.32 (m, 1H), 7.28 (t, 1H), 6.09 (br. s, 2H, NH).

### Ex. No. 4-02

¹H-NMR (400 MHz, CDCl₃), δ 8.83 (d, 2H), 7.44 (t, 1H), 7.28 (m, 2H), 7.19 (s, 1H), 7.12 (dd, 1H), 6.38-6.75 (t, 1H), 6.07 (s, 2H, NH).

### Ex. No. 4-03

¹H-NMR (400 MHz, CDCl₃), δ 8.83 (d, 2H), 7.42 (d, 2H), 7.27 (t, 1H), 7.20 (d, 2H). 6.38-6.76 (t, 1H), 6.02 (s, 2H, NH).

### Ex. No. 4-05

¹H-NMR (400 MHz, CDCl₃), δ 8.82 (d, 2H), 7.47 (t, 1H), 7.35 (d, 1H), 7.19-7.32 (m, 3H), 6.09 (s, 2H, NH).

### Ex. No. 4-06

¹H-NMR (400 MHz, CD₃CN, δ, ppm): 8.84 (d, 2H), 7.50 (d, 2H), 7.38-7.36 (m, 3H), 6.24 (broad s, 2H, NH₂).

### Ex. No. 4-07

¹H-NMR (400 MHz, CDCl₃), δ 8.84 (d, 2H), 7.46 (d, 2H), 7.32 (d, 2H), 7.28 (t, 1H), 66.05 (br. s, 2H, NH).

### Ex. No. 4-10

¹H-NMR (400 MHz, d₆-DMSO), δ 8.96 (d, 2H), 7.56 (t, 1H), 7.45 (d, 2H), 7.36 (d, 2H), 7.26-7.44 (dt, 1H), 7.01 (br. s, 2H, NH).

### Ex. No. 4-16

¹H-NMR (400 MHz, CDCl₃), δ 8.84 (d, 2H), 7.46 (t, 1H), 7.37 (d, 1H), 7.27 (t, 1H), 7.24 (m, 1H), 7.19 (m, 1H), 6.09 (br. s, 2H, NH), 4.93-5.16 (double sext., 1H).

### Ex. No. 4-18

¹H-NMR (400 MHz, CDCl₃), δ 8.84 (d, 2H), 7.28-7.41 (m, 4H), 6.06 (s, 2H, NH).

### Ex. No. 4-19

¹H-NMR (400 MHz, d₆-DMSO), δ 8.95 (d, 2H), 7.64 (dt, 1H), 7.56 (t, 1H), 7.45 (dd, 1H), 7.29 (d, 1H), 7.17 (s, 2H, NH).

### Ex. No. 4-21

¹H-NMR (400 MHz, d₆-DMSO), δ 8.96 (d, 2H), 7.78 (d, 1H), 7.55 (t, 1H), 7.43 (m, 2H), 7.16 (s, 2H, NH).

### Ex. No. 4-23

¹H-NMR (400 MHz, d₆-DMSO), δ 8.95 (d, 2H), 7.58 (m, 1H), 7.56 (t, 1H), 7.47 (m, 1H), 7.28 (s, 1H), 7.21 (s, 2H, NH).

### Ex. No. 4-34

¹H-NMR (400 MHz, CDCl₃), δ 8.84 (d, 2H), 7.46 (m, 2H), 7.39 (m, 1H), 7.30 (t, 1H), 6.16 (s, 2H, NH).

### Ex. No. 4-43

¹H-NMR (400 MHz, d₆-DMSO), δ 8.95 (d, 2H), 7.53 (t, 1H), 7.44 (m, 2H), 7.37 (dd, 1H), 7.16 (s, 2H, NH).

### Ex. No. 4-55

¹H-NMR (400 MHz, d₆-DMSO), δ 8.97 (d, 2H), 7.60 (dd, 1H), 7.56 (t, 1H), 7.42 (d, 1H), 7.36 (dd, 1H), 7.29 (s, 1H), 7.08 (s, 2H, NH).

### Ex. No. 5-01

H-NMR (400 MHz, CD₃CN, δ, ppm): 8.84 (d, 2H), 7.49-7.35 (m, 6H), 6.17 (broad s, 2H, NH₂).

### Ex. No. 5-02

¹H-NMR (400 MHz, CD₃CN, δ, ppm): 8.83 (d, 2H), 7.35 (t, 1H), 7.28 (s, 4H), 6.14 (broad s, 2H, NH₂), 2.38 (s, 3H).

### Ex. No. 5-22

¹H-NMR (400 MHz, CD₃CN, δ, ppm): 8.83 (d, 2H), 7.35 (t, 1H), 7.30 (d, 2H), 7.02 (d, 2H), 6.10 (broad s, 2H, NH₂), 3.83 (s, 3H).

### Ex. No. 5-58

¹H-NMR (400 MHz, d₆-DMSO), δ 8.95 (d, 2H), 7.56 (t, 1H), 7.35 (d, 2H), 7.27 (d, 2H), 6.91 (s, 2H, NH), 2.48 (s, 3H).

### Ex. No. 5-59

¹H-NMR (400 MHz, CDCl₃), δ 8.94 (d, 2H), 7.72 (d, 2H), 7.58 (d, 2H), 7.30 (t, 1H), 6.22 (s, 2H, NH), 2.80 (s, 3H).

### Ex. No. 5-60

¹H-NMR (400 MHz, CDCl₃), δ 8.84 (d, 2H), 7.99 (d, 2H), 7.62 (d, 2H), 7.32 (t, 1H), 6.23 (s, 2H, NH), 3.11 (s, 3H).

### Ex. No. 5-62

¹H-NMR (400 MHz, CD₃CN, δ, ppm): 8.84 (d, 2H), 7.78 (d, 2H), 7.54 (d, 2H), 7.38 (t, 1H), 6.33 (broad s, 2H, NH₂).

### Ex. No. 5-71

¹H-NMR (400 MHz, CD₃CN, δ, ppm): 8.84 (d, 2H), 7.80 (d, 2H), 7.58 (d, 2H), 7.38 (t, 1H), 6.37 (broad s, 2H, NH₂).

### Ex. No. 5-89

¹H-NMR (400 MHz, CDCl₃), δ 8.80 (d, 2H), 7.28 (d, 2H), 7.23 (t, 1H), 6.79 (d, 2H), 4.92 (br. s, 2H, NH), 3.00 (s, 6H).

### Ex. No. 6-01

¹H-NMR (400 MHz, CDCl₃), δ 8.82 (d, 2H), 7.32 (t, 1H), 7.25 (t, 1H), 7.17 (m, 3H), 6.03 (br. s, 2H, NH), 2.41 (s, 3H).

### Ex. No. 6-61

¹H-NMR (400 MHz, CDCl₃), δ 8.83 (d, 2H), 7.75 (s, 1H), 7.66 (d, 1H), 7.50 (m, 2H), 7.28 (t, 1H), 6.08 (s, 2H, NH).

### Ex. No. 6-70

¹H-NMR (400 MHz, CDCl₃), δ 8.84 (d, 2H), 7.71 (s, 1H), 7.63 (m, 2H), 7.57 (m, 1H), 7.29 (t, 1H), 6.20 (br. s, 2H, NH).

### Ex. No. 6-72

¹H-NMR (400 MHz, CDCl₃), δ 8.83 (d, 2H), 8.15 (d, 2H), 7.49 (d, 2H), 7.28 (t, 1H), 6.14 (br. s, 2H, NH), 3.96 (s, 3H).

### Ex. No. 6-78

¹H-NMR (400 MHz, CDCl₃), δ 8.84 (d, 2H), 8.32 (m, 1H), 8.22 (dd, 1H), 7.76 (d, 1H), 7.64 (dd, 1H), 7.33 (t, 1H), 6.19 (br. s, 2H, NH).

### Ex. No. 6-77

¹H-NMR (400 MHz, CDCl₃), δ 8.82 (d, 2H), 7.32 (dd, 1H), 7.25 (t, 1H), 6.72 (m, 3H), 6.03 (br. s, 2H, NH), 2.98 (s, 6H).

### Ex. No. 7-01

¹H-NMR (400 MHz, d₆-DMSO, δ, ppm): 8.92 (d, 2H), 7.50 (t, 1H), 7.33-7.09 (m, 4H), 6.51 (broad s, 2H, NH₂), 2.17 (s, 3H).

### Ex. No. 8-05

¹H-NMR (400 MHz, d₆-DMSO), δ 8.95 (d, 2H), 7.53 (t, 1H), 7.22 (d, 1H), 7.14 (d, 1H), 7.07 (dd, 1H), 6.80 (s, 2H, NH), 2.26 (s, 6H).

### Ex. No. 8-06

¹H-NMR (400 MHz, d₆-DMSO, δ, ppm): 8.93 (d, 2H), 7.51 (t, 1H), 6.99 (s, 1H), 6.96 (s, 2H), 6.62 (broad s, 2H, NH₂), 2.31 (s, 6H).

### Ex. No. 8-25

¹H-NMR (400 MHz, d₆-DMSO), δ 8.95 (d, 2H), 7.56 (t, 1H), 7.25 (dd, 1H), 7.17 (dd, 1H), 7.10 (d, 1H), 6.93 (s, 2H, NH), 3.87 (s, 3H).

### Ex. No. 8-26

¹H-NMR (400 MHz, CDCl₃), δ 8.83 (d, 2H), 7.42 (m, 1H), 7.27 (d, 1H), 7.25 (t, 1H), 7.01 (d, 1H), 5.98 (br. s, 2H, NH), 3.95 (s, 3H).

### Ex. No. 8-27

¹H-NMR (400 MHz, CDCl₃), δ 8.82 (d, 2H), 7.40 (d, 1H), 7.32 (dd, 1H), 7.27 (t, 1H), 6.97 (d, 1H), 6.02 (br. s, 2H, NH), 3.95 (s, 3H).

### Ex. No. 8-33

¹H-NMR (400 MHz, d₆-DMSO), δ 8.94 (d, 2H), 7.56 (t, 1H), 7.08 (m, 4H, Ar—H+NH), 3.97 (s, 3H).

### Ex. No. 8-41

¹H-NMR (400 MHz, CDCl₃), δ 8.81 (d, 2H), 7.36 (m, 2H), 7.25 (m, 2H), 7.09 (m, 3H), 7.02 (d, 2H), 6.02 (s, 2H, NH).

### Ex. No. 8-42

¹H-NMR (400 MHz, d₆-DMSO), δ 8.96 (d, 2H), 7.55 (t, 1H), 7.04 (d, 1H), 7.01 (m, 3H, NH+Ar—H), 6.95 (d, 1H), 2.38 (s, 3H).

### Ex. No. 8-43

¹H-NMR (400 MHz, d₆-DMSO), δ 8.94 (d, 2H), 7.54 (t, 1H), 7.36 (t, 1H), 7.08 (m, 2H), 6.97 (s, 2H, NH), 2.36 (s, 3H).

### Ex. No. 8-44

¹H-NMR (400 MHz, CDCl₃), δ 8.81 (d, 2H), 7.43 (m, 2H), 7.25 (t, 1H), 6.86 (d, 1H), 6.02 (s, 2H, NH), 3.81 (s, 3H).

### Ex. No. 8-45

¹H-NMR (400 MHz, d₆-DMSO), δ 8.96 (d, 2H), 7.55 (t, 1H), 7.21 (d, 1H), 6.89 (m, 3H, NH+Ar—H), 6.83 (d, 1H), 3.82 (s, 3H), 2.19 (s, 3H).

### Ex. No. 8-48

¹H-NMR (400 MHz, CDCl₃), δ 8.79 (d, 2H), 7.29 (m, 2H), 7.25 (t, 1H), 6.91 (d, 1H), 6.03 (s, 2H, NH), 3.81 (s, 3H).

### Ex. No. 8-54

¹H-NMR (400 MHz, CDCl₃), δ 8.82 (d, 2H), 7.39 (s, 1H), 7.36 (s, 1H), 7.28 (t, 1H), 5.93 (s, 2H, NH), 2.41 (s, 3H).

### Ex. No. 8-62

¹H-NMR (400 MHz, CDCl₃), δ 8.82 (d, 2H), 7.25 (t, 1H), 7.09 (d, 1H), 6.89 (d, 1H), 5.88 (br. s, 2H, NH), 3.94 (s, 3H), 3.93 (s, 3H).

### Ex. No. 8-65

¹H-NMR (400 MHz, d₆-DMSO), δ 8.94 (d, 2H), 7.56 (t, 1H), 7.14 (d, 1H), 7.11 (d, 1H), 7.04 (s, 2H, NH), 3.93 (s, 3H).

### Ex. No. 8-66

¹H-NMR (400 MHz, d₆-DMSO), δ 8.97 (d, 2H), 7.57 (t, 1H), 7.25 (d, 1H), 7.10 (d, 1H), 7.02 (s, 2H, NH), 6.92 (dd, 1H), 6.86-7.37 (t, 1H), 3.85 (s, 3H).

### Ex. No. 8-88

¹H-NMR (400 MHz, CDCl₃), δ 8.82 (d, 2H), 7.40 (d, 1H), 7.28 (t, 1H), 7.24 (dd, 1H), 6.98 (d, 1H), 5.99 (br. s, 2H, NH), 4.03 (t, 2H), 1.86 (sext, 2H), 1.09 (t, 3H).

### Ex. No. 8-96

¹H-NMR (400 MHz, CDCl₃), δ 8.82 (d, 2H), 7.41 (d, 1H), 7.27 (t, 1H), 7.21 (dd, 1H), 6.97 (d, 1H), 5.98 (br. s, 2H, NH), 4.07 (t, 2H), 1.83 (m, 2H), 1.52 (m, 2H), 1.01 (t, 3H).

### Ex. No. 8-107

¹H-NMR (400 MHz, CDCl₃), δ 8.83 (d, 2H), 7.27 (t, 1H), 6.89 (d, 1H), 6.78 (d, 1H), 6.09 (s, 2H), 6.02 (br. s, 2H, NH).

### Ex. No. 8-108

¹H-NMR (400 MHz, CDCl₃), δ 8.82 (d, 2H), 7.27 (t, 1H), 7.20 (m, 2H), 6.97 (6, 1H), 6.05 (br. s, 2H, NH), 3.85 (s, 3H).

### Ex. No. 8-111

¹H-NMR (400 MHz, CDCl₃), δ 8.85 (d, 2H), 7.30 (t, 1H), 7.27 (m, 1H), 7.13 (m, 2H), 6.11 (br. s, 2H, NH), 3.88 (s, 3H).

### Ex. No. 8-112

¹H-NMR (400 MHz, CDCl₃), δ 8.84 (d, 2H), 7.29 (t, 1H), 7.24 (m, 1H), 7.11 (m, 2H), 6.09 (br. s, 2H, NH), 4.08 (q, 2H), 1.43 (t, 3H).

### Ex. No. 8-113

¹H-NMR (400 MHz, CDCl₃), δ 8.73 (d, 2H), 7.27 (t, 1H), 6.74 (m, 1H), 6.70 (m, 1H), 6.59 (m, 1H), 6.09 (br. s, 2H, NH), 4.05 (q, 2H), 1.44 (t, 3H).

### Ex. No. 9-06

¹H-NMR (400 MHz, CDCl₃), δ 8.82 (d, 2H), 7.25 (t, 1H), 6.88 (m, 1H), 6.85 (m, 2H), 6.02 (s, 2H), 5.82 (br. s, 2H, NH).

### Ex. No. 9-09

¹H-NMR (400 MHz, CDCl₃), δ 8.82 (d, 2H), 7.24 (t, 1H), 6.94 (m, 2H), 6.86 (m, 1H), 5.94 (br. s, 2H, NH), 4.29 (s, 4H).

### Ex. No. 9-14

¹H-NMR (400 MHz, CDCl₃), δ 8.83 (d, 2H), 7.28 (t, 1H), 7.12 (m, 3H), 6.03 (br. s, 2H, NH).

### Ex. No. 10-13

¹H-NMR (400 MHz, CDCl₃), δ 8.95 (d, 2H), 7.58 (d, 2H), 7.41 (t, 1H), 7.22 (m, 6H), 7.02 (m, 4H), 6.55 (d, 2H), 3.98 (s, 4H).

### Ex. No. 10-21

¹H-NMR (400 MHz, CDCl₃), δ 8.96 (d, 2H), 7.45 (m, 1H), 7.36 (d, 1H), 7.22 (m, 6H), 7.19 (m, 1H), 6.98 (m, 4H), 6.78 (dd, 1H), 4.02 (s, 4H).

### Ex. No. 10-22

¹H-NMR (400 MHz, CDCl₃), δ 8.97 (d, 2H), 7.43 (t, 1H), 7.22 (m, 6H), 7.04 (m, 4H), 6.24 (m, 1H), 6.21 (m, 1H), 4.04 (s, 3H), 4.00 (s, 4H).

### Ex. No. 10-23

¹H-NMR (400 MHz, CDCl₃), δ 8.95 (d, 2H), 7.42 (t, 1H), 7.22 (m, 7H), 7.11 (dd, 1H), 7.01 (m, 4H), 6.78 (d, 1H), 6.63 (s, 1H), 6.17-6.66 (t, 1H), 4.01 (s, 4H).

### Ex. No. 10-24

¹H-NMR (400 MHz, CDCl₃), δ 8.94 (d, 2H), 7.41 (t, 1H), 7.21 (m, 8H), 7.04 (m, 4H), 6.93 (m, 1H), 6.63 (m, 1H), 6.44 (dd, 1H), 4.00 (s, 4H), 3.94 (s, 3H).

### Ex. No. 10-25

¹H-NMR (400 MHz, CDCl₃), δ 8.94 (d, 2H), 7.39 (t, 1H), 7.19 (m, 6H), 7.15 (d, 2H), 7.03 (m, 4H), 6.80 (d, 2H), 4.00 (s, 4H), 2.52 (s, 3H).

### Ex. No. 10-26

¹H-NMR (400 MHz, CDCl₃), δ 8.96 (d, 2H), 7.44 (t, 1H), 7.35 (d, 1H), 7.22 (m, 6H), 7.02 (m, 4H), 6.86 (d, 1H), 6.65 (dd, 1H), 4.01 (s, 4H).

### Ex. No. 10-27

¹H-NMR (400 MHz, CDCl₃), δ 8.93 (d, 2H), 7.40 (m, 1H), 7.38 (t, 1H), 7.17 (m, 6H), 6.99 (m, 4H), 6.94 (d, 1H), 6.48 (m, 1H), 4.03 (s, 4H).

### Ex. No. 10-28

¹H-NMR (400 MHz, CDCl₃), δ 8.95 (d, 2H), 7.41 (t, 1H), 7.20 (m, 6H), 7.00 (m, 6H), 6.81 (m, 2H), 6.31-6.80 (t, 1H), 4.00 (s, 4H).

### Ex. No. 10-29

¹H-NMR (400 MHz, CDCl₃), δ 8.98 (d, 2H), 7.43 (t, 1H), 7.22 (m, 6H), 7.02 (m, 4H), 6.98 (m, 1H), 6.63 (m, 2H), 4.01 (s, 4H).

### Ex. No. 11-07

¹H-NMR (400 MHz, CDCl₃), δ 8.79 (d, 2H), 7.42 (br. S, 1H, NH), 7.35 (s, 4H), 7.25 (t, 1H), 2.59 (s, 3H).

### Ex. No. 11-82

¹H-NMR (400 MHz, CDCl₃), δ 8.82 (d, 2H), 7.44 (br. s, 1H, NH), 7.37 (m, 1H), 7.32 (m, 2H), 7.25 (t, 1H), 2.62 (s, 3H).

### Ex. No. 12-07

¹H-NMR (400 MHz, CDCl₃), δ 8.81 (d, 2H), 7.53 (br. s, NH), 7.36 (s, 4H), 7.24 (t, 1H), 2.82 (q, 2H), 1.05 (t, 3H).

### Ex. No. 12-12

¹H-NMR (400 MHz, CDCl₃), δ 8.81 (d, 2H), 8.03 (br. s, 1H, NH), 7.79 (m, 1H), 7.68 (m, 1H), 7.38 (m, 1H), 7.25 (t, 1H), 7.09 (m, 1H), 2.82 (q, 2H), 1.04 (t, 3H).

### Ex. No. 12-18

¹H-NMR (400 MHz, CDCl₃), δ 8.82 (d, 2H), 7.92 (br. s, 1H, NH), 7.41 (m, 1H), 7.24 (m, 2H). 7.10 (dt, 1H), 2.84 (q, 2H), 1.06 (t, 3H).

### Ex. No. 12-80

¹H-NMR (400 MHz, CDCl₃), δ 8.82 (d, 2H), 8.07 (br. s, 1H, NH), 7.46 (dd, 1H), 7.25 (m, 2H), 7.14 (dd, 1H), 2.83 (q, 2H), 1.03 (t, 3H).

### Ex. No. 12-82

¹H-NMR (400 MHz, CDCl₃), δ 8.82 (d, 2H), 7.62 (br. s, NH), 7.37 (m, 1H), 7.30 (m, 2H), 7.27 (t, 1H), 2.84 (q, 2H), 1.08 (t, 3H).

### Ex. No. 12-98

^{H-NMR (}400 MHz, CDCl₃), δ 8.82 (d, 2H), 8.05 (br. s, 1H, NH), 7.25 (t, 1H), 6.97 (s, 1H), 6.94 (s, 1H), 4.06 (s, 3H), 2.87 (q, 2H), 1.07 (t, 3H).

### Ex. No. 12-99

¹H-NMR (400 MHz, CDCl₃), δ 8.84 (d, 2H), 8.11 (br. s, 1H, NH), 7.76 (m, 1H), 7.53 (m, 2H), 7.26 (t, 1H), 2.79 (q, 2H), 1.05 (t, 3H).

### Ex. No. 12-100

¹H-NMR (400 MHz, CDCl₃), δ 8.82 (d, 2H), 8.04 (br. s, 1H, NH), 7.32 (d, 2H), 7.23 (m, 3H), 2.84 (q, 2H), 2.52 (s, 3H), 1.03 (t, 3H).

### Ex. No. 12-101

¹H-NMR (400 MHz, CDCl₃), δ 8.81 (d, 2H), 8.01 (br. s, 1H, NH), 7.27 (t, 1H), 7.10 (m, 2H), 6.93 (m, 1H), 3.94 (s, 3H), 2.83 (q, 2H), 1.04 (t, 3H).

### Ex. No. 12-102

¹H-NMR (400 MHz, CDCl₃), δ 8.83 (d, 2H), 7.92 (br. s, 1H, NH), 7.36 (dd, 1H), 7.24 (m, 2H), 7.18 (m, 1H), 7.09 (m, 1H), 6.27-6.79 (t, 1H), 2.82 (q, 2H), 1.02 (t, 3H).

### Ex. No. 12-103

¹H-NMR (400 MHz, CDCl₃), δ 8.81 (d, 2H), 8.14 (br. s, 1H, NH), 7.40 (d, 2H), 7.25 (t, 1H), 7.11 (d, 2H), 6.34-6.82 (t, 1H), 2.81 (q, 2H), 1.02 (t, 3H).

### Ex. No. 12-104

¹H-NMR (400 MHz, CDCl₃), δ 8.79 (d, 2H), 7.28 (s, 1H, NH), 7.25 (t, 1H), 7.11 (d, 1H), 6.87 (dd, 1H), 6.84 (d, 1H), 3.82 (s, 3H), 2.88 (q, 2H), 2.25 (s, 3H), 1.03 (t, 3H).

### Ex. No. 13-19

¹H-NMR (400 MHz, CDCl₃), δ 8.91 (d, 2H), 7.38 (m, 2H), 7.28 (m, 2H), 2.65 (s, 6H),

### Ex. No. 13-31

¹H-NMR (400 MHz, CDCl₃), δ 8.88 (d, 2H), 7.78 (m, 1H), 7.49 (m, 1H), 7.38 (t, 1H), 7.37 (m, 1H), 7.14 (t, 1H), 2.88 (q, 4H), 0.91 (t, 6H).

### Ex. No. 13-32

¹H-NMR (400 MHz, CDCl₃), δ 8.87 (d, 2H), 7.76 (d, 2H), 7.37 (t, 1H), 7.13 (d, 2H), 2.86 (q, 4H), 0.89 (t, 6H).

### Ex. No. 13-115

¹H-NMR (400 MHz, CDCl₃), δ 8.88 (d, 2H), 7.45 (dd, 1H), 7.38 (t, 1H), 7.24 (m, 1H), 7.17 (m, 1H), 2.88 (q, 4H), 0.90 (t, 6H).

### Ex. No. 13-116

¹H-NMR (400 MHz, CDCl₃), δ 8.88 (d, 2H), 7.52 (d, 1H), 7.41 (m, 1H), 7.38 (m, 1H), 7.30 (s, 1H), 7.27 (t, 1H), 2.88 (q, 4H), 0.92 (t, 6H).

### Ex. No. 13-117

¹H-NMR (400 MHz, CDCl₃), δ 8.87 (d, 2H), 7.38 (t, 1H), 6.97 (m, 1H), 6.93 (m, 1H), 4.06 (s, 3H), 2.91 (q, 4H), 0.94 (t, 6H).

### Ex. No. 13-118

¹H-NMR (400 MHz, CDCl₃), δ 8.80 (d, 2H), 7.39 (m, 2H), 7.31 (m, 2H), 3.08 (m, 2H), 2.74 (m, 2H), 1.47 (m, 2H), 1.33 (m, 1H), 1.12 (m, 2H), 0.88 (t, 3H).

### Ex. No. 14-18

¹H-NMR (400 MHz, CD₃CN, δ, ppm): 8.95 (d, 2H), 7.66-7.63 (s+d, 2H), 7.59 (t, 1H), 7.41 (d,1H).

### Ex. No. 14-37

¹H-NMR (400 MHz, CD₃CN, δ, ppm): 8.96 (d, 2H), 7.66-7.59 (m, 3H),7.39 (dd, 1H).

### Ex. No. 15-07

¹H-NMR (400 MHz, CD₃CN, δ, ppm): 8.82 (d, 2H), 7.45 (m, 4H), 7.42 (t, 1H), 6.83-6.56 (t, ³J=54 Hz, CHF₂), 6.24 (broad s, 2H, NH₂).

### Ex. No. 15-19

¹H-NMR (400 MHz, CDCl₃), δ 8.80 (d, 2H), 7.40 (m, 2H), 7.32 (m, 1H), 7.22 (t, 1H), 6.18 (s, 2H, NH), 5.26 (s, 2H).

### Ex. No. 15-38

¹H-NMR (400 MHz, d₆-DMSO), δ 8.94 (d, 2H), 7.54 (m, 1H), 7.50 (t, 1H), 7.42 (m, 2H), 7.18 (s, 2H, NH), 6.86-7.15 (t, 1H).

### Ex. No. 15-45

¹H-NMR (400 MHz, CDCl₃), δ 8.84 (d, 2H), 7.43 (d, 2H), 7.37 (d, 2H), 7.28 (t, 1H), 5.98 (br. s, 2H, NH).

### Ex. No. 15-48

¹H-NMR (400 MHz, CDCl₃), δ 8.82 (d, 2H), 7.58 (d, 2H), 7.30 (d, 2H), 7.24 (t, 1H), 5.97 (br. s, 2H, NH).

### Ex. No. 15-51

¹H-NMR (400 MHz, CDCl₃), δ 8.82 (d, 2H), 7.78 (d, 2H), 7.24 (t, 1H), 7.17 (d, 2H), 5.96 (br. s, 2H, NH).

### Ex. No. 15-57

¹H-NMR (400 MHz, CDCl₃), δ 8.84 (d, 2H), 7.38 (m, 1H), 7.35 (m, 2H), 7.28 (t, 1H), 6.05 (s, 2H, NH).

### Ex. No. 15-64

¹H-NMR (400 MHz, CD₃CN, δ, ppm): 8.84 (d, 2H), 7.45 (d, 2H), 7.39-7.34 (m, 3H), 6.15 (broad s, 2H, NH₂).

### Ex. No. 15-95

¹H-NMR (400 MHz, d₆-DMSO), δ 8.92 (d, 2H), 7.56 (m, 1H), 7.49 (t, 1H), 7.48-7.62 (t, 1H), 7.44 (m, 2H), 7.14 (s, 2H, NH).

### Ex. No. 15-105

¹H-NMR (400 MHz, CDCl₃), δ 8.82 (d, 2H), 7.59 (d, 2H), 7.32 (d, 2H), 7.25 (t, 1H), 5.96 (br. s, 2H, NH).

### Ex. No. 15-108

¹H-NMR (400 MHz, CDCl₃), δ 8.82 (d, 2H), 7.59 (d, 2H), 7.27 (t, 1H), 7.18 (d, 2H), 5.98 (br. s, 2H, NH).

### Ex. No. 15-114

¹H-NMR (400 MHz, CDCl₃), δ 8.84 (d, 2H), 7.38 (m, 1H), 7.36 (m, 2H), 7.27 (t, 1H), 6.35 (s, 2H, NH).

### Ex. No. 15-153

¹H-NMR (400 MHz, CDCl₃), δ 8.82 (d, 2H), 7.56 (d, 1H), 7.43 (d, 1H), 7.31 (dd, 1H), 7.28 (t, 1H), 6.05 (br. s, 2H, NH).

### Ex. No. 15-154

¹H-NMR (400 MHz, CDCl₃), δ 84 (d, 2H), 7.57 (d, 1H), 7.45 (d, 1H), 7.32 (dd, 1H), 7.27 (t, 1H), 6.03 (br. s, 2H, NH).

### Ex. No. 15-155

¹H-NMR (400 MHz, CDCl₃), δ 8.84 (d, 2H), 7.77 (m, 1H), 7.56 (m, 2H), 7.27 (t, 1H), 6.04 (br. s, 2H, NH).

### Ex. No. 15-156

¹H-NMR (400 MHz, CDCl₃), δ 8.82 (d, 2H), 7.79 (m, 1H), 7.58 (m, 2H), 7.28 (t, 1H), 6.02 (br. s, 2H, NH).

### Ex. No. 15-157

¹H-NMR (400 MHz, CDCl₃), δ 8.83 (d, 2H), 7.67 (m, 1H), 7.54 (m, 2H), 7.27 (t, 1H), 6.07 (br. s, 2H, NH).

### Ex. No. 15-158

¹H-NMR (400 MHz, CDCl₃), δ 8.84 (d, 2H), 7.68 (m, 1H), 7.57 (m, 2H), 7.28 (t, 1H), 6.06 (br. s, 2H, NH).

### Ex. No. 15-159

¹H-NMR (400 MHz, CDCl₃), δ 8.83 (d, 2H), 7.39 (m, 1H), 7.29 (t, 1H), 7.24 (m, 2H), 6.10 (s, 2H, NH).

### Ex. No. 15-160

¹H-NMR (400 MHz, CDCl₃), δ 8.83 (d, 2H), 7.69 (d, 1H), 7.53 (d, 1H), 7.27 (t, 1H), 7.19 (dd, 1H), 6.05 (br. s, 2H, NH).

### Ex. No. 15-161

¹H-NMR (400 MHz, CDCl₃), δ 8.84 (d, 2H), 7.63 (m, 2H), 7.61 (m, 1H), 7.29 (t, 1H), 6.09 (br. s, 2H, NH).

### Ex. No. 15-162

¹H-NMR (400 MHz, CDCl₃), δ 8.85 (d, 2H), 7.64 (m, 1H), 7.62 (m, 2H), 7.29 (t, 1H), 6.09 (br. s, 2H, NH).

### Ex. No. 15-163

¹H-NMR (400 MHz, CDCl₃), δ 8.83 (d, 2H), 7.92 (m, 2H), 7.89 (m, 1H), 7.31 (t, 1H), 6.11 (br. s, 2H, NH).

### Ex. No. 15-164

¹H-NMR (400 MHz, CDCl₃), δ 8.85 (d, 2H), 7.94 (m, 2H), 7.88 (m, 1H), 7.32 (t, 1H), 6.10 (br. s, 2H, NH).

### Ex. No. 15-165

¹H-NMR (400 MHz, CDCl₃), δ 8.83 (d, 2H), 7.53 (m, 1H), 7.33 (m, 2H), 7.29 (t, 1H), 6.11 (br. s, 2H, NH).

### Ex. No. 15-166

¹H-NMR (400 MHz, CDCl₃), δ 8.84 (d, 2H), 7.55 (m, 1H), 7.38 (m, 1H), 7.33 (m, 1H), 7.28 (t, 1H), 6.09 (br. s, 2H, NH).

### Ex. No. 15-167

¹H-NMR (400 MHz, CDCl₃), δ 8.81 (d, 2H), 7.67 (m, 2H), 7.56 (m, 1H), 7.22 (t, 1H), 6.19 (br. s, 2H, NH), 5.46 (m, 1H), 5.33 (m, 1H).

### Ex. No. 15-168

¹H-NMR (400 MHz, CDCl₃), δ 8.83 (d, 2H), 7.69 (d, 1H), 7.56 (d, 1H), 7.25 (t, 1H), 7.19 (dd, 1H), 6.02 (br. s, 2H, NH).

### Ex. No. 15-169

¹H-NMR (400 MHz, CDCl₃), δ 8.83 (d, 2H), 7.41 (m, 1H), 7.20-7.31 (m, 3H), 6.10 (br. s, 2H), NH).

### Ex. No. 16-04

¹H-NMR (400 MHz, CD₃CN, δ, ppm): 8.74 (d, 2H), 7.42-7.38 (m, 2H), 7.23-7.16 (m, 3H), 6.15 (broad s, 2H, NH₂)-the pyrazole-3-methyl proton signal is under the solvent peak.

### Ex. No. 16-05

¹H-NMR (400 MHz, CD₃CN, δ, ppm): 8.74 (d, 2H), 7.54 (d, 1H), 7.39-7.33 (m, 3H), 7.22 (t, 1H), 6.01 (broad s, 2H, NH₂), 2.10 (s, 3H).

### Ex. No. 16-06

¹H-NMR (400 MHz, CD₃CN, δ, ppm): 8.74 (d, 2H), 7.43-7.26 (m, 4H), 7.21 (t, 1H), 6.15 (broad s, 2H, NH₂), 2.20 (s, 3H).

### Ex. No. 16-07

¹H-NMR (400 MHz, CD₃CN, δ, ppm): 8.75 (d, 2H), 7.43 (d, 2H), 7.40 (d, 7.22 (t, 1H), 6.21 (broad s, 2H, NH₂)-the pyrazole-3-methyl proton signal is under the solvent peak.

### Ex. No. 16-15

¹H-NMR (400 MHz, d₆-DMSO, δ, ppm): 8.81 (d, 2H), 7.65 (s, 1H), 7.45-7.36 (dd, 2H), 7.32 (t, 1H), 6.45 (broad s, 2H, NH₂), 1.99 (s, 3H).

### Ex. No. 16-39

¹H-NMR (400 MHz, CDCl₃), δ 8.76 (d, 2H), 7.27 (m, 3H), 7.15 (t, 1H), 6.03 (br. s, 2H, NH). 2.69 (q, 2H), 1.18 (t, 3H) .

### Ex. No. 16-134

¹H-NMR (400 MHz, CDCl₃), δ 8.77 (d, 2H), 7.27 (m, 1H), 7.25 (m, 2H), 7.16 (t, 1H), 6.01 (br. s, 2H, NH), 2.59 (d, 2H), 1.86 (nonette, 1H), 0.95 (d, 6H).

### Ex. No. 18-07

¹H-NMR (400 MHz, d₆-DMSO), δ 8.91 (d, 2H), 7.49 (d, 2H), 7.46 (t, 1H), 7.42 (d, 2H), 6.82 (s, 2H, NH), 5.56-5.71 (dq, 1H), 1.56 (dd, 3H).

### Ex. No. 18-19

¹H-NMR (400 MHz, d₆-DMSO), δ 8.92 (d, 2H), 7.52 (m, 1H), 7.47 (t, 1H), 7.43 (m, 2H), 7.05 (s, 2H, NH), 5.59-5.76 (dq, 1H), 1.58 (dd, 3H).

### Ex. No. 18-26

¹H-NMR (400 MHz, d₆-DMSO), δ 8.93 (d, 2H), 7.48 (t, 1H), 7.45 (d, 2H), 7.39 (d, 2H), 6.90 (s, 2H, NH), 1.91-2.05 (t, 3H).

### Ex. No. 18-32

¹H-NMR (400 MHz, d₆-DMSO), δ 8.93 (d, 2H), 7.77 (d, 2H), 7.48 (t, 1H), 7.19 (d, 2H), 6.81 (s, 2H, NH), 1.92-2.04 (t, 3H).

### Ex. No. 18-34

¹H-NMR (400 MHz, CDCl₃), δ 8.82 (d, 2H), 7.52 (d, 1H), 7.38 (m, 1H), 7.31 (m, 1H), 7.22 (t, 1H), 5.79 (br. s, 2H, NH), 1.96 (t, 3H).

### Ex. No. 18-38

¹H-NMR (400 MHz, CDCl₃), δ 8.84 (d, 2H), 7.38 (m, 1H), 7.36 (m, 2H), 7.27 (t, 1H), 6.35 (s, 2H, NH).

### Ex. No. 18-48

¹H-NMR (400 MHz, CDCl₃), δ 8.84 (d, 2H), 7.57 (d, 2H), 7.25 (t, 1H), 7.22 (d, 2H), 6.01 (br. s, 2H, NH).

### Ex. No. 18-51

¹H-NMR (400 MHz, CDCl₃), δ 8.82 (d, 2H), 7.76 (d, 2H), 7.27 (t, 1H), 7.10 (d, 2H), 5.94 (br. s, 2H, NH).

### Ex. No. 18-57

¹H-NMR (400 MHz, CDCl₃), δ 8.82 (d, 2H), 7.37 (m, 1H), 7.29 (t, 1H), 7.25 (m, 2H), 6.03 (s, 2H, NH).

### Ex. No. 18-190

¹H-NMR (400 MHz, CDCl₃), δ 8.80 (d, 2H), 7.39 (s, 2H), 7.32 (s, 1H), 7.19 (t, 1H), 6.08 (br. s, 2H, NH), 5.39-5.54 (ddd, 1H), 2.00 (m, 1H), 1.74 (m, 1H), 0.93 (t, 3H).

### Ex. No. 18-209

¹H-NMR (400 MHz, CDCl₃), δ 8.79 (d, 2H), 7.37 (s, 2H), 7.31 (s, 1H), 7.20 (t, 1H), 6.06 (br. s, 2H, NH), 5.46-5.62 (ddd, 1H), 2.01 (m, 1H), 1.68 (m, 1H), 1.47 (m, 1H), 1.34 (m, 1H), 0.89 (t, 3H).

### Ex. No. 18-229

¹H-NMR (400 MHz, CDCl₃), δ 8.81 (d, 2H), 7.36 (m, 1H), 7.26 (m, 2H), 7.23 (t, 1H), 5.95 (br. s, 2H, NH), 3.59 (s, 3H).

### Ex. No. 18-230

¹H-NMR (400 MHz, CDCl₃), δ 8.83 (d, 2H), 7.64 (m, 1H), 7.57 (m, 2H), 7.23 (t, 1H), 6.04 (br. s, 2H, NH), 2.03 (t, 3H).

### Ex. No. 18-231

¹H-NMR (400 MHz, CDCl₃), δ 8.82 (d, 2H), 7.99 (s, 2H), 7.82 (s, 1H), 7.24 (t, 1H), 6.09 (br. s, 2H, NH), 5.63-5.85 (dq, 1H), 1.58 (dd, 3H).

### Ex. No. 18-232

¹H-NMR (400 MHz, CDCl₃), δ 8.80 (d, 2H), 7.69 (s, 1H), 7.67 (s, 1H), 7.56 (s, 1H), 7.21 (t, 1H), 6.08 (br. s, 2H, NH), 5.65-5.81 (dq, 1H), 1.60 (dd, 3H).

### Ex. No. 18-233

¹H-NMR (400 MHz, CDCl₃), δ 8.82 (d, 2H), 7.95 (m, 2H), 7.83 (m, 1H), 7.27 (t, 1H), 6.07 (br. s, 2H, NH), 2.09 (t, 3H).

### Ex. No. 18-234

¹H-NMR (400 MHz, CDCl₃), δ 8.78 (d, 2H), 7.90 (m, 1H), 7.85 (m, 2H), 7.33 (t, 1H), 6.06 (br. s, 2H, NH).

### Ex. No. 18-235

¹H-NMR (400 MHz, CDCl₃), δ 8.83 (d, 2H), 7.64 (m, 2H), 7.57 (m, 1H), 7.24 (t, 1H), 6.05 (br. s, 2H, NH), 2.04 (t, 3H).

### Ex. No. 18-236

¹H-NMR (400 MHz, CDCl₃), δ 8.86 (d, 2H), 7.62 (m, 1H), 7.54 (m, 2H), 7.30 (t, 1H), 6.03 (br. s, 2H, NH).

### Ex. No. 18-237

¹H-NMR (400 MHz, CDCl₃), δ 8.85 (d, 2H), 7.44 (m, 1H), 7.29 (t, 1H), 7.27-7.36 (m, 2H), 6.04 (br. s, 2H, NH).

### Ex. No. 18-240

¹H-NMR (400 MHz, CDCl₃), δ 8.83 (d, 2H), 7.56 (s, 1H), 7.40 (m, 1H), 7.27 (m, 1H), 7/24 (t, 1H), 6.06 (br. s, 2H, NH), 2.08 (t, 3H).

### Ex. No. 18-241

¹H-NMR (400 MHz, CDCl₃), δ 8.84 (d, 2H), 7.32 (m, 1H), 7.29 (t, 1H), 7.25 (m, 1H), 7.17 (m, 1H), 6.05 (br. s, 2H, NH).

### Ex. No. 18-242

¹H-NMR (400 MHz, CDCl₃), δ 8.84 (d, 2H), 7.68 (m, 1H), 7.45 (m, 2H), 7.28 (t, 1H), 6.04 (br. s, 2H, NH).

### Ex. No. 18-243

¹H-NMR (400 MHz, CDCl₃), δ 8.79 (d, 2H), 7.62 (m, 1H), 7.59 (m, 2H), 7.20 (t, 1H), 6.06 (br. s, 2H, NH), 5.62 (dq, 1H), 1.60 (dd, 3H).

### Ex. No. 18-244

¹H-NMR (400 MHz, CDCl₃), δ 8.83 (d, 2H), 7.70 (s, 1H), 7.58 (d, 1H), 7.46 (d, 1H), 7.29 (t, 1H), 5.98 (br. s, 2H, NH).

### Ex. No. 18-245

¹H-NMR (400 MHz, CDCl₃), δ 8.79 (d, 2H), 7.83 (d, 1H), 7.61 (d, 1H), 7.57 (s, 1H), 7.20 (t, 1H), 6.02 (br. s, 2H, NH), 5.62 (dq, 1H), 1.56 (dd, 3H).

### Ex. No. 18-246

¹H-NMR (400 MHz, CDCl₃), δ 8.84 (d, 2H), 7.67 (d, 1H), 7.56 (d, 1H), 7.28 (t, 1H), 7.12 (dd, 1H), 6.00 (br. s, 2H, NH).

### Ex. No. 19-26

¹H-NMR (400 MHz, CD₃CN, δ, ppm): 8.66 (s, 2H), 7.45 (d, 2H), 7.39 (d, 2H), 6.16 (broad s, 2H, NH₂), 2.34 (s, 3H).

### Ex. No. 19-37

¹H-NMR (400 MHz, CD₃CN, δ, ppm): 8.66 (s, 2H), 7.58 (d, 1H), 7.55 (s, 1H), 7.33 (d, 1H), 6.25 (broad s, 2H, NH₂), 2.35 (s, 3H).

### Ex. No. 19-45

¹H-NMR (400 MHz, CD₃CN, δ, ppm): 8.77 (s, 2H), 7.47 (d, 2H), 7.40 (d, 2H), 6.22 (broad s, 2H, NH₂).

### Ex. No. 19-56

¹H-NMR (400 MHz, CD₃CN, δ, ppm): 8.75 (s, 2H), 7.61 (d, 1H), 7.56 (s, 1H), 7.33 (d, 1H), 6.17 (broad s, 2H, NH₂).

### Ex. No. 19-64

¹H-NMR (400 MHz, CD₃CN, δ, ppm): 8.81 (s, 2H), 7.46 (d, 2H), 7.39 (d, 2H), 6.13 (broad s, 2H, NH₂).

### Ex. No. 19-75

¹H-NMR (400 MHz, CD₃CN, δ, ppm): 8.82 (s, 2H), 7.59 (d, 1H), 7.55 (s, 1H), 7.33 (d, 1H), 6.24 (broad s, 2H, NH₂).

### Ex. No. 19-121

¹H-NMR (400 MHz, CD₃CN, δ, ppm): 9.10 (d, 1H), 7.74 (d, 1H), 7.46 (d, 2H), 7.39 (d, 1H), 6.18 (broad s, 2H, NH₂).

### Ex. No. 19-195

¹H-NMR (400 MHz, CDCl₃), δ 9.18 (d, 1H), 7.68 (d, 1H), 7.61 (d, 1H), 7.55 (d, 1H), 7.28 (dd, 1H), 5.97 (br. s, 2H, NH).

### Ex. No. 19-196

¹H-NMR (400 MHz, CDCl₃), δ 9.17 (d, 1H), 7.71 (d, 1H), 7.60 (d, 1H), 7.51 (d, 1H), 7.16 (dd, 1H), 5.96 (br. s, 2H, NH).

### Ex. No. 19-197

¹H-NMR (400 MHz, CDCl₃), δ 9.19 (d, 1H), 7.88 (m, 3H), 7.62 (d, 1H), 6.04 (br. s, 2H, NH).

### Ex. No. 24-06

¹H-NMR (400 MHz, CD₃CN, δ, ppm): 8.72 (d, 2H), 7.64 (broad NH), 7.39-7.28 (m, 4H), 7.19 (t, 1H), 2.59 (d, 3H), 2.08 (s, 3H).

### Ex. No. 25-07

¹H-NMR (400 MHz, CD₃CN, δ, ppm): 8.74 (d, 2H), 7.70 (broad d, 1H, NH), 7.42-7.35 (2d, 4H), 7.22 (t, 1H), 2.88 (m, 2H), 0.97 (t, 3H)-the pyrazole-3-methyl proton signal is under the solvent peak.

### Ex. No. 25-34

¹H-NMR (400 MHz, CD₃CN, δ, ppm): 8.71 (d, 2H), 7.76 (broad NH), 7.57 (s, 1H), 7.40-7.34 (d+d, 2H), 7.19 (t, 1H), 2.84-2.79 (m, 2H), the pyrazole-3-methyl proton signal is under the solvent peak, 0.98 (t, 3H).

### Ex. No. 26-23

¹H-NMR (400 MHz, CD₃CN, δ, ppm): 8.75 (d, 2H), 7.51 (broad d, 1H, NH), 7.43 (m, 2H), 7.22 (t, 1H), 7.15 (m, 2H), 3.25-3.17 (m, 1H), 2.09 (s, 3H), 0.96 (d, 6H).

### Ex. No. 26-24

¹H-NMR (400 MHz, CD₃CN, δ, ppm): 8.81 (d, 2H), 7.61-7.35 (m, 6H), 3.21 (m, 1H), 2.08 (s, 3H), 1.06-0.98 (d+d, 6H).

### Ex. No. 26-26

¹H-NMR (400 MHz, CD₃CN, δ, ppm): 8.75 (d, 2H), 7.53 (broad s, 1H, NH), 7.40 (m, 4H), 7.23 (t, 1H), 3.25 (m, 1H), 0.97 (d, 6H)-the pyrazole-3-methyl proton signal is under the solvent peak.

### Ex. No. 26-46

¹H-NMR (400 MHz, CD₃CN, δ, ppm): 8.71 (d, 2H), 8.14 (broad s, 1H, NH), 7.35 (d, 2H), 7.29 (d, 2H), 7.21-7.16 (m, 3H), 6.99-6.97 (m, 2H), 4.07 (d, 2H), 2.05 (s, 3H).

### Ex. No. 26-65

¹H-NMR (400 MHz, CD₃CN, δ, ppm): 8.73 (d, 2H), 7.71 (broad s, 1H, NH), 7.39-7.33 (d+d, 4H), 7.20 (t, 1H), 2.69 (broad dd, 2H), 2.08 (s, 3H), 0.84 (m, 1H), 0.34 (m, 2H), 0.00 (m, 2H).

### Ex. No. 27-19

¹H-NMR (400 MHz, CDCl₃), δ 8.83 (d, 2H), 8.00 (br. t, 1H, NH), 7.38 (m, 1H), 7.32 (m, 2H), 7.25 (t, 1H), 2.78 (m, 2H), 1.42 (sext, 2H), 0.78 (t, 3H).

### Ex. No. 27-26

¹H-NMR (400 MHz, CDCl₃), δ 8.82 (d, 2H), 7.64 (br. S, NH), 7.36 (s, 4H), 7.24 (t, 1H), 3.17 (sept, 1H), 0.99 (d, 6H).

### Ex. No. 27-38

¹H-NMR (400 MHz, CDCl₃), δ 8.82 (d, 2H), 7.72 (br. d, 1H, NH), 7.34 (m, 2H), 7.27 (t, 1H), 7.24 (m, 1H), 3.18 (m, 1H), 1.03 (d, 6H).

### Ex. No. 27-45

¹H-NMR (400 MHz, CDCl₃), δ 8.79 (d, 2H), 8.37 (br. S, 1H, NH), 7.21 (m, 8H), 6.98 (m, 2H) 4.07 (s, 2H).

### Ex. No. 27-50

¹H-NMR (400 MHz, CDCl₃), δ 8.80 (d, 2H), 8.06 (br. t, 1H, NH), 7.63 (m, 2H), 7.23 (m, 5H), 7.03 (d, 1H), 6.98 (m, 2H), 4.06 (s, 2H).

### Ex. No. 27-51

¹H-NMR (400 MHz, CDCl₃), δ 8.79 (d, 2H), 8.11 (br. t, 1H, NH), 7.62 (d, 2H), 7.22 (m, 5H), 6.98 (m, 3H), 4.05 (s, 2H).

### Ex. No. 27-76

¹H-NMR (400 MHz, CDCl₃), δ 8.82 (d, 2H), 8.02 (br. s, 1H, NH), 7.35 (m, 1H), 7.30 (m, 2H), 7.26 (t, 1H), 2.68 (d, 2H), 0.89 (m, 1H), 0.45 (m, 2H), 0.22 (m, 2H).

### Ex. No. 27-114

¹H-NMR (400 MHz, CDCl₃), δ 8.83 (d, 2H), 8.12 (br. t, 1H, NH), 7.38 (m, 1H), 7.30 (m, 2H), 7.25 (t, 1H), 5.63 (m, 1H), 5.06 (m, 2H), 3.48 (m, 2H).

### Ex. No. 27-133

¹H-NMR (400 MHz, CDCl₃), δ 8.82 (d, 2H), 8.13 (br. t, 1H, NH), 7.37 (m, 1H), 7.29 (m, 2H), 7.24 (t, 1H), 2.63 (t, 2H), 1.64 (nonette, 1H), 0.78 (d, 6H).

### Ex. No. 27-140

¹H-NMR (400 MHz, CDCl₃), δ 8.81 (d, 2H), 8.39 (br. S, 1H, NH), 7.26 (m, 5H), 7.18 (d, 2H), 6.88 (d, 2H), 4.07 (s, 2H).

### Ex. No. 27-153

¹H-NMR (400 MHz, CDCl₃), δ 8.87 (d, 2H), 8.62 (br. t, 1H, NH), 7.51 (m, 1H), 7.33 (m, 2H), 7.08 (m, 2H), 7.02 (m, 2H), 4.14 (d, 2H).

### Ex. No. 27-154

¹H-NMR (400 MHz, CDCl₃), δ 8.80 (d, 2H), 7.99 (br. d, 1H, NH), 7.35 (m, 1H), 7.31 (m, 2H), 7.27 (t, 1H), 3.42 (m, 1H), 1.55 (m, 4H), 1.42 (m, 4H).

### Ex. No. 27-155

¹H-NMR (400 MHz, CDCl₃), δ 8.81 (d, 2H), 8.22 (br. t, 1H, NH), 7.33 (m, 1H), 7.19 (m, 4H), 7.12 (m, 1H), 7.02 (m, 1H), 6.98 (m, 2H), 4.09 (s, 2H).

### Ex. No. 27-156

¹H-NMR (400 MHz, CDCl₃), δ 8.79 (d, 2H), 8.23 (br. t, 1H, NH), 7.23 (m, 6H), 7.01 (d, 2H), 6.97 (m, 2H), 6.28-6.79 (t, 1H), 4.05 (s, 2H).

### Ex. No. 27-157

¹H-NMR (400 MHz, CDCl₃), δ 8.81 (d, 2H), 8.04 (br. t, 1H, NH), 7.37 (m, 1H), 7.22 (m, 5H), 7.08 (m, 1H), 6.98 (m, 2H), 4.12 (s, 2H).

### Ex. No. 27-158

¹H-NMR (300 MHz, CDCl₃), δ 8.84 (d, 2H), 8.05 (br. t, 1H, NH), 7.51 (m, 1H), 7.19-7.40 (m, 6H), 6.92 (m, 2H), 4.08 (s, 2H).

### Ex. No. 27-159

¹H-NMR (400 MHz, CDCl₃), δ 8.79 (d, 2H), 8.42 (br. t, 1H, NH), 7.21 (m, 4H), 7.08 (d, 1H), 7.02 (m, 2H), 6.85 (dd, 1H), 6.68 (d, 1H), 4.11 (s, 2H), 3.69 (s, 3H), 2.24 (s, 3H).

### Ex. No. 27-160

¹H-NMR (400 MHz, CDCl₃), δ 8.80 (d, 2H), 8.18 (br. t, 1H, NH), 7.36 (d, 1H), 7.20 (m, 5H), 7.14 (dd, 1H), 6.95 (m, 2H), 4.09 (s, 2H).

### Ex. No. 27-161

¹H-NMR (400 MHz, CDCl₃), δ 8.79 (d, 2H), 8.12 (br. t, 1H, NH), 7.19 (m, 8H), 7.01 (m, 2H), 4.07 (s, 2H), 2.52 (s, 3H).

### Ex. No. 27-162

¹H-NMR (400 MHz, CDCl₃), δ 8.80 (d, 2H), 8.09 (br. t, 1H, NH), 7.17-7.34 (m, 5H), 7.05 (d, 1H), 6.97 (m, 4H), 6.14-6.63 (t, 1H), 4.08 (s, 2H).

### Ex. No. 27-163

¹-NMR (400 MHz, CDCl₃), δ 8.79 (d, 2H), 8.17 (br. t, 1H, NH), 7.22 (m, 4H), 7.01 (m, 2H), 6.88 (m, 2H), 4.14 (s, 2H), 4.03 (s, 3H).

### Ex. No. 28-07

¹H-NMR (400 MHz, CD₃CN, δ, ppm): 8.79 (d, 2H), 7.41-7.38 (2d, 4H), 7.31 (t, 1H), 2.29 (s, 3H)-the pyrazole-3-methyl proton signal is under the solvent peak.

### Ex. No. 29-07

¹H-NMR (400 MHz, CD₃CN, δ, ppm): 8.87 (d, 2H), 8.68 (broad NH), 7.49-7.30 (m, 5H).

### Ex. No. 29-12

¹H-NMR (400 MHz, CDCl₃), δ 9.38 (br. s, 1H, NH), 8.84 (d, 2H), 7.77 (m, 1H), 7.68 (d, 1H), 7.38 (d, 1H), 7.36 (t, 1H), 7.17 (m, 1H), 2.08 (s, 3H).

### Ex. No. 29-19

¹H-NMR (400 MHz, CDCl₃), δ 9.62 (br. s, 1H, NH), 8.91 (d, 2H), 7.42 (t, 1H), 7.37 (m, 1H), 7.29 (m, 2H), 2.08 (s, 3H).

### Ex. No. 29-26

¹H-NMR (400 MHz, CD₃CN, δ, ppm): 8.84 (d, 2H), 7.50-7.47 (m, 3H), 7.32 (d, 2H), 2.19 (s, 6H).

### Ex. No. 29-153

¹H-NMR (400 MHz, CDCl₃), δ 9.41 (br. s, 1H, NH), 8.89 (d, 2H), 7.40 (t, 1H), 6.99 (m, 1H), 6.95 (m, 1H), 4.04 (s, 3H), 2.09 (s, 3H).

### Ex. No. 29-154

¹H-NMR (400 MHz, CDCl₃), δ 8.87 (d, 2H), 8.79 (br. s, 1H, NH), 7.38 (t, 1H), 7.16 (d, 1H), 6.88 (m, 2H), 3.82 (s, 3H), 2.27 (s, 3H), 1.98 (s, 3H).

### Ex. No. 29-155

¹H-NMR (400 MHz, CDCl₃), δ 9.58 (br. s, 1H, NH), 8.92 (d, 2H), 7.54 (d, 1H), 7.41 (t, 1H), 7.35 (m, 2H), 2.07 (s, 3H).

### Ex. No. 29-156

¹H-NMR (400 MHz, CDCl₃), δ 9.18 (br. s, 1H, NH), 8.91 (d, 2H), 7.39 (m, 3H), 7.16 (d, 2H), 6.34-6.82 (t, 1H), 2.01 (s, 3H).

### Ex. No. 29-157

¹H-NMR (400 MHz, CDCl₃), δ 9.32 (br. s, 1H, NH), 8.89 (d, 2H), 7.40 (m, 2H), 7.28 (m, 1H), 7.17 (t, 1H), 2.05 (s, 3H).

### Ex. No. 29-158

¹H-NMR (400 MHz, CDCl₃), δ 8.83 (d, 2H), 7.39 (t, 1H), 6.87 (m, 1H), 6.84 (m, 1H), 4.08 (s, 3H), 2.27 (s, 6H).

### Ex. No. 29-159

¹H-NMR (400 MHz, CDCl₃), δ 8.82 (d, 2H), 7.36 (t, 1H), 7.18 (d, 1H), 6.79 (dd, 1H), 6.63 (d, 1H), 3.78 (s, 3H), 2.25 (s, 6H), 2.24 (s, 3H).

### Ex. No. 29-160

¹H-NMR (400 MHz, CDCl₃), δ 8.83 (d, 2H), 7.39 (t, 1H), 7.35 (d, 1H), 6.92 (dd, 1H), 6.77 (d, 1H), 2.27 (s, 6H).

### Ex. No. 29-161

¹H-NMR (400 MHz, CDCl₃), δ 8.82 (d, 2H), 7.38 (t, 1H), 7.32 (d, 2H), 7.18 (d, 2H), 6.35-6.82 (t, 1H), 2.24 (s, 6H).

### Ex. No. 29-162

¹H-NMR (400 MHz, CDCl₃), δ 8.83 (d, 2H), 7.36 (m, 2H), 7.18 (m, 2H), 2.25 (s, 6H).

### Ex. No. 29-163

¹H-NMR (400 MHz, CDCl₃), δ 9.87 (br. s, 1H, NH), 8.94 (d, 2H), 7.65 (m, 3H), 7.44 (t, 1H), 2.07 (s, 3H).

### Ex. No. 29-164

¹H-NMR (400 MHz, CDCl₃), δ 9.78 (br. s, 1H, NH), 8.92 (d, 2H), 7.60 (m, 2H), 7.55 (m, 1H), 7.43 (t, 1H), 2.08 (s, 3H).

### Ex. No. 29-165

¹H-NMR (400 MHz, CDCl₃), δ 8.84 (d, 2H), 7.68 (m, 1H), 7.61 (m, 2H), 7.58 (br. s, 1H, NH), 7.38 (t, 1H), 4.17 (m, 1H), 3.99 (m, 1H), 2.05 (s, 1H).

¹H-NMR (400 MHz, CDCl₃), δ 8.80 (d, 2H), 7.63 (m, 1H), 7.56 (m, 2H), 7.31 (t, 1H), 4.78 (dd, 2H), 4.59 (dd, 2H), 2.42 (m, 2H), 2.11 (t, 3H).

### Ex. No. 29-167

¹H-NMR (400 MHz, CDCl₃), δ 8.84 (d, 2H), 8.00 (m, 2H), 7.78 (m, 1H), 7.32 (t, 1H), 4.96 (s, 2H), 4.83 (s, 2H), 2.82 (s, 2H).

### Ex. No. 32-03

¹H-NMR (400 MHz, CDCl₃), δ 8.85 (d, 2H), 8.44 (d, 1H), 7.73 (dd, 1H), 7.44 (d, 1H), 7.31 (t, 1H), 6.51 (br. s, 2H, NH).

### Ex. No. 32-12

¹H-NMR (400 MHz, CDCl₃), δ 8.82 (d, 2H), 8.21 (d, 1H), 7.61 (dd, 1H), 7.29 (t, 1H), 6.85 (d, 1H), 6.02 (br. s, 2H, NH), 3.99 (s, 3H).

### Ex. No. 32-17

¹H-NMR (400 MHz, CDCl₃), δ 8.86 (d, 2H), 8.81 (d, 1H), 7.94 (dd, 1H), 7.77 (d, 1H), 7.34 (t, 1H), 6.25 (br. s, 2H, NH).

### Ex. No. 32-20

¹H-NMR (400 MHz, d₆-DMSO, δ, ppm): 8.82 (d, 2H), 8,41 (d, 1H), 7.83-7.81 (dd, 1H), 7.52 (d, 1H), 7.35 (t, 1H), 6.81 (broad s, 2H, NH₂), 2.16 (s, 3H).

### Ex. No. 32-37

¹H-NMR (400 MHz, d₆-DMSO, δ, ppm): 8.83 (d, 2H), 8.39 (d, 1H), 7.82-7.79 (dd, 1H), 7.52 (d, 1H), 7.35 (t, 1H), 6.75 (broad s, 2H, NH₂), 2.59-2.53 (q, 2H), 1.13-1.09 (t, 3H).

### Ex. No. 32-52

¹H-NMR (400 MHz, CDCl₃), δ 8.82 (d, 2H), 8.20 (d, 1H), 7.46 (dd, 1H), 7.25 (t, 1H), 6.61 (d, 1H), 5.94 (br. s, 2H, NH), 3.14 (s, 6H).

### Ex. No. 32-53

¹H-NMR (400 MHz, CDCl₃), δ 8.83 (d, 2H), 8.20 (d, 1H), 7.62 (dd, 1H), 7.27 (t, 1H), 6.79 (d, 1H), 6.04 (br. s, 2H, NH), 5.35 (sept, 1H), 1.39 (d, 6H).

### Ex. No. 32-54

¹H-NMR (400 MHz, CDCl₃), δ 8.84 (d, 2H), 8.11 (d, 1H), 7.68 (d, 1H), 7.29 (t, 1H), 6.05 (br. s, 2H, NH), 4.07 (s, 3H).

### Ex. No. 33-63

¹H-NMR (400 MHz, d₆-DMSO, δ, ppm): 8.82 (d, 2H), 8.63 (s, 1H), 8.18 (s, 1H), 7.34 (t, 1H), 6.69 (broad s, 2H, NH₂), under the solvent peak (q, 2H), 1.04 (t, 3H).

### Ex. No. 35-20

¹H-NMR (400 MHz, CDCl₃), δ 8.74 (d, 2H), 7.39 (m, 2H), 7.24 (m, 1H), 7.11 (t, 1H), 6.07 (br. s, 2H, NH), 1.78 (m, 1H), 1.12 (m, 2H), 0.91 (m, 2H).

### Ex. No. 35-39

¹H-NMR (400 MHz, CDCl₃), δ 8.79 (d, 2H), 7.52 (m, 2H), 7.30 (m, 1H), 7.19 (t, 1H), 6.11 (br. s, 2H, NH), 1.39 (m, 4H).

### Ex. No. 35-77

¹H-NMR (400 MHz, CDCl₃), δ 8.88 (d, 2H), 7.25 (m, 1H), 7.22 (m, 2H), 7.17 (t, 1H), 6.05 (br. s, 2H, NH), 3.48 (quint, 1H), 2.46 (m, 2H), 2.21 (m, 2H), 1.98 (m, 1H), 1.82 (m, 1H).

### Ex. No. 35-134

¹H-NMR (400 MHz, CDCl₃), δ 8.89 (d, 2H), 7.46 (m, 2H), 7.28 (m, 1H), 7.21 (t, 1H), 6.07 (br. s, 2H, NH), 1.31 (m, 2H), 1.19 (m, 2H).

### Ex. No. 42-20

¹H-NMR (400 MHz, CDCl₃), δ 8.79 (d, 2H), 7.96 (br. s, 1H, NH), 7.37 (m, 2H), 7.31 (m, 1H), 7.22 (t, 1H), 5.19-5.37 (d, 2H), 2.93 (q, 2H), 1.08 (t, 3H).

### Ex. No. 42-39

¹H-NMR (400 MHz, CDCl₃), δ 8.77 (d, 2H), 7.92 (br. s, 1H, NH), 7.29 (m, 1H), 7.21 (m, 6H), 7.03 (m, 2H), 5.17-5.35 (d, 2H), 4.18 (s, 2H).

### Ex. No. 42-58

¹H-NMR (400 MHz, CDCl₃), δ 8.95 (d, 2H), 7.37 (t, 1H), 7.21 (m, 7H), 7.07 (m, 4H), 6.67 (d, 2H), 5.14-5.29 (d, 2H), 4.07 (s, 4H).

### Ex. No. 42-77

¹H-NMR (400 MHz, CDCl₃), δ 8.88 (d, 2H), 7.42 (m, 2H), 7.35 (m, 2H), 6.55-6.90 (t, 1H), 2.95 (q, 4H), 0.96 (t, 6H).

### Ex. No. 42-96

¹H-NMR (400 MHz, CDCl₃), δ 8.79 (d, 2H), 7.22 (m, 8H), 7.00 (m, 2H), 6.44-6.82 (t, 1H), 4.12 (s, 2H).

### Ex. No. 42-115

¹H-NMR (400 MHz, CDCl₃), δ 8.96 (d, 2H), 7.42 (t, 1H), 7.31 (m, 7H), 7.06 (m, 4H), 6.67 (m, 2H), 6.43-6.78 (t, 1H), 4.04 (s, 4H).

### Ex. No. 42-135

¹H-NMR (400 MHz, CDCl₃), δ 9.66 (br. s, 1H, NH), 8.87 (d, 2H), 7.36 (m, 3H), 7.31 (m, 1H), 6.61-6.97 (t, 1H), 2.09 (s, 3H).

### Ex. No. 43-02

¹H-NMR (400 MHz, CDCl₃), δ 8.70 (s, 2H), 7.74 (d, 1H), 7.69 (d, 1H), 7.50 (dd, 1H), 5.92 (br. s, 2H, NH).

### Ex. No. 43-03

¹H-NMR (400 MHz, CDCl₃), δ 8.70 (s, 2H), 7.63 (m, 2H), 7.61 (s, 1H), 5.90 (br. s, 2H, NH).

### Ex. No. 43-04

¹H-NMR (400 MHz, CDCl₃), δ 8.71 (s, 2H), 7.62 (m, 2H), 7.59 (s, 1H), 5.91 (br. s, 2H, NH).

### Ex. No. 43-05

### ¹H-NMR (400 MHz, CDCl₃), δ 8.72 (s, 2H), 7.60 (m, 2H), 7.57 (s, 1H), 5.96 (br. s, 2H, NH).

### Ex. No. 43-06

¹H-NMR (400 MHz, CDCl₃), δ 8.69 (s, 2H), 7.63 (m, 2H), 7.58 (s, 1H), 5.85 (br. s, 2H, NH), 2.02 (t, 3H).

### Detailled Synthesis Examples

### Example 1-79

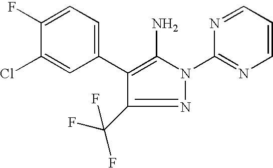

Step 1: Under reflux, 1.43 g of sodium (62 mmol) were dissolved in 15 ml of ethanol. At 40° C., a solution of 10 g (59 mmol) of 3-chloro-4-fluorobenzonitrile and 8.8 g (62 mmol) of ethyl trifluoroacetate in 10 ml of ethanol was added dropwise, and the reaction mixture was then stirred under reflux for 12 hours. After cooling, the reaction mixture was concentrated on a rotary evaporator, dissolved in ethyl acetate and washed twice with 100 ml of 1N hydrochloric acid and once with water. The organic phase was separated off, dried over sodium sulphate and filtered. On a rotary evaporator, under reduced pressure, the solvent was carefully distilled off from the filtrate. The residue was triturated with a solvent mixture of petroleum ether/dichloromethane (vol.: 1:1). The colourless solid that separated off was filtered off with suction over a glass frit and dried. This gave 12 g (72.8% of theory) of 2-(3-chloro-4-fluorophenyl)-4,4,4-trifluoro-3-oxobutanenitrile as a mixture of tautomers.

¹H-NMR (400 MHz, d₆-DMSO, δ, ppm): tautomer mixture in a ratio of about 3:2, 8.22 (A-1, dd, 1H), 7.69 (A-1, m, 1H), 7.62 (B-1, dd, 1H), 7.47 (B-1, m, 1H), 7.42 (B-1, t, 1H), 7.17 (A-1, t, 1H), 4.61 (B-1, s, 1H).

Step 2: 0.5 g (1.86 mmol) of 2-(3-chloro-4-fluorophenyl)-4,4,4-trifluoro-3-oxobutanenitrile and 0.2 g (1.86 mmol) of 2-pyrimidinylhydrazine were dissolved in 5 ml of toluene. After addition of catalytic amounts of para-toluenesulphonic acid, the reaction mixture was stirred in a microwave synthesis system (manufacturer: CEM) at 170° C. (5 bar) for 12 minutes. After cooling, the reaction mixture was concentrated and the residue was worked up by column chromatography (silica gel, cyclohexane/ethyl acetate, vol.: 7:1 to 3:1). This gave 0.16 g (24% of theory) of 4-(3-chloro-4-fluorophenyl)-1-pyrimidin-2-y1-3-(trifluoromethyl)-1H-pyrazole-5-amine as a colourless solid.

¹H-NMR (400 MHz, d₆-DMSO, δ, ppm): 8.93 (d, 2H), 7.51 (m, 2H), 7.47 (t, 1H), 7.35 (m, 1H), 6.87 (broad s, 2H, NH₂).

### Example 1-87

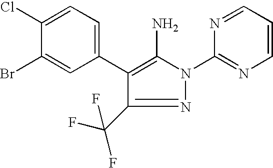

Step 1: 3-Bromo-4-chlorotoluene (1.0 equiv, 3.0 g, 14.6 mmol) and N-bromosuccinimide (1.2 equiv., 3.1 g, 17.5 mmol) were dissolved in CCl₄, AIBN (0.01 equiv., 23.9 mg, 0.15 mmol) was added and the mixture was stirred under reflux for 4 h. After cooling to room temperature, the mixture was washed with sat. NaHCO₃ solution and with water, the organic phase was concenrtated and the residue was purified by column chromatography (EtOAc/heptane gradient). This gave 2.0 g (47% of theory) of 2-bromo-4-bromomethyl-1-chlorobenzene.

Step 2: At room temperature, 2-bromo-4-bromomethyl-1-chlorobenzene (1.0 equiv, 2.0 g, 7.0 mmol) and potassium cyanide (1.3 equiv., 0.59 g, 9.1 mmol) were dissolved in a mixture of ethanol and water (2:1 parts by volume) and stirred at room temperature for 6 h. The reaction mixture was then concentrated, and ethyl acetate was added. The organic phase was washed with water, and the aqueous phase was extracted with ethyl acetate. The organic phase was dried and concentrated, and the residue was purified by column chromatography (EtOAc/heptane gradient). This gave 1.3 g (80% of theory) of (3-bromo-4-chlorophenyl)-acetonitrile.

Step 3: Sodium methoxide (1.2 equiv., 1.46 g, 6.8 mmol) was added to a solution of (3-bromo-4-chlorophenyl)acetonitrile (1.0 equiv, 1.3 g, 5.6 mmol) in abs. tetrahydrofuran, and the resulting reaction solution was stirred at 50° C. for 1 hour. Ethyl trifluoroacetate (1.2 equiv, 0.9 g, 6.8 mmol) in 2 ml tetrahydrofuran was then added dropwise, and the reaction mixture was then stirred under reflux for 6 hours. After cooling, the reaction mixture was concentrated under reduced pressure, dissolved in ethyl acetate and washed twice with 100 ml of 1N hydrochloric acid and once with water. The organic phase was separated off, dried over sodium sulphate and filtered. Under reduced pressure, the solvent was carefully distilled off from the filtrate and the residue was triturated with a solvent mixture of petroleum ether/dichloromethane (vol.: 1:1). The colourless solid that precipitated out was filtered off with suction over a glass frit and dried. This gave 1.7 g (92% of theory) of 2-(3-bromo-4-chlorophenyl)-4,4,4-trifluoro-3-oxobutyronitrile as a mixture of tautomers.

Step 4: 2-(3-Bromo-4-chlorophenyl)-4,4,4-trifluoro-3-oxobutyronitrile (1.0 equiv, 1.7 g, 5.2 mmol) and 2-pyrimidinylhydrazine (1.0 equiv, 0.57 g, 5 2 mmol) were dissolved in 10 ml of toluene. After addition of catalytic amounts of acetic acid, the reaction mixture was stirred under reflux for 6 hours. After cooling to room temperature, the reaction mixture was concentrated and the residue was purified by column chromatography (EtOAc/heptane gradient). This gave 0.19 g (9% of theory) of 4-(3-bromo-4-chlorophenyl)-2-pyrimidin-2-yl-5-(trifluoromethyl)-2H-pyrazol-3-ylamine as a colourless solid.

¹H-NMR (400 MHz, d₆-DMSO, δ, ppm): 8.97 (d, 2H), 7.72 (d, 1H), 7.69 (d, 1H), 7.56 (t, 1H), 7.38 (dd, 1H), 7.13 (s, 2H, NH₂).

### Ex. No. 3-25

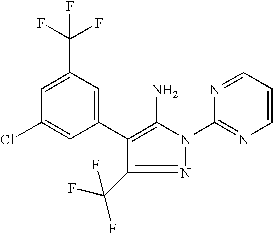

Step 1: 838 mg (2,655 mmol) of 2-(3-chloro-5-trifluoromethylphenyl)-4,4,4-trifluoro-3-oxo-butyronitrile were initially charged in 2.036 g (13.276 mmol) of phosphoryl chloride, and 0.37 ml (2.655 mmol) of triethylamine were added. The reaction mixture was stirred at 80-100° C. for 3 h and carefully stirred ito warm water. After extraction with ethyl acetate, the organic phase was dried with magnesium sulphate, filtered and concentrated under reduced pressure. This gave 758 mg (85%) of 3-chloro-2-(3-chloro-5-trifluoromethylphenyl)-4,4,4-trifluorobut-2-enenitrile.

Step 2: 750 mg (2.245 mmol) of 3-chloro-2-(3-chloro-5-trifluoromethylphenyl)-4,4,4-trifluoro-but-2-enenitrile were initially charged in 10 ml of ethanol, and 247 mg (2.245 mmol) of pyrimidin-2-ylhydrazine and 227 mg (2.245 mmol) of triethylamine were added. The mixture was heated under reflux for 10 h. After cooling, 75 ml of water were added, and the solid formed was filtered off with suction. Purification of the solid by column chromatography gave 500 mg (54%) of 4-(3-chloro-5-trifluoromethylphenyl)-2-pyrimidin-2-yl-5-trifluoromethyl-2H-pyrazol-3-ylamine.

¹H-NMR (400 MHz, CDCl₃), δ 8.84 (d, 2H), 7.61 (s, 2H), 7.58 (s, 1H), 6.18 (s, 2H, NH).

### Ex. No. 8-92

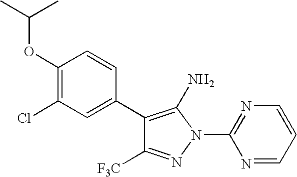

Step 1: Aminocrotonitrile (7150 mg, 52 5 mmol, 1 equiv) was initially charged in aqueous HCl, and after 5 min of stirring 2-hydrazinopyrimidine (5786 mg, 52.54 mmol, 1 equiv) was added. The reaction solution was stirred under reflux for 4 h and, after cooling, poured into 2N NaOH. The aqueous phase was then extracted repeatedly with ethyl acetate. The combined organic phases were dried over sodium sulphate, filtered off and concentrated. The residue was recrystallized from ethyl acetate, giving 2-pyrimidin-2-y1-5-trifluoromethyl-2H-pyrazol-3-ylamine (5002 mg, 42% of theor. yield).

Step 2: 2-Pyrimidin-2-yl-5-trifluoromethyl-2H-pyrazol-3-ylamine (1000 mg, 4.36 mmol, 1 equiv) was added to acetic acid, and after 5 min of stirring at room temperature, a solution of bromine (0.22 ml, 4.36 mmol, 1 equiv) in acetic acid was added dropwise. The reaction mxiture was stirred at room temperature for 4 h, water was then added and the mxiture was extracted repeatedly with dichloromethane. The combined organic phases were dried over sodium sulphate and concentrated. The resulting residue was purified by column chromatography, giving 1120 mg of 4-bromo-2-pyrimidin-2-yl-5-trifluoromethyl-2H-pyrazol-3-ylamine (83% of theor. yield).

Step 3: Under argon, DMF (4.5 ml) was added to 4-bromo-2-pyrimidin-2-yl-5-trifluoromethyl-2H-pyrazol-3-ylamine (200 mg, 0.65 mmol, 1.0 equiv) and 3-chloro-4-isopropoxyphenyl-boronic acid (181 mg, 0.84 mmol, 1.3 equiv) in a round-bottom flask which had been dried by heating, and the mixture was stirred for 5 min. Tetrakis(triphenylphosphine)palladium(0) (60 mg, 0.05 mmol, 0.08 equiv) and sodium carbonate solution (2M, 0.5 ml) were then added. The resulting reaction mxiture was stirred at 80° C. for 4 h. After cooling to room temperature, water and dichloromethane were added. After repeated extraction of the aqueous phase with dichloromethane, the combined organic phases were dried over sodium sulphate and concentrated. Purification of the resulting residue by prep. HPLC gave 4-(3-chloro-4-isopropoxyphenyl)-2-pyrimidin-2-yl-5-trifluoromethyl-2H-pyrazol-3-ylamine (20 mg, 7%).

¹H-NMR (400 MHz, CDCl₃), δ 8.83 (d, 2H), 7.41 (d, 1H), 7.27 (t, 1H), 7.21 (d, 1H), 7.00 (d, 1H), 5.98 (br. s, 2H, NH), 4.58 (sept, 1H), 1.42 (d, 6H).

### Example 11-10

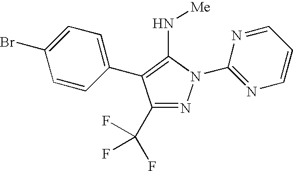

0.30 g (0.78 mmol) of 4-(4-bromophenyl)-1-pyrimidin-2-yl-3-(trifluoromethyl)-1H-pyrazole-5-amine (Example 01-09) were initially charged in 6 ml of DMF and covered with nitrogen. 0.041 g (1 mmol) of sodium hydride (60%) and 0.12 g (0.86 mmol) of methyl iodide were added, and the mixture was heated at 40° C. for 5 hours. After extractive work-up with heptane/ethyl acetate (vol. 1:1) and water, the product was isolated by preparative HPLC separation. This gave 0.060 g of 4-(4-bromophenyl)-N-methyl-1-pyrimidin-2-yl-3-(trifluoromethyl)-1H-pyrazole-5-amine (11-10) as a colourless solid.

¹H-NMR (300 MHz, CDCl3, δ, ppm): 8.81, 2H, d; 7.50, 2H, d; 7.29, 2H, d; 7.26, 1H, tr; 2.60, 3H, s.

### Example 12-07

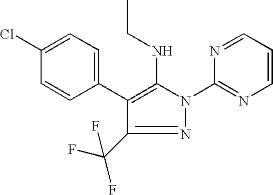

0.3 g (0 9 mmol) of 4-(4-chlorophenyl)-1-pyrimidin-2-yl-3-(trifluoromethyl)-1H-pyrazole-5-amine (Table 1, substance 1-06) was dissolved in 1.5 ml of DMF, and 0.07 g (1 8 mmol, 60% dispersion in mineral oil) of sodium hydride was added at room temperature. 0.28 g (1 8 mmol) of ethyl iodide were dissolved in 0.5 ml of DMF and added dropwise. The reaction mixture was stirred at 55° C. for 12 hours and then concentrated to ten percent of its volume. The residue was stirred vigorously with 1N hydrochloric acid and dichloromethane, the dichloromethane phase was separated in a phase separation cartridge (manufacturer: Whatman) and on a rotary evaporator the solvent was carefully distilled off from the filtrate. The residue was chromatographed by preparative HPLC.

This gave 0.092 g (24.2% of theory) of 4-(4-chlorophenyl)-N-ethyl-l-pyrimidin-2-yl-3-(trifluoromethyl)-1H-pyrazole-5-amine as an amorphous residue.

¹H-NMR (400 MHz, CD₃CN, δ, ppm): 8.83 (d, 2H), 7.77 (broad s, 1H, NH), 7.41 (s, 4H), 7.37 (t, 1H), 2.84 (broad m, 2H), 0.96 (t, 3H).

### Example 14-07

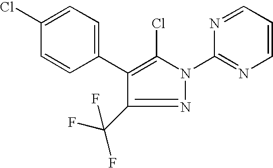

1.5 g (4 4 mmol) of 4-(4-chlorophenyl)-1-pyrimidin-2-yl-3-(trifluoromethyl)-1H-pyrazole-5-amine (Table 1, substance 1-06) were dissolved in 10 ml of concentrated hydrochloric acid. After one hour of stirring at room temperature, a poorly soluble precipitate was formed. 0.72 g (5 2 mmol) of copper(II) chloride and a further 10 ml of concentrated hydrochloric acid were added. 0.54 g (7.9 mmol) of sodium nitrite were then added a little at a time (evolution of gas). The reaction mixture was stirred at room temperature for 12 hours and then poured on ice and subsequently extracted with ethyl acetate. The organic phase was separated off, dried over sodium sulphate and filtered, and the solvent was carefully distilled off on a rotary evaporator. The residue was worked up by column chromatography (silica gel, dichloromethane/aceto-nitrile, Vol.: 9:1).

This gave 0.28 g (11.3% of theory) of 245-chloro-4-(4-chlorophenyl)-3-(trifluoromethyl)-1H-pyrazol-1-yl]pyrimidine as a yellow-orange solid.

¹H-NMR (400 MHz, d₆-DMSO, δ, ppm): 8.95 (d, 2H), 7.58 (t, 1H), 7.51 (d, 2H), 7.46 (d, 2H).

### Example 14-29

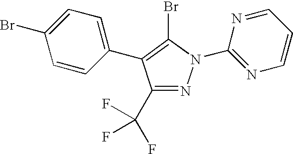

1.50 g (3.90 mmol) of 4-(4-bromophenyl)-1-pyrimidin-2-yl-3-(trifluoromethyl)-1H-pyrazole-5-amine (Example 01-09) were initially charged in 8 ml (23.15 g, 91.6 mmol) of bromoform. 0.60 g (5.86 mmol) of t-butyl nitrite were added at 0-10° C., and the mixture was heated at 70° C. for 2 h. After concentration on a rotary evaporator, the product was isolated by column chromatography. This gave 0.82 g of 5-bromo-4-(4-bromophenyl)-N-methyl-1-pyrimidin-2-yl-3-(trifluoromethyl)-1H-pyrazole (Example 14-29) as a solid.

¹H-NMR (400 MHz, CDCl3, δ, ppm): 8.94, 2H, d; 7.61, 2H, d; 7.48, 1H, tr; 7.30, 2H, d; 19F-NMR (CDCl3): −61.23 ppm.

### Example 29-37

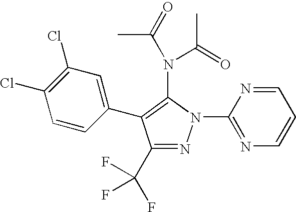

0.50 g (1.34 mmol) of 4-(3,4-dichlorophenyl)-1-pyrimidin-2-yl-3-(trifluoromethyl)-1H-pyrazole-5-amine (Example 01-80) were initially charged in 3 ml of dioxane. 0.27 g (2.67 mmol) of acetic anhydride and 0.016 g (0.13 mmol) of DMAP were added, and the mixture was heated at 100° C. for 6 h. After extractive work-up with heptane/ethyl acetate (vol. 1:1) and water, the product was isolated by column chromatography. This gave 0.190 g of N,N-diacetyl-4-(3,4-dichlorophenyl)-1-pyrimidin-2-yl-3-(trifluoromethyl)-1H-pyrazole-5-amine (Example 29-37) as a colourless solid.

¹H-NMR (300 MHz, CDCl3, δ, ppm): 8.84, 2H, d; 7.54, 1H, d; 7.39, 3H, d +tr; 7.16, 1H, dd; 2.27, 6H, s; ¹⁹F-NMR (300 MHz, CDCl3): −61.19 ppm.

### Example 31-29

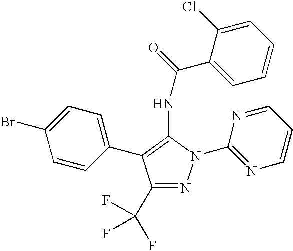

0.10 g (0.26 mmol) of 4-(4-bromophenyl)-1-pyrimidin-2-yl-3-(trifluoromethyl)-1H-pyrazole-5-amine (Example 01-09) was initially charged in 3 ml of dioxane. 0.050 g (0.286 mmol) of 2-chlorobenzoyl chloride, 0.050 g (0.39 mmol) of diisopropylethylamine and 0.006 g (0.05 mmol) of DMAP were added, and the mixture was heated at 100° C. for 6 h. After extractive work-up with heptane/ethyl acetate (vol. 1:1) and water, the product was isolated by column chromatography. This gave 0.100 g of N-(2-chlorobenzoyl)-4-(3,4-dichlorophenyl)-1-pyrimidin-2-yl-3-(trifluoromethyl)-1H-pyrazole-5-amine (Example 31-29) as a colourless solid.

¹H-NMR (300 MHz, CDCl3, δ, ppm): 10.16, 1H, s; 8.86, 2H, d; 7.55, 2H, d; 7.37, 1H, tr; 7.48-7.32, 6H, m; ¹⁹F-NMR (300 MHz, CDCl3): -61.25 ppm.

### Example 36-10 <

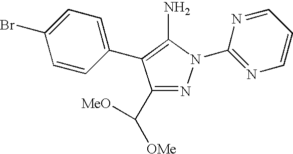

0.40 g (1.34 mmol) of 2-(4-bromophenyl)-4,4-dimethoxy-3-oxobutyronitrile were initially charged in 6 ml of methanol. 0.148 g (1.34 mmol) of 2-pyrimidinylhydrazine and 0.32 g (5.36 mmol) of acetic acid were added, and the mixture was heated at 65° C. for 2 h. After concentration on a rotary evaporator, the residue was triturated with heptane/ethyl acetate (vol. 4:1). This gave 0.29 g of 4-(4-bromophenyl)-3-(dimethoxymethyl)-1-pyrimidin-2-yl-1H-pyrazole-5-amine (Example 36-10) as a colourless solid.

¹H-NMR (400 MHz, d6DMSO, δ, ppm): 8.87, 2H, d; 7.55, 2H, d; 7.44, 3H, d+tr; 6.77, 2H, s, br; 5.22, 1H, s; 3.27, 6H, s.

### Example 38-10

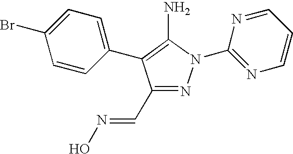

0.15 g (0.38 mmol) of 4-(4-bromophenyl)-3-(dimethoxymethyl)-1-pyrimidin-2-yl-1H-pyrazole-5-amine (36-10) was initially charged in 4 ml of dioxane and 1 ml of water. 0.053 g (0.769 mmol) of hydroxylammonium chloride and 0.38 ml (0.769 mmol) of 2N hydrichloric acid were added, and the mixture was heated at 100° C. for 8 h. On stirring with 40 ml of water, the oxime product precipitated out. This gave 0.089 g of 4-(4-bromophenyl)-1-pyrimidin-2-yl-1H-pyrazole-5-amine-3-carbaldehyde oxime (Example 38-10) as a colourless solid.

¹H-NMR (400 MHz, d6DMSO, δ, ppm): 11.13, 1H, s; 8.88, 2H, d; 7.96, 1H, s; 7.57, 2H, d; 7.46, 1H, tr; 7.32, 2H, d; 6.74, 2H, s, br.

### Example 37-10

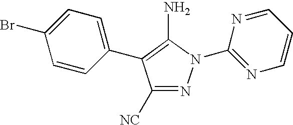

0.089 g (0.248 mmol) of 4-(4-bromophenyl)-1-pyrimidin-2-yl-1H-pyrazol-5-amin-3-carbaldehyde oxime (Example 38-10) was initially charged in 2 ml of 1,2-dichloroethane. 0.114 g (0.743 mmol) od phosphorus oxychloride were added, and the mxiture was stirred at 25° C. for 20 h. The mixture was concentrated on a rotary evaporator and the residue was stirred water water, and the solid that remained was then filtered off with suction. This gave 0.033 g of 4-(4-bromophenyl)-3-cyano-1-pyrimidin-2-yl-1H-pyrazole-5-amine (Example 37-10) as a colourless solid.

¹H-NMR (400 MHz, d6DMSO, δ, ppm): 8.95, 2H, d; 7.71, 2H, d; 7.59, 1H, tr; 7.48, 2H, d; 7.28, 2H, s, br.

### Biological Examples

### Example A

Phaedon Test (PHAECO Spray Treatment)

| | |
|---|---|
| Solvent: | 78.0 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of chinese cabbage (Brassica pekinensis) are sprayed with an active compound preparation of the desired concentration and, after drying, populated with larvae of the mustard beetle (Phaedon cochleariae).

After the desired period of time, the effect in % is determined 100% means that all beetle larvae have been killed; 0% means that none of the beetle larvae have been killed.

In this test, for example, the following compounds of the Preparation Examples show, at an application rate of 500 g/ha after 7 d, an activity of at least 80%:

Ex. No. 1-3, 1-5, 1-6, 1-8, 1-9, 1-11, 1-12, 1-14, 1-17, 1-18, 1-20, 1-22, 1-23, 1-30, 1-32, 1-33, 1-34, 1-71, 1-73, 1-74, 1-75, 1-79, 1-80, 1-81, 1-82, 1-83, 1-87, 1-88, 1-89, 2-4, 2-5, 2-6, 2-12, 2-13, 2-15, 2-16, 2-17, 3-2, 3-3, 3-11, 3-14, 3-15, 3-20, 3-23, 3-24, 3-25, 3-26, 3-31, 3-32, 3-33, 3-34, 4-2, 4-3, 4-5, 4-6, 4-7, 4-18, 4-19, 4-21, 4-34, 4-36, 5-2, 5-5, 5-9, 5-22, 5-23, 5-25, 5-33, 5-50, 5-58, 5-59, 5-60, 5-62, 5-80, 5-89, 6-1, 6-61, 6-70, 6-78, 8-5, 8-6, 8-11, 8-24, 8-25, 8-26, 8-27, 8-30, 8-31, 8-42, 8-43, 8-54, 8-62, 8-65, 8-66, 8-68, 8-69, 8-73, 8-88, 8-89, 8-92, 8-107, 8-108, 8-109, 8-110, 8-111, 8-112, 9-4, 9-06, 9-09, 9-14, 11-7, 11-10, 11-82, 12-7, 12-12, 12-80, 12-103, 12-104, 13-19, 13-31, 13-32, 13-115, 13-116, 14-7, 14-18, 15-7, 15-19, 15-38, 15-45, 15-48, 15-51, 15-57, 15-95, 15-105, 15-108, 15-114, 15-154, 15-155, 15-156, 15-157, 15-158, 15-159, 15-160, 15-161, 15-162, 15-163, 15-164, 15-165, 15-166, 15-167, 15-168, 15-169, 16-4, 16-6, 16-7, 16-15, 16-18, 16-19, 16-27, 16-30, 16-38, 16-39, 16-49, 16-87, 16-134, 16-162, 17-37, 17-48, 17-67, 17-124, 17-143, 18-7, 18-10, 18-19, 18-26, 18-32, 18-34, 18-38, 18-45, 18-48, 18-51, 18-57, 18-124, 18-143, 18-181, 18-190, 18-200, 18-209, 18-219, 18-229, 18-230, 18-231, 18-232, 18-233, 18-234, 18-235, 18-236, 18-237, 18-238, 18-240, 18-241, 18-242, 18-243, 18-244, 18-245, 18-246, 19-56, 19-57, 19-121, 25-31, 25-07, 26-7, 26-37, 26-46, 26-62, 26-65, 26-84, 26-102, 26-103, 26-116, 26-125, 26-139, 26-140, 27-19, 27-26, 27-38, 27-45, 27-50, 27-51, 27-76, 27-114, 27-140, 27-153, 27-155, 27-156, 27-157, 27-158, 27-159, 27-160, 27-163, 28-7, 29-26, 29-37, 29-64, 29-156, 29-158, 29-159, 29-161, 29-162, 29-163, 29-164, 29-165, 30-10, 32-52, 32-54, 35-07, 39-10, 39-48, 39-86, 39-105, 39-124, 39-162, 39-219, 39-200, 40-10, 40-29, 40-124, 41-86, 42-39, 42-96, 43-02, 43-03, 43-04, 43-05, 43-06, 43-09, 43-10, 43-11

### Example B

Tetranychus Test, OP-Resistant (TETRUR Spray Treatment)

| | |
|---|---|
| Solvent: | 78.0 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of bean leaves (Phaseolus vulgaris) which are infested by all stages of the greenhouse red spidermite (Tetranychus urticae) are sprayed with an active compound preparation of the desired concentration.

After the desired period of time, the effect in % is determined 100% means that all spidermites have been killed; 0% means that none of the spidermites have been killed.

In this test, for example, the following compounds of the Preparation Examples show, at an application rate of 100 g/ha after 5 d, an activity of at least 80%:

### Ex. No. 15-108, 15-154, 19-57, 27-114, 18-242

In this test, for example, the following compounds of the Preparation Examples show, at an application rate of 500 g/ha after 5 d, an activity of at least 80%:

Ex. No. 1-9, 1-22, 1-24, 1-71, 1-73, 1-81, 1-83, 2-4, 2-12, 2-6, 2-15, 3-11, 3-14, 3-24, 3-25, 3-32, 3-33, 8-107, 8-108, 13-116, 12-82, 15-57, 15-105, 15-108, 15-154, 15-155, 15-156, 15-158, 15-159, 15-160, 15-162,15-163, 15-164, 15-165, 15-166, 15-168, 15-169, 16-38, 16-134, 18-10, 18-38, 18-57, 18-232, 18-233, 18-234, 18-235, 18-236, 18-237, 18-238, 18-241, 18-244, 16-134, 25-7, 25-31, 26-37, 26-62, 27-153, 27-158, 27-160, 29-37, 29-165, 43-02

### Example C

Spodoptera frugiperda Test (SPODFR Spray Treatment)

| | |
|---|---|
| Solvent: | 78.0 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of maize leaves (Zea mays) are sprayed with an active compound preparation of the desired concentration and, after drying, populated with caterpillars of the army worm (Spodoptera frugiperda).

After the desired period of time, the effect in % is determined 100% means that all caterpillars have been killed; 0% means that none of the caterpillars have been killed.

In this test, for example, the following compounds of the Preparation Examples show, at an application rate of 500 g/ha after 7 d, an activity of ≧80%:

Ex. No. 1-5, 1-6, 1-9, 1-12, 1-13, 1-17, 1-18, 1-22, 1-23, 1-32, 1-33, 1-71, 1-73, 1-74, 1-75, 1-79, 1-80, 1-81, 1-82, 1-83, 1-87, 1-88, 1-89, 1-32, 1-89, 2-2, 2-6, 2-12, 2-13, 2-16, 2-17, 3-11, 3-14, 3-20, 3-24, 3-25, 3-26, 3-31, 3-32, 3-34, 4-3, 4-21, 4-23, 4-34, 4-36, 5-33, 6-70, 6-78, 8-5, 8-11, 8-24, 8-26, 8-27, 8-31, 8-44, 8-48, 8-66, 8-68, 8-69, 8-73, 8-107, 9-06, 9-09, 11-82, 12-7, 12-80, 12-82, 12-99, 12-103, 13-19, 13-31, 13-32, 13-115, 13-116, 14-7, 14-18, 15-19, 15-38, 15-45, 15-48, 15-51, 15-57, 15-95, 15-108, 15-114, 15-154, 15-155, 15-156, 15-157, 15-158, 15-159, 15-160, 15-161, 15-162, 15-163, 15-164, 15-165, 15-166, 15-167, 15-168, 15-169, 16-38, 16-39, 16-134, 18-10, 18-19, 18-26, 18-32, 18-38, 18-45, 18-48, 18-51, 18-57, 18-124, 18-181, 18-190, 18-200, 18-209, 18-229, 18-230, 18-231, 18-232, 18-233, 18-234, 18-235, 18-236, 18-237, 18-238, 18-239, 18-240, 18-241, 18-242, 18-243, 18-244,18-245,18-246, 19-56, 19-57, 27-19, 27-26, 27-38, 27-76, 27-114, 27-153, 27-155, 27-158, 27-160, 29-19, 29-29, 29-37, 29-143, 29-156, 29-157, 29-161, 29-162, 29-163, 29-164, 29-165, 30-10, 42-77, 42-96, 42-135, 43-01, 43-02, 43-03, 43-04, 43-05, 43-06, 43-11.

### Example D

Myzus Test (MYZUPE Spray Treatment)

| | |
|---|---|
| Solvents: | 78.0 parts by weight of acetone |
| | 1.5 parts by weight of dimethylformamide |
| Emulsifier: | 0.5 part by weight of alkylaryl polyglycol ether |

To produce a suitable preparation of active compound, 1 part by weight of active compound is mixed with the stated amounts of solvents and emulsifier, and the concentrate is diluted with emulsifier-containing water to the desired concentration.

Discs of chinese cabbage (Brassica pekinensis) which are infested by all stages of the green peach aphid (Myzus persicae) are sprayed with an active compound preparation of the desired concentration.

After the desired period of time, the effect in % is determined 100% means that all aphids have been killed; 0% means that none of the aphids have been killed.

In this test, for example, the following compounds of the Preparation Examples show, at an application rate of 500 g/ha after 5 d, an activity of at least 80%:

Ex. No. 1-6, 1-9, 1-11, 1-14, 1-17, 1-22, 1-71, 1-74, 1-79, 1-81, 1-89, 3-14, 3-20, 3-25, 3-26, 3-32, 4-5, 6-61, 8-6, 8-27, 8-44, 8-48, 8-68, 8-69, 8-107, 12-103, 12-104, 13-19, 13-32, 13-116, 15-19, 15-38, 15-45, 15-57, 15-95, 15-105, 15-108, 15-114, 15-154, 15-155, 15-156, 15-158, 15-159, 15-161, 15-162, 15-165, 15-169, 16-15, 16-19, 16-27, 16-39, 16-65, 16-134, 17-37, 18-19, 18-26, 18-32, 18-34, 18-38, 18-57, 18-229, 18-232, 18-235, 18-236, 18-239, 18-242, 19-38, 19-45, 19-48, 19-56, 19-57, 19-76, 19-152, 19-191, 25-07, 26-6, 26-23, 26-124, 26-139, 27-19, 27-26, 27-38, 27-45, 27-76, 27-114, 27-140, 27-153, 27-159, 27-160, 27-163, 35-07, 29-165, 42-96, 43-09, 43-10, 43-11.

### Example E

Lucilia cuprina Test (LUCICU)

Solvent: dimethyl sulphoxide

To produce a suitable preparation of active compound, 1 part by weight of active compound is mixed with the stated amount of water, and the concentrate is diluted with water to the desired concentration.

Containers containing horse meat treated with the active compound preparation of the desired concentration are populated with Lucilia cuprina larvae.

After the desired period of time, the kill in % is determined 100% means that all larvae have been killed; 0% means that none of the larvae have been killed.

In this test, for example, the following compounds of the Preparation Examples show, at an application rate of 100 ppm after 2 d, an activity of ≧80%:

Ex. No. 1-3, 1-5, 1-6, 1-9, 1-11, 1-12, 1-18, 1-22, 1-33, 1-71, 1-73, 1-79, 1-80, 1-81, 1-82, 1-83, 1-89, 2-13, 2-4, 2-6, 2-12, 2-15, 3-14, 3-20, 3-24, 3-25, 3-31, 3-32, 4-3, 4-6, 4-19, 4-21, 4-23, 4-36, 5-2, 5-22, 5-33, 5-62, 8-24, 8-31, 8-68, 8-108, 12-7, 12-82, 13-116, 14-7, 14-18, 15-38, 15-45, 15-51, 15-57, 15-95, 15-108, 15-157, 15-158, 15-159, 15-160, 15-161, 15-162, 15-163, 15-165, 15-166, 15-168, 16-6, 16-7, 16-19, 16-134, 18-7, 18-10, 18-19, 18-38, 18-45, 18-230, 18-233, 18-234, 18-232, 18-235, 18-236, 18-237, 18-244, 19-56, 26-46, 26-65, 26-84, 26,-139, 27-114, 27-160

### Example F

Musca domestica Test (MUSCDO)

Solvent: dimethyl sulphoxide

To produce a suitable preparation of active compound, 1 part by weight of active compound is mixed with the stated amount of water, and the concentrate is diluted with water to the desired concentration.

Containers containing a sponge treated with the active compound preparation of the desired concentration were populated with adult Musca domestica.

After the desired period of time, the kill in % is determined 100% means that all flies have been killed; 0% means that none of the flies have been killed.

In this test, for example, the following compounds of the Preparation Examples show, at an application rate of 20 ppm after 2 d, an activity of ≧80%:

### Ex. No. 1-6, 1-9, 1-12, 1-18, 1-22, 1-33, 1-73, 1-77, 1-79, 1-80, 1-81, 2-13, 3-24, 4-3, 4-21, 14-18, 15-45, 15-51,15-159, 15-160, 15-168, 18-234, 18-244, 18-236, 18-237

### Example G

Boophilus microplus Test (BOOPMI Injection)

Solvent: dimethyl sulphoxide

To produce a suitable preparation of active compound, 1 part by weight of active compound is mixed with the stated amount of solvent, and the concentrate is diluted with solvent to the desired concentration.

The active compound solution is injected into the abdomen (Boophilus microplus) and the animals are transferred into dishes and stored in a climatized room.

After the desired period of time the activity in % is determined 100% means that none of the ticks has laid fertile eggs.

In this test, for example, the following compounds of the Preparation Examples show, at an application rate of 20 μg/animal after 7 d, an activity of ≧80%:

Ex. No. 1-5, 1-6, 1-9, 1-11, 1-12, 1-18, 1-22, 1-71, 1-73, 1-79, 1-80, 1-81, 1-82, 1-83, 1-89, 2-12, 2-13, 3-2, 3-14, 3-20, 3-24, 3-25, 3-31, 3-32, 4-3, 4-21, 4-36, 5-2, 5-22, 5-33, 5-62, 8-31, 15-57, 2-13, 3-20, 3-31, 8-11, 8-24, 8-54, 8-68, 8-69, 15-45, 15-51, 15-108, 15-157, 15-158, 15-160, 15-161, 15-162, 15-163, 15-164, 15-165, 15-166, 15-168, 16-19, 16-30, 16-38, 16-134, 17-37, 18-7, 18-10, 18-19, 18-38, 18-45,18-230, 18-232, 18-233, 18-234, 18-235, 18-236, 18-237, 18-244, 26-84, 27-114

### Example H

Ctenocephalides fells; Oral (CTECFE)

Solvent: dimethyl sulphoxide

To produce a suitable preparation of active compound, 1 part by weight of active compound is mixed with the stated amount of water. Part of the concentrate is diluted with citrated cattle blood, and the desired concentration is prepared.

20 unfed adult fleas (Ctenocephalides felis) are placed into a chamber whose top and bottom ends are closed with gauze. A metal cylinder whose bottom end is closed with parafilm is placed onto the chamber. The cylinder contains the blood/active compound preparation, which can be taken up by the fleas through the parafilm membrane. The blood is warmed to 37° C., but the flea chamber is at room temperature.

After the desired period of time, the kill in % is determined 100% means that all fleas have been killed; 0% means that none of the fleas have been killed.

In this test, for example, the following compounds of the Preparation Examples show, at an application rate of 20 ppm after 2 d, an activity of ≧80%:

Ex. No. 1-5, 1-9, 1-12, 1-18, 1-22, 1-80, 1-81, 1-83, 1-89, 3-14, 3-20, 3-25, 3-31, 4-3, 8-24, 8-68, 1-81, 12-82, 15-57, 15-95, 15-157, 15-158, 15-161, 15-162, 15-163, 18-19, 18-38, 18-230, 18-232, 18-234, 18-235, 18-236, 18-237

1. A compound of formula (I) 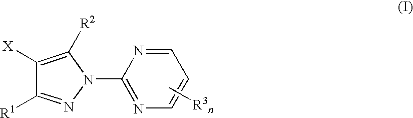
in which
X represents phenyl, 2-pyridyl, 3-pyridyl or 2-pyrimidinyl, in each case substituted by one or more substituents selected from the group consisting of halogen, alkyl, haloalkyl, alkoxy, alkoxyalkyl, alkoxyalkoxy, cycloalkyl, alkenoxy, alkynoxy, benzyloxy, cycloalkylalkoxy, haloalkoxy, haloalkoxyalkyl, alkylsulphanyl, haloalkylsulphanyl, alkylsulphinyl, haloalkylsulphinyl, alkylsulphonyl, haloalkylsulphonyl, cyano, alkylcarbonyl, alkoxycarbonyl, alkoxycarbonylalkyl, carboxyl, carboxamide, dialkylcarboxamide, trialkylsilyl, nitro, amino, alkylamino, dialkylamino, alkylsulphonylamino, dialkylsulphonylamino, —CH═NO—H, —CH═NO-alkyl, —CH═NO-halolkyl, —C(CH₃)═NO—H, —C(CH₃)═NO-alkyl, —C(CH₃)═NO-haloalkyl, formyl, benzyl (for its part optionally substituted by one or more halogen atoms), phenoxy or pyrid-2-yloxy (the latter two optionally substituted by one or more substituents selected from the group consisting of alkyl, halogen and haloalkyl) and phenyl (optionally substituted by one or more halogen atoms), where vicinal alkyl, haloalkyl, alkoxy and/or haloalkoxy groups together with the carbon atoms to which they are attached may form a five- to six-membered cyclic system which contains 0 to 2 oxygen atoms and whose alkyl moiety may optionally be substituted by one or more fluorine atoms,
R¹ represents alkyl (optionally mono- or disubstituted by substituents independently of one another selected from the group consisting of alkoxy, haloalkoxy, alkylsulphanyl, haloalkylsulphanyl, alkylsulphinyl, haloalkylsulphinyl, alkylsulphonyl, haloalkylsulphonyl, alkylcarbonyl, alkoxycarbonyl, hydroxyl, cycloalkyl, phenyl (for its part optionally substituted by one or more halogen atoms) and heterocyclyl), cycloalkyl (optionally substituted by one or more haloalkyl groups and/or halogen atoms), haloalkyl (optionally substituted by alkoxy), cyano, formyl, —CH═NO—H, —CH═NO-alkyl, —CH═NO-haloalkyl, —C(CH₃)═NO—H, —C(CH₃)═NO-alkyl, —C(CH₃)═NO-haloalkyl or benzyl (optionally mono- or polysubstituted by one or more substituents independently of one another selected from the group consisting of halogen, alkyl, haloalkyl and alkoxy),
R² represent amino and
R² furthermore represents amino which is monosubstituted by alkyl, alkylcarbonyl, cycloalkyl, cycloalkylalkyl, alkenyl, alkoxycarbonyl, alkenoxycarbonyl, alkynoxycarbonyl, alkoxycarbonylalkyl, alkoxycarbonylcarbonyl, benzyl or phenylcarbonyl (where the phenyl ring in benzyl and phenylcarbonyl for its part is optionally substituted by one or more halogen atoms) and
R² furthermore represents amino which is disubstituted by two substituents independently of one another selected from the group consisting of benzyl, alkyl, alkoxycarbonyl, alkenoxycarbonyl, alkynoxycarbonyl and alkylcarbonyl,
R² furthermore represents piperidine, pyrrolidine or morpholine which are optionally mono- or disubstituted by alkyl and
R² furthermore represents halogen,
R³ represents halogen, alkyl, haloalkyl, alkoxy or dialkylamino and n represents 0 or 1,
as well as N-oxides and/or salts thereof.

2. A compound of formula (I) according to claim 1 in which
X represents phenyl, 2-pyridyl, 3-pyridyl or 2-pyrimidinyl steht, in each case substituted by one or more substituents selected from the group consisting of fluorine, chlorine, bromine, iodine, C₁-C₆-alkyl, C₁-C₆-haloalkyl, C₁-C₆-alkoxy-C₁-C₆-alkyl, C₁-C₆-alkoxy, C₁-C₆-haloalkoxy, C₁-C₆-haloalkoxy-C₁-C₆-alkyl, C₁-C₆-alkylsulphanyl, halo-C₁-C₆-alkylsulphanyl, C₁-C₆-alkylsulphinyl, halo-C₁-C₆-alkylsulphinyl, C₁-C₆-alkylsulphonyl, halo-C₁-C₆-alkylsulphonyl, cyano, C₁-C₆-alkylcarbonyl, C₁-C₆-alkoxycarbonyl, C₁-C₆-alkoxycarbonyl-C₁-C₆-alkyl, nitro, amino, C₁-C₆-alkylamino, di(C₁-C₆-alkyl)amino, C₁-C₆-alkylsulphonylamino, di(C₁-C₆-alkyl)sulphonylamino, —CH═NO—H, —CH═NO—C₁-C₆-alkyl, —CH═NO-halo-C₁-C₆-alkyl, —C(CH₃)═NO—H, —C(CH₃)═NO—C₁-C₆-alkyl, —C(CH₃)═NO-halo-C₁-C₆-alkyl, formyl, benzyl (for its part optionally substituted by one or more halogen atoms), phenoxy or pyrid-2-yloxy (the latter two optionally substituted by one or more halogen atoms and/or halo-C₁-C₆-alkyl groups) and phenyl (optionally substituted by one or more halogen atoms), where vicinal alkyl, haloalkyl, alkoxy and/or haloalkoxy groups together with the carbon atoms to which they are attached may form a five- to six-membered cyclic system which contains 0 to 2 oxygen atoms and whose alkyl moiety may optionally be substituted by one or more fluorine atoms,
R¹ represents C₁-C₆-alkyl (optionally mono- or disubstituted by substituents independently of one another selected from the group consisting of C₁-C₆-alkoxy, halo-C₁-C₆-alkoxy, C₁-C₆-alkylsulphanyl, halo-C₁-C₆-alkylsulphanyl, C₁-C₆-alkylsulphinyl, halo-C₁-C₆-alkylsulphinyl, C₁-C₆-alkylsulphonyl, halo-C₁-C₆-alkylsulphonyl, C₁-C₆-alkylcarbonyl, C₁-C₆-alkoxycarbonyl, hydroxyl, C₃-C₆-cycloalkyl, phenyl and heterocyclyl), C₃-C₆-cycloalkyl (optionally substituted by one or more halogen atoms), halo-C₁-C₆-alkyl (optionally substituted by C₁-C₆-alkoxy), cyano, formyl, —CH═NO—H, —CH═NO—C₁-C₆-alkyl, —CH═NO-halo-C₁-C₆-alkyl, —C(CH₃)═NO—H, —C(CH₃)═NO—C₁-C₆-alkyl, —C(CH₃)═NO-halo-C₁-C₆-alkyl or benzyl (optionally mono- or polysubstituted by one or more substituents independently of one another selected from the group consisting of halogen, C₁-C₆-alkyl and C₁-C₆-alkoxy),
R² represents amino which is optionally monosubstituted by C₁-C₆-alkyl, C₁-C₆-alkylcarbonyl, C₁-C₆-alkoxycarbonyl-C₁-C₆-alkyl, C₃-C₅-cycloalkyl, C₃-C₅-cycloalkyl-C₁C₃-alkyl, C₁-C₆-alkenyl, C₁-C₆-alkoxycarbonyl, C₃-C₆-alkenoxycarbonyl, C₃-C₆-alkynoxycarbonyl, C₁-C₆-alkoxycarbonylcarbonyl, benzyl or phenylcarbonyl (where the phenyl ring in benzyl and phenylcarbonyl for its part is optionally substituted by one or more halogen atoms),
R² furthermore represents amino which is disubstituted by two substituents independently of one another selected from the group consisting of benzyl, C₁-C₆-alkyl and C₁-C₆-alkylcarbonyl, C₁-C₆-alkoxycarbonyl, C₃-C₆-alkenoxycarbonyl and C₃-C₆-alkynoxycarbonyl,
R² furthermore represents piperidine, pyrrolidine or morpholine which are optionally substituted by one or two C₁-C₆-alkyl,
R² furthermore represents fluorine, chlorine, bromine or iodine,
R³ represents fluorine, chlorine, bromine, C₁-C₆-alkyl, halo-C₁-C₆-alkyl, C₁-C₆-alkoxy or di-C₁-C₆-alkylamino and
n represents 0 or 1.

3. A compound of formula (I) according to claim 1, wherein if X represents phenyl substituted by C₁-C₅-alkyl, Cl, F, Br, CF₃, C₂-C₃-alkenyl, C₂-C₃-alkynyl, C₁-C₄ alkoxy, alkoxyalkyl, phenyl, benzyl, O-phenyl or O-benzyl, and R² represents NH₂ and R¹ represents CF₃, C₁-C₃-alkyl or C₁-C₃-alkoxy-C₁-C₃-alkyl and n represents 0, then X is at least disubstituted, and if X is substituted by two identical substituents, said two substituents are located neither in positions 2 and 4 nor in positions 3 and 4.

4. A compound of formula (I), wherein the compounds are selected from Tables 1 to 43, and further wherein
a) if the compounds are selected from Table 1, then, if X is monosubstituted, the substituent of X does not represent F, Cl or Br, and, if X is substituted by two identical substituents selected from the group consisting of F, Cl or Br, then said two substituents are located neither in positions 2 and 4 nor in positions 3 and 4;
b) if the compounds are selected from Table 3, then, if X is monosubstituted, the substituent is not CF₃, and, if X is disubstituted by CF₃, the two substituents are located neither in positions 2 and 4 nor in positions 3 and 4;
c) if the compounds are selected from Table 8, then, if X is substituted by two identical substituents selected from the group consisting of methyl and methoxy, the two substituents are located neither in positions 2 and 4 nor in positions 3 and 4;
d) if the compounds are selected from Table 16 where R¹ is C₁-C₃-alkyl, then, if X is monosubstituted, the substituent of X does not represent F, Cl or Br, and, if X is disubstituted by Cl, the two substituents are located neither in positions 2 and 3 nor in positions 2 and 4 nor in positions 3 and 4; and
e) if the compounds are selected from Table 17 where R¹ is CH₂OMe, CH₂OEt or CH(OMe)Me, then, if X is monosubstituted, the substituent of X does not represent F, Cl or Br, and, if X is disubstituted by two identical substituents selected from the group consisting of F, Cl and Br, then the two substituents are located neither in positions 2 and 4 nor in positions 3 and 4.

5. A process for the synthesis of a compound of formula (IA-1), which is a compound of formula (I), said process 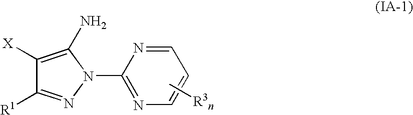
comprising reacting a compound of formula (V) 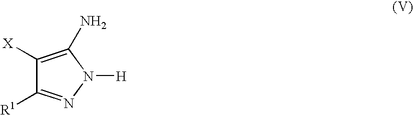
with a compound of the formula (VI) 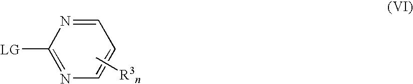
where LG represents halogen or alkylsulphonyl.

6. A process for preparing a compound of formula (IA-3) 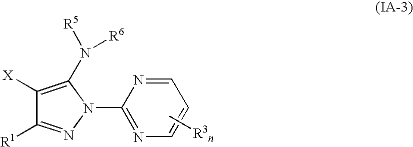
where
R⁵ represents hydrogen, alkyl, alkylcarbonyl, cycloalkyl, cycloalkylalkyl, alkenyl, alkoxycarbonyl, alkenoxycarbonyl, alkynoxycarbonyl, alkoxycarbonylcarbonyl, benzyl or phenylcarbonyl (where the phenyl ring in benzyl and phenylcarbonyl for its turn is optionally substituted by one or more fluorine, bromine and/or chlorine atoms),
R⁶ represents hydrogen,
R⁶ furthermore, if R⁵ represents benzyl, alkyl, alkoxycarbonyl, alkenoxycarbonyl, alkynoxycarbonyl or alkylcarbonyl, may also represent benzyl, alkyl, alkoxycarbonyl, alkenoxycarbonyl, alkynoxycarbonyl or alkylcarbonyl,
comprising reacting a compound of the formula (IA-1), which is a compound of claim 1, 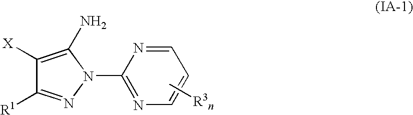
with one or two halogenating agents R⁵-LG and/or R⁶-LG, where LG represents halogen or alkylsulphonyl.

7. A process for preparing a compound of formula (IB) 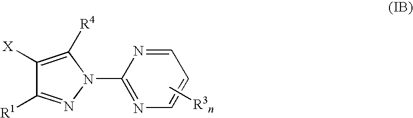
where represents a halogen, comprising reacting a compound of formula (IA-1), which is a compound of claim 1, 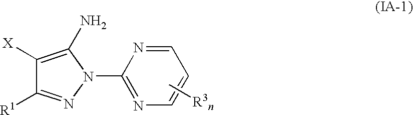
in the presence of nitrosyl ions with a halide.

8. A process for synthesis of a compound of formula (IA-1-2), which is a compound of claim 1, 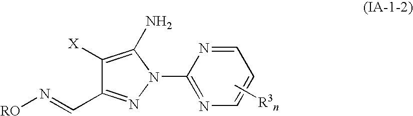
wherein R represents alkyl, said process comprising reacting a compound of formula (IA-1-1) 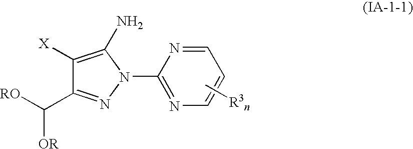
in the presence of an acid with an O-alkyl hydroxylamine R—O—NH₂.

9. A process for synthesis of a compound of formula (IA-1-3), which is a compound of claim 1, said process comprising reacting 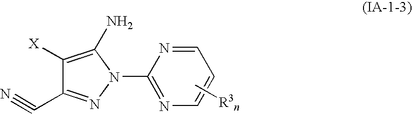
a compound of formula (IA-1-1) 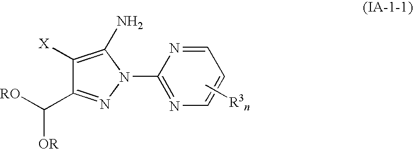
in the presence of an acid with hydroxylamine to give a compound of the formula (IA-1-4) 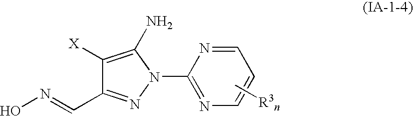
and dehydrating to give a compound of formula (IA-1-3).

10. A process for synthesis of a compound of formula IA-1, which is a compound of claim 1, said process comprising reacting 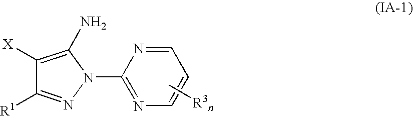
a compound of formula (IIA) 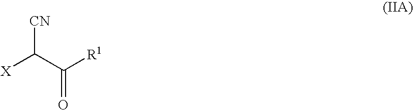
and/or a compound of formula (IIB) 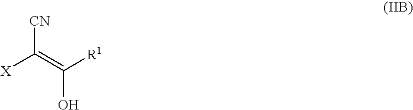
and/or a compound of formula (IIC) 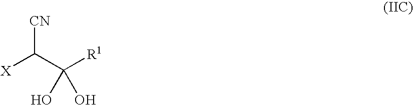
with a chlorinating agent to give a compound of formula (VII) 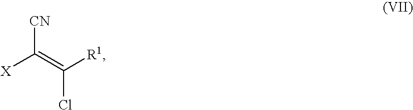
and subsequently condensing with a compound of formula (III) 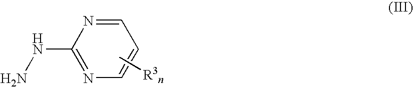
in a suitable organic solvent in the presence of basic auxiliary reagents to give the compound of formula IA-1.

11. A process for synthesis of a compound of formula IA-1, which is a compound of claim 1, said process comprising reacting 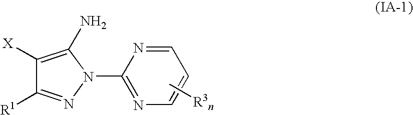
a compound of formula (IA-1-5) 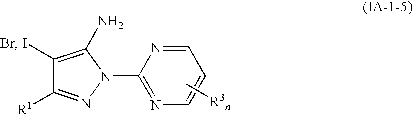
with a compound of formula (VIII)
X—B(OR)₂ (VIII)
in the presence of a suitable palladium catalyst and/or base to give a compound of the formula (IA-1).

12. A method for controlling animal pests comprising using a compound of claim 1.

13. A method for controlling animal pests, comprising allowing a compound of formula (I) according to claim 1 to act on an animal pest and/or a habitat thereof and/or on seed.

14. A process for preparing an agrochemical composition comprising mixing a compound of formula (I) according to claim 1 with an extender and/or a surfactant.

15. A composition for controlling animal pests comprising a compound of claim 1 and at least one extender and/or surfactant.

16. A composition for controlling animal pests comprising a compound of claim 2 and at least one extender and/or surfactant.

17. A composition for controlling animal pests comprising a compound of claim 3 and at least one extender and/or surfactant.

18. A composition for controlling animal pests comprising a compound of claim 4 and at least one extender and/or surfactant.

19. A method for controlling animal pests comprising using a compound of claim 2.

20. A method for controlling animal pests comprising using a compound of claim 3.
